(19) United States
(12) Patent Application Publication
(10) Pub. No.: US 2026/0199367 A1
(43) Pub. Date: Jul. 16, 2026

(54) PLK1 DEGRADATION INDUCING COMPOUNDS WITH INCREASED RIGIDITY

(71) Applicant: UPPTHERA, INC., Incheon (KR)

(72) Inventors: Soo Hee Ryu, Incheon (KR); Seong Hoon Kim, Incheon (KR); Jun Kyu Lee, Incheon (KR); Keum Young Kang, Incheon (KR); Gibbeum Lee, Incheon (KR); Sang Youn Kim, Incheon (KR); Solhee Noh, Incheon (KR)

(73) Assignee: UPPTHERA, INC., Incheon (KR)

(21) Appl. No.: 19/133,468

(22) PCT Filed: Nov. 29, 2023
(86) PCT No.: PCT/KR2023/019484

§ 371 Date: May 28, 2025

(30) Foreign Application Priority Data
Nov. 29, 2022 (KR) 10-2022-0163312
Jan. 19, 2023 (KR) 10-2023-0007899
Jul. 27, 2023 (KR) 10-2023-0097913

(51) Int. Cl. A61K 31/551; A61K 31/519; A61K 47/68; C07D 475/00; C07D 487/04

(52) U.S. Cl. CPC A61K 31/551; A61K 31/519; A61K 47/6803; C07D 475/00; C07D 487/04

(57) ABSTRACT

Novel dihydropteridone or pyrimidodiazepinone derivative based PLK1 degraders are disclosed. The present PLK1 degraders are proteolysis targeting chimeras (PROTACs) that recruit PLK1 protein into CRBN E3 ubiquitin ligase via optimized rigid linkers. The compounds may induce PLK1 degradation with improved drug properties and may be utilized for example, as a payload of antibody-drug conjugated (ADC) for the treatment of cancer.

### TECHNICAL FIELD

The present disclosure relates to a novel PLK1 degradation inducing compound, a method for preparing the same, and the use thereof.

### BACKGROUND ART

Polo-like kinase 1 (PLK1) is a serine/threonine kinase that is widely distributed in eukaryotic cells and plays important roles in several phases of the cell cycle. PLK1 has long been considered a potential drug target for cancer treatment and several PLK1 inhibitors have been evaluated in clinical trials. Amongst others, BI-2536 is a first-in-class highly selective ATP-competitive small molecule based upon a dihydropteridone derivative that accomplished inhibiting the enzymatic activity of PLK1 by blocking the ATP-binding site. BI-2536 has been found to be a potent inhibitor of BRD4 as well, implicating a single polypharmacological targeting compound. However, the conventional PLK1 inhibitors do not sufficiently inhibit PLK1 activity at concentrations that are clinically safe and most of them have failed or were stopped in clinical trial stage. It shows that pharmacological mechanism that follows the method of inhibiting enzyme activity by binding to the active site of PLK1 like the small molecule compound inhibitors is not sufficiently effective in the development of new drugs intended to derive anticancer effects by inhibiting PLK1 activity of cancer cells.

Proteolysis targeting chimeras (PROTACs) are bispecific molecules containing a target protein binder and an E3 ubiquitin ligase binder connected by a linker. By recruiting a ubiquitin ligase to a target protein, PROTACs may promote proteasomal degradation of the target protein. Since depletion of oncogenic proteins in cancer cells may achieve better therapeutic efficacy than inhibiting the same protein, PROTACs have been developed as new therapeutic modalities in the fields of anticancer therapeutics.

Regarding PLK1-degrading PROTACs, CN109879877A discloses PLK1/BRD4-dual targeting PROTACs. The inventors focused that BRD4 and PLK1 were both attractive therapeutic targets in acute myeloid leukemia (AML) and also demonstrated the PROTACs induced the degradation of both BRD4 and PLK1 proteins, and proliferation inhibition activity in human acute leukemia cells (Mu, Xupeng, et al. “Protein targeting chimeric molecules specific for dual bromodomain 4 (BRD4) and Polo-like kinase 1 (PLK1) proteins in acute myeloid leukemia cells.” Biochemical and biophysical research communications 521.4 (2020): 833-839.).

However, CN109879877A only discloses limited PROTAC designs wherein dihydropteridone derivatives are tethered to thalidomide derivatives which are cereblon (CRBN) binders via alkyl or PEG linkers. PROTAC field is currently undergoing an important shift from synthetically tractable alkyl and polyethylene glycol to optimized rigid and functional linkers such that degrader potency is maintained while flexibility or degrees of freedom of the ternary complex are reduced (Troup, Robert I., Charlene Fallan, and Matthias G J Baud. “Current strategies for the design of PROTAC linkers: a critical review.” Exploration of Targeted Anti-tumor Therapy 1.5 (2020): 273.).

It is currently accepted that the linker is not just a mere connector that links protein of interest (POI) and E3 warheads. Instead, it plays a specific role in the PROTAC's degradation potency and efficacy by participating in ternary complex formation. Furthermore, the physicochemical profiles of PROTAC (e.g., potency, solubility, or stability) may vary depending on the linker employed. Given this, modifications to the linker structure, including the substitution of early-stage alkyl/PEG linkers, are necessary to develop therapeutically effective PROTAC compounds. However, the discovery of PROTAC linkers that form target-specific stable ternary complex leading to sufficient target protein degradation effects remains an extremely challenging work and requires extensive empirical trials and errors within the fields.

The applicant previously developed PLK1-targeting PROTACs with target degradation effects, utilizing different PLK1 binding moieties (WO2021/194318 A1, WO2021/194319 A1, WO2021/194320 A1, WO2021/194321 A1). However, predicting the combination of anchor/linker/warhead that leads to the optimal degradation is a formidable challenge due to the structural complexity and dynamics of the ternary complex. Under the circumstances, by introduce of amine and ring conformation-based PROTAC linkers with rigid properties, the inventors have succeeded in synthesis of novel optimized PLK1 degraders with efficacious PLK1 degradation that may have improved pharmacological profiles for PROTAC-based drugs.

### DISCLOSURE OF INVENTION

### Technical Problem

A main objective of the invention is to provide alternative novel PLK1 degraders with increased rigidity via linker optimization.

### Solution to Problem

To accomplish this, the present invention provides novel PROTAC compounds with a rigid linker by insertion of two saturated 6-membered rings optionally mediated by short chains with limited variations.

### Design of Novel PLK1 Degraders

In one general aspect, the present disclosure provides a novel compound represented by the following Formula I:

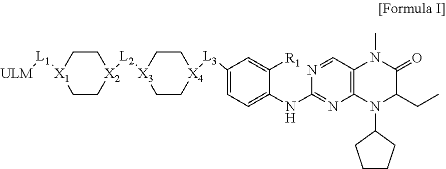

wherein:
L₁ is a covalent bond or —(X₅)₁₋₃— wherein X₅ is each independently —CH₂— or —NH— provided that —NH— is absent or present once at most in L₁;
L₂ is a covalent bond or —(X₆)₁₋₃— wherein X₆ is each independently —CH₂—, —C(O)—, —NH— or —CH(NH₂)— provided that —C(O)—, —NH—, —N(CH₃)— and —CH(NH₂) are each independently absent or present once at most in L₂;
L₃ is

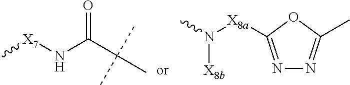

wherein L₃ is covalently bonded into X₄ through [symbol] ;
X₁, X₂, X₃ and X₄ are each independently CH or N;
X₇ and X_{8d} are each independently a covalent bond or —CH₂—;
X_{8b} is —H or —CH₃; and
R₁ is —OCH₃ or —OCF₃;
wherein ULM is a moiety represented by the following Formula II-1, II-2 or II-3:

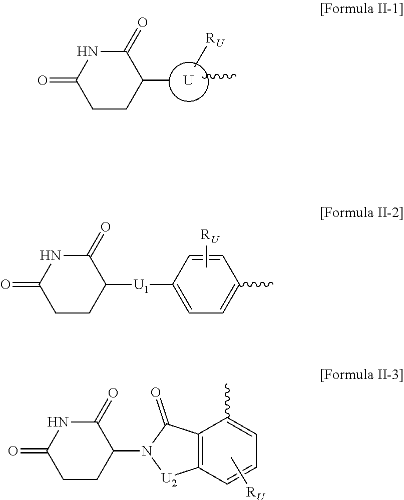

wherein:
Ring U is phenyl or 5- or 6-membered heteroaryl;
U₁ is —NH—, —NHCH₂—, —NHCH₂CH₂—, —CH₂NH—, —CH₂CH₂NH—, —NHCO—, —CONH— or —O—;
U₂ is CH₂ or C(O); and
R_{U} is halo.

In formula 1,

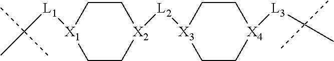

is a PROTAC linker characterized in that two saturated 6-membered rings

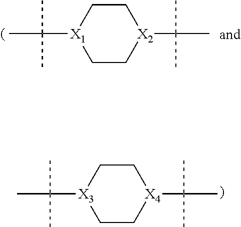

are tethering ULM and PTM in para-position, optionally being mediated by a short chains L₁, L₂ and L₃. The rigid properties of the PROTAC linker may contribute to more stable conformation in CRBN E3 ligase-PLK1 interactions and presentation of PLK1 within the zone of ubiquitylation.

In one embodiment, one to three of X₁ to X₄ is N. The invention has attention that piperidine or piperazine rings bear a basic center by protonable amino group, therefore may have the pivotal role of the linker moiety and improve rigidity and solubility upon protonation.

In Formula 1, L₁, L₂ and L₃ mediate two saturated 6-membered rings described above and may contribute to overall favorable rigidity and solubility of the PROTAC linker within Formula 1.

In L₁ of Formula 1, —(X₅)₁₋₃— denotes —(X₅)— unit is sequentially connected one to three times wherein each unit —(X₅)— may be same or different.

In one embodiment, L₁ is a covalent bond wherein ULM is connected to X₁ by a covalent bond.

In one embodiment, L₁ is —(CH₂)₁₋₃—, for example, —CH₂—, —CH₂CH₂— or —CH₂CH₂CH₂— wherein —NH— is absent in L₁.

In one embodiment, L₁ is —NH—, —NHCH₂—, —CH₂NH—, —NHCH₂CH₂—, —CH₂NHCH₂— or —CH₂CH₂NH—, wherein —NH— is present once in L₁.

In L₂ of Formula 1, —(X₆)₁₋₃— denotes —(X₆)— unit is sequentially connected one to three times wherein each unit —(X₆)— may be same or different.

In one embodiment, L₂ is a covalent bond wherein two 6-membered rings are connected by a covalent bond.

In one embodiment, L₂ is —(CH₂)₁₋₃—, for example, —CH₂—, —CH₂CH₂— or —CH₂CH₂CH₂— wherein functional group is absent in L₂.

In one embodiment, L₂ is —(Rₓ)—, —(Rₓ)—CH₂—, —CH₂—(Rₓ)—, —(Rₓ)—(Rₓ)—, —(Rₓ)—CH₂—CH₂—, —CH₂—(Rₓ)—CH₂—, —CH₂—CH₂—(Rₓ)—, —(Rₓ)—(Rₓ)—CH₂—, —(Rₓ)—CH₂—(Rₓ)—, —CH₂—(Rₓ)—(Rₓ)— or —(Rₓ)—(Rₓ)—(Rₓ)—, wherein Rₓ is —C(O)—, —NH—, —N(CH₃)— or —CH(NH₂); and each unit —(Rₓ)— is different. Therefore, —C(O)—, —NH—, —N(CH₃)— or —CH(NH₂) is present once in L₂, if any.

In one embodiment, L₃ is

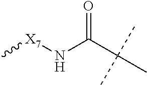

wherein the amide group may mediate a network of interactions that involve residues from both the N- and C-lobes of PLK1, with the NH and CO forming hydrogen bonds with PLK1 key amino acid residues, for example, the main chain carbonyl of LEU 59 residue of the glycine-rich loop and the side chain of ARG 57 residue, respectively.

In one embodiment, L₃ is

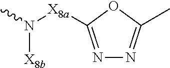

wherein oxadiazole group may further improve PROTAC properties including metabolic stability.

The PROTAC linker design speculated in Formula 1 is optimized for anchor/linker/warhead, therefore may lead to improved physicochemical properties with efficacious potency.

ULM represented by Formula II-1 or II-2 is CRBN ligand, wherein a glutarimide is conjugated to phenyl or heteroaryl ring directly or mediated by a short chain moiety.

In Formula II-1, Ring U may be phenyl or 5- or 6-membered heteroaryl including pyrrolyl, furanyl, thienyl, oxazolyl, isoxazolyl, thiazolyl, imidazolyl, triazolyl, tetrazolyl, pyridinyl, pyrimidinyl, pyrazinyl, pyridazinyl or pyrazolyl. In one embodiment, Ring U is phenyl or pyridinyl.

In one embodiment, Formula II-1 is selected from the following:

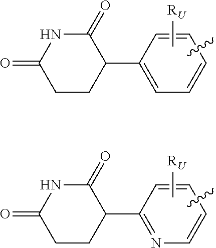

In Formula II-1, L₁ and a glutarimide moiety within Formula II-1 may be connected to Ring U in ortho-, meta-, or para-position. In one embodiment, they are connected in para-position.

In one embodiment, Formula II-1 is selected from the following:

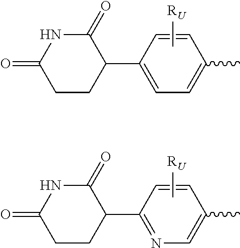

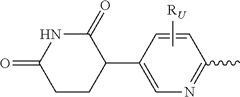

In Formula II-2, U₁ may be —NH—, —NHCH₂—, —NHCH₂CH₂—, —CH₂NH—, —CH₂CH₂NH—, —NHCO—, —CONH— or —O—. In one embodiment, U₁ is —NH—.

In one embodiment, Formula II-2 is following:

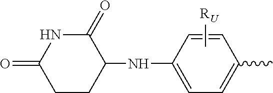

In Formula II-1, II-2 and II-3, R_{U} may be fluoro, chloro, bromo or iodo. In one embodiment, R_{U} is fluoro.

In one embodiment of the present disclosure, Formula I is the following Formula I-1:

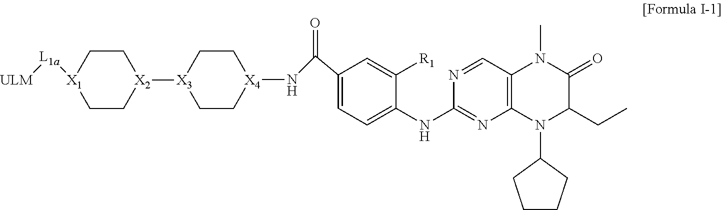

wherein L₁ₐ is —CH₂—, —NH—, —CH₂CH₂—, —NHCH₂—, —CH₂NH—, —CH₂CH₂CH₂—, —NHCH₂CH₂—, —CH₂NHCH₂— or —CH₂CH₂NH—.

In one embodiment of the present disclosure, Formula I is the following Formula I-2:

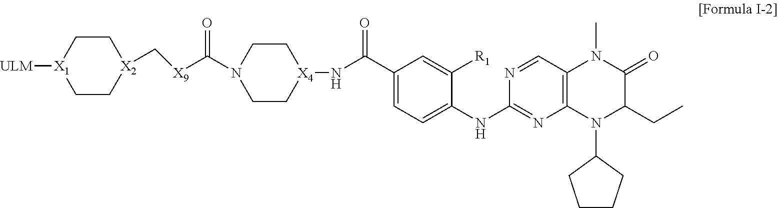

wherein:
X₉ is —CH₂— or —CH(NH₂)—.

In one embodiment of the present disclosure, Formula I is the following Formula I-3:

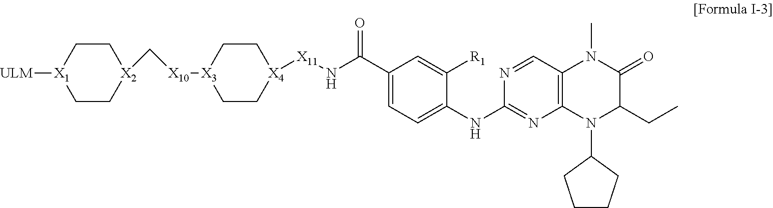

wherein X₁₀ is a covalent bond, —NH— or —CH(NH₂)—; and
X₁₁ is a covalent bond or —CH₂—.

In one embodiment of the present disclosure, Formula I is the following Formula I-4:

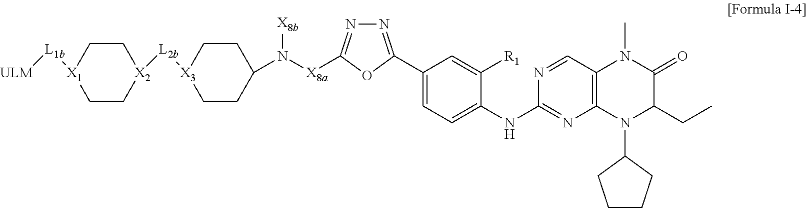

wherein:
L_{1b} is a covalent bond or —CH₂NH—;
L_{2b} is a covalent bond; —CH₂NH—, —CH₂N(CH₃)— or —CH₂CH(NH₂)C(O)—;
X_{8d} is a covalent bond or —CH₂—; and
X_{8b} is —H or —CH₃.

In one embodiment of the present disclosure, the compound represented by Formula I is selected from the group consisting of Compounds 1 to 25.

TABLE 1

| Com- | |
|---|---|
| pound | Structure |
| 1 | 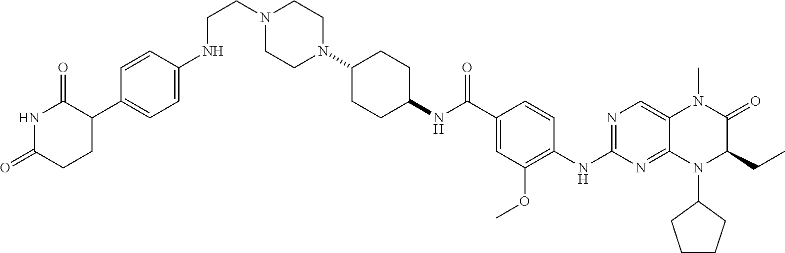 |
| 2 | 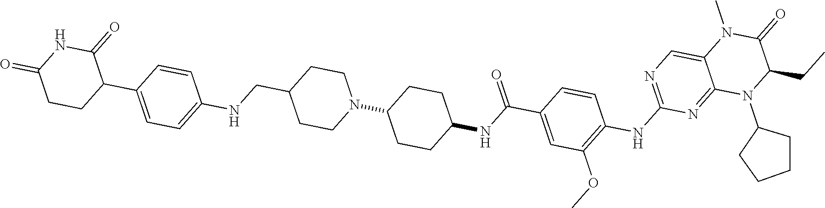 |
| 3 | 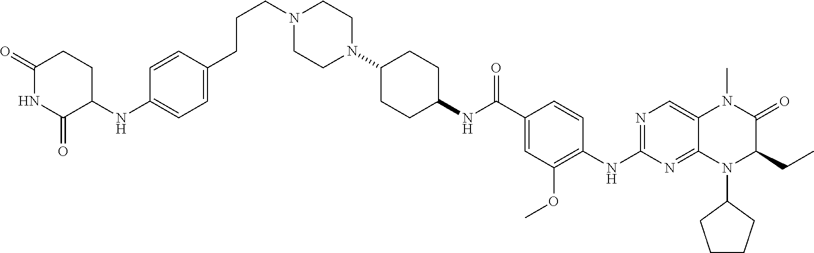 |
| 4 | 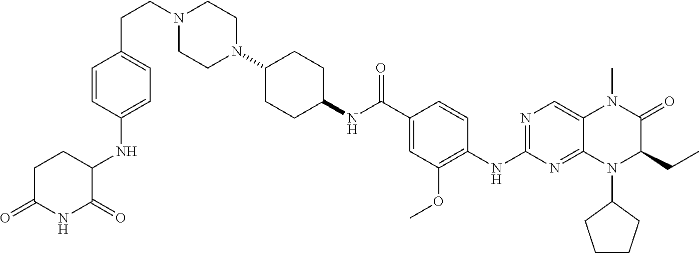 |
| 5 | 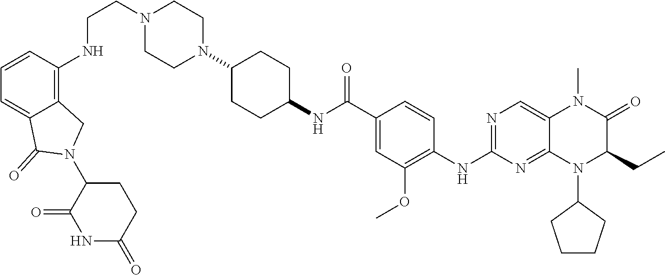 |
| 6 | 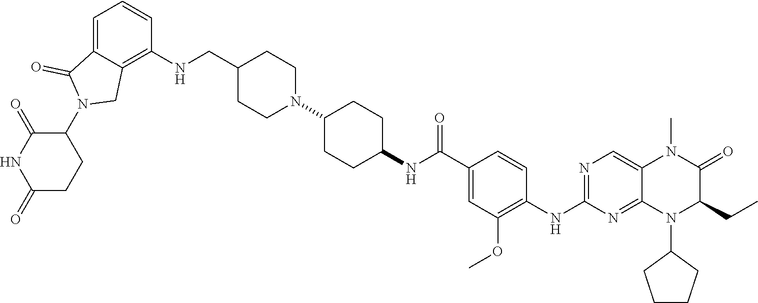 |
| 7 | 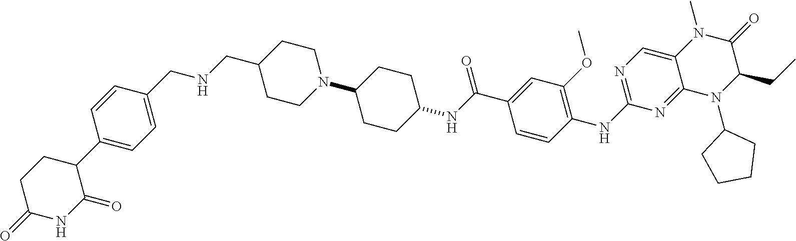 |
| 8 | 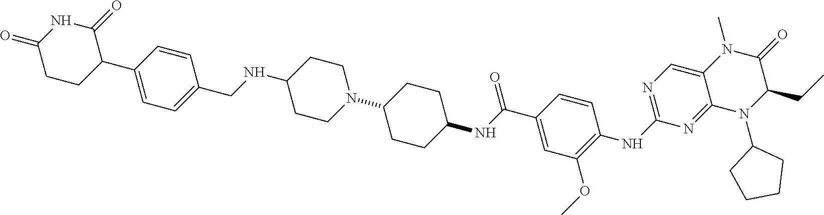 |
| 9 | 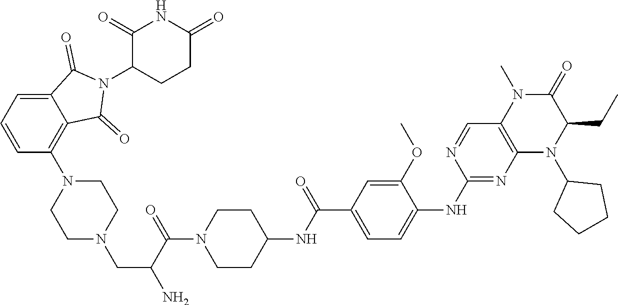 [SMILES: CC[C@@H]1C(=O)N(C)c2cnc(Nc3ccc(C(=O)NC4CCN(C(=O)C(N)CN5CCN(c6cccc7c6C(=O)N(C6CCC(=O)NC6=O)C7=O)CC5)CC4)cc3OC)nc2N1C1CCCC1] |
| 10 | 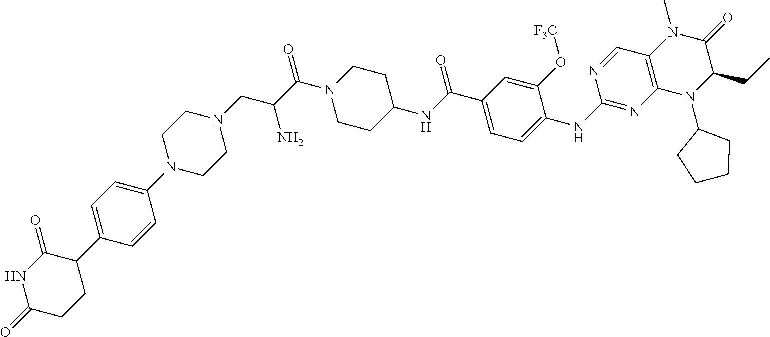 [SMILES: CC[C@@H]1C(=O)N(C)c2cnc(Nc3ccc(C(=O)NC4CCN(C(=O)C(N)CN5CCN(c6ccc(C7CCC(=O)NC7=O)cc6)CC5)CC4)cc3OC(F)(F)F)nc2N1C1CCCC1] |
| 11 | 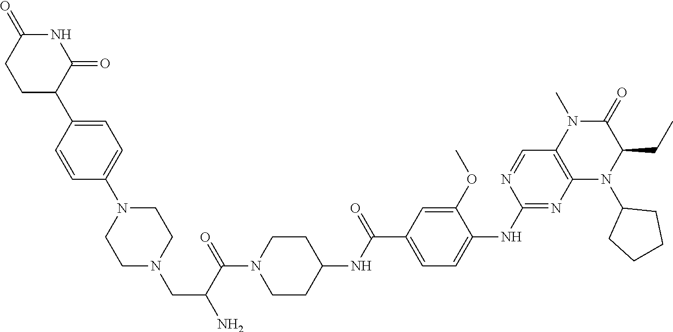 [SMILES: CC[C@@H]1C(=O)N(C)c2cnc(Nc3ccc(C(=O)NC4CCN(C(=O)C(N)CN5CCN(c6ccc(C7CCC(=O)NC7=O)cc6)CC5)CC4)cc3OC)nc2N1C1CCCC1] |
| 12 | 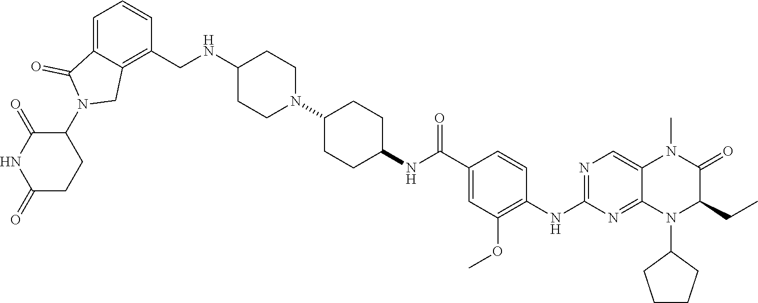 |
| 13 | 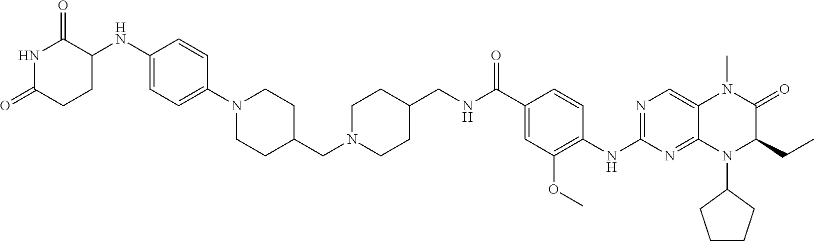 [SMILES: CC[C@@H]1C(=O)N(C)c2cnc(Nc3ccc(C(=O)NCC4CCN(CC5CCN(c6ccc(NC7CCC(=O)NC7=O)cc6)CC5)CC4)cc3OC)nc2N1C1CCCC1] |
| 14 | 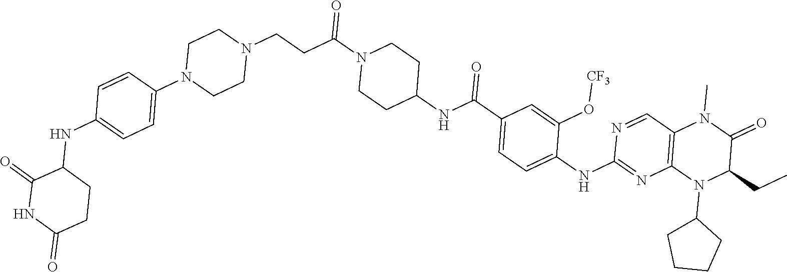 [SMILES: CC[C@@H]1C(=O)N(C)c2cnc(Nc3ccc(C(=O)NC4CCN(C(=O)CCN5CCN(c6ccc(NC7CCC(=O)NC7=O)cc6)CC5)CC4)cc3OC(F)(F)F)nc2N1C1CCCC1] |
| 15 | 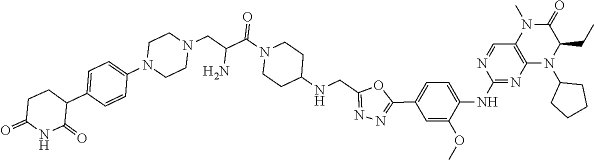 [SMILES: CC[C@@H]1C(=O)N(C)c2cnc(Nc3ccc(-c4nnc(CNC5CCN(C(=O)C(N)CN6CCN(c7ccc(C8CCC(=O)NC8=O)cc7)CC6)CC5)o4)cc3OC)nc2N1C1CCCC1] |
| 16 | 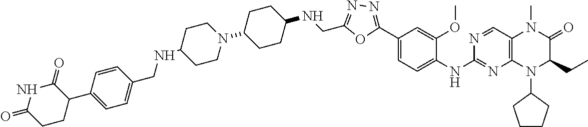 |
| 17 | 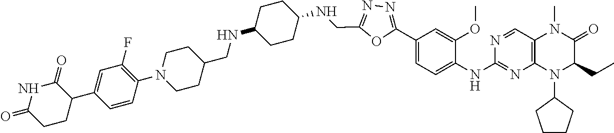 |
| 18 | 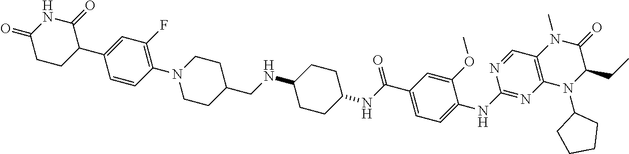 |
| 19 | 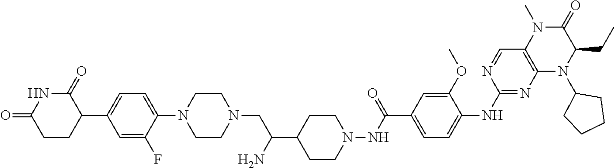 [SMILES: CC[C@@H]1C(=O)N(C)c2cnc(Nc3ccc(C(=O)NN4CCC(C(N)CN5CCN(c6ccc(C7CCC(=O)NC7=O)cc6F)CC5)CC4)cc3OC)nc2N1C1CCCC1] |
| 20 | 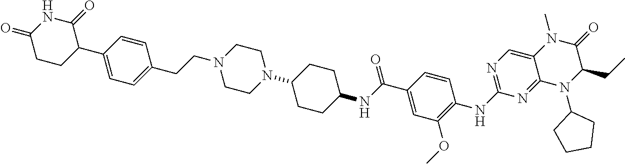 |
| 21 | 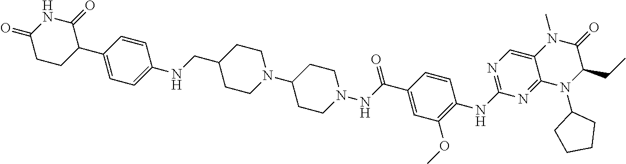 [SMILES: CC[C@@H]1C(=O)N(C)c2cnc(Nc3ccc(C(=O)NN4CCC(N5CCC(CNc6ccc(C7CCC(=O)NC7=O)cc6)CC5)CC4)cc3OC)nc2N1C1CCCC1] |
| 22 | 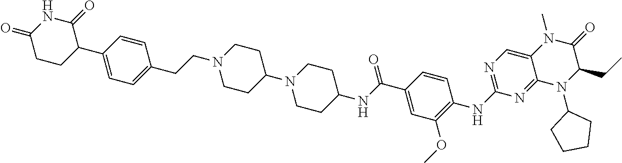 [SMILES: CC[C@@H]1C(=O)N(C)c2cnc(Nc3ccc(C(=O)NC4CCN(C5CCN(CCc6ccc(C7CCC(=O)NC7=O)cc6)CC5)CC4)cc3OC)nc2N1C1CCCC1] |
| 23 | 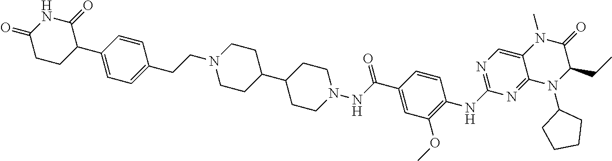 [SMILES: CC[C@@H]1C(=O)N(C)c2cnc(Nc3ccc(C(=O)NN4CCC(C5CCN(CCc6ccc(C7CCC(=O)NC7=O)cc6)CC5)CC4)cc3OC)nc2N1C1CCCC1] |
| 24 | 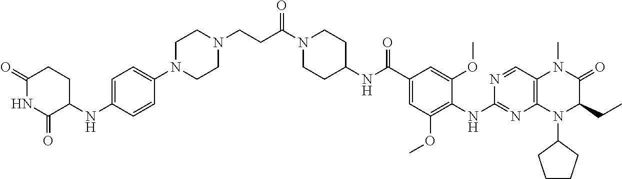 [SMILES: CC[C@@H]1C(=O)N(C)c2cnc(Nc3c(OC)cc(C(=O)NC4CCN(C(=O)CCN5CCN(c6ccc(NC7CCC(=O)NC7=O)cc6)CC5)CC4)cc3OC)nc2N1C1CCCC1] |
| 25 | 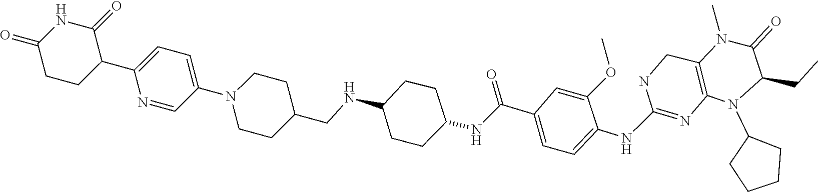 |

In another general aspect, there is provided a compound represented by the following Formula III:

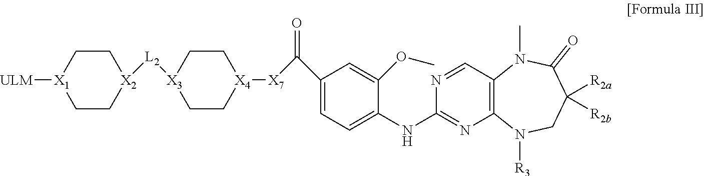

wherein:
L₂ is —CH₂—, —NHCH₂—, —CH₂NH—, —CH(NH₂)CH₂—, —CH₂CH(NH₂)— or —CH₂CH₂C(O)—;
X₁, X₂, X₃ and X₄ are each independently CH or N;
X₇ is a covalent bond or —CH₂—;
R_{2d} and R_{2b} are -halo; or R₂ₐ and R_{2b} are linked each other to form 3- to 6-membered ring, for example, cyclopropane ring; and
R₃ is —C₁₋₃alkyl or 3- to 7-membered cycloalkyl; for example, isopropyl or cyclopentyl,
wherein ULM is a moiety represented by the following Formula II-4:

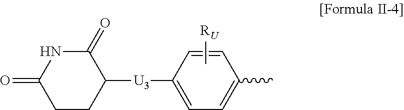

wherein U₃ is a covalent bond or —NH—; and R_{U} is halo.

In one embodiment of the present disclosure, the compound represented by Formula III is selected from the group consisting of Compound 26 to 31.

TABLE 2

| Compound | Structure |
|---|---|
| 26 | 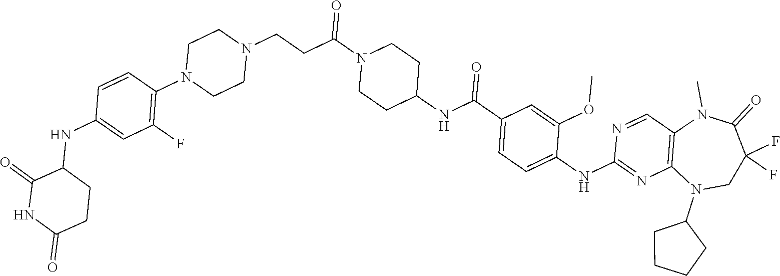 [SMILES: COc1cc(C(=O)NC2CCN(C(=O)CCN3CCN(c4ccc(NC5CCC(=O)NC5=O)cc4F)CC3)CC2)ccc1Nc1ncc2c(n1)N(C1CCCC1)CC(F)(F)C(=O)N2C] |
| 27 | 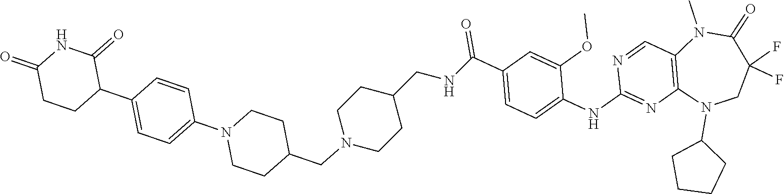 [SMILES: COc1cc(C(=O)NCC2CCN(CC3CCN(c4ccc(C5CCC(=O)NC5=O)cc4)CC3)CC2)ccc1Nc1ncc2c(n1)N(C1CCCC1)CC(F)(F)C(=O)N2C] |
| 28 | 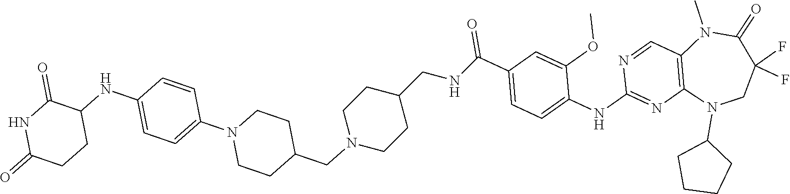 [SMILES: COc1cc(C(=O)NCC2CCN(CC3CCN(c4ccc(NC5CCC(=O)NC5=O)cc4)CC3)CC2)ccc1Nc1ncc2c(n1)N(C1CCCC1)CC(F)(F)C(=O)N2C] |
| 29 | 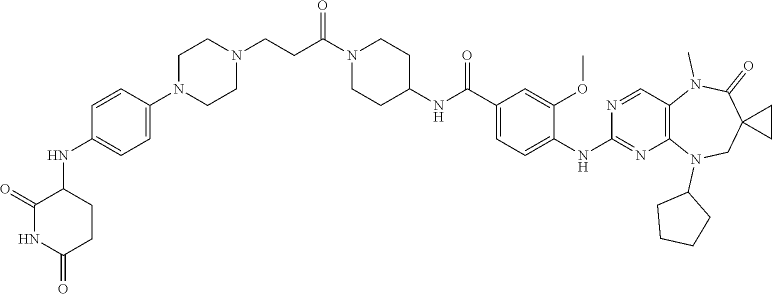 [SMILES: COc1cc(C(=O)NC2CCN(C(=O)CCN3CCN(c4ccc(NC5CCC(=O)NC5=O)cc4)CC3)CC2)ccc1Nc1ncc2c(n1)N(C1CCCC1)CC1(CC1)C(=O)N2C] |
| 30 | 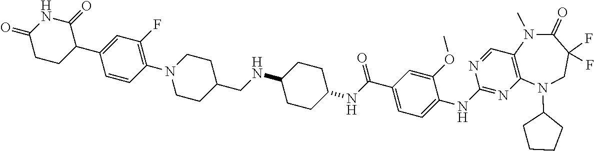 |
| 31 | 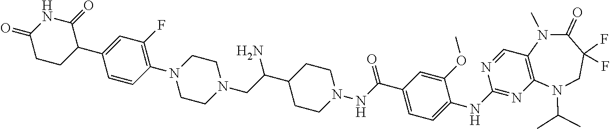 [SMILES: COc1cc(C(=O)NN2CCC(C(N)CN3CCN(c4ccc(C5CCC(=O)NC5=O)cc4F)CC3)CC2)ccc1Nc1ncc2c(n1)N(C(C)C)CC(F)(F)C(=O)N2C] |

PROTAC linkers may include multiple types of functional groups including amines, amides, ethers, alkylamines, single, multiple C—C bonds and the like. One of the alternative groups, cyclic scaffolds including piperidine or piperazine, may impart some rigidity to the linkers, which may be further modulated by additional groups (Desantis, Jenny, et al. “PROTACs bearing piperazine-containing linkers: what effect on their protonation state.” RSC advances 12.34 (2022): 21968-21977).

While the basic properties of these functional groups may be starting points towards optimized PROTAC design, achieving a successful PROTAC linker design is extremely challenging because it is unpredictable to determine which combination of anchor, linker, and warhead would lead to target protein degradation even at basic levels (Troup, Robert I., Charlene Fallan, and Matthias G J Baud. “Current strategies for the design of PROTAC linkers: a critical review.” Exploration of Targeted Antitumor Therapy 1.5 (2020): 273.).

Under the circumstances, the inventors revealed newly designed PROTACs with rigid linkers exhibiting the sufficient level of PLK1 degradation efficiency throughout the entire scope denoted by Formula I or III. In the fields of PROTACs, these are surprising results that are not expected from early stage PROTACs with flexible PEG or alkyl linkers.

In one embodiment, the compound of the present disclosure may be in the form of a salt, preferably pharmaceutically acceptable salts. In the present disclosure, a pharmaceutically acceptable salt refers to any organic or inorganic acid addition salt with a concentration that is relatively non-toxic, is harmless, and has effective action to patients, wherein side effects caused by this salt does not deteriorate beneficial efficacy of the novel compounds of the present disclosures.

In one embodiment, the compound of the present disclosure may be in the form of a racemate, enantiomer, rotamer, tautomer, N-oxide, or any stereoisomer as if each is specifically described unless specifically excluded by context.

In one embodiment, the compound of the present disclosure may be in the form of a hydrate or solvate thereof,

In one embodiment, the compound of the present disclosure may be in the form of a chimeric molecule by conjugation to a functional macromolecule via a chemical linker. In certain embodiment, the macromolecule is a biomolecule including a nucleic acid, an aptamer, a carbohydrate, a peptide or fragment thereof, an antibody or fragment thereof.

### Synthesis of Novel PLK1 Degraders

In one embodiment, the novel compound of the present disclosure may be prepared by for example, the following Reaction Schemes 1 to 3, by a synthetic method known in the field of organic chemistry or a modification technique apparent to those skilled in the art.

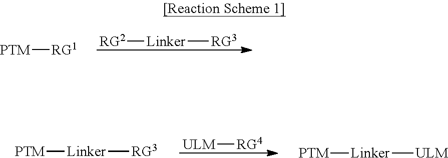

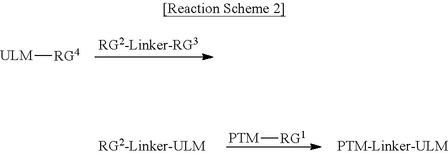

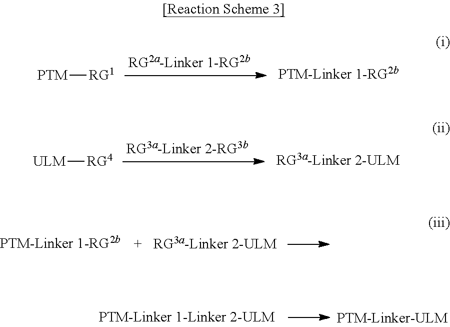

In the Reaction Schemes, PTM is

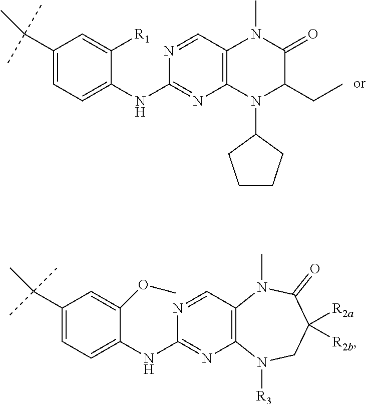

### Linker is

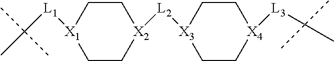

and ULM is same as defined above. RG¹, RG², RG^{2a}, RG^{2b}, RG³, RG^{3a}, RG^{3b} and RG⁴ are moieties including a suitable reactive group capable of linking together with an intermediate of PROTACs through formation of the covalent bond in the field of organic synthesis. The formation of the covalent bond may be achieved by synthetic reactions such as amide formation, ester formation, carbamate formation, urea formation, ether formation, amine formation, and single bonds, double bond formation between various carbons, click chemistry and the like, depending on specific reaction groups, but is not limited thereto.

Each step in the above Reaction Scheme may include one or multiple synthesis steps. Isolation and purification of the product may be accomplished by standard procedures known to those skilled in the art of organic chemistry.

### Use of Novel PLK1 Degraders

In one embodiment, the novel compound of the present disclosure is a PLK1 degrader that induces PLK1 protein degradation in cells.

PLK-1 is an enzyme that in humans is encoded by the PLK1 (polo-like kinase 1) gene, wherein the protein sequence is known in the fields (e.g., NCBI Reference Sequence NP_005021.). The structure of the PLK1 protein contains an N-terminal Ser/Thr kinase domain and a C-terminal repeat of the polo-box domain (PBD), whose phosphorylation is directly related to the enzymatic activity of PLK1.

The PROTACs of the invention may induce proteasomal degradation of PLK1 in cells, by recruiting CRBN ubiquitin ligase to PLK1 that are supported by the experimental examples of the present disclosure. For example, the present disclosure reveals Compounds 1 to 31 have PLK1 degradation activity evidenced by luciferase assay on HeLa LgBit cell system; leading to inhibitory activity on cancer cell viability evidenced by cell viability assay for small cell lung cancer (SCLC) cell lines H69 and H526.

In one embodiment, the PROTACs of the invention has therapeutic efficacy better than small PLK1 inhibitors from which the PROTACs originate, by depletion of target protein than inhibiting the same. Accordingly, the PROTACs of the invention may utilized for the treatment of PLK1-related disorder or condition, wherein abnormal expression of PLK1 proteins involves in onset and/or progress of disease.

In the present disclosure, PLK1-related disorder or condition refer to any disease or condition capable of being treated, delayed, inhibited or prevented from induction of degradation or inhibition of activity of PLK1. PLK1-related disorder or condition include, but not limited thereto, a cancer, a benign tumor or a neurological disease, etc.

In one embodiment, the PROTACs of the inventions may have anticancer activity on PLK1 expressing cancer cells by depletion of PLK1, a key oncogenic regulator of the cell cycle. The cancer includes all cancers capable of exhibiting prophylactic or therapeutic efficacy due to inhibition of PLK1 activity, and may be solid cancer or blood cancer. For example, the cancer may be one or more selected from the group consisting of squamous cell carcinoma, small cell lung cancer, non-small cell lung cancer, lung adenocarcinoma, lung squamous cell carcinoma, peritoneal cancer, skin cancer, skin or intraocular melanoma, rectal cancer, anal muscle cancer, esophageal cancer, small intestine cancer, endocrine cancer, parathyroid cancer, adrenal cancer, soft tissue sarcoma, urethral cancer, chronic or acute leukemia, lymphocytic lymphoma, hepatocellular carcinoma, gastrointestinal cancer, gastric cancer, pancreatic cancer, glioblastoma, neuroblastoma, glioma, cervical cancer, ovarian cancer, liver cancer, bladder cancer, liver tumor, breast cancer, colon cancer, colorectal cancer, endometrial or uterine cancer, salivary gland cancer, kidney cancer, prostate cancer, vulvar cancer, thyroid cancer, head and neck cancer, brain cancer, osteosarcoma, bone cancer, large cell lymphoma, adrenocorticoid tumor, t cell lymphoma/leukemia, neuroendocrine cancer, neuroendocrine tumor, cholangiocarcinoma, and the like, but is not limited thereto. The cancer includes not only primary cancer but also metastatic cancer.

The benign tumors include all benign tumors capable of exhibiting prophylactic or therapeutic efficacy due to the inhibition of PLK1 activity, such as benign tumors in pre-cancer stages, and may be solid tumors or blood tumors. For example, the tumor may be one or more selected from the group consisting of Barrett's esophagus, colon adenoma and polyp, breast fibroadenoma and cyst, monoclonal gammopathy of undetermined significance (MGUS), monoclonal lymphocytosis, and the like, but is not limited thereto.

The neurological diseases include all neurological diseases capable of exhibiting prophylactic or therapeutic efficacy due to the inhibition of PLK1 activity, and specifically, may be one or more selected from the group consisting of central nervous system disease, neurodegenerative disease, Alzheimer's disease, Parkinson's disease, multiple sclerosis, Huntington's disease, senile dementia, epilepsy, Lou Gehrig, stroke, and nerve damage and axonal degeneration-related disorders following brain or spinal cord injury, but is not limited thereto.

To improve drug activities, any known strategies in the fields of PROTACs may be applied to the PROTACs of the present, including photochemically controllable PROTACs (PHOTACs), hypoxia-activated PROTACs, folate-caged PROTACs, antibody-PROTAC conjugates (Ab-PROTACs) and aptamer-PROTAC conjugates (APCs), and BCL-XL PROTACs, and the like (Zhao, Chunlong, and Frank J. Dekker. “Novel Design Strategies to Enhance the Efficiency of Proteolysis Targeting Chimeras.” ACS Pharmacology & Translational Science 5.9 (2022): 710-723.).

In one embodiment, the PROTACs of the invention may be utilized as a payload of antibody-drug conjugates (ADCs), for example, of antibody-PROTAC conjugates (Ab-PROTACs). ADCs enable the delivery of a cytotoxic payload specifically to cancer cells, enabling one to achieve a maximal effect on cancer cells whereas undesired effects in noncancer cells can be minimized. Therefore, Ab-PROTACs utilizing the PROTACs of the invention may be a strategy to improve the tissue and cell-type selectivity of PROTACs.

In one embodiment, the present disclosure provides a pharmaceutical composition comprising the PROTACs of the invention, wherein the compound is conjugated to an antibody or an antigen binding fragment thereof via a linker.

In one embodiment, the present disclosure provides an antibody-drug conjugate comprising an antibody or an antigen binding fragment thereof and the PROTACs of the invention, wherein the compound is conjugated to the antibody or antigen binding fragment thereof via a linker.

In certain embodiment, the linker chemically connects the antibody or an antigen binding fragment thereof and the PROTACs of the invention wherein a functional group within the compound is modified to create a covalent bond with the linker moiety. In certain embodiment, the functional group is an amine group within PROTAC linker or E3L binder moiety of the compounds.

In certain embodiment, the antibody or antigen binding fragment thereof is a cancer cell-specific and bearing one or more molecules of the compounds of Formula I or III. In certain embodiment, the linker is a cleavable or non-cleavable linker.

The present disclosure also provides a pharmaceutical composition comprising the PROTACs of the invention, and at least one pharmaceutically acceptable carrier. In one embodiment, the pharmaceutical composition comprises an effective amount of at least one PROTAC compound of the invention, and optionally one or more of other active ingredient(s) in effective amounts for combination therapy. In one embodiment, the pharmaceutical composition comprises more than one pharmaceutically acceptable amount of additive or excipient.

In another embodiment of the present disclosure is a method of degrading PLK1 by administering the PROTACs of the invention to a sample in vitro. The sample may include a cell, a cell culture, a body fluid or tissue of a mammal including a human, but is not limited thereto.

### Advantageous Effects of Invention

The novel compounds of the present disclosure may induce PLK1 degradation in cells with improved PROTACs properties for example potency, target selectivity, solubility, lipophilicity, permeability, stability and toxicity; therefore, they may be effectively utilized for treatment of PLK1-related disorder or conditions.

### BEST MODE FOR CARRYING OUT THE INVENTION

Unless otherwise defined, all technical and scientific terms used herein have the same meaning as commonly understood by one of ordinary skill in the art to which this disclosure belongs. The terminology used in the description is for describing particular embodiments only and is not intended to be limiting of the disclosure.

The present disclosure provides synthetic methods and results of bioactivity of Compounds 1 to 31.

The compounds of the present invention were purified according to the following method and the structure was analyzed.

### Instruments

LCMS: Shimadzu LCMS-2020
HPLC: Agilent 1260 II LC, Agilent 1200/G6410B
NMR: BRUKER AVANCE/400 MHZ
SFC: SHIMADZU LC-30ADsf

### LCMS Analysis

LCMS data were recorded with Shimadzu LCMS-2020 equipped with an ESI (Electron Spray Ionization) device. 0.0375% TFA in water (solvent A) and 0.01875% TFA in ACN (solvent B) were used as mobile phases. As a column, Kinetex EVO C18 (2.1×30 mm, 5 μm) or HALO C18 (3.0×30 mm, 5 μm) were used.

### HPLC Analysis

In HPLC analysis, Agilent 1260 II LC or Agilent 1200/G6410B were used. 0.0375% TFA in water (solvent A) and 0.01875% TFA in ACN (solvent B) were used as the mobile phase. As a column, Zobrax Eclipse Plus C18 (4.6×150 mm, 3.5 μm) or YMC ODS A (4.6×150 mm, 3 μm) were used.

### NMR Analysis

¹H NMR spectrum was recorded with Bruker AVANCE III 400 MHz/5 mm Probe (BBO).

### SFC Analysis

In SFC analysis, SHIMADZU LC-30ADsf was used. CO₂ (solvent A) and 0.05% DEA in IPA+ACN (solvent B) were used as the mobile phase. As a column, Chiralpak AD-3 (50×4.6 mm, 3 μm) was used.

### EXAMPLES

### Example 1. Synthesis of 4-(((R)-8-cyclopentyl-7-ethyl-5-methyl-6-oxo-5,6,7,8-tetrahydropteridin-2-yl)amino)-N-((1r,4R)-4-(4-(2-((4-(2,6-dioxopiperidin-3-yl)phenyl)amino)ethyl)piperazin-1-yl)cyclohexyl)-3-methoxybenzamide (Compound 1)

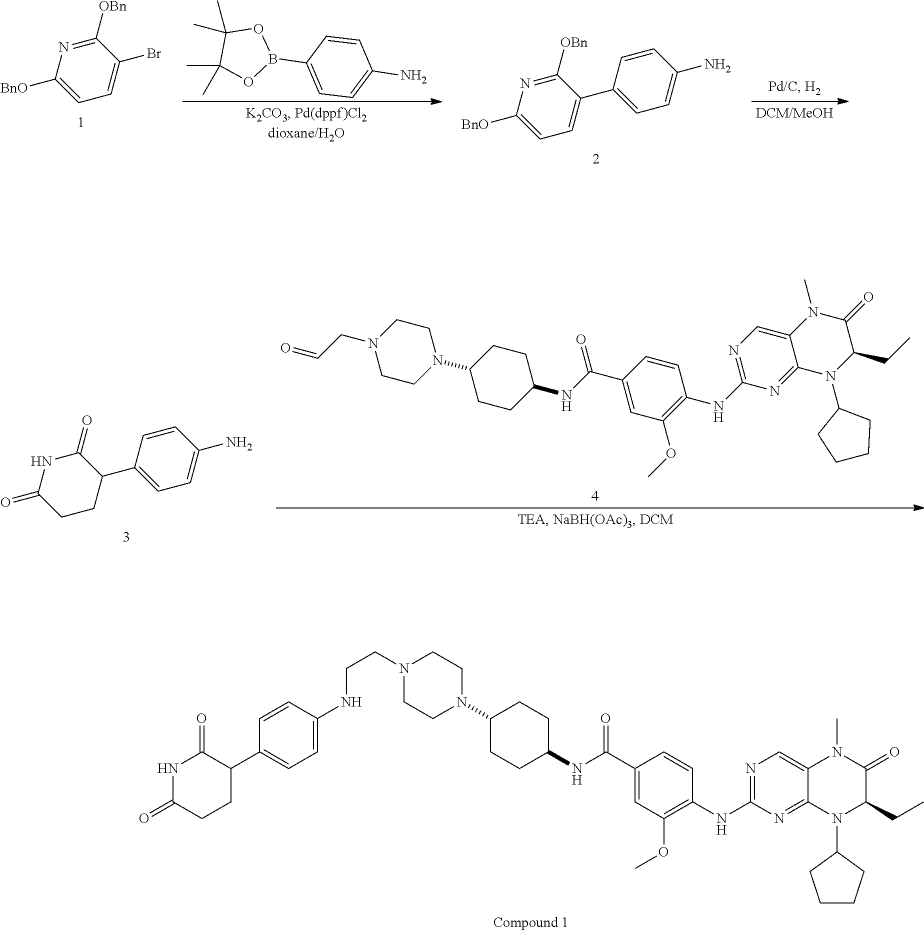

### Step 1. Synthesis of 4-(2,6-bis(benzyloxy)pyridin-3-yl)aniline (2)

To a solution of 2, 6-dibenzyloxy-3-bromo-pyridine (6 g, 16.21 mmol) and 4-(4,4,5,5-tetramethyl-1,3,2-dioxaborolan-2-yl) aniline (4.62 g, 21.07 mmol) in dioxane (90 mL) and H₂O (9 mL) were added K₂CO₃ (6.72 g, 48.62 mmol) and Pd(dppf)Cl₂ (1.19 g, 1.62 mmol) at 20° C. under N₂ and the resulting mixture was stirred at 110° C. for 6 h. LCMS showed starting material was consumed completely and 57% peak with desired mass. The reaction mixture was concentrated in vacuum. The residue was purified by flash silica gel chromatography (80 g SepaFlash® Silica Flash Column, Eluent of 0~20% EtOAc/Petroleum ether gradient @200 mL/min) to afford 4-(2,6-bis(benzyloxy)pyridin-3-yl)aniline (6.3 g, 16.14 mmol, 99.61% yield, 98% purity) as a yellow oil. MS(M+H)⁺=383.2

### Step 2. Synthesis of 3-(4-aminophenyl)piperidine-2,6-dione (3)

To a solution of 4-(2,6-bis(benzyloxy)pyridin-3-yl)aniline (6 g, 15.69 mmol) in DCM (50 mL) and MeOH (50 mL) was added Pd/C (1 g, 10% purity) under N₂ atmosphere. The suspension was degassed and purged with H₂ for 3 times. The mixture was stirred under H₂ (15 Psi) at 20° C. for 12 h. LCMS showed 44% of starting material remained and 37% peak with desired mass and the reaction mixture was stirred at 20° C. for another 12 h. LCMS showed starting material was consumed completely and peak with desired mass was detected. The reaction mixture was diluted with MeOH (100 mL) and filtered. The filtrate was concentrated in vacuum to afford 3-(4-aminophenyl)piperidine-2,6-dione (2.4 g, 11.75 mmol, 74.91% yield) as a green solid. MS(M+H)⁺=205.1

### Step 3. Synthesis of 4-(((R)-8-cyclopentyl-7-ethyl-5-methyl-6-oxo-5,6,7,8-tetrahydropteridin-2-yl)amino)-N-((1r,4R)-4-(4-(2-((4-(2,6-dioxopiperidin-3-yl)phenyl)amino)ethyl)piperazin-1-yl)cyclohexyl)-3-methoxybenzamide (Compound 1)

To a solution of 4-(((R)-8-cyclopentyl-7-ethyl-5-methyl-6-oxo-5,6,7,8-tetrahydropteridin-2-yl)amino)-3-methoxy-N-((1r,4R)-4-(4-(2-oxoethyl)piperazin-1-yl)cyclohexyl)benzamide (208.00 mg, 328.70 μmol) in DCM (10 mL) was added TEA (99.78 mg, 986.10 μmol, 137.25 μL) and 3-(4-aminophenyl)piperidine-2,6-dione (80.55 mg, 394.44 μmol) at 20° C. After stirring 10 min. Then NaBH(OAc)₃ (209.00 mg, 986.10 μmol) was slowly added at 20° C. and the resulting mixture was stirred at 20° C. for 2 h. LCMS showed all starting material was consumed completely and 30% peak with desired mass. The reaction mixture was diluted with H₂O (20 mL) and extracted with DCM (12 mL×3). The organic layer was washed saturated NaHCO₃ (12 mL×3), dried over Na₂SO₄, filtered and concentrated. The residue was purified by prep-TLC (SiO₂, DCM:MeOH=10:1) to afford 86 mg of product with 95% purity by LCMS and HPLC. The product was dissolved in mixture solution (20 mL, ACN:H₂O=1:3) and lyophilized to afford 4-(((R)-8-cyclopentyl-7-ethyl-5-methyl-6-oxo-5,6,7,8-tetrahydropteridin-2-yl)amino)-N-((1r,4R)-4-(4-(2-((4-(2,6-dioxopiperidin-3-yl)phenyl)amino)ethyl)piperazin-1-yl)cyclohexyl)-3-methoxybenzamide (53 mg, 59.39 μmol, 18.07% yield, 92% purity) as a yellow solid. MS(M+H)⁺=821.2

¹H NMR (400 MHz, DMSO-d₆) δ=10.73 (s, 1H), 8.44-8.36 (m, 1H), 8.03 (d, J=7.6 Hz, 1H), 7.84 (s, 1H), 7.59 (s, 1H), 7.50-7.45 (m, 2H), 6.91 (d, J=8.5 Hz, 2H), 6.53 (d, J=8.5 Hz, 2H), 5.37-5.24 (m, 1H), 4.35 (q, J=7.9 Hz, 1H), 4.27-4.19 (m, 1H), 3.93 (s, 3H), 3.78-3.68 (m, 1H), 3.64 (dd, J=5.0, 10.4 Hz, 1H), 3.24 (s, 3H), 3.09 (d, J=3.6 Hz, 2H), 2.66-2.56 (m, 4H), 2.48-2.39 (m, 7H), 2.29-2.21 (m, 1H), 2.14-1.95 (m, 4H), 1.93-1.84 (m, 6H), 1.80-1.72 (m, 4H), 1.68-1.59 (m, 3H), 1.40-1.28 (m, 4H), 0.76 (t, J=7.4 Hz, 3H)

### Example 2. Synthesis of 4-(((R)-8-cyclopentyl-7-ethyl-5-methyl-6-oxo-5,6,7,8-tetrahydropteridin-2-yl)amino)-N-((1r,4R)-4-(4-(((4-(2,6-dioxopiperidin-3-yl)phenyl)amino)methyl)piperidin-1-yl)cyclohexyl)-3-methoxybenzamide (Compound 2)

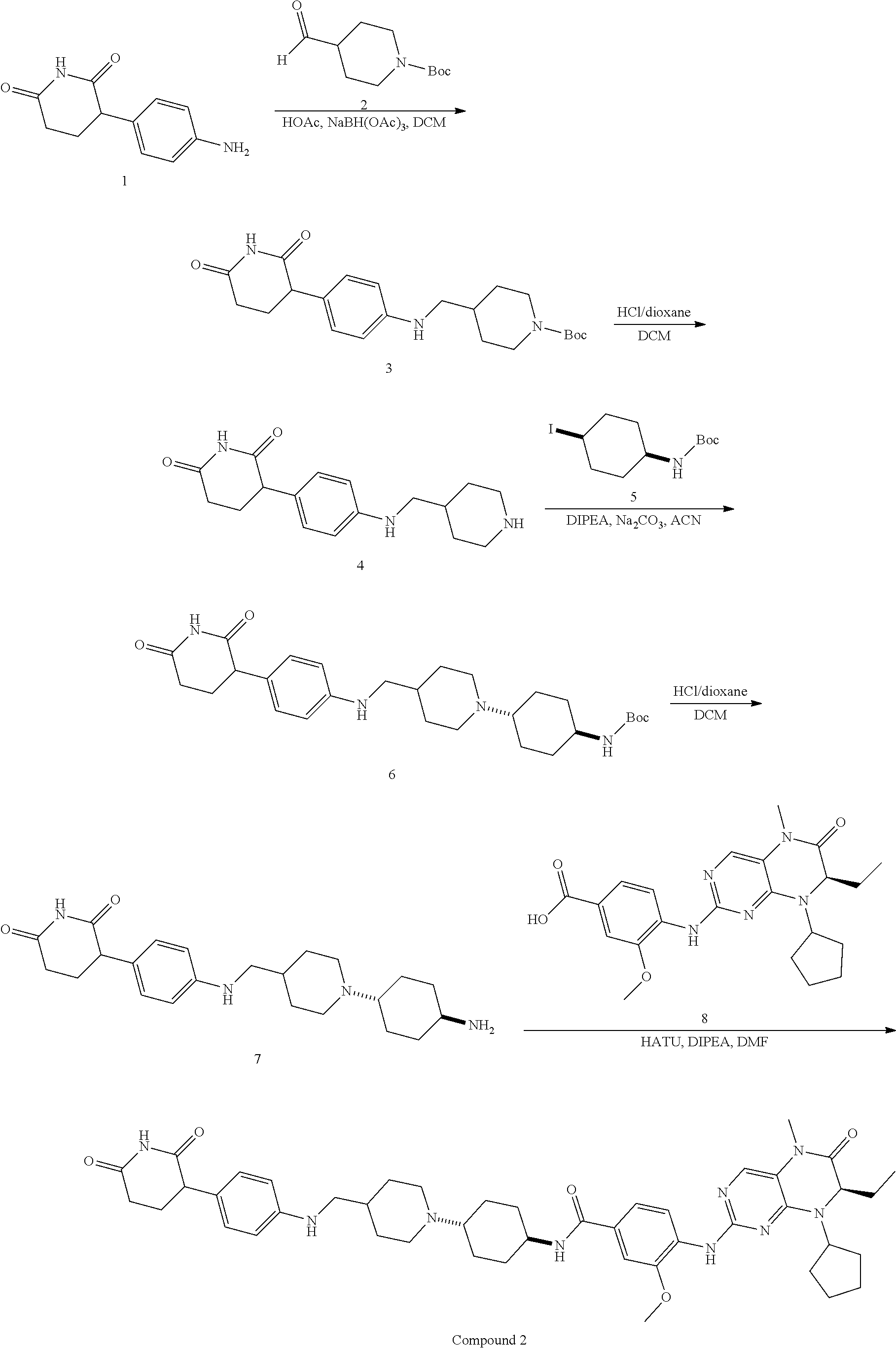

### Step 1. Synthesis of tert-butyl 4-(((4-(2,6-dioxopiperidin-3-yl)phenyl)amino)methyl)piperidine-1-carboxylate (3)

A mixture of 3-(4-aminophenyl)piperidine-2,6-dione (700 mg, 3.43 mmol), tert-butyl 4-formylpiperidine-1-carboxylate (657.91 mg, 3.08 mmol) and AcOH (20.58 mg, 342.76 μmol, 19.62 L) in DCM (20 mL) was stirred at 15° C. for 0.5 hours, then NaBH(OAc)₃ (3.3 g, 15.57 mmol) was added, the resulting mixture was stirred at 15° C. for 12 hours. LCMS showed a peak (84%) with desired mass. The mixture was diluted with H₂O (20 mL), then treated with saturated NaHCO₃ solution to adjust pH>7. The mixture was extracted with DCM (50 mL×2). The combined organic layers were dried over MgSO₄, filtered and concentrated in vacuum. The residue was purified by flash silica gel chromatography (20 g SepaFlash® Silica Flash Column, Eluent of 20~45% EtOAc/Petroleum ether gradient @200 mL/min), the eluent was concentrated in vacuum, the residue was diluted with EtOAc (30 mL), treated with HCl solution (1 M) to adjust pH<7. The aqueous phase was washed with EtOAc (30 mL×2), the EtOAc layer was discarded. The aqueous phase was treated with saturated Na₂CO₃ solution to adjust pH>7, extracted with EtOAc (50 mL×3), the combined organic layers were dried over Na₂SO₄, filtered and concentrated in vacuum to afford tert-butyl 4-(((4-(2,6-dioxopiperidin-3-yl)phenyl)amino)methyl)piperidine-1-carboxylate (360 mg, 896.64 μmol, 26.16% yield) as a light blue solid. MS(M+Na)⁺=424.1.

### Step 2. Synthesis of 3-(4-((piperidin-4-ylmethyl)amino)phenyl)piperidine-2,6-dione (4)

To a solution of tert-butyl 4-(((4-(2,6-dioxopiperidin-3-yl)phenyl)amino)methyl)piperidine-1-carboxylate (360 mg, 896.64 μmol) in DCM (10 mL) was added HCl/dioxane (4 M, 15 mL), the mixture was stirred at 15° C. for 1 hour. LCMS showed the starting material was consumed completely. The mixture was concentrated in vacuum to afford 3-(4-((piperidin-4-ylmethyl)amino)phenyl)piperidine-2,6-dione (340 mg, HCl salt) as a white solid, which was used in the next step directly. MS(M+H)⁺=302.4.

### Step 3. Synthesis of tert-butyl ((1r,4r)-4-(4-(((4-(2,6-dioxopiperidin-3-yl)phenyl)amino)methyl)piperidin-1-yl)cyclohexyl)carbamate(trans) (6)

A mixture of 3-(4-((piperidin-4-ylmethyl)amino)phenyl)piperidine-2,6-dione (380 mg, 1.12 mmol, HCl salt), tert-butyl ((1s,4s)-4-iodocyclohexyl)carbamate(cis) (475.49 mg, 1.46 mmol), Na₂CO₃ (357.64 mg, 3.37 mmol) and DIPEA (436.11 mg, 3.37 mmol, 587.75 μL) in ACN (10 mL) was stirred at 80° C. for 12 hours. LCMS showed 23% of 3-(4-((piperidin-4-ylmethyl)amino)phenyl)piperidine-2,6-dione remained and 6% of the desired mass was detected. To the mixture were added Na₂CO₃ (357.64 mg, 3.37 mmol), DIPEA (436.11 mg, 3.37 mmol, 587.75 μL) and tert-butyl ((1s,4s)-4-iodocyclohexyl)carbamate(cis) (182.88 mg, 562.39 μmol) and stirred at 80° C. for 2 hours, additional tert-butyl ((1s,4s)-4-iodocyclohexyl)carbamate(cis) (182.88 mg, 562.39 μmol) was added and the resulting mixture was stirred at 80° C. for further 14 hours, LCMS showed 11% of 3-(4-((piperidin-4-ylmethyl)amino)phenyl)piperidine-2,6-dione remained and 60% of the desired mass was detected, to the mixture was added tert-butyl ((1s,4s)-4-iodocyclohexyl)carbamate(cis) (91.44 mg, 281.20 μmol), the mixture was stirred at 80° C. for 2 hours, then tert-butyl ((1s,4s)-4-iodocyclohexyl)carbamate(cis) (91.44 mg, 281.20 μmol) was added, the resulting mixture was stirred at 80° C. for 2 hours, LCMS showed 3-(4-((piperidin-4-ylmethyl)amino)phenyl)piperidine-2,6-dione remained and 61% of the desired mass was detected, to the mixture was added tert-butyl ((1s,4s)-4-iodocyclohexyl)carbamate (cis) (365.76 mg, 1.12 mmol), the resulting mixture was stirred at 80° C. for 12 hours, LCMS showed trace of 3-(4-((piperidin-4-ylmethyl)amino)phenyl)piperidine-2,6-dione remained and ~50% of the desired mass was detected. The mixture was filtered and the filter cake was washed with ACN (40 mL), the filtrate was treated with AcOH to adjust pH<7, the resulting mixture was concentrated in vacuum, the residue was diluted with DCM (40 mL) and washed with H₂O (30 mL×2), the organic phase was dried over MgSO₄, filtered and concentrated in vacuum. The residue was purified by prep-TLC (SiO₂, DCM:MeOH=7:1) to afford tert-butyl ((1r,4r)-4-(4-(((4-(2,6-dioxopiperidin-3-yl)phenyl)amino)methyl)piperidin-1-yl)cyclohexyl)carbamate(trans) (63 mg, 126.34 μmol, 11.23% yield) as a brown solid. MS(M+H)⁺=499.3.

### Step 4. Synthesis of 3-(4-(((1-((1r,4r)-4-aminocyclohexyl)piperidin-4-yl)methyl)amino)phenyl) piperidine-2,6-dione(trans) (7)

A mixture of tert-butyl ((1r,4r)-4-(4-(((4-(2,6-dioxopiperidin-3-yl)phenyl)amino)methyl)piperidin-1-yl)cyclohexyl)carbamate(trans) (55 mg, 110.30 μmol), HCl/dioxane (4 M, 3 mL) in DCM (3 mL) was stirred at 15° C. for 1 hour. LCMS showed the starting material was consumed completely and desired mass was detected. The mixture was concentrated in vacuum to afford 3-(4-(((1-((1r,4r)-4-aminocyclohexyl)piperidin-4-yl)methyl)amino)phenyl)piperidine-2,6-dione(trans) (50 mg, HCl salt) as a brown solid, which was used in the next step directly. MS(M+H)⁺=399.3.

### Step 5. Synthesis of 4-(((R)-8-cyclopentyl-7-ethyl-5-methyl-6-oxo-5,6,7,8-tetrahydropteridin-2-yl)amino)-N-((1r,4R)-4-(4-(((4-(2,6-dioxopiperidin-3-yl)phenyl)amino)methyl)piperidin-1-yl)cyclohexyl)-3-methoxybenzamide(trans) (Compound 2)

To a solution of (R)-4-((8-cyclopentyl-7-ethyl-5-methyl-6-oxo-5,6,7,8-tetrahydropteridin-2-yl)amino)-3-methoxybenzoic acid (35 mg, 82.26 μmol) in DMF (2 mL) were added HATU (46.92 mg, 123.39 μmol) and DIPEA (63.79 mg, 493.56 μmol, 85.97 μL), the mixture was stirred at 15° C. for 15 minutes, then 3-(4-(((1-((1r,4r)-4-aminocyclohexyl)piperidin-4-yl)methyl)amino)phenyl)piperidine-2,6-dione(trans) (50 mg, 114.94 μmol, HCl salt) was added, the resulting mixture was stirred at 15° C. for 1 hour. LCMS showed 3-(4-(((1-((1r,4r)-4-aminocyclohexyl)piperidin-4-yl)methyl)amino)phenyl)piperidine-2,6-dione (trans) was consumed completely and a peak (65%) with desired mass. The mixture was diluted with H₂O (40 mL) and extracted with EtOAc (30 mL×3). The combined organic layers were washed with brine (80 mL×5), dried over Na₂SO₄, filtered and concentrated in vacuum. The residue was purified by prep-TLC (SiO₂, DCM:MeOH=7:1), the eluent was concentrated in vacuum. The residue was purified by prep-HPLC (column: Phenomenex luna C18 150*25 mm*10 um; mobile phase: [water (TFA)-ACN]; B %: 31%-51%, 7 min), the eluent contained DMF residual, the eluent was concentrated in vacuum to remove most of the ACN, the residue was treated with NaHCO₃ solution (0.5 mL) to adjust pH>7, and extracted with DCM (10 mL×3). The organic phase was washed with brine (10 mL×4), dried over MgSO₄, filtered and concentrated in vacuum, the residue was diluted with H₂O (10 mL) and ACN (5 mL) and freeze-dried to afford 4-(((R)-8-cyclopentyl-7-ethyl-5-methyl-6-oxo-5,6,7,8-tetrahydropteridin-2-yl)amino)-N-((1r,4R)-4-(4-(((4-(2,6-dioxopiperidin-3-yl)phenyl)amino)methyl)piperidin-1-yl)cyclohexyl)-3-methoxybenzamide (trans) (15.2 mg, 18.10 μmol, 22.01% yield, 96% purity) as a white solid. MS(M+H)⁺=806.4.

¹H NMR (400 MHz, DMSO-d₆) δ=10.73 (s, 1H), 8.41 (d, J=8.9 Hz, 1H), 8.16-8.03 (m, 1H), 7.84 (s, 1H), 7.60 (s, 1H), 7.52-7.40 (m, 2H), 6.90 (d, J=8.6 Hz, 2H), 6.53 (br d, J=8.4 Hz, 2H), 5.77-5.59 (m, 1H), 4.42-4.30 (m, 1H), 4.27-4.19 (m, 1H), 3.94 (s, 3H), 3.84-3.69 (m, 1H), 3.63 (dd, J=5.1, 10.8 Hz, 1H), 3.25 (s, 3H), 2.94-2.88 (m, 2H), 2.65-2.58 (m, 2H), 2.47-2.45 (m, 1H), 2.43 (br d, J=4.4 Hz, 1H), 2.06 (br d, J=4.5 Hz, 2H), 2.05-1.99 (m, 3H), 1.99-1.93 (m, 3H), 1.93-1.84 (m, 4H), 1.83-1.72 (m, 5H), 1.69-1.52 (m, 5H), 1.50-1.33 (m, 4H), 1.28-1.21 (m, 2H), 0.76 (t, J=7.4 Hz, 3H).

### Example 3. Synthesis of 4-(((R)-8-cyclopentyl-7-ethyl-5-methyl-6-oxo-5,6,7,8-tetrahydropteridin-2-yl)amino)-N-((1r,4R)-4-(4-(3-(4-((2,6-dioxopiperidin-3-yl)amino)phenyl)propyl)piperazin-1-yl)cyclohexyl)-3-methoxybenzamide (Compound 3)

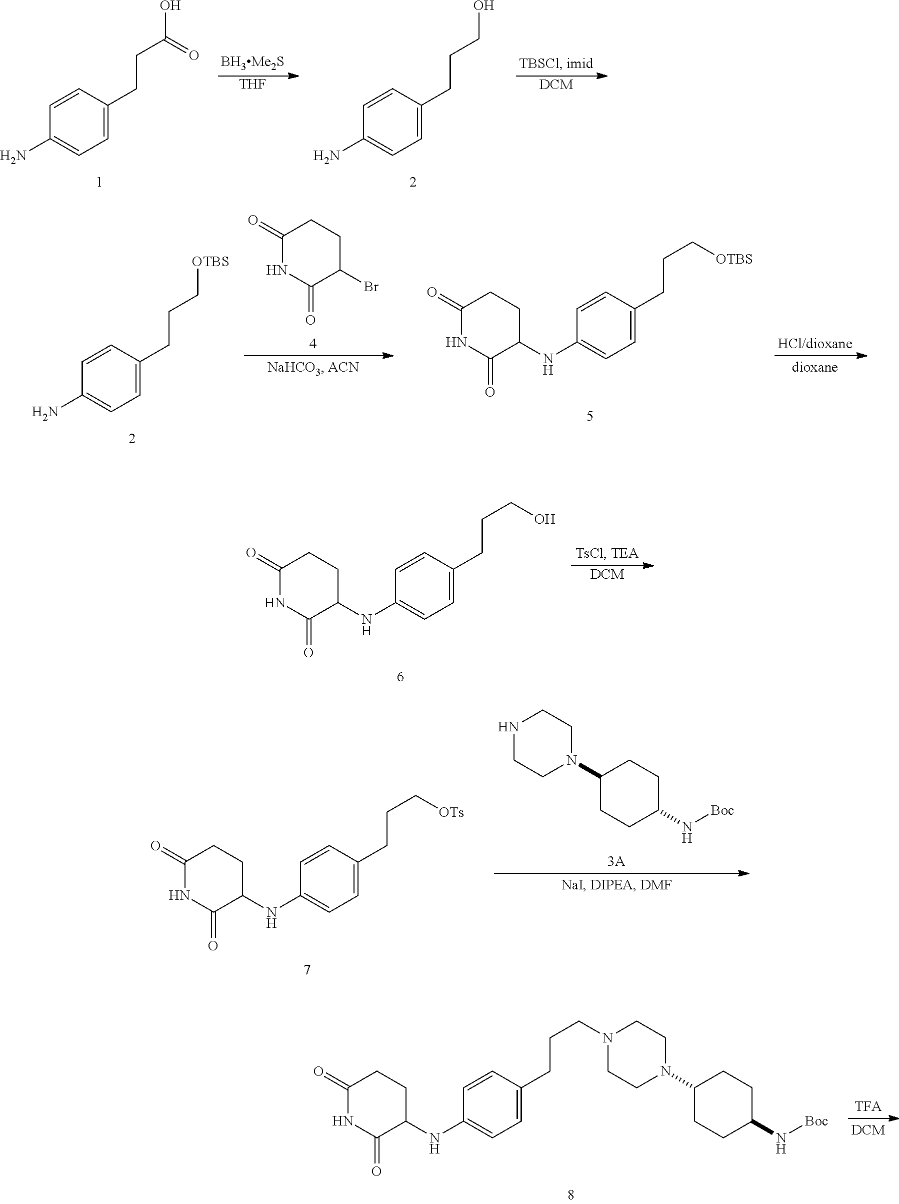

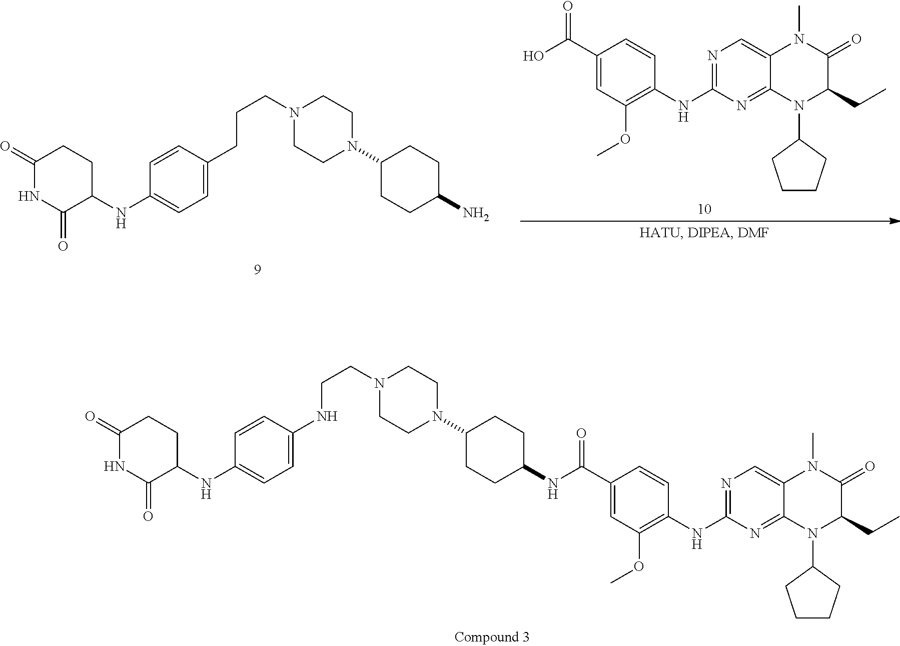

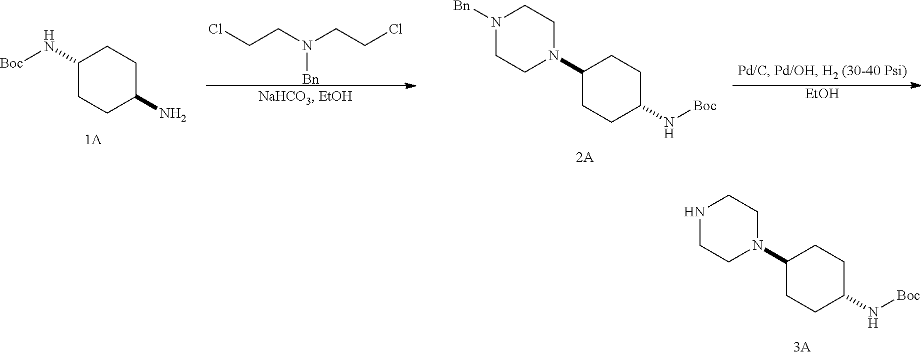

### Step 1. Synthesis of tert-butyl ((1r,4r)-4-(4-benzylpiperazin-1-yl)cyclohexyl)carbamate (trans) (2A)

To a solution of tert-butyl ((1r,4r)-4-aminocyclohexyl)carbamate (trans) (2 g, 9.33 mmol) and N-benzyl-2-chloro-N-(2-chloroethyl)ethan-1-amine (2.27 g, 8.46 mmol, HCl) in EtOH (20 mL) was added NaHCO₃ (3.92 g, 46.66 mmol, 1.81 mL). The mixture was stirred at 85° C. for 16 h. LCMS showed tert-butyl ((1r,4r)-4-aminocyclohexyl)carbamate (trans) was consumed completely and desired mass was detected. The reaction mixture was quenched with H₂O (30 mL) at 25° C., and then extracted with EtOAc (160 mL). The organic layer was dried over Na₂SO₄, filtered and concentrated under reduced pressure to give a residue. The residue was triturated with EtOAc at 25° C. for 30 min to afford tert-butyl ((1r,4r)-4-(4-benzylpiperazin-1-yl)cyclohexyl)carbamate (trans) (2.1 g, 5.34 mmol, 57.23% yield, 95% purity) as a white solid. MS(M+H)⁺=374.4.

### Step 2. Synthesis of tert-butyl ((1r,4r)-4-(piperazin-1-yl)cyclohexyl)carbamate (trans) (3A)

To a solution of tert-butyl ((1r,4r)-4-(4-benzylpiperazin-1-yl)cyclohexyl)carbamate(trans) (2.1 g, 5.62 mmol) in EtOH (20 mL) was added Pd/C (500 mg, 10% purity) and Pd(OH)₂/C (500 mg, 20% purity) under N₂ atmosphere. The suspension was degassed and purged with H₂ for 3 times. The mixture was stirred under H₂ (30-40 Psi) at 25° C. for 16 h. TLC (DCM:MeOH=10:1) indicated tert-butyl ((1r,4r)-4-(4-benzylpiperazin-1-yl)cyclohexyl)carbamate (trans) was consumed completely and one new spot formed. The mixture was filtered and the filtrate was concentrated under reduced pressure to afford tert-butyl ((1r,4r)-4-(piperazin-1-yl)cyclohexyl)carbamate (trans) (1.52 g, 5.36 mmol, 95.40% yield) as a white solid.

### Step 3. Synthesis of 3-(4-aminophenyl)propan-1-ol (2)

To a solution of 3-(4-aminophenyl)propanoic acid (2 g, 12.11 mmol) in THF (30 mL) was added BH₃-Me₂S (10 M, 4.84 mL) at 0° C. The mixture was stirred at 25° C. for 2 hr. LCMS showed 3-(4-aminophenyl)propanoic acid was consumed completely and the desired mass was detected. The reaction mixture was poured into 40 mL of HCl (1N) solution at 0° C. The mixture was adjusted the pH value to 7.4 with saturated NaHCO₃ solution and extracted with EtOAc (120 mL). The organic layers were dried over Na₂SO₄, filtered and concentrated under reduced pressure to afford 3-(4-aminophenyl)propan-1-ol (1.82 g, 12.04 mmol, 99.42% yield) as a brown oil. The crude product was used into the next step without further purification. MS (M+H)⁺=152.0.

### Step 4. Synthesis of 4-(3-((tert-butyldimethylsilyl)oxy)propyl)aniline (3)

To a solution of 3-(4-aminophenyl)propan-1-ol (1.82 g, 12.04 mmol) in DCM (20 mL) was added TBSCl (2.18 g, 14.44 mmol, 1.78 mL) and Imid (1.64 g, 24.07 mmol). The mixture was stirred at 25° C. for 16 h. LCMS showed 3-(4-aminophenyl)propan-1-ol was consumed completely and the desired mass was detected. The reaction mixture was poured into 50 mL of HCl (1N) solution at 0° C., and then extracted with EtOAc (100 mL). The combined organic layers were dried over Na₂SO₄, filtered and concentrated under reduced pressure to give a residue. The residue was purified by flash silica gel chromatography (ISCO®; 40 g SepaFlash® Silica Flash Column, Eluent of 0~20% EtOAc/Petroleum ether gradient @50 mL/min) to afford 4-(3-((tert-butyldimethylsilyl)oxy)propyl)aniline (2.9 g, 10.92 mmol, 90.76% yield) as a brown oil. MS (M+H)⁺=266.1.

### Step 5. Synthesis of 3-((4-(3-((tert-butyldimethylsilyl)oxy)propyl)phenyl)amino)piperidine-2,6-dione (5)

To a solution of 4-(3-((tert-butyldimethylsilyl)oxy)propyl)aniline (2.7 g, 10.17 mmol) and 3-bromopiperidine-2,6-dione (4.88 g, 25.43 mmol) in ACN (15 mL) was added NaHCO₃ (4.96 g, 58.99 mmol, 2.30 mL). The mixture was stirred at 80° C. for 14 h. LCMS showed 4-(3-((tert-butyldimethylsilyl)oxy)propyl)aniline was consumed completely and the desired mass was detected. The reaction mixture was filtered and the filtrate was concentrated under reduced pressure to give a residue. The residue was purified by flash silica gel chromatography (ISCO®; 20 g SepaFlash® Silica Flash Column, Eluent of 0~17% EtOAc/Petroleum ether gradient @120 mL/min) to afford 3-((4-(3-((tert-butyldimethylsilyl)oxy)propyl)phenyl)amino)piperidine-2,6-dione (0.78 g, 2.07 mmol, 20.37% yield) as a blue solid. MS (M+H)⁺=377.4.

### Step 6. Synthesis of 3-((4-(3-hydroxypropyl)phenyl)amino)piperidine-2,6-dione (6)

To a solution of 3-((4-(3-((tert-butyldimethylsilyl)oxy)propyl)phenyl)amino)piperidine-2,6-dione (0.78 g, 2.07 mmol) in dioxane (8 mL) was added HCl/dioxane (4 M, 16 mL). The mixture was stirred at 25° C. for 1 h. LCMS showed 3-((4-(3-((tert-butyldimethylsilyl)oxy)propyl)phenyl)amino)piperidine-2,6-dione was consumed completely and the desired mass was detected. The mixture was concentrated under reduced pressure to afford 3-((4-(3-hydroxypropyl)phenyl)amino)piperidine-2,6-dione (0.54 g, 2.06 mmol, 99.39% yield) as a blue solid. The product was used into the next step without further purification. MS (M+H)⁺=263.3.

### Step 7. Synthesis of 3-(4-((2,6-dioxopiperidin-3-yl)amino)phenyl)propyl 4-methylbenzenesulfonate (7)

To a solution of 3-((4-(3-hydroxypropyl)phenyl)amino)piperidine-2,6-dione (0.54 g, 2.06 mmol) in DCM (6 mL) was added TEA (624.95 mg, 6.18 mmol, 859.63 μL) and TosCl (588.72 mg, 3.09 mmol). The mixture was stirred at 20° C. for 12 h. LCMS showed 3-((4-(3-hydroxypropyl)phenyl)amino)piperidine-2,6-dione was consumed completely and the desired mass was detected. The mixture was concentrated under reduced pressure to give a residue. The residue was purified by flash silica gel chromatography (ISCO®; 40 g SepaFlash® Silica Flash Column, Eluent of 0~50% EtOAc/Petroleum ether gradient @80 mL/min) to afford 3-(4-((2,6-dioxopiperidin-3-yl)amino)phenyl)propyl 4-methylbenzenesulfonate (710 mg, 1.62 mmol, 78.67% yield, 95% purity) as a blue solid. MS (M+H)⁺=417.1.

### Step 8. Synthesis of tert-butyl ((1r,4r)-4-(4-(3-(4-((2,6-dioxopiperidin-3-yl)amino)phenyl)propyl)piperazin-1-ylcyclohexyl)carbamate (trans) (8)

To a solution of 3-(4-((2,6-dioxopiperidin-3-yl)amino)phenyl)propyl 4-methylbenzenesulfonate (0.4 g, 960.41 μmol) and tert-butyl ((1r,4r)-4-(piperazin-1-yl)cyclohexyl)carbamate (trans) (0.45 g, 1.59 mmol) in DMF (4 mL) was added DIPEA (744.76 mg, 5.76 mmol, 1.00 mL) and NaI (28.79 mg, 192.08 mol). The mixture was stirred at 25° C. for 16 h. LCMS showed 3-(4-((2,6-dioxopiperidin-3-yl)amino)phenyl)propyl 4-methylbenzenesulfonate was consumed completely and the desired mass was detected. The reaction mixture was quenched with H₂O (20 mL) at 25° C., and then extracted with EtOAc (120 mL). The combined organic layers were washed with brine (20 mL), dried over Na₂SO₄, filtered and concentrated under reduced pressure to give a residue. The residue was purified by flash silica gel chromatography (ISCO®; 20 g SepaFlash® Silica Flash Column, Eluent of 0~20% MeOH/EtOAc @50 mL/min) to afford tert-butyl ((1r,4r)-4-(4-(3-(4-((2,6-dioxopiperidin-3-yl)amino)phenyl)propyl)piperazin-1-yl)cyclohexyl)carbamate (trans) (0.227 g, 430.17 μmol, 44.79% yield) as a yellow solid. MS (M+H)⁺=528.3.

### Step 9. Synthesis of 3-((4-(3-(4-((1r,4r)-4-aminocyclohexyl)piperazin-1-yl)propyl)phenyl)amino)piperidine-2,6-dione (trans) (9)

To a solution of tert-butyl ((1r,4r)-4-(4-(3-(4-((2,6-dioxopiperidin-3-yl)amino)phenyl)propyl)piperazin-1-yl)cyclohexyl)carbamate (trans) (0.1 g, 189.50 μmol) in DCM (0.3 mL) was added TFA (153.50 mg, 1.35 mmol, 0.1 mL). The mixture was stirred at 25° C. for 1 hr. LCMS showed tert-butyl ((1r,4r)-4-(4-(3-(4-((2,6-dioxopiperidin-3-yl)amino)phenyl)propyl)piperazin-1-yl)cyclohexyl)carbamate (trans) was consumed completely and desired mass was detected. The reaction mixture was concentrated under reduced pressure to afford 3-((4-(3-(4-((1r,4r)-4-aminocyclohexyl)piperazin-1-yl)propyl)phenyl)amino)piperidine-2,6-dione (trans) (0.1 g, 184.64 μmol, 97.43% yield, TFA) as a yellow oil. The product was used into the next step without further purification. MS(M+H)⁺=428.5.

### Step 10. Synthesis of 4-(((R)-8-cyclopentyl-7-ethyl-5-methyl-6-oxo-5,6,7,8-tetrahydropteridin-2-yl)amino)-N-((1r,4R)-4-(4-(3-(4-((2,6-dioxopiperidin-3-yl)amino)phenyl)propyl)piperazin-1-yl)cyclohexyl)-3-methoxybenzamide (trans) (Compound 3)

To a solution of (R)-4-((8-cyclopentyl-7-ethyl-5-methyl-6-oxo-5,6,7,8-tetrahydropteridin-2-yl)amino)-3-methoxybenzoic acid (78.56 mg, 184.64 μmol) in DMF (1 mL) were added HATU (105.31 mg, 276.95 μmol) and DIPEA (119.31 mg, 923.18 μmol, 160.80 μL). The mixture was stirred at 25° C. for 0.5 hr. Then to the mixture was added 3-((4-(3-(4-((1r,4r)-4-aminocyclohexyl)piperazin-1-yl)propyl)phenyl)amino)piperidine-2,6-dione (trans) (0.1 g, 184.64 μmol, TFA) and stirred at 25° C. for another 16 hr. LCMS showed 3-((4-(3-(4-((1r,4r)-4-aminocyclohexyl)piperazin-1-yl)propyl)phenyl)amino)piperidine-2,6-dione (trans) was consumed completely and the desired mass was detected. The reaction mixture was quenched with H₂O (10 mL) at 25° C., and then extracted with EtOAc (80 mL). The combined organic layers were washed with brine (10 mL), dried over Na₂SO₄, filtered and concentrated under reduced pressure to give a residue. The residue was purified by flash silica gel chromatography (ISCO®; 20 g SepaFlash® Silica Flash Column, Eluent of 0~30% MeOH/EtOAc 50 mL/min) to afford 4-(((R)-8-cyclopentyl-7-ethyl-5-methyl-6-oxo-5,6,7,8-tetrahydropteridin-2-yl)amino)-N-((1r,4R)-4-(4-(3-(4-((2,6-dioxopiperidin-3-yl)amino)phenyl)propyl)piperazin-1-yl)cyclohexyl)-3-methoxybenzamide (trans) (91.6 mg, 104.21 μmol, 56.44% yield, 95% purity) as a white solid. MS (M+H)⁺=835.4.

¹H NMR (400 MHz, DMSO-d₆) δ=10.77 (s, 1H), 8.45-8.36 (m, 1H), 8.03 (d, J=7.6 Hz, 1H), 7.84 (s, 1H), 7.59 (s, 1H), 7.52-7.42 (m, 2H), 6.91 (d, J=8.3 Hz, 2H), 6.59 (d, J=8.3 Hz, 2H), 5.62 (d, J=7.3 Hz, 1H), 4.36 (t, J=8.0 Hz, 1H), 4.30-4.19 (m, 2H), 3.93 (s, 3H), 3.77-3.66 (m, 1H), 3.24 (s, 3H), 2.71-2.60 (m, 1H), 2.63-2.55 (m, 4H), 2.45-2.37 (m, 5H), 2.31-2.23 (m, 3H), 2.18-1.98 (m, 3H), 1.96-1.84 (m, 7H), 1.80-1.70 (m, 4H), 1.68-1.58 (m, 5H), 1.44-1.23 (m, 5H), 0.76 (t, J=7.4 Hz, 3H)

### Example 4. Synthesis of 4-(((R)-8-cyclopentyl-7-ethyl-5-methyl-6-oxo-5,6,7,8-tetrahydropteridin-2-yl)amino)-N-((1r,4R)-4-(4-(4-((2,6-dioxopiperidin-3-yl)amino)phenethyl)piperazin-1-yl)cyclohexyl)-3-methoxybenzamide (Compound 4)

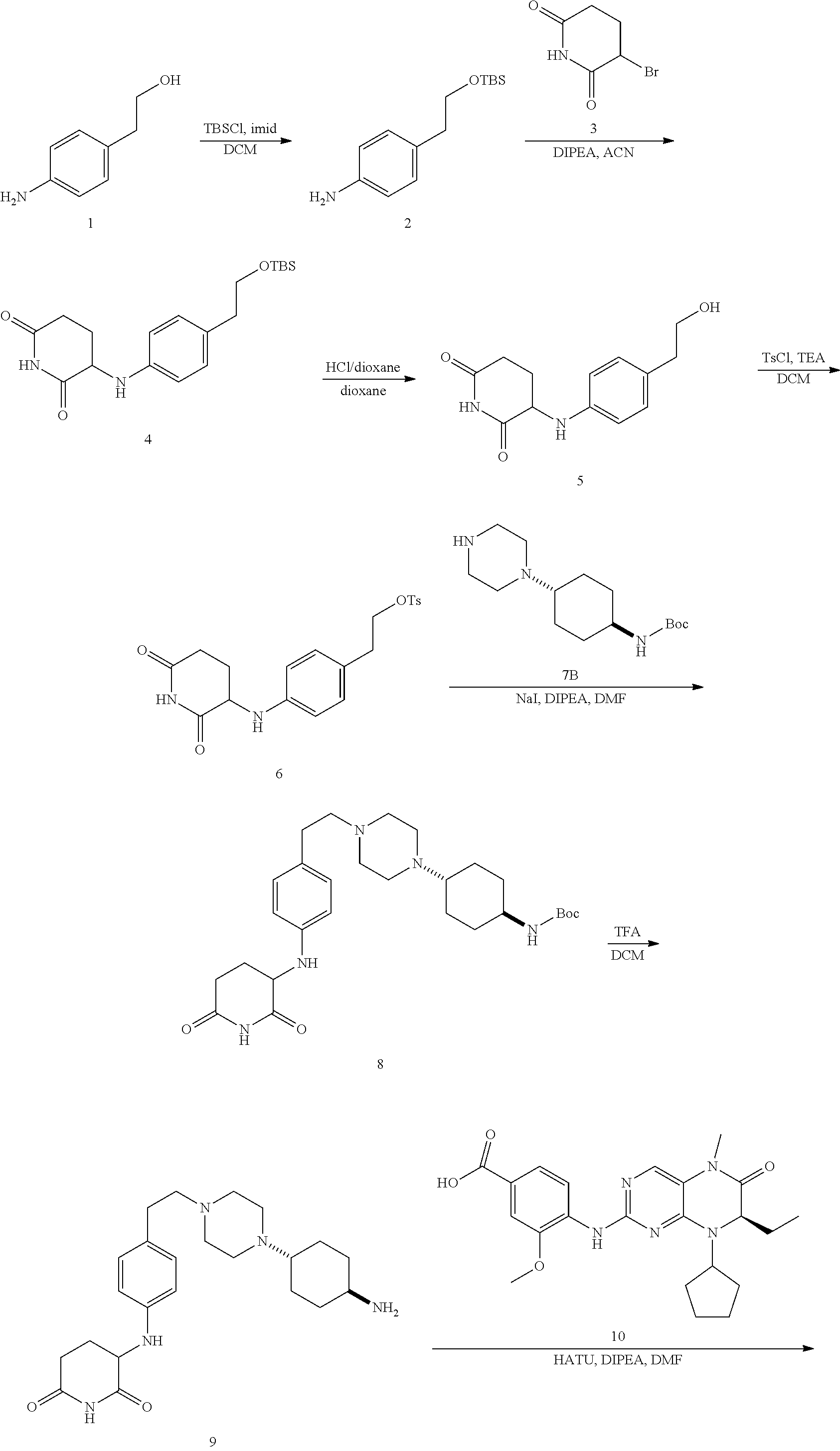

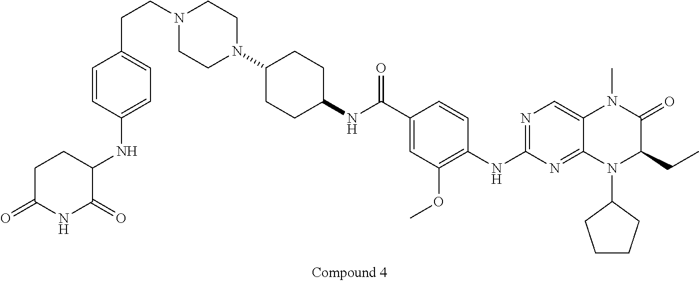

### Step 1. Synthesis of 4-(2-((tert-butyldimethylsilyl)oxy)ethyl)aniline (2)

To a solution of 2-(4-aminophenyl)ethan-1-ol (2 g, 14.58 mmol) in DCM (20 mL) were added TBSCl (2.7 g, 17.91 mmol, 2.2 mL) and imidazole (1.2 g, 17.63 mmol) and the mixture was stirred at 25° C. for 14 h. TLC (Petroleum ether:EtOAc=5:1) showed a new spot. The mixture was diluted with water (20 mL) and extracted with EtOAc (20 mL×2). The combined organic layer was concentrated under reduced pressure. The reside was purified by flash silica gel chromatography (20 g SepaFlash® Silica Flash Column, Eluent of 20~30% EtOAc/MeOH gradient @50 mL/min) to afford 4-(2-((tert-butyldimethylsilyl)oxy)ethyl)aniline (4.27 g, crude) as a yellow oil. MS(M+H)⁺=252.4

### Step 2. Synthesis of 3-((4-(2-((tert-butyldimethylsilyl)oxy)ethyl)phenyl)amino)piperidine-2,6-dione (4)

To a solution of 4-[2-[tert-butyl(dimethyl)silyl]oxyethyl]aniline (2 g, 7.95 mmol) and 3-bromopiperidine-2,6-dione (2.29 g, 11.93 mmol) in ACN (40 mL) was added DIPEA (3.08 g, 23.86 mmol, 4.16 mL) at 20° C. and the reaction mixture was stirred at 80° C. for 12 h. LCMS showed 44% of 4-(2-((tert-butyldimethylsilyl)oxy)ethyl)aniline remained and 38% of peak with desired mass. The reaction mixture was concentrated in vacuum. The residue was purified by flash silica gel chromatography (ISCO®; 20 g SepaFlash® Silica Flash Column, Eluent of 0~25% EtOAc/Petroleum ether gradient @200 mL/min) to afford 3-((4-(2-((tert-butyldimethylsilyl)oxy)ethyl)phenyl)amino)piperidine-2,6-dione (806 mg, 2.22 mmol, 27.95% yield) as a brown solid. MS(M+H)⁺=363.3

### Step 3. Synthesis of 3-((4-(2-hydroxyethyl)phenyl)amino)piperidine-2,6-dione (5)

To a solution of 3-[4-[2-[tert-butyl(dimethyl)silyl]oxyethyl]anilino]piperidine-2,6-dione (400 mg, 1.10 mmol) in dioxane (5 mL) was added HCl/dioxane (4 M, 10 mL) at 20° C. and the mixture was stirred at 20° C. for 1 h. LCMS showed starting material was consumed completely and the desired mass was detected. The reaction mixture was combined with another batch (200 mg). The reaction mixture was concentrated in vacuum to afford 3-((4-(2-hydroxyethyl)phenyl)amino)piperidine-2,6-dione (600 mg, crude) as a brown solid. MS(M+H)⁺=249.2

### Step 4. Synthesis of 4-((2,6-dioxopiperidin-3-yl)amino)phenethyl 4-methylbenzenesulfonate (6)

To a solution of 3-((4-(2-hydroxyethyl)phenyl)amino)piperidine-2,6-dione (600 mg, 2.42 mmol) in DCM (8 mL) were added TEA (733.62 mg, 7.25 mmol, 1.01 mL) and TosCl (691.09 mg, 3.62 mmol) at 20° C. The mixture was stirred at 20° C. for 12 h. LCMS showed starting material was consumed completely and 58% of peak with desired mass. The reaction mixture was concentrated in vacuum. The residue was purified by flash silica gel chromatography (12 g SepaFlash® Silica Flash Column, Eluent of 0~50% EtOAc/Petroleum ether gradient @100 mL/min) to afford 4-((2,6-dioxopiperidin-3-yl)amino)phenethyl 4-methylbenzenesulfonate (332 mg, 824.92 μmol, 34.13% yield) as a blue solid. MS(M+H)⁺=403.2

### Step 5. Synthesis of tert-butyl ((1r,4r)-4-(4-(4-((2,6-dioxopiperidin-3-yl)amino)phenethyl)piperazin-1-yl)cyclohexyl) carbamate (trans) (8)

To a solution of 4-((2,6-dioxopiperidin-3-yl)amino)phenethyl 4-methylbenzenesulfonate (332 mg, 824.92 μmol) and tert-butyl ((1r,4r)-4-(piperazin-1-yl)cyclohexyl)carbamate (trans) (467.58 mg, 1.65 mmol) in DMF (10 mL) were added DIPEA (639.68 mg, 4.95 mmol, 862.10 μL) and NaI (24.73 mg, 164.98 μmol) at 20° C. The mixture was stirred at 40° C. for 12 h. LCMS showed 26% of 4-((2,6-dioxopiperidin-3-yl)amino)phenethyl 4-methylbenzenesulfonate remained and 25% of peak with desired mass. The reaction mixture was diluted with H₂O (40 mL) and extracted with EtOAc (20 mL×3). The organic layer was washed with brine (15 mL×3), dried over Na₂SO₄, filtered and concentrated in vacuum. The residue was purified by prep-TLC (SiO₂, DCM:MeOH=10:1) and re-purified by reversed-phase HPLC (column: Phenomenex luna C18 150*25 mm*10 um; mobile phase: [water(TFA)-ACN]; B %: 14%-44%, 9 min) and lyophilized to afford tert-butyl ((1r,4r)-4-(4-(4-((2,6-dioxopiperidin-3-yl)amino)phenethyl)piperazin-1-yl)cyclohexyl) carbamate (trans) (220 mg, 332.96 μmol, 40.36% yield, 95% purity, TFA). MS(M+H)⁺=514.5

### Step 6. Synthesis of 3-((4-(2-(4-((1r,4r)-4-aminocyclohexyl)piperazin-1-yl)ethyl)phenyl)amino)piperidine-2,6-dione (trans) (9)

To a solution of tert-butyl ((1r,4r)-4-(4-(4-((2,6-dioxopiperidin-3-yl)amino)phenethyl)piperazin-1-yl)cyclohexyl) carbamate (trans) (220 mg, 428.29 μmol, TFA) in DCM (3 mL) was added TFA (460.50 mg, 4.04 mmol, 0.3 mL) at 20° C., the mixture was stirred at 20° C. for 1 h. LCMS showed starting material was consumed completely and 45% of peak with desired mass. The reaction mixture was concentrated in vacuum to afford 3-((4-(2-(4-((1r,4r)-4-aminocyclohexyl)piperazin-1-yl)ethyl)phenyl)amino)piperidine-2,6-dione (trans) (220 mg, crude, TFA) as a black green oil. MS(M+H)⁺=414.4

### Step 7. Synthesis of 4-(((R)-8-cyclopentyl-7-ethyl-5-methyl-6-oxo-5,6,7,8-tetrahydropteridin-2-yl)amino)-N-((1r,4R)-4-(4-(4-((2,6-dioxopiperidin-3-yl)amino)phenethyl)piperazin-1-yl)cyclohex yl)-3-methoxybenzamide (trans) (Compound 4)

To a solution of (R)-4-((8-cyclopentyl-7-ethyl-5-methyl-6-oxo-5,6,7,8-tetrahydropteridin-2-yl)amino)-3-methoxybenzoic acid (169.36 mg, 398.05 μmol) in DMF (4 mL) were added HATU (166.48 mg, 437.85 μmol) and DIPEA (308.67 mg, 2.39 mmol, 415.99 μL) at 20° C., after stirring 10 min, then a solution of 3-((4-(2-(4-((1r,4r)-4-aminocyclohexyl)piperazin-1-yl)ethyl)phenyl)amino)piperidine-2,6-dione (trans) (210 mg, 398.05 μmol, TFA) in DMF (4 mL) and DIPEA (308.67 mg, 2.39 mmol, 415.99 L) was added at 20° C. The reaction mixture was stirred at 20° C. for 1 h. LCMS showed starting material was consumed completely and 37% of peak with desired mass. The reaction mixture was diluted with H₂O (40 mL) and extracted with EtOAc (15 mL×3), the organic layer was washed with brine (12 mL×3), dried over Na₂SO₄, filtered and concentrated in vacuum. The crude product was purified by prep-TLC (SiO₂, DCM:MeOH=10:1) and the product was dissolved in mixture solution (20 mL, ACN:H₂O=1:3) and lyophilized to afford 4-(((R)-8-cyclopentyl-7-ethyl-5-methyl-6-oxo-5,6,7,8-tetrahydropteridin-2-yl)amino)-N-((1r,4R)-4-(4-(4-((2,6-dioxopiperidin-3-yl)amino)phenethyl)piperazin-1-yl)cyclohex yl)-3-methoxybenzamide (trans) (82.6 mg, 96.38 μmol, 24.21% yield, 95.8% purity) as a white solid. MS(M+H)⁺=821.5

¹H NMR (400 MHz, DMSO-d₆) δ=10.77 (s, 1H), 8.41 (d, J=8.9 Hz, 1H), 8.11-8.03 (m, 1H), 7.84 (s, 1H), 7.60 (s, 1H), 7.50-7.44 (m, 2H), 6.95 (d, J=8.4 Hz, 2H), 6.61 (d, J=8.3 Hz, 2H), 5.78-5.62 (m, 1H), 4.41-4.31 (m, 1H), 4.31-4.21 (m, 2H), 3.94 (s, 3H), 3.79-3.68 (m, 1H), 3.25 (s, 3H), 2.82-2.68 (m, 4H), 2.65-2.52 (m, 9H), 2.15-1.70 (m, 15H), 1.69-1.58 (m, 3H), 1.48-1.31 (m, 4H), 0.76 (t, J=7.4 Hz, 3H)

### Example 5. Synthesis of 4-(((R)-8-cyclopentyl-7-ethyl-5-methyl-6-oxo-5,6,7,8-tetrahydropteridin-2-yl)amino)-N-((1r,4R)-4-(4-(2-((2-(2,6-dioxopiperidin-3-yl)-1-oxoisoindolin-4-yl)amino)eth yl)piperazin-1-yl)cyclohexyl)-3-methoxybenzamide (Compound 5)

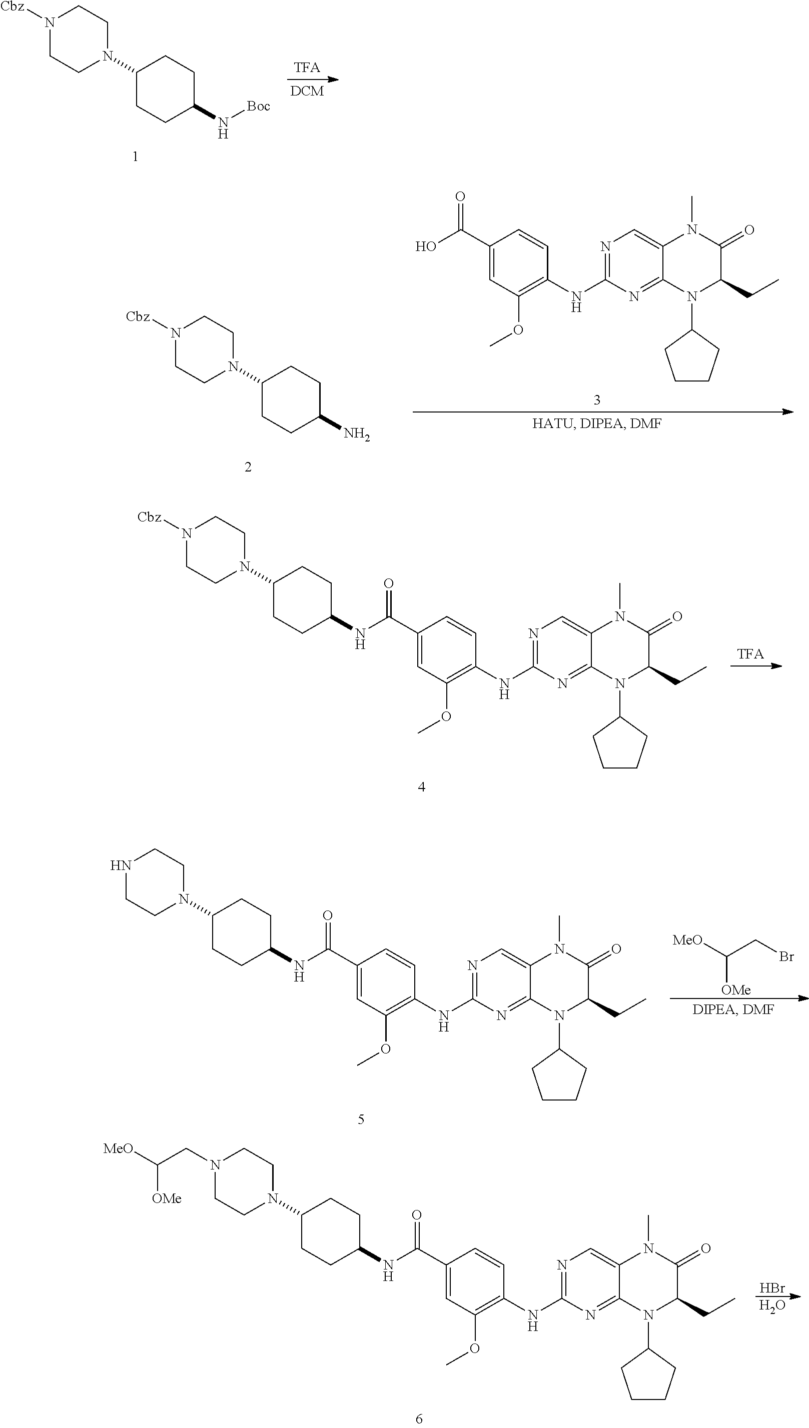

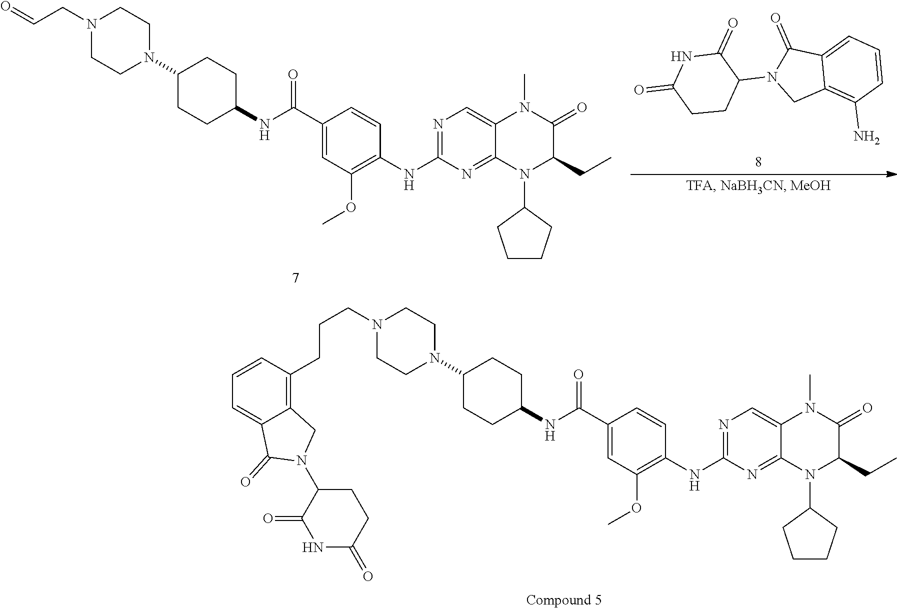

### Step 1. Synthesis of benzyl 4-((1r,4r)-4-aminocyclohexyl)piperazine-1-carboxylate (trans) (2)

To a solution of benzyl 4-((1r,4r)-4-((tert-butoxycarbonyl)amino)cyclohexyl)piperazine-1-carboxylate (trans) (1 g, 2.39 mmol) in DCM (5 mL) was added TFA (1.69 g, 14.86 mmol, 1.1 mL) at 20° C. and the resulting mixture was stirred at 20° C. for 1 h. LCMS showed starting material remained and desired mass was detected. TFA (1.37 g, 11.97 mmol, 886.63 μL) was added to this reaction mixture at 20° C. and the resulting mixture was stirred at 20° C. for 2 h. LCMS showed starting material remained and a peak with desired mass. The reaction mixture was stirred at 20° C. for 12 h. LCMS showed starting material was consumed completely and a main peak with desired mas. The reaction mixture was concentrated in vacuum to afford benzyl 4-((1r,4r)-4-aminocyclohexyl)piperazine-1-carboxylate (trans) (1.03 g, crude, TFA) as a yellow oil. MS(M+H)⁺=318.2

### Step 2. Synthesis of benzyl 4-((1R,4r)-4-(4-(((R)-8-cyclopentyl-7-ethyl-5-methyl-6-oxo-5,6,7,8-tetrahydropteridin-2-yl)amino)-3-methoxybenzamido)cyclohexyl)piperazine-1-carboxylate (trans) (4)

To a solution of (R)-4-((8-cyclopentyl-7-ethyl-5-methyl-6-oxo-5,6,7,8-tetrahydropteridin-2-yl)amino)-3-methoxybenzoic acid (trans) (0.8 g, 1.88 mmol) in DMF (6 mL) were added HATU (786.41 mg, 2.07 mmol) and DIPEA (729.02 mg, 5.64 mmol, 982.51 L) at 20° C. After stirring 10 min, a solution of benzyl 4-((1r,4r)-4-aminocyclohexyl)piperazine-1-carboxylate (1.03 g, crude, TFA) in DMF (6 mL) and DIPEA (1.46 g, 11.28 mmol, 1.97 mL) was added at 20° C. and the resulting mixture was stirred at 20° C. for 1 h. LCMS showed all starting material was consumed completely and 78% peak with desired mass. The reaction mixture was diluted with H₂O (20 mL) and extracted with EtOAc (20 mL×3). The organic layer was washed with brine (20 mL×3), dried over Na₂SO₄, filtered and concentrated. The residue was purified by flash silica gel chromatography (25 g SepaFlash® Silica Flash Column, Eluent of 0~100% EtOAc/Petroleum ether to 0~15% DCM/MeOH gradient @200 mL/min) to afford benzyl 4-((1R,4r)-4-(4-(((R)-8-cyclopentyl-7-ethyl-5-methyl-6-oxo-5,6,7,8-tetrahydropteridin-2-yl)amino)-3-methoxybenzamido)cyclohexyl)piperazine-1-carboxylate (trans) (1.2 g, 1.64 mmol, 87.17% yield, 99% purity) as an off-white solid. MS(M+H)⁺=725.4

### Step 3. Synthesis of 4-(((R)-8-cyclopentyl-7-ethyl-5-methyl-6-oxo-5,6,7,8-tetrahydropteridin-2-yl)amino)-3-methoxy-N-((1r,4R)-4-(piperazin-1-yl)cyclohexyl)benzamide (trans) (5)

A solution of benzyl 4-((1R,4r)-4-(4-(((R)-8-cyclopentyl-7-ethyl-5-methyl-6-oxo-5,6,7,8-tetrahydropteridin-2-yl)amino)-3-methoxybenzamido)cyclohexyl)piperazine-1-carboxylate (trans) (600.00 mg, 827.71 μmol) in TFA (7.68 g, 67.31 mmol, 5 mL) was stirred at 50° C. for 12 h. LCMS showed starting material was consumed completely and 84% peak with desired mass. The reaction mixture was concentrated in vacuum to afford 4-(((R)-8-cyclopentyl-7-ethyl-5-methyl-6-oxo-5,6,7,8-tetrahydropteridin-2-yl)amino)-3-methoxy-N-((1r,4R)-4-(piperazin-1-yl)cyclohexyl)benzamide (trans) (583 mg, crude, TFA) as a brown oil. MS(M+H)⁺=591.4

### Step 4. Synthesis of 4-(((R)-8-cyclopentyl-7-ethyl-5-methyl-6-oxo-5,6,7,8-tetrahydropteridin-2-yl)amino)-N-((1r,4R)-4-(4-(2,2-dimethoxyethyl)piperazin-1-yl)cyclohexyl)-3-methoxybenzamide (trans) (6)

To a solution of 4-(((R)-8-cyclopentyl-7-ethyl-5-methyl-6-oxo-5,6,7,8-tetrahydropteridin-2-yl)amino)-3-methoxy-N-((1r,4R)-4-(piperazin-1-yl)cyclohexyl)benzamide (trans) (523.00 mg, crude, TFA) in DMF (10 mL) were added DIPEA (575.43 mg, 4.45 mmol, 775.52 μL) and 2-bromo-1,1-dimethoxy-ethane (224.51 mg, 1.33 mmol, 155.91 μL) at 20° C. and the resulting mixture was stirred at 100° C. for 12 h. LCMS showed 3% of starting material remained and 27% peak with desired mass. The reaction mixture was diluted with H₂O (20 mL) and extracted with EtOAc (15 mL×3). The organic layer was washed with brine (15 mL×3), dried over Na₂SO₄, filtered and concentrated. The residue was purified by flash silica gel chromatography (10 g SepaFlash® Silica Flash Column, Eluent of 0~100% EtOAc/Petroleum ether gradient @150 mL/min) to afford 4-(((R)-8-cyclopentyl-7-ethyl-5-methyl-6-oxo-5,6,7,8-tetrahydropteridin-2-yl)amino)-N-((1r,4R)-4-(4-(2,2-dimethoxyethyl)piperazin-1-yl)cyclohexyl)-3-methoxybenzamide (trans) (283 mg, 416.87 μmol, 56.18% yield) as a yellow solid. MS(M+H)⁺=679.4

### Step 5. Synthesis of 4-(((R)-8-cyclopentyl-7-ethyl-5-methyl-6-oxo-5,6,7,8-tetrahydropteridin-2-yl)amino)-3-methoxy-N-((1r,4R)-4-(4-(2-oxoethyl)piperazin-1-yl)cyclohexyl)benzamide (trans) (7)

To a solution of 4-(((R)-8-cyclopentyl-7-ethyl-5-methyl-6-oxo-5,6,7,8-tetrahydropteridin-2-yl)amino)-N-((1r,4R)-4-(4-(2,2-dimethoxyethyl)piperazin-1-yl)cyclohexyl)-3-methoxybenzamide (trans) (283 mg, 416.87 μmol) in H₂O (5 mL) was added HBr (1.49 g, 7.37 mmol, 1 mL, 40% purity) was added at 20° C. and the resulting mixture was stirred at 40° C. for 12 h. LCMS showed 42% of starting material remained and 28% peak with desired mass and the reaction mixture was stirred at 40° C. for 12 h. LCMS showed starting material was consumed completely and 91% peak with desired mass. The reaction mixture was concentrated in vacuum to afford 4-(((R)-8-cyclopentyl-7-ethyl-5-methyl-6-oxo-5,6,7,8-tetrahydropteridin-2-yl)amino)-3-methoxy-N-((1r,4R)-4-(4-(2-oxoethyl)piperazin-1-yl)cyclohexyl)benzamide (trans) (264 mg, crude) as a yellow oil. MS(M+H₂O+H)⁺=651.3

### Step 6. Synthesis of 4-(((R)-8-cyclopentyl-7-ethyl-5-methyl-6-oxo-5,6,7,8-tetrahydropteridin-2-yl)amino)-N-((1r,4R)-4-(4-(2-((2-(2,6-dioxopiperidin-3-yl)-1-oxoisoindolin-4-yl)amino)ethyl)piperazin-1-yl)cyclohexyl)-3-methoxybenzamide (trans) (Compound 5)

To a solution of 4-(((R)-8-cyclopentyl-7-ethyl-5-methyl-6-oxo-5,6,7,8-tetrahydropteridin-2-yl)amino)-3-methoxy-N-((1r,4R)-4-(4-(2-oxoethyl)piperazin-1-yl)cyclohexyl)benzamide (trans) (174 mg, 274.97 μmol) and 3-(4-amino-1-oxo-isoindolin-2-yl) piperidine-2, 6-dione (71.29 mg, 274.97 μmol) in MeOH (5 mL) was added TFA (31.35 mg, 274.97 μmol, 20.43 μL) at 20° C., then NaBH₃CN (51.84 mg, 824.91 μmol) was slowly added at 20° C. and the resulting mixture was stirred at 20° C. for 1 h. LCMS showed starting material was consumed completely and 55% peak with desired mass. The reaction mixture was concentrated in vacuum. The residue was diluted with H₂O (15 mL) and extracted with EtOAc (10 mL×3). The organic layer was washed with saturated NaHCO₃ (10 mL×3), dried over Na₂SO₄, filtered and concentrated. The residue was purified by prep-TLC (SiO₂, DCM:MeOH=10:1) to afford product with 85% purity by LCMS. The product was re-purified by prep-HPLC (column: Phenomenex luna C18 150*40 mm*15 um; mobile phase: [water (TFA)-ACN]; B %: 15%-45%, 10 min) and lyophilized to afford 4-(((R)-8-cyclopentyl-7-ethyl-5-methyl-6-oxo-5,6,7,8-tetrahydropteridin-2-yl)amino)-N-((1r,4R)-4-(4-(2-((2-(2,6-dioxopiperidin-3-yl)-1-oxoisoindolin-4-yl)amino)ethyl)piperazin-1-yl)cyclohexyl)-3-methoxybenzamide (trans) (23.1 mg, 18.58 μmol, 6.76% yield, 98% purity, 3TFA) as a white solid. MS(M+H)⁺=876.4

¹H NMR (400 MHz, DMSO-d₆) δ=11.02 (s, 1H), 9.04-8.69 (m, 1H), 8.27-8.20 (m, 1H), 8.05-7.97 (m, 1H), 7.78 (s, 1H), 7.55-7.48 (m, 2H), 7.33 (t, J=7.7 Hz, 1H), 7.00 (d, J=7.3 Hz, 1H), 6.85 (d, J=8.2 Hz, 1H), 5.13 (dd, J=5.1, 13.4 Hz, 1H), 4.43-4.38 (m, 1H), 4.27-4.10 (m, 3H), 3.91 (s, 3H), 3.81-3.76 (m, 1H), 3.44-3.42 (m, 14H), 3.23 (s, 3H), 2.96-2.92 (m, 1H), 2.66-2.58 (m, 2H), 2.46-2.38 (m, 1H), 2.31-2.23 (m, 1H), 2.13-2.02 (m, 3H), 2.02-1.90 (m, 4H), 1.88-1.78 (m, 3H), 1.65-1.55 (m, 3H), 1.54-1.36 (m, 5H), 0.75 (t, J=7.5 Hz, 3H)

### Example 6. Synthesis of 4-(((R)-8-cyclopentyl-7-ethyl-5-methyl-6-oxo-5,6,7,8-tetrahydropteridin-2-yl)amino)-N-((1r,4R)-4-(4-(((2-(2,6-dioxopiperidin-3-yl)-1-oxoisoindolin-4-yl)amino)meth yl)piperidin-1-yl)cyclohexyl)-3-methoxybenzamide (Compound 6)

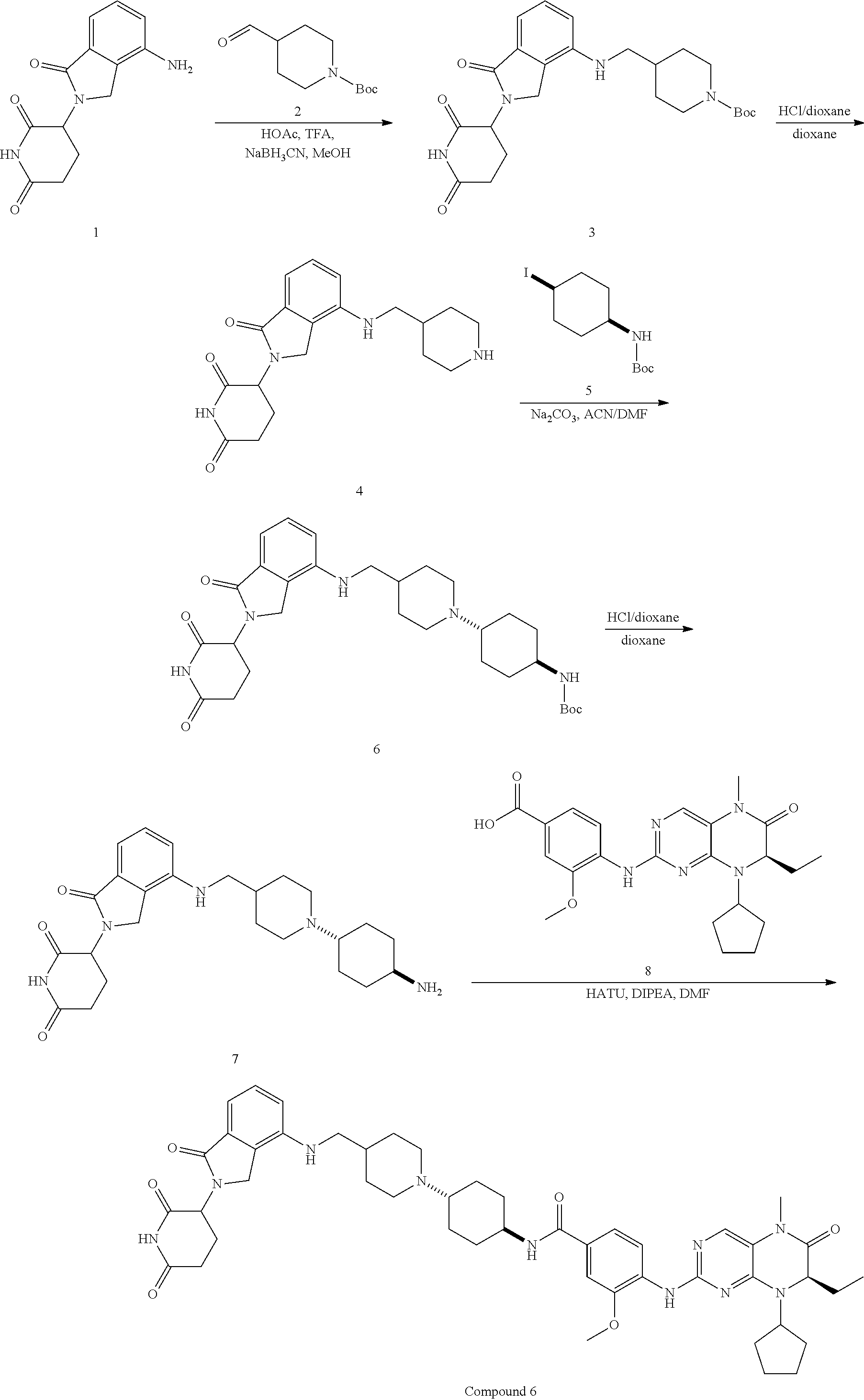

### Step 1. Synthesis of tert-butyl 4-(((2-(2,6-dioxopiperidin-3-yl)-1-oxoisoindolin-4-yl)amino)methyl)piperidine-1-carboxylate (3)

To a solution of 3-(4-amino-1-oxoisoindolin-2-yl)piperidine-2,6-dione (1 g, 3.86 mmol) and tert-butyl 4-formylpiperidine-1-carboxylate (904.88 mg, 4.24 mmol) in MeOH (20 mL) was added HOAc (115.81 mg, 1.93 mmol, 110.40 L), TFA (87.96 mg, 771.43 μmol, 57.30 L) and NaBH₃CN (727.17 mg, 11.57 mmol). The mixture was stirred at 25° C. for 16 hr. LCMS showed 45% of 3-(4-amino-1-oxoisoindolin-2-yl)piperidine-2,6-dione remained, several new peaks were shown on LCMS and a peak (44%) with desired mass. The reaction mixture was quenched with saturated aqueous solution of NaHCO₃ (30 mL) at 0° C., and then extracted with EtOAc (50 mL×2). The combined organic layers were washed with brine (30 mL), dried over Na₂SO₄, filtered and concentrated under reduced pressure to afford a residue which was triturated with EtOAc at 25° C. for 2 hours, and followed triturated with MeOH at 25° C. for 16 hours to afford tert-butyl 4-(((2-(2,6-dioxopiperidin-3-yl)-1-oxoisoindolin-4-yl)amino)methyl)piperidine-1-carboxylate (780 mg, 1.25 mmol, 32.34% yield, 73% purity) as a white solid. MS(M+H-100)⁺=357.3

### Step 2. Synthesis of 3-(1-oxo-4-((piperidin-4-ylmethyl)amino)isoindolin-2-yl)piperidine-2,6-dione (4)

To a solution of tert-butyl 4-(((2-(2,6-dioxopiperidin-3-yl)-1-oxoisoindolin-4-yl)amino)methyl)piperidine-1-carboxylate (780 mg, 1.25 mmol) in dioxane (2 mL) was added HCl/dioxane (4 M, 3.12 mL). The mixture was stirred at 20° C. for 2 hr. LCMS showed tert-butyl 4-(((2-(2,6-dioxopiperidin-3-yl)-1-oxoisoindolin-4-yl)amino)methyl)piperidine-1-carboxylate was consumed completely and one peak (65%) with desired mass. The reaction mixture was concentrated under reduced pressure to remove solvent. The residue was purified by prep-HPLC (column: YMC Triart C18 150*25 mm*5 um; mobile phase: [water (HCl)-ACN]; B %: 4%-34%, 10 min) followed by lyophilization to afford 3-(1-oxo-4-((piperidin-4-ylmethyl)amino)isoindolin-2-yl)piperidine-2,6-dione (360 mg, 916.31 μmol, 73.47% yield, HCl) as a white solid. MS(M+H)⁺=357.1

### Step 3. Synthesis of tert-butyl ((1r,4r)-4-(4-(((2-(2,6-dioxopiperidin-3-yl)-1-oxoisoindolin-4-yl)amino)methyl)piperidin-1-yl)cyclohexyl)carbamate (6)

To a solution of 3-(1-oxo-4-((piperidin-4-ylmethyl)amino)isoindolin-2-yl)piperidine-2,6-dione (340 mg, 865.41 μmol, HCl) and tert-butyl ((1s,4s)-4-iodocyclohexyl)carbamate (562.83 mg, 1.73 mmol) in ACN (3 mL) and DMF (3 mL) was added Na₂CO₃ (550.35 mg, 5.19 mmol). The mixture was stirred at 80° C. for 16 hours. LCMS showed 38% of 3-(1-oxo-4-((piperidin-4-ylmethyl)amino)isoindolin-2-yl)piperidine-2,6-dione remained, several new peaks were shown on LCMS and a peak (28%) with desired mass. Then tert-butyl ((1s,4s)-4-iodocyclohexyl)carbamate (281.42 mg, 865.41 μmol) was added. The mixture was stirred at 80° C. for another 32 hours. LCMS showed 16% of 3-(1-oxo-4-((piperidin-4-ylmethyl)amino)isoindolin-2-yl)piperidine-2,6-dione remained and a peak (30%) with desired mass. The reaction mixture was quenched with water (10 mL) at 0° C., and then extracted with EtOAc (50 mL×2). The combined organic layers were washed with brine (15 mL), dried over Na₂SO₄, filtered and concentrated under reduced pressure to afford a residue which was purified by prep-HPLC (column: Phenomenex luna C18 150*40 mm*15 um; mobile phase: [water(FA)-ACN]; gradient: 8%-38% B over 15 min) followed by lyophilization to afford tert-butyl ((1r,4r)-4-(4-(((2-(2,6-dioxopiperidin-3-yl)-1-oxoisoindolin-4-yl)amino)methyl)piperidin-1-yl)cyclohexyl)carbamate (60 mg, 105.11 μmol, 12.15% yield, 97% purity) as a white solid. MS(M+H)⁺=554.5

### Step 4. Synthesis of 3-(4-(((1-((1r,4r)-4-aminocyclohexyl)piperidin-4-yl)methyl)amino)-1-oxoisoindolin-2-yl)piperidine-2,6-dione (7)

To a solution of tert-butyl ((1r,4r)-4-(4-(((2-(2,6-dioxopiperidin-3-yl)-1-oxoisoindolin-4-yl)amino)methyl)piperidin-1-yl)cyclohexyl)carbamate (40 mg, 72.24 μmol) in dioxane (0.2 mL) was added HCl/dioxane (4 M, 180.61 L). The mixture was stirred at 20° C. for 0.5 hr. LCMS showed the starting material was consumed completely and one main peak with desired mass. The reaction mixture was concentrated under reduced pressure to remove solvent to afford 3-(4-(((1-((1r,4r)-4-aminocyclohexyl)piperidin-4-yl)methyl)amino)-1-oxoisoindolin-2-yl)piperidine-2,6-dione (35 mg, 71.42 μmol, 98.87% yield, HCl) as a white solid. MS(M+H)⁺=454.3

### Step 5. Synthesis of 4-(((R)-8-cyclopentyl-7-ethyl-5-methyl-6-oxo-5,6,7,8-tetrahydropteridin-2-yl)amino)-N-((1r,4R)-4-(4-(((2-(2,6-dioxopiperidin-3-yl)-1-oxoisoindolin-4-yl)amino)methyl)piperidin-1-yl)cyclohexyl)-3-methoxybenzamide (Compound 6)

To a solution of 3-(4-(((1-((1r,4r)-4-aminocyclohexyl)piperidin-4-yl)methyl)amino)-1-oxoisoindolin-2-yl)piperidine-2,6-dione (35 mg, 71.42 μmol, HCl) and (R)-4-((8-cyclopentyl-7-ethyl-5-methyl-6-oxo-5,6,7,8-tetrahydropteridin-2-yl)amino)-3-methoxybenzoic acid (27.35 mg, 64.28 μmol) in DMF (2 mL) were added HATU (40.74 mg, 107.13 μmol) and DIPEA (46.15 mg, 357.12 μmol, 62.20 L). The mixture was stirred at 20° C. for 16 hr. LCMS showed the starting material was consumed completely and one peak (60%) with desired mass. The reaction mixture was quenched with water (15 mL) at 0° C., and then extracted with EtOAc (20 mL×2). The combined organic layers were washed with brine (10 mL), dried over Na₂SO₄, filtered and concentrated under reduced pressure to afford a residue which was purified by prep-HPLC (column: Phenomenex luna C18 150*25 mm*10 um; mobile phase: [water (TFA)-ACN]; gradient: 30%-60% B over 10 min) followed by lyophilization to afford 4-(((R)-8-cyclopentyl-7-ethyl-5-methyl-6-oxo-5,6,7,8-tetrahydropteridin-2-yl)amino)-N-((1r,4R)-4-(4-(((2-(2,6-dioxopiperidin-3-yl)-1-oxoisoindolin-4-yl)amino)methyl)piperidin-1-yl)cyclohexyl)-3-methoxybenzamide (29.0 mg, 25.83 μmol, 36.16% yield, 97% purity, 2TFA) as a white solid. MS(M+H)⁺=861.5

¹H NMR (400 MHz, DMSO-d₆) δ=11.02 (s, 1H), 9.01-8.83 (m, 1H), 8.22 (d, J=8.1 Hz, 1H), 8.13-7.95 (m, 1H), 7.78 (s, 1H), 7.60-7.42 (m, 2H), 7.30 (t, J=7.8 Hz, 1H), 6.94 (d, J=7.5 Hz, 1H), 6.81 (d, J=8.1 Hz, 1H), 5.89-5.71 (m, 1H), 5.13 (dd, J=5.0, 13.2 Hz, 1H), 4.40-4.39 (m, 1H), 4.27-4.18 (m, 2H), 4.15-4.08 (m, 1H), 3.92 (s, 3H), 3.83-3.74 (m, 2H), 3.66-3.61 (m, 2H), 3.23 (s, 3H), 3.10-3.08 (m, 2H), 3.03-2.89 (m, 3H), 2.69-2.62 (m, 1H), 2.57-2.54 (m, 1H), 2.30-2.26 (m, 1H), 2.11-1.95 (m, 7H), 1.94-1.84 (m, 4H), 1.83-1.70 (m, 3H), 1.68-1.55 (m, 4H), 1.52-1.35 (m, 5H), 0.76 (t, J=7.3 Hz, 3H).

### Example 7. Synthesis of 4-(((R)-8-cyclopentyl-7-ethyl-5-methyl-6-oxo-5,6,7,8-tetrahydropteridin-2-yl)amino)-N-((1r,4R)-4-(4-(((4-(2,6-dioxopiperidin-3-yl)benzyl)amino)methyl)piperidin-1-yl)cyclohexyl)-3-methoxybenzamide (Compound 7)

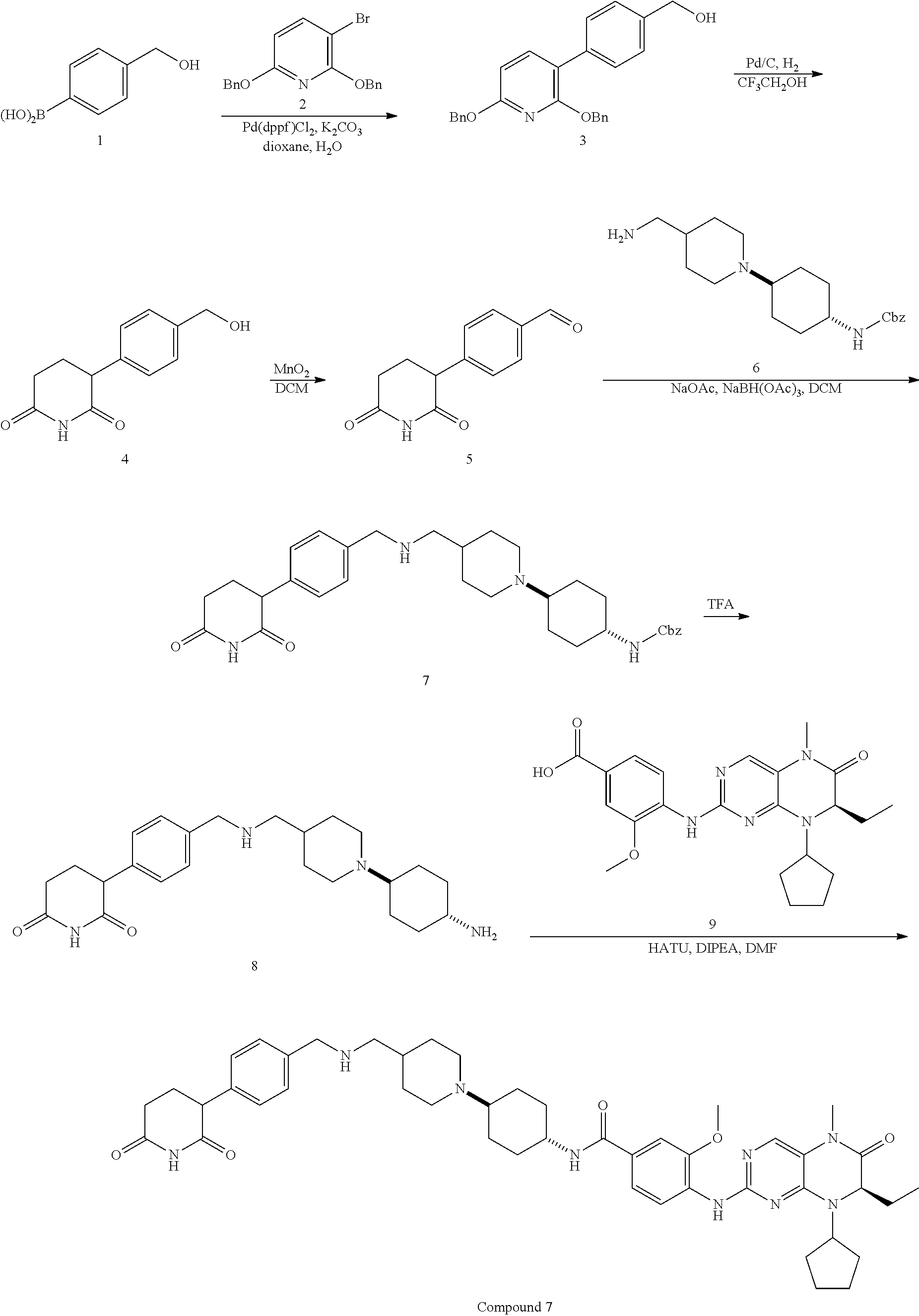

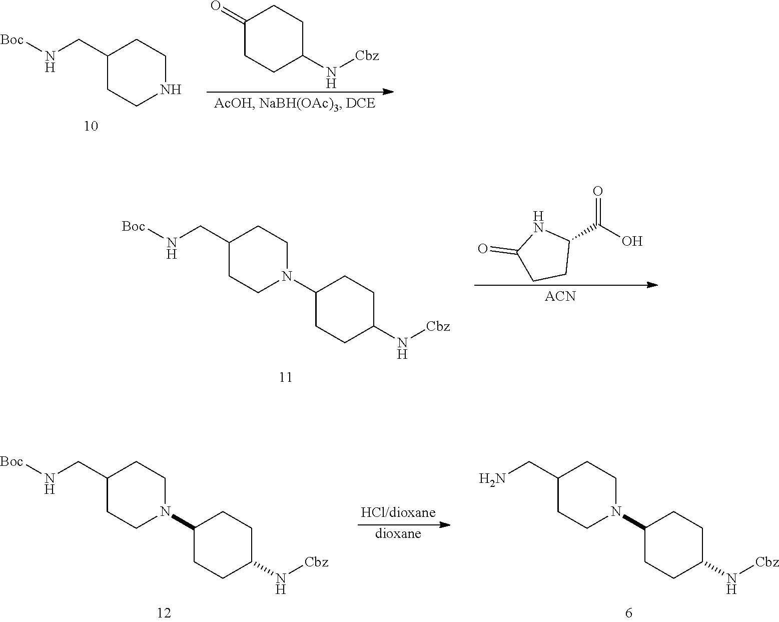

### Step 1. Synthesis of (4-(2,6-bis(benzyloxy)pyridin-3-yl)phenyl)methanol (3)

To a solution of 2,6-bis(benzyloxy)-3-bromopyridine (2 g, 5.40 mmol), (4-(hydroxymethyl)phenyl)boronic acid (902.93 mg, 5.94 mmol) in dioxane (20 mL), H₂O (2 mL) were added Pd(dppf)Cl₂ (395.26 mg, 540.19 μmol), K₂CO₃ (2.24 g, 16.21 mmol), the mixture was stirred at 100° C. for 16 h under N₂ atmosphere. LCMS showed a main peak with desired mass. The mixture was filtered and the filtrate was concentrated in vacuum. The residue was purified by flash silica gel chromatography (20 g SepaFlash® Silica Flash Column, Eluent of 0~50% EtOAc/Petroleum ether gradient @40 mL/min) to afford (4-(2,6-bis(benzyloxy)pyridin-3-yl)phenyl)methanol (1.8 g, 4.53 mmol, 83.84% yield) as a yellow oil. MS(M+H)⁺=398.2.

### Step 2. Synthesis of 3-(4-(hydroxymethyl)phenyl)piperidine-2,6-dione (4)

A mixture of (4-(2,6-bis(benzyloxy)pyridin-3-yl)phenyl)methanol (1 g, 2.52 mmol) and Pd/C (200 mg, 2.52 mmol, 10% purity) in CF₃CH₂OH (10 mL) was degassed and purged with H₂ for 3 times, then the mixture was stirred at 30° C. for 16 h under H₂ (50 Psi) atmosphere. TLC (Petroleum ether:EtOAc=1:2) indicated the starting material was consumed completely, and one major new spot with larger polarity was detected. The mixture was filtered and the filter cake was washed with CF₃CH₂OH (100 mL). The filtrate was concentrated in vacuum. The residue was purified by flash silica gel chromatography (Biotage; 10 g SepaFlash® Silica Flash Column, Eluent of 0~50% EtOAc/Petroleum ether gradient @80 mL/min) to afford 3-(4-(hydroxymethyl)phenyl)piperidine-2,6-dione (300 mg, 1.37 mmol, 54.63% yield) as a white solid. MS(M+H)⁺=219.3.

### Step 3. Synthesis of 4-(2,6-dioxopiperidin-3-yl)benzaldehyde (5)

To a solution of 3-(4-(hydroxymethyl)phenyl)piperidine-2,6-dione (100 mg, 458.19 mol) in DCM (2 mL) was added MnO₂ (199.17 mg, 2.29 mmol), the mixture was stirred at 20° C. for 16 h. TLC (Petroleum ether:EtOAc=1:2) indicated the starting material was consumed completely, and one major new spot was detected. The mixture was filtered and the filter cake was washed with DCM (20 mL). The filtrate was concentrated in vacuum to afford 4-(2,6-dioxopiperidin-3-yl)benzaldehyde (50 mg, 230.18 mol, 50.24% yield) as a yellow solid. MS(M+H)⁺=218.3.

### Step 4. Synthesis of benzyl (4-(4-(((tert-butoxycarbonyl)amino)methyl)piperidin-1-yl)cyclohexyl)carbamate (11)

To a solution of tert-butyl (piperidin-4-ylmethyl)carbamate (3 g, 14.00 mmol) in DCE (80 mL) was added benzyl (4-oxocyclohexyl)carbamate (3.5 g, 14.15 mmol) and HOAc (840.66 mg, 14.00 mmol, 801.39 μL) at 20° C. After stirring at 25° C. for 2 h, then NaBH(OAc)₃ (11.87 g, 56.00 mmol) was added and the resulting mixture was stirred at 25° C. for 14 h. LCMS showed a main peak with desired mass. The reaction mixture was quenched by addition of NaHCO₃ (200 mL) and extracted with EtOAc (200 mL×3). The combined organic layers were washed with brine (500 mL×2), dried over Na₂SO₄, filtered and concentrated in vacuum. The residue was purified by reversed-phase HPLC (Column₃₃₀ g Flash Column Welch Ultimate XB_C₁₈ 20-40 μm; 120 A; Solvent for sample dissolution about 15.00 grams of sample dissolved in 20 mL of DMF; Flow rate 100 mL/min; Mobile phase MeCN/H₂O; Gradient B % 10-100% 50 min; % min; Instrument TELEDYNE ISCO CombiFlashRf₁₅₀), the eluent was lyophilized to afford benzyl (4-(4-(((tert-butoxycarbonyl)amino)methyl)piperidin-1-yl)cyclohexyl)carbamate (2 g, 4.49 mmol, 32.06% yield) as a yellow solid. MS(M+H)⁺=446.3.

### Step 5. Synthesis of benzyl ((1r,4r)-4-(4-(((tert-butoxycarbonyl)amino)methyl)piperidin-1-yl)cyclohexyl)carbamate(trans) (12)

To a solution of benzyl (4-(4-(((tert-butoxycarbonyl)amino)methyl)piperidin-1-yl)cyclohexyl)carbamate (0.5 g, 1.12 mmol) in CH₃CN (15 mL) was added (S)-5-oxopyrrolidine-2-carboxylic acid (75.00 mg, 580.88 μmol) at 25° C. The mixture was stirred at 70° C. for 2 h, then the mixture was stirred at 25° C. for 16 h. LCMS showed a main peak with desired mass. The reaction mixture was filtered. The filter cake was diluted with EtOAc (30 mL) and H₂O (15 mL), the mixture was adjust to pH=8 with NaOH (1M, 0.5 mL) and extracted with EtOAc (40 mL×2). The combined organic layers were washed with brine (30 mL×2), dried over anhydrous Na₂SO₄, filtered and concentrated in vacuum to afford benzyl ((1r,4r)-4-(4-(((tert-butoxycarbonyl)amino)methyl)piperidin-1-yl)cyclohexyl)carbamate(trans) (180 mg, 403.96 μmol, 36.00% yield) as a white solid. MS(M+H)⁺=446.3.

### Step 6. Synthesis of benzyl ((1r,4r)-4-(4-(aminomethyl)piperidin-1-yl)cyclohexyl)carbamate(trans) (6)

To a solution of benzyl ((1r,4r)-4-(4-(((tert-butoxycarbonyl)amino)methyl)piperidin-1-yl)cyclohexyl)carbamate (trans) (180 mg, 403.96 μmol) in dioxane (1 mL) was added HCl/dioxane (4 M, 1 mL), the mixture was stirred at 20° C. for 1 h. LCMS showed a main peak with desired mass. The mixture was concentrated in vacuum to afford benzyl ((1r,4r)-4-(4-(aminomethyl)piperidin-1-yl)cyclohexyl)carbamate (trans) (150 mg, crude, HCl salt) as a white solid. MS(M+H)⁺=346.2.

### Step 7. Synthesis of benzyl ((1r,4r)-4-(4-(((4-(2,6-dioxopiperidin-3-yl)benzyl)amino)methyl)piperidin-1-yl)cyclohexyl)carbamate (trans) (7)

To a solution of 4-(2,6-dioxopiperidin-3-yl)benzaldehyde (50 mg, 230.18 μmol), benzyl ((1r,4r)-4-(4-(aminomethyl)piperidin-1-yl)cyclohexyl)carbamate (trans) (87.92 mg, 230.18 μmol, HCl salt) in DCM (2 mL) was added NaOAc (18.88 mg, 230.18 mol) at 20° C. After stirring for 0.5 h, then NaBH(OAc)₃ (146.35 mg, 690.54 μmol) was added, the mixture was stirred at 20° C. for 16 h. LCMS showed a main peak with desired mass. The mixture was diluted with water (10 mL) and extracted with EtOAc (10 mL×3). The combined organic layers were washed with saturated NaHCO₃ (10 mL×2), dried over Na₂SO₄, filtered and concentrated in vacuum. The residue was purified by reversed-phase HPLC (column: YMC Triart C18 150*25 mm*5 um; mobile phase: [water (HCl)-ACN]; gradient: 2%-32% B over 8 min). The eluent was lyophilized to afford benzyl ((1r,4r)-4-(4-(((4-(2,6-dioxopiperidin-3-yl)benzyl)amino)methyl)piperidin-1-yl)cyclohexyl)carbamate (trans) (60 mg, 109.75 μmol, 47.68% yield) as a white solid. MS(M+H)⁺=547.4.

### Step 8. Synthesis of 3-(4-((((1-((1r,4r)-4-aminocyclohexyl)piperidin-4-yl)methyl)amino)methyl)phenyl)piperidine-2,6-dione (trans) (8)

A mixture of benzyl ((1r,4r)-4-(4-(((4-(2,6-dioxopiperidin-3-yl)benzyl)amino)methyl)piperidin-1-yl)cyclohexyl)carbamate (trans) (60 mg, 109.75 μmol) in TFA (1 mL) was stirred at 60° C. for 4 h. LCMS showed a main peak with desired mass. The mixture was concentrated in vacuum to afford 3-(4-((((1-((1r,4r)-4-aminocyclohexyl)piperidin-4-yl)methyl)amino)methyl)phenyl)piperidine-2,6-dione (trans) (57 mg, 108.24 μmol, 98.63% yield, TFA salt) as a brown oil. MS(M+H)⁺=413.3.

### Step 9. Synthesis of 4-(((R)-8-cyclopentyl-7-ethyl-5-methyl-6-oxo-5,6,7,8-tetrahydropteridin-2-yl)amino)-N-((1r,4R)-4-(4-(((4-(2,6-dioxopiperidin-3-yl)benzyl)amino)methyl)piperidin-1-yl)cyclohexyl)-3-methoxybenzamide(trans) (Compound 7)

To a solution of (R)-4-((8-cyclopentyl-7-ethyl-5-methyl-6-oxo-5,6,7,8-tetrahydropteridin-2-yl)amino)-3-methoxybenzoic acid (40.40 mg, 94.95 μmol) in DMF (1 mL) were added HATU (54.15 mg, 142.43 μmol), DIPEA (61.36 mg, 474.75 μmol, 82.69 μL) at 20° C. After stirring for 0.5 h, then 3-(4-((((1-((1r,4r)-4-aminocyclohexyl)piperidin-4-yl)methyl)amino)methyl)phenyl)piperidine-2,6-dione (trans) (50 mg, 94.95 μmol, TFA salt) was added and the resulting mixture was stirred at 20° C. for 16 h. LCMS showed a main peak with desired mass. The mixture was diluted with water (10 mL) and extracted with EtOAc (10 mL×3). The combined organic layers were washed with brine (20 mL×2), dried over Na₂SO₄, filtered and concentrated in vacuum. The residue was purified by reversed-phase HPLC (column: Phenomenex luna C18 150*25 mm*10 um; mobile phase: [water (TFA)-ACN]; gradient: 22%-52% B over 10 min). The eluent was lyophilized to afford 4-(((R)-8-cyclopentyl-7-ethyl-5-methyl-6-oxo-5,6,7,8-tetrahydropteridin-2-yl)amino)-N-((1r,4R)-4-(4-(((4-(2,6-dioxopiperidin-3-yl)benzyl)amino)methyl)piperidin-1-yl)cyclohexyl)-3-methoxybenzamide (trans) (42.4 mg, 37.62 μmol, 39.62% yield, 93% purity, 2 TFA salt) as a white solid. MS(M+H)⁺=820.5.

¹H NMR (400 MHz, DMSO-d₆) δ=10.87 (s, 1H), 9.48-9.22 (m, 1H), 9.00-8.78 (m, 2H), 8.31-8.18 (m, 1H), 8.04-7.93 (m, 1H), 7.79 (s, 1H), 7.56-7.39 (m, 4H), 7.36-7.25 (m, 2H), 4.43-4.37 (m, 1H), 4.23-4.14 (m, 3H), 3.93-3.88 (m, 4H), 3.83-3.77 (m, 1H), 3.56-3.47 (m, 2H), 3.32-3.10 (m, 5H), 3.07-2.82 (m, 4H), 2.75-2.64 (m, 1H), 2.27-2.17 (m, 1H), 2.11-1.74 (m, 14H), 1.69-1.54 (m, 4H), 1.52-1.36 (m, 6H), 0.76 (t, J=7.4 Hz, 3H).

### Example 8. Synthesis of 4-(((R)-8-cyclopentyl-7-ethyl-5-methyl-6-oxo-5,6,7,8-tetrahydropteridin-2-yl)amino)-N-((1r,4R)-4-(4-((4-(2,6-dioxopiperidin-3-yl)benzyl)amino)piperidin-1-yl)cyclohexyl)-3-methoxybenzamide (Compound 8)

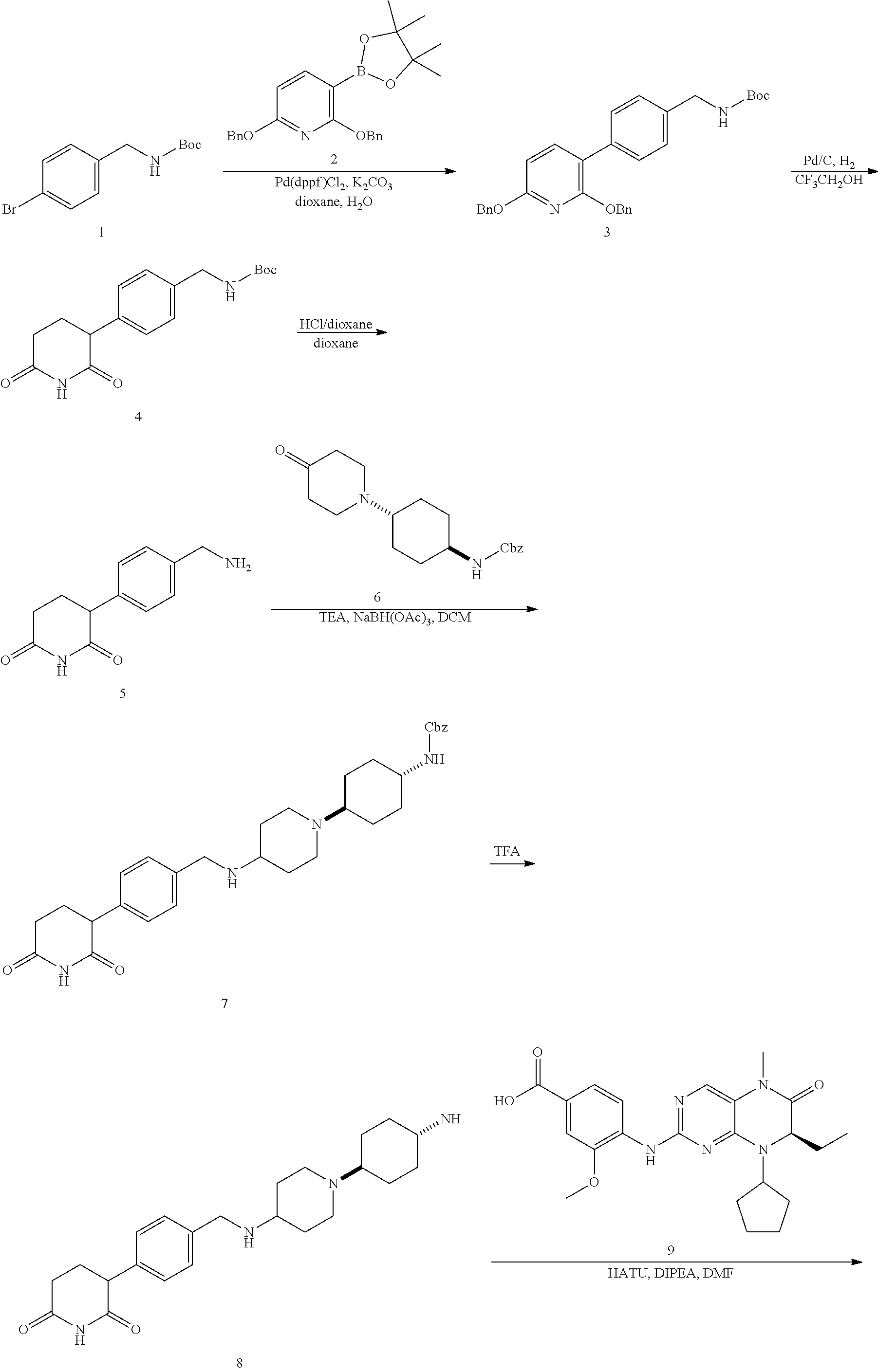

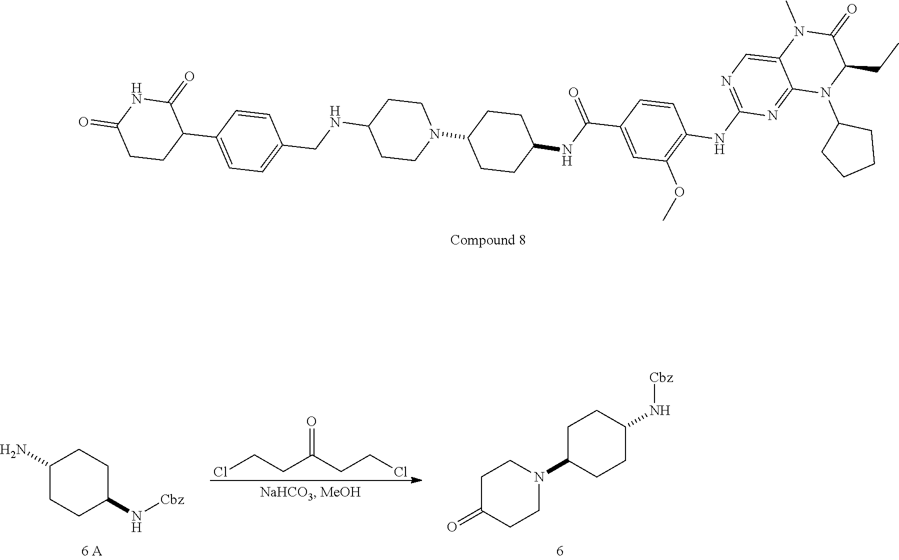

### Step 1. Synthesis of tert-butyl (4-(2,6-bis(benzyloxypyridin-3-yl)benzyl)carbamate

To a solution of tert-butyl (4-bromobenzyl)carbamate (450 mg, 1.57 mmol), 2,6-bis(benzyloxy)-3-(4,4,5,5-tetramethyl-1,3,2-dioxaborolan-2-yl)pyridine (656.22 mg, 1.57 mmol) in dioxane (5 mL) and H₂O (0.5 mL) were added Pd(dppf)Cl₂ (230.12 mg, 314.50 μmol), K₂CO₃ (651.99 mg, 4.72 mmol). The mixture was stirred at 100° C. for 16 h under N₂ atmosphere. LCMS showed a peak (53%) with desired mass. The mixture was filtered through a pad of celite. The filtrate was concentrated in vacuum to give a residue. The residue was purified by flash silica gel chromatography (Biotage; 10 g SepaFlash® Silica Flash Column, Eluent of 0~20% EtOAc/Petroleum ether gradient @80 mL/min) to afford tert-butyl (4-(2,6-bis(benzyloxy)pyridin-3-yl)benzyl)carbamate (600 mg, 1.12 mmol, 71.46% yield, 93% purity) as a yellow solid. MS(M+H)⁺=497.2.

### Step 2. Synthesis of tert-butyl (4-(2,6-dioxopiperidin-3-yl)benzyl)carbamate (4)

A mixture of tert-butyl (4-(2,6-bis(benzyloxy)pyridin-3-yl)benzyl)carbamate (600 mg, 1.21 mmol) and Pd/C (100 mg, 10% purity) in CF₃CH₂OH (10 mL) was degassed and purged with H₂ for 3 times, the mixture was stirred at 20° C. for 16 h under H₂ (50 Psi) atmosphere. TLC (Petroleum ether:EtOAc=3:1) indicated the starting material was consumed completely and one major new spot with lower polarity was detected. The mixture was diluted with CH₃CH₂OH (30 mL), filtered through a pad of celite, washed with CH₃CH₂OH (30 mL). The filtrate was concentrated in vacuum to afford tert-butyl (4-(2,6-dioxopiperidin-3-yl)benzyl)carbamate (380 mg, 1.19 mmol, 98.79% yield) was a yellow oil. MS(M+H)⁺=319.4.

### Step 3. Synthesis of 3-(4-(aminomethyl)phenyl)piperidine-2,6-dione (5)

To a solution of tert-butyl (4-(2,6-dioxopiperidin-3-yl)benzyl)carbamate (280 mg, 879.49 μmol) in dioxane (3 mL) was added HC/dioxane (4 M, 2.80 mL), the mixture was stirred at 20° C. for 1 h. TLC (Petroleum ether:EtOAc=3:1) indicated the starting material was consumed completely and one major new spot with lower polarity was detected. The mixture was concentrated in vacuum to afford 3-(4-(aminomethyl)phenyl)piperidine-2,6-dione (220 mg, crude, HCl) as a white solid. MS(M+H)⁺=219.3.

### Step 4. Synthesis of benzyl ((1r,4r)-4-(4-oxopiperidin-1-yl)cyclohexyl)carbamate (6)

To a solution of benzyl ((1r,4r)-4-aminocyclohexyl)carbamate (500 mg, 1.76 mmol, HCl) in MeOH (5 mL) were added 1,5-dichloropentan-3-one (272.18 mg, 1.76 mmol), NaHCO₃ (737.47 mg, 8.78 mmol, 341.58 μL), the mixture was stirred at 75° C. for 16 h. LCMS showed multiple peaks and desired mass. TLC (DCM:MeOH=10:1) indicated the starting material was consumed completely and a new spot was detected. The reaction mixture was diluted with H₂O (10 mL), extracted with EtOAc (10 mL×3). The combined organic layers were washed with brine (20 mL×2), dried over Na₂SO₄, filtered. The filtrate was concentrated in vacuum to afford a residue. The residue was purified by flash silica gel chromatography (Biotage; 10 g SepaFlash® Silica Flash Column, Eluent of 50~100% EtOAc/Petroleum ether to 10% MeOH/EtOAc gradient @80 mL/min) to afford benzyl ((1r,4r)-4-(4-oxopiperidin-1-yl)cyclohexyl)carbamate (400 mg, 1.21 mmol, 68.95% yield) as a yellow oil. MS(M+H)⁺=331.1.

### Step 5. Synthesis of benzyl ((1r,4r)-4-(4-((4-(2,6-dioxopiperidin-3-yl)benzyl)amino)piperidin-1-yl)cyclohexyl)carb amate (7)

To a solution of 3-(4-(aminomethyl)phenyl)piperidine-2,6-dione (80 mg, crude, HCl), benzyl ((1r,4r)-4-(4-oxopiperidin-1-yl)cyclohexyl)carbamate (103.78 mg, 314.08 mol) in DCM (1 mL) was added TEA (158.91 mg, 1.57 mmol, 218.58 μL), the mixture was stirred at 20° C. for 0.5 h, NaBH(OAc)₃ (199.70 mg, 942.24 μmol) was added, the mixture was stirred at 20° C. for 16 h. LCMS showed a main peak with desired mass. The mixture was filtered and the filter cake was wash twice with EtOAc (10 mL) and water (10 mL), dry in vacuum to afford benzyl ((1r,4r)-4-(4-((4-(2,6-dioxopiperidin-3-yl)benzyl)amino)piperidin-1-yl)cyclohexyl)carb amate (150 mg, crude) as a white solid. MS(M+H)⁺=533.4.

### Step 6. Synthesis of 3-(4-(((1-((1r,4r)-4-aminocyclohexyl)piperidin-4-yl)amino)methyl)phenyl)piperidine-2,6-dione (8)

A mixture of benzyl ((1r,4r)-4-(4-((4-(2,6-dioxopiperidin-3-yl)benzyl)amino)piperidin-1-yl)cyclohexyl)carb amate (100 mg, crude) in TFA (0.5 mL) was stirred at 60° C. for 4 h. LCMS showed a main peak with desired mass. The mixture was concentrated in vacuum to afford 3-(4-(((1-((1r,4r)-4-aminocyclohexyl)piperidin-4-yl)amino)methyl)phenyl)piperidine-2,6-dione (90 mg, crude, TFA) as a brown oil. MS(M+H)⁺=399.3.

### Step 7. Synthesis of 4-(((R)-8-cyclopentyl-7-ethyl-5-methyl-6-oxo-5,6,7,8-tetrahydropteridin-2-yl)amino)-N-((1r,4R)-4-(4-((4-(2,6-dioxopiperidin-3-yl)benzyl)amino)piperidin-1-yl)cyclohexyl)-3-methoxybenzamide (Compound 8)

To a solution of (R)-4-((8-cyclopentyl-7-ethyl-5-methyl-6-oxo-5,6,7,8-tetrahydropteridin-2-yl)amino)-3-methoxybenzoic acid (74.71 mg, 175.59 μmol) in DMF (2 mL) were added HATU (80.12 mg, 210.71 μmol), DIPEA (68.08 mg, 526.76 μmol, 91.75 μL), the mixture was stirred at 20° C. for 0.5 h, 3-(4-(((1-((1r,4r)-4-aminocyclohexyl)piperidin-4-yl)amino)methyl)phenyl)piperidine-2,6-dione (90 mg, crude, TFA) was added, the mixture was stirred at 20° C. for 16 h. LCMS showed a peak (47%) with desired mass. The mixture was diluted with water (10 mL) extracted with EtOAc (10 mL×3). The combined organic layers were washed with brine (20 mL×2), dried over Na₂SO₄, filtered. The filtrate was concentrated in vacuum to give a residue. The residue was purified by prep-HPLC (column: Phenomenex luna C18 150*25 mm*10 um; mobile phase: [water (TFA)-ACN]; gradient: 23%-53% B over 10 min) and re-purified by prep-HPLC (column: Phenomenex Luna C18 150*25 mm*10 um; mobile phase: [water (TFA)-ACN]; gradient: 22%-52% B over 10 min) The eluent was lyophilized to afford 4-(((R)-8-cyclopentyl-7-ethyl-5-methyl-6-oxo-5,6,7,8-tetrahydropteridin-2-yl)amino)-N-((1r,4R)-4-(4-((4-(2,6-dioxopiperidin-3-yl)benzyl)amino)piperidin-1-yl)cyclohexyl)-3-methoxybenzamide (41.5 mg, 40.13 μmol, 22.86% yield, 98% purity, 2TFA) as a white solid. MS(M+H)⁺=806.5.

¹H NMR (400 MHz, DMSO-d₆) δ=10.87 (s, 1H), 9.86-9.70 (m, 1H), 9.31-9.13 (m, 2H), 9.07-8.90 (m, 1H), 8.26 (d, J=7.6 Hz, 1H), 7.98 (d, J=7.5 Hz, 1H), 7.79 (s, 1H), 7.56-7.46 (m, 4H), 7.33 (d, J=8.1 Hz, 2H), 4.45-4.37 (m, 1H), 4.26-4.17 (m, 3H), 3.94-3.88 (m, 5H), 3.62-3.57 (m, 3H), 3.43-3.34 (m, 2H), 3.25-3.20 (m, 4H), 3.16-3.05 (m, 2H), 2.76-2.64 (m, 1H), 2.56-2.51 (m, 2H), 2.41-2.31 (m, 2H), 2.28-2.18 (m, 1H), 2.13-1.96 (m, 5H), 1.94-1.74 (m, 7H), 1.64-1.53 (m, 3H), 1.50-1.42 (m, 3H), 0.75 (t, J=7.5 Hz, 3H).

### Example 9. Synthesis of N-(1-(2-amino-3-(4-(2-(2,6-dioxopiperidin-3-yl)-1,3-dioxoisoindolin-4-yl)piperazin-1-yl)propanoyl)piperidin-4-yl)-4-(((R)-8-cyclopentyl-7-ethyl-5-methyl-6-oxo-5,6,7,8-tetrahydropteridin-2-yl)amino)-3-methoxybenzamide (Compound 9)

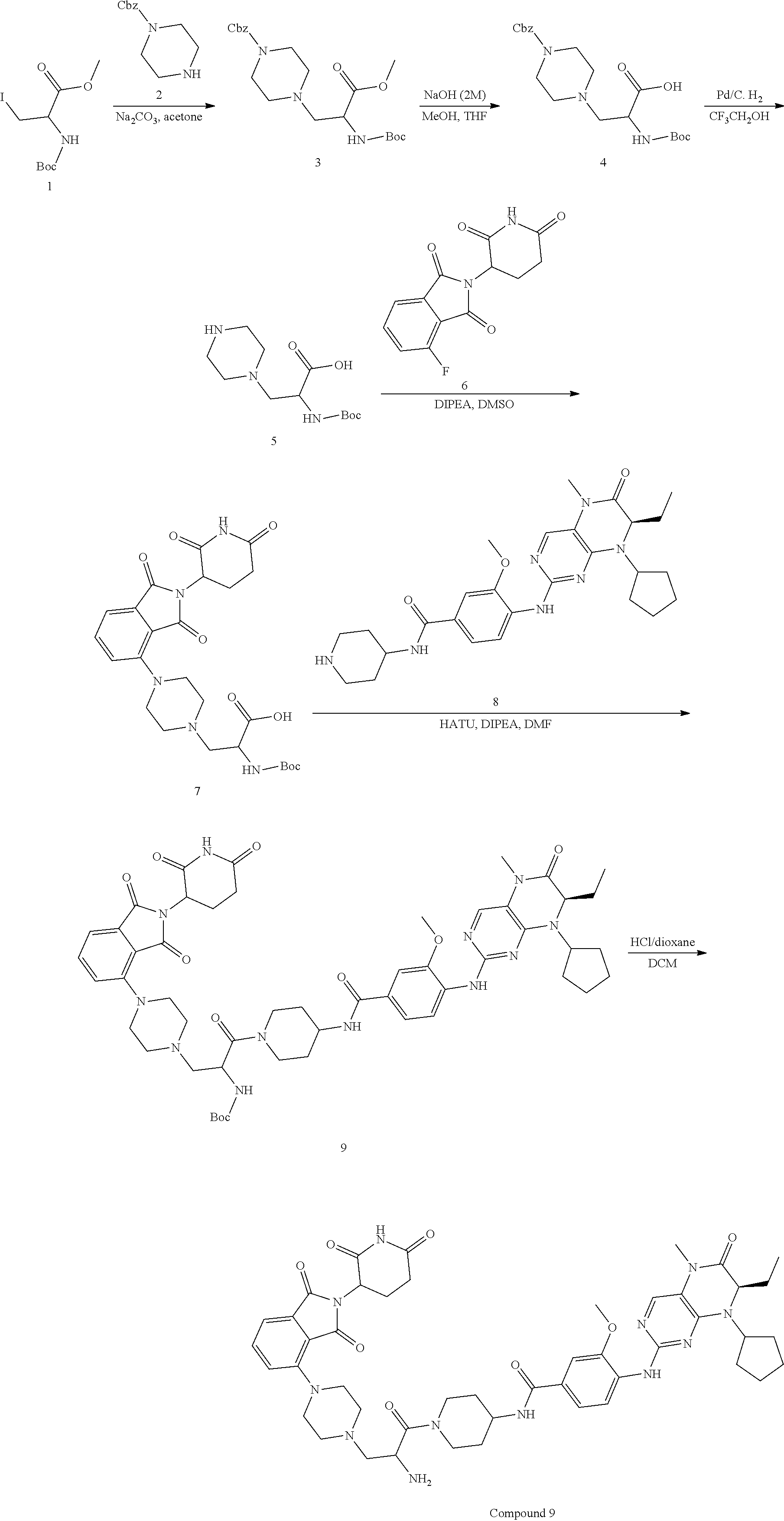

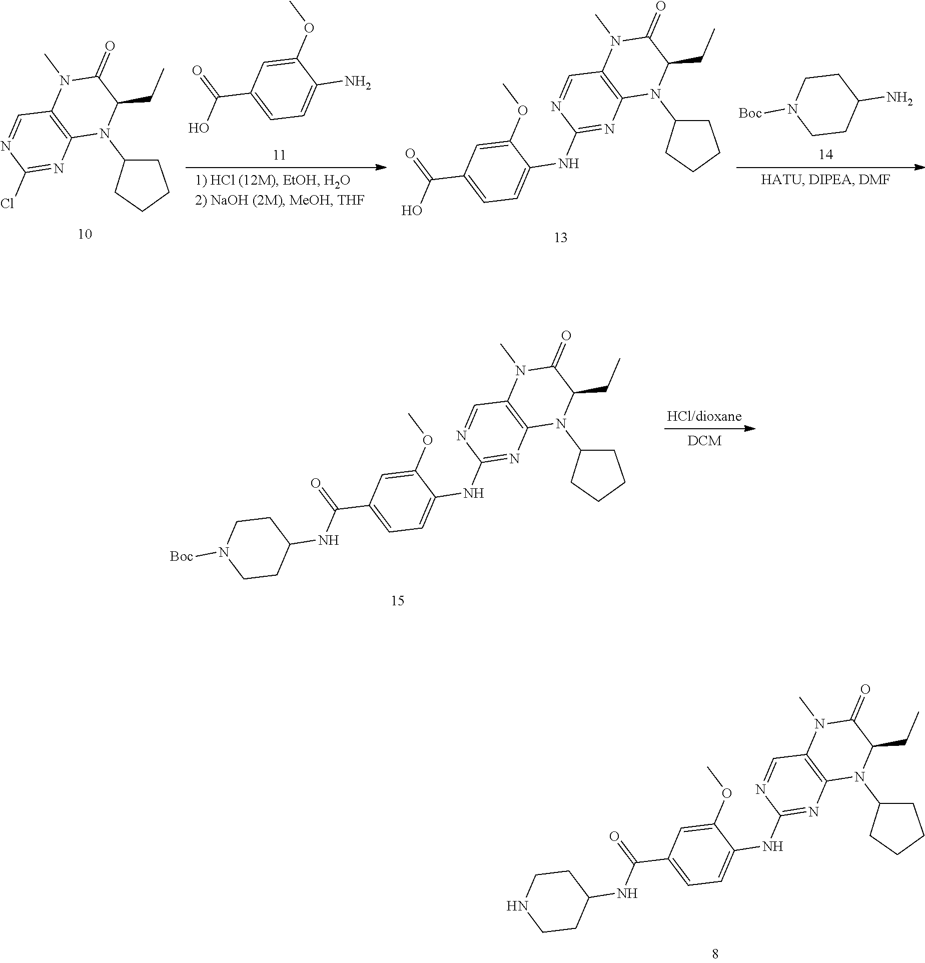

### Step 1. Synthesis of (R)-4-((8-cyclopentyl-7-ethyl-5-methyl-6-oxo-5,6,7,8-tetrahydropteridin-2-yl)amino)-3-methoxybenzoic acid (13)

3 batches: A mixture of (R)-2-chloro-8-cyclopentyl-7-ethyl-5-methyl-7,8-dihydropteridin-6(5H)-one (550 mg, 1.87 mmol), 4-amino-3-methoxybenzoic acid (374.27 mg, 2.24 mmol) and HCl (12 M, 550.00 μL) in EtOH (2 mL) and H₂O (8 mL) was stirred at 105° C. for 12 hours. LCMS showed (R)-2-chloro-8-cyclopentyl-7-ethyl-5-methyl-7,8-dihydropteridin-6(5H)-one was consumed completely. Three batches of the mixture were concentrated in vacuum to afford a mixture (R)-ethyl 4-((8-cyclopentyl-7-ethyl-5-methyl-6-oxo-5,6,7,8-tetrahydropteridin-2-yl)amino)-3-methoxybenzoate and (R)-4-((8-cyclopentyl-7-ethyl-5-methyl-6-oxo-5,6,7,8-tetrahydropteridin-2-yl)amino)-3-methoxybenzoic acid (2.5 g) as a brown gum. To a solution of the brown gum (2.5 g) in MeOH (20 mL) and THF (20 mL) was added NaOH (2 M, 20 mL), the mixture was stirred at 25° C. for 16 hours. LCMS showed the starting material was consumed completely and 65% of desired mass. The mixture was concentrated in vacuum to remove most of the solvent. The residue was triturated with H₂O (30 mL) and EtOAc (30 mL) for 10 minutes, the suspension was filtered and the filter cake was washed with H₂O (30 mL) and EtOAc (30 mL), the filter cake was diluted with EtOH (30 mL) and HCl solution (20 mL), the mixture was concentrated in vacuum at 70° C., the residue was triturated with a mixture (EtOAc:EtOH:DMF=4:2:1, 7 mL) for 30 minutes, the suspension was filtered and the filter cake was washed with EtOAc (20 mL). And it was triturated again with a mixture (EtOAc:EtOH:DMF=4:2:1, 10 mL) for 30 minutes, the suspension was filtered and the filter cake was washed with a mixture (EtOAc:EtOH:DMF=4:2:1, 3 mL), the filter cake was collected and dried to afford (R)-4-((8-cyclopentyl-7-ethyl-5-methyl-6-oxo-5,6,7,8-tetrahydropteridin-2-yl)amino)-3-methoxybenzoic acid (950 mg, 2.23 mmol, 78.51% yield) as a gray solid. MS(M+H)⁺=426.2.

¹H NMR (400 MHz, DMSO-d₆) δ=13.31-12.55 (m, 1H), 9.94-9.42 (m, 1H), 7.98 (d, J=8.1 Hz, 1H), 7.91 (s, 1H), 7.72-7.47 (m, 2H), 4.47 (dd, J=3.2, 6.4 Hz, 1H), 4.26-4.15 (m, 1H), 3.90 (s, 3H), 3.30-3.17 (m, 3H), 2.02-1.86 (m, 3H), 1.85-1.70 (m, 3H), 1.61-1.38 (m, 4H), 0.75 (t, J=7.4 Hz, 3H).

### Step 2. Synthesis of tert-butyl (R)-4-(4-((8-cyclopentyl-7-ethyl-5-methyl-6-oxo-5,6,7,8-tetrahydropteridin-2-yl)amino)-3-methoxybenzamido)piperidine-1-carboxylate (15)

To a solution of (R)-4-((8-cyclopentyl-7-ethyl-5-methyl-6-oxo-5,6,7,8-tetrahydropteridin-2-yl)amino)-3-methoxybenzoic acid (400 mg, 940.12 μmol) in DMF (5 mL) were added HATU (428.95 mg, 1.13 mmol) and DIPEA (364.51 mg, 2.82 mmol), the mixture was stirred at 25° C. for 15 minutes, then tert-butyl 4-aminopiperidine-1-carboxylate (188.28 mg, 940.12 μmol) was added, the resulting mixture was stirred at 25° C. for 1 hour. LCMS showed (R)-4-((8-cyclopentyl-7-ethyl-5-methyl-6-oxo-5,6,7,8-tetrahydropteridin-2-yl)amino)-3-methoxybenzoic acid was consumed completely and 75% of desired mass. The mixture was treated with AcOH to adjust pH=6-7, the resulting mixture was diluted with H₂O (50 mL) and extracted with EtOAc (50 mL×3). The combined organic layers were washed with brine (100 mL×3), dried over Na₂SO₄, filtered and concentrated in vacuum. The residue was purified by prep-TLC (SiO₂, Petroleum ether:EtOAc=1:2) to afford tert-butyl (R)-4-(4-((8-cyclopentyl-7-ethyl-5-methyl-6-oxo-5,6,7,8-tetrahydropteridin-2-yl)amino)-3-methoxybenzamido)piperidine-1-carboxylate (375 mg, 617.04 μmol, 65.63% yield) as an off-white solid. MS(M+H)⁺=608.3.

### Step 3. Synthesis of (R)-4-((8-cyclopentyl-7-ethyl-5-methyl-6-oxo-5,6,7,8-tetrahydropteridin-2-yl)amino)-3-methoxy-N-(piperidin-4-yl)benzamide (8)

To a solution of tert-butyl (R)-4-(4-((8-cyclopentyl-7-ethyl-5-methyl-6-oxo-5,6,7,8-tetrahydropteridin-2-yl)amino)-3-methoxybenzamido)piperidine-1-carboxylate (400 mg, 658.17 μmol) in DCM (10 mL) was added HCl/dioxane (4 M, 10 mL), the mixture was stirred at 25° C. for 1 hour. LCMS showed the starting material was consumed completely and 89% of desired mass. The reaction mixture was concentrated in vacuum to afford (R)-4-((8-cyclopentyl-7-ethyl-5-methyl-6-oxo-5,6,7,8-tetrahydropteridin-2-yl)amino)-3-methoxy-N-(piperidin-4-yl)benzamide (395 mg, HCl salt) as an off-white solid. MS(M+H)⁺=508.2.

### Step 4. Synthesis of benzyl 4-(2-((tert-butoxycarbonyl)amino)-3-methoxy-3-oxopropyl)piperazine-1-carboxylate (3)

A mixture of methyl 2-((tert-butoxycarbonyl)amino)-3-iodopropanoate (2.5 g, 7.60 mmol), benzyl piperazine-1-carboxylate (1.71 g, 7.75 mmol, 1.50 mL) and Na₂CO₃ (1.61 g, 15.19 mmol) in acetone (50 mL) was stirred at 25° C. for 12 hours. LCMS showed methyl 2-((tert-butoxycarbonyl)amino)-3-iodopropanoate was consumed completely and 33% of desired mass. The suspension was filtered and the filter cake was washed with EtOAc (50 mL), the filtrate was concentrated in vacuum. The residue was purified by flash silica gel chromatography (20 g SepaFlash® Silica Flash Column, Eluent of 14~26% EtOAc/Petroleum ether gradient @100 mL/min) to afford benzyl 4-(2-((tert-butoxycarbonyl)amino)-3-methoxy-3-oxopropyl)piperazine-1-carboxylate (1.07 g, 2.54 mmol, 33.42% yield) as a yellow oil. MS(M+H)⁺=422.2.

### Step 5. Synthesis of 3-(4-((benzyloxy)carbonyl)piperazin-1-yl)-2-((tert-butoxycarbonyl)amino)propanoic acid (4)

To a solution of benzyl 4-(2-((tert-butoxycarbonyl)amino)-3-methoxy-3-oxopropyl)piperazine-1-carboxylate (1.07 g, 2.54 mmol) in MeOH (5 mL) and THF (5 mL) was added NaOH (2 M, 5 mL), the mixture was stirred at 25° C. for 12 hours. LCMS showed the starting material was consumed completely and 53% of desired mass. The mixture was concentrated in vacuum to remove most of the organic solvent. The residue was treated with a mixture of AcOH (580 μL) in H₂O (5 mL) at 0° C. to adjust pH=5-6. The mixture was extracted with EtOAc (10 mL×2), the combined organic layers were concentrated in vacuum to afford 3-(4-((benzyloxy)carbonyl)piperazin-1-yl)-2-((tert-butoxycarbonyl)amino)propanoic acid (850 mg) as a brown oil, which was used directly. MS(M+H)⁺=408.2.

### Step 6. Synthesis of 2-((tert-butoxycarbonyl)amino)-3-(piperazin-1-yl)propanoic acid (5)

To a solution of 3-(4-((benzyloxy)carbonyl)piperazin-1-yl)-2-((tert-butoxycarbonyl)amino)propanoic acid (850 mg, 2.09 mmol) in CF₃CH₂OH (15 mL) was added Pd/C (200 mg, 10% purity) under N₂. The suspension was degassed under vacuum and purged with H₂ several times. The mixture was stirred under H₂ (15 psi) at 25° C. for 12 hours. LCMS showed the starting material was consumed completely. The mixture was filtered and the filter cake was washed with CF₃CH₂OH (50 mL), the filtrate was concentrated in vacuum to afford 2-((tert-butoxycarbonyl)amino)-3-(piperazin-1-yl)propanoic acid (590 mg, crude) as a light brown solid. MS(M+H)⁺=274.1.

### Step 7. Synthesis of 2-((tert-butoxycarbonyl)amino)-3-(4-(2-(2,6-dioxopiperidin-3-yl)-1,3-dioxoisoindolin-4-yl)piperazin-1-yl)propanoic acid (7)

A mixture of 2-(2,6-dioxopiperidin-3-yl)-4-fluoroisoindoline-1,3-dione (600 mg, 2.17 mmol), 2-((tert-butoxycarbonyl)amino)-3-(piperazin-1-yl)propanoic acid (600.00 mg, 2.20 mmol) and DIPEA (890.40 mg, 6.89 mmol, 1.20 mL) in DMSO (10 mL) was stirred at 100° C. for 12 hours. LCMS showed 2-(2,6-dioxopiperidin-3-yl)-4-fluoroisoindoline-1,3-dione was consumed completely and 74% of desired mass. To the mixture was added AcOH to until pH=7, the resulting mixture was filtered. The filtrate was purified by prep-HPLC (column: Phenomenex luna C18 150*40 mm*15 um; mobile phase: [water(FA)-ACN]; B %: 10%-40%, 15 min), the eluent was freeze-dried to afford 2-((tert-butoxycarbonyl)amino)-3-(4-(2-(2,6-dioxopiperidin-3-yl)-1,3-dioxoisoindolin-4-yl)piperazin-1-yl)propanoic acid (460 mg, 868.68 μmol, 39.99% yield) as an orange solid. MS(M+H)⁺=530.2.

### Step 8. Synthesis of tert-butyl (1-(4-(4-(((R)-8-cyclopentyl-7-ethyl-5-methyl-6-oxo-5,6,7,8-tetrahydropteridin-2-yl)amino)-3-methoxybenzamido)piperidin-1-yl)-3-(4-(2-(2,6-dioxopiperidin-3-yl)-1,3-dioxoisoindolin-4-yl)piperazin-1-yl)-1-oxopropan-2-yl)carbamate (9)

To a solution of 2-((tert-butoxycarbonyl)amino)-3-(4-(2-(2,6-dioxopiperidin-3-yl)-1,3-dioxoisoindolin-4-yl)piperazin-1-yl)propanoic acid (180 mg, 339.92 μmol), HATU (181.70 mg, 477.86 mol) and DIPEA (285.05 mg, 2.21 mmol, 384.16 μL) in DMF (5 mL) was stirred at 25° C. for 15 minutes, then (R)-4-((8-cyclopentyl-7-ethyl-5-methyl-6-oxo-5,6,7,8-tetrahydropteridin-2-yl)amino)-3-methoxy-N-(piperidin-4-yl)benzamide (200 mg, 367.59 μmol, HCl salt) was added, the resulting mixture was stirred at 25° C. for 2 hours, LCMS showed trace of 2-((tert-butoxycarbonyl)amino)-3-(4-(2-(2,6-dioxopiperidin-3-yl)-1,3-dioxoisoindolin-4-yl)piperazin-1-yl)propanoic acid remained and 67% of the desired mass. The mixture was diluted with H₂O (15 mL) and extracted with EtOAc (10 mL×3). The combined organic layers were washed with brine (15 mL×5), dried over Na₂SO₄, filtered and concentrated in vacuum. The residue was purified by prep-TLC (SiO₂, DCM:MeOH=15:1, twice) to afford tert-butyl (1-(4-(4-(((R)-8-cyclopentyl-7-ethyl-5-methyl-6-oxo-5,6,7,8-tetrahydropteridin-2-yl)amino)-3-methoxybenzamido)piperidin-1-yl)-3-(4-(2-(2,6-dioxopiperidin-3-yl)-1,3-dioxoisoindolin-4-yl)piperazin-1-yl)-1-oxopropan-2-yl)carbamate (320 mg, 313.99 μmol, 85.42% yield) as a yellow solid. MS(M+H)⁺=1019.4.

### Step 9. Synthesis of N-(1-(2-amino-3-(4-(2-(2,6-dioxopiperidin-3-yl)-1,3-dioxoisoindolin-4-yl)piperazin-1-yl) propanoyl)piperidin-4-yl)-4-(((R)-8-cyclopentyl-7-ethyl-5-methyl-6-oxo-5,6,7,8-tetrahydropteridin-2-yl)amino)-3-methoxybenzamide (Compound 9)

To a solution of tert-butyl (1-(4-(4-(((R)-8-cyclopentyl-7-ethyl-5-methyl-6-oxo-5,6,7,8-tetrahydropteridin-2-yl)amino)-3-methoxybenzamido)piperidin-1-yl)-3-(4-(2-(2,6-dioxopiperidin-3-yl)-1,3-dioxoisoindolin-4-yl)piperazin-1-yl)-1-oxopropan-2-yl)carbamate (320 mg, 313.99 μmol) in DCM (5 mL) was added HCl/dioxane (4 M, 5 mL), the mixture was stirred at 25° C. for 1 hour. LCMS showed the starting material was consumed completely and 75% of desired mass. The mixture was concentrated in vacuum. The residue was diluted with DMF (2.5 mL), the mixture was treated with DIPEA to adjust pH>7. The resulting mixture was purified by prep-HPLC (column: Waters xbridge 150*25 mm 10 um; mobile phase: [water(NH₄HCO₃)-ACN]; B %: 35%-65%, 10 min), the eluent was freeze-dried to afford N-(1-(2-amino-3-(4-(2-(2,6-dioxopiperidin-3-yl)-1,3-dioxoisoindolin-4-yl)piperazin-1-yl) propanoyl)piperidin-4-yl)-4-(((R)-8-cyclopentyl-7-ethyl-5-methyl-6-oxo-5,6,7,8-tetrahydropteridin-2-yl)amino)-3-methoxybenzamide (127.0 mg, 129.34 μmol, 41.19% yield, 93.6% purity) as a yellow solid. MS(M+H)⁺=919.5.

¹H NMR (400 MHz, CD₃OD) δ=8.54-8.42 (m, 1H), 7.78 (d, J=1.5 Hz, 1H), 7.70-7.59 (m, 1H), 7.52-7.42 (m, 2H), 7.41-7.25 (m, 2H), 5.14-5.05 (m, 1H), 4.59-4.42 (m, 2H), 4.32-4.25 (m, 1H), 4.25-4.04 (m, 3H), 4.00 (d, J=8.5 Hz, 3H), 3.47-3.35 (m, 4H), 3.35-3.33 (m, 3H), 2.96-2.80 (m, 3H), 2.80-2.70 (m, 4H), 2.70-2.58 (m, 2H), 2.58-2.46 (m, 1H), 2.24-2.10 (m, 3H), 2.07-1.92 (m, 3H), 1.92-1.78 (m, 5H), 1.77-1.68 (m, 3H), 1.67-1.42 (m, 2H), 0.86 (br t, J=7.4 Hz, 3H).

### Example 10. Synthesis of N-(1-(2-amino-3-(4-(4-(2,6-dioxopiperidin-3-yl)phenyl)piperazin-1-yl)propanoyl)piperidin-4-yl)-4-(((R)-8-cyclopentyl-7-ethyl-5-methyl-6-oxo-5,6,7,8-tetrahydropteridin-2-yl)amino)-3-(trifluoromethoxy)benzamide (Compound 10)

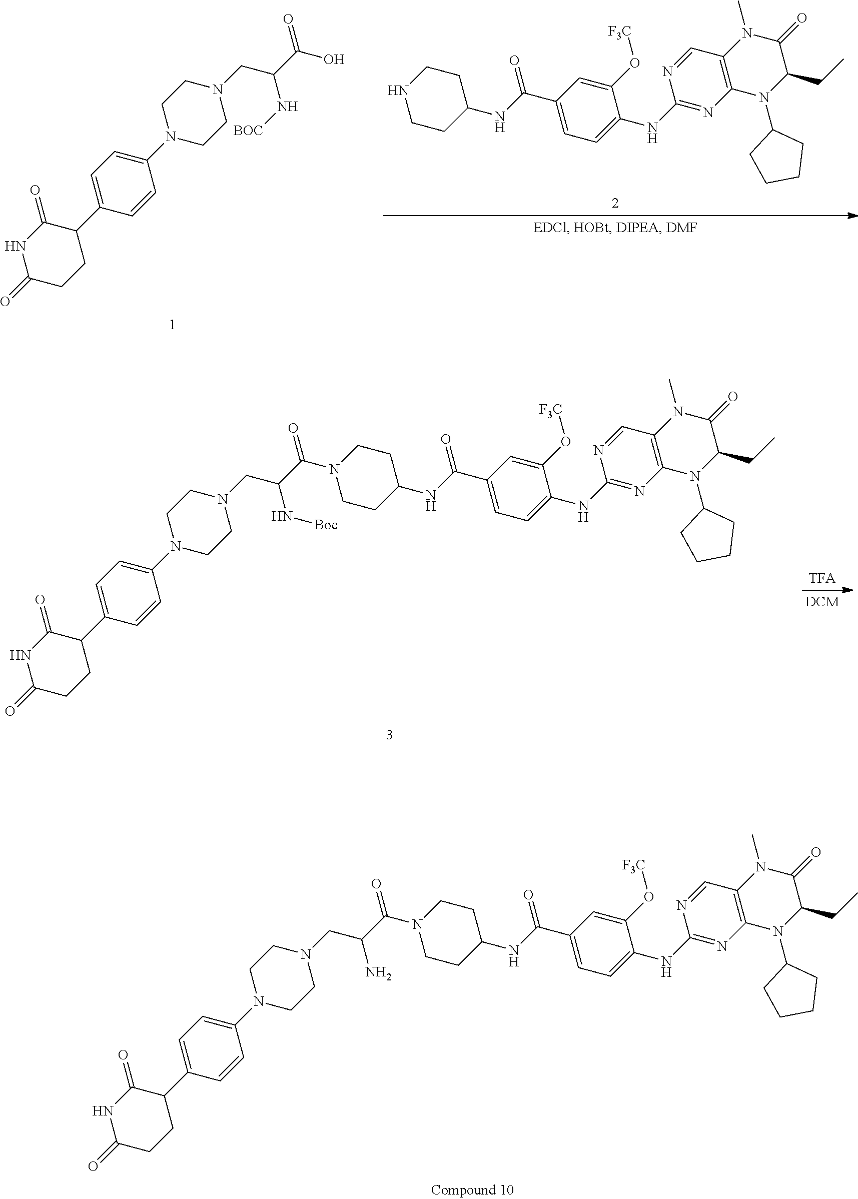 [SMILES: CC(C)(C)OC(=O)NC(CN1CCN(c2ccc(C3CCC(=O)NC3=O)cc2)CC1)C(=O)O ; CC[C@@H]1C(=O)N(C)c2cnc(Nc3ccc(C(=O)NC4CCNCC4)cc3OC(F)(F)F)nc2N1C1CCCC1 ; CC[C@@H]1C(=O)N(C)c2cnc(Nc3ccc(C(=O)NC4CCN(C(=O)C(CN5CCN(c6ccc(C7CCC(=O)NC7=O)cc6)CC5)NC(=O)OC(C)(C)C)CC4)cc3OC(F)(F)F)nc2N1C1CCCC1 ; CC[C@@H]1C(=O)N(C)c2cnc(Nc3ccc(C(=O)NC4CCN(C(=O)C(N)CN5CCN(c6ccc(C7CCC(=O)NC7=O)cc6)CC5)CC4)cc3OC(F)(F)F)nc2N1C1CCCC1]

### Step 1. Synthesis of tert-butyl (1-(4-(4-(((R)-8-cyclopentyl-7-ethyl-5-methyl-6-oxo-5,6,7,8-tetrahydropteridin-2-yl)amino)-3-(trifluoromethoxy)benzamido)piperidin-1-yl)-3-(4-(4-(2,6-dioxopiperidin-3-yl)phenyl)piperazin-1-yl)-1-oxopropan-2-yl)carbamate (3)

To a solution of 2-((tert-butoxycarbonyl)amino)-3-(4-(4-(2,6-dioxopiperidin-3-yl)phenyl)piperazin-1-yl)propanoic acid (200 mg, 342.22 μmol, 78.8% purity) in DMF (6 mL) were added EDCI (78.72 mg, 410.66 μmol) and HOBt (55.49 mg, 410.66 μmol), DIPEA (132.69 mg, 1.03 mmol, 178.82 L) and (R)-4-((8-cyclopentyl-7-ethyl-5-methyl-6-oxo-5,6,7,8-tetrahydropteridin-2-yl)amino)-N-(piperidin-4-yl)-3-(trifluoromethoxy)benzamide (204.67 mg, 342.22 μmol, HCl salt). The mixture was stirred at 25° C. for 1 h. LCMS showed ~60% of desired mass. The reaction mixture was diluted with water (30 mL) and extracted with EtOAc (20 mL×3). The combined organic layers were washed with saturated brine (50 mL), dried over Na₂SO₄, filtered and concentrated under reduced pressure to give a residue. The residue was purified by flash silica gel chromatography (ISCO®; 12 g SepaFlash® Silica Flash Column, eluent of 0~40% petroleum ether:EtOAc/EtOH (v/v=5/1) gradient @80 mL/min) to afford tert-butyl (1-(4-(4-(((R)-8-cyclopentyl-7-ethyl-5-methyl-6-oxo-5,6,7,8-tetrahydropteridin-2-yl)amino)-3-(trifluoromethoxy)benzamido)piperidin-1-yl)-3-(4-(4-(2,6-dioxopiperidin-3-yl)phenyl)piperazin-1-yl)-1-oxopropan-2-yl)carbamate (180 mg, 169.23 μmol, 49.45% yield, 94.4% purity) as yellow oil. MS(M+H)⁺=1004.5.

### Step 2. Synthesis of N-(1-(2-amino-3-(4-(4-(2,6-dioxopiperidin-3-yl)phenyl)piperazin-1-yl)propanoyl)piperidin-4-yl)-4-(((R)-8-cyclopentyl-7-ethyl-5-methyl-6-oxo-5,6,7,8-tetrahydropteridin-2-yl)amino)-3-(trifluoromethoxy)benzamide (Compound 10)

To a solution of tert-butyl (1-(4-(4-(((R)-8-cyclopentyl-7-ethyl-5-methyl-6-oxo-5,6,7,8-tetrahydropteridin-2-yl)amino)-3-(trifluoromethoxy)benzamido)piperidin-1-yl)-3-(4-(4-(2,6-dioxopiperidin-3-yl)phenyl)piperazin-1-yl)-1-oxopropan-2-yl)carbamate (180 mg, 179.26 μmol) in DCM (2 mL) was added TFA (3.08 g, 27.01 mmol, 2 mL) at 0° C. The mixture was stirred at 25° C. for 2 h. LCMS showed ~72.7% of desired mass. The mixture was concentrated under reduced pressure to give a residue. The residue was purified by prep-HPLC (column: Phenomenex luna C18 150×40 mmx 15 μm; mobile phase: [water (TFA)-ACN]; B %: 15%-45%, 10 min) followed by lyophilization to afford N-(1-(2-amino-3-(4-(4-(2,6-dioxopiperidin-3-yl)phenyl)piperazin-1-yl)propanoyl)piperidin-4-yl)-4-(((R)-8-cyclopentyl-7-ethyl-5-methyl-6-oxo-5,6,7,8-tetrahydropteridin-2-yl)amino)-3-(trifluoromethoxy)benzamide (56 mg, 54.46 μmol, 30.38% yield, 99% purity, TFA salt) as a white solid. MS(M+H)⁺=904.4.

¹H NMR (400 MHz, DMSO-d₆) δ=10.77 (d, J=2.9 Hz, 1H), 9.57-9.36 (m, 1H), 8.45 (dd, J=7.6, 17.7 Hz, 1H), 8.22-7.97 (m, 3H), 7.95-7.88 (m, 2H), 7.79 (d, J=2.1 Hz, 1H), 7.10-7.02 (m, 2H), 6.92 (dd, J=4.8, 8.5 Hz, 2H), 4.72-4.62 (m, 1H), 4.39-4.37 (m, 1H), 4.10-4.07 (m, 2H), 3.95-3.86 (m, 4H), 3.75-3.70 (m, 1H), 3.23-3.14 (m, 6H), 2.95-2.72 (m, 5H), 2.66-2.57 (m, 6H), 2.23-2.04 (m, 2H), 1.95-1.72 (m, 8H), 1.62-1.53 (m, 1H), 1.46-1.34 (m, 5H), 0.78-0.70 (m, 3H).

### Example 11. Synthesis of N-(1-(2-amino-3-(4-(4-(2,6-dioxopiperidin-3-yl)phenyl)piperazin-1-yl)propanoyl)piperidin-4-yl)-4-(((R)-8-cyclopentyl-7-ethyl-5-methyl-6-oxo-5,6,7,8-tetrahydropteridin-2-yl)amino)-3-methoxybenzamide (Compound 11)

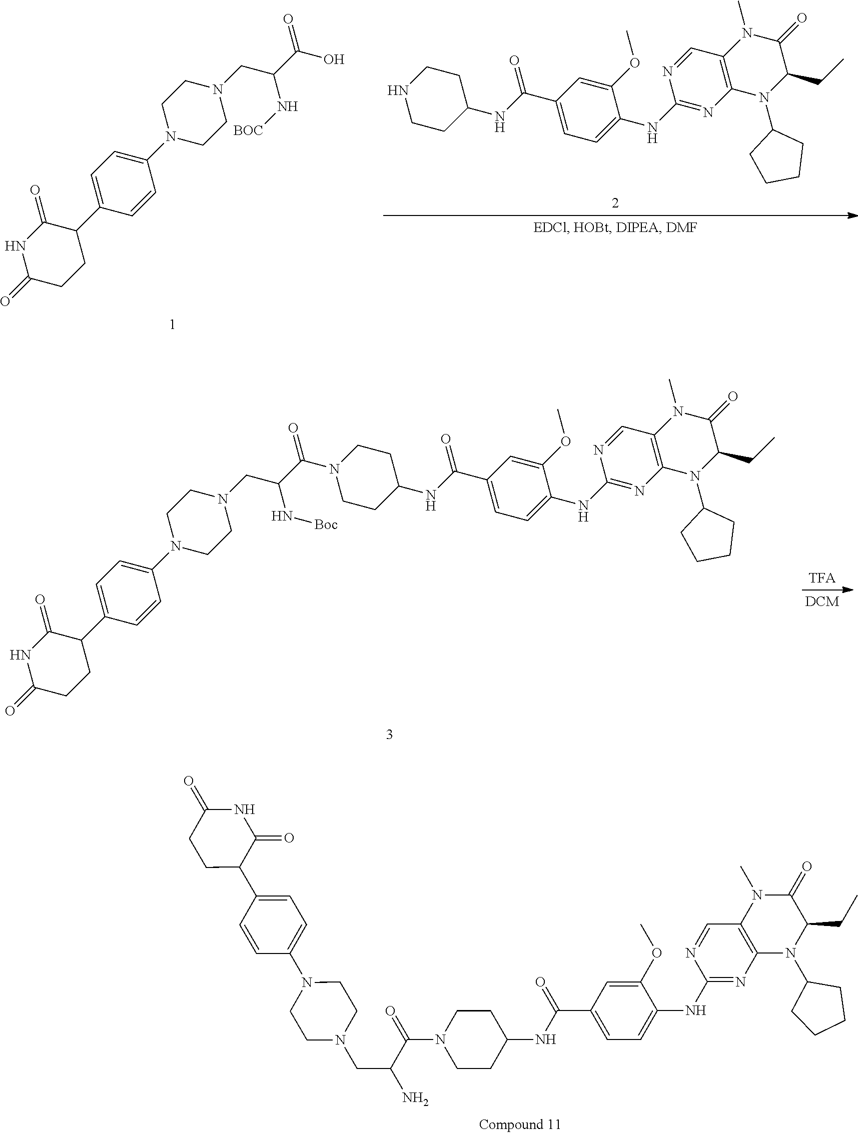 [SMILES: CC(C)(C)OC(=O)NC(CN1CCN(c2ccc(C3CCC(=O)NC3=O)cc2)CC1)C(=O)O ; CC[C@@H]1C(=O)N(C)c2cnc(Nc3ccc(C(=O)NC4CCNCC4)cc3OC)nc2N1C1CCCC1 ; CC[C@@H]1C(=O)N(C)c2cnc(Nc3ccc(C(=O)NC4CCN(C(=O)C(CN5CCN(c6ccc(C7CCC(=O)NC7=O)cc6)CC5)NC(=O)OC(C)(C)C)CC4)cc3OC)nc2N1C1CCCC1 ; CC[C@@H]1C(=O)N(C)c2cnc(Nc3ccc(C(=O)NC4CCN(C(=O)C(N)CN5CCN(c6ccc(C7CCC(=O)NC7=O)cc6)CC5)CC4)cc3OC)nc2N1C1CCCC1]

### Step 1. Synthesis of tert-butyl (1-(4-(4-(((R)-8-cyclopentyl-7-ethyl-5-methyl-6-oxo-5,6,7,8-tetrahydropteridin-2-yl)amino)-3-methoxybenzamido) piperidin-1-yl)-3-(4-(4-(2,6-dioxopiperidin-3-yl)phenyl)piperazin-1-yl)-1-oxopropan-2-yl) carbamate (3)

To a solution of 2-((tert-butoxycarbonyl)amino)-3-(4-(4-(2,6-dioxopiperidin-3-yl)phenyl)piperazin-1-yl)propanoic acid (200 mg, 342.22 μmol, 78.8% purity) in DMF (5 mL) were added EDCI (78.73 mg, 410.66 μmol), (R)-4-((8-cyclopentyl-7-ethyl-5-methyl-6-oxo-5,6,7,8-tetrahydropteridin-2-yl)amino)-3-methoxy-N-(piperidin-4-yl)benzamide (186.20 mg, 342.22 μmol, HCl salt), DIPEA (132.69 mg, 1.03 mmol, 178.83 L) and HOBt (55.49 mg, 410.66 μmol). The mixture was stirred at 25° C. for 2 h. LCMS showed ~60% of desired mass. The reaction mixture was diluted with water (30 mL) and extracted with EtOAc (20 mL×3). The combined organic layers were washed with saturated brine (50 mL), dried over Na₂SO₄, filtered and concentrated under reduced pressure to give a residue, which was purified by flash silica gel chromatography (ISCO®; 12 g SepaFlash® Silica Flash Column, Eluent of 0~50% petroleum ether:EtOAc/EtOH (v/v=5/1) gradient @80 mL/min) to afford tert-butyl (1-(4-(4-(((R)-8-cyclopentyl-7-ethyl-5-methyl-6-oxo-5,6,7,8-tetrahydropteridin-2-yl)amino)-3-methoxybenzamido)piperidin-1-yl)-3-(4-(4-(2,6-dioxopiperidin-3-yl)phenyl)piperazin-1-yl)-1-oxopropan-2-yl)carbamate (180 mg, 159.51 μmol, 46.61% yield, 84.2% purity) as a yellow solid. MS(M+H)⁺=950.4.

### Step 2 Synthesis of N-(1-(2-amino-3-(4-(4-(2,6-dioxopiperidin-3-yl)phenyl)piperazin-1-yl)propanoyl)piperidin-4-yl)-4-(((R)-8-cyclopentyl-7-ethyl-5-methyl-6-oxo-5,6,7,8-tetrahydropteridin-2-yl)amino)-3-methoxybenzamide (Compound 11)

To a solution of tert-butyl (1-(4-(4-(((R)-8-cyclopentyl-7-ethyl-5-methyl-6-oxo-5,6,7,8-tetrahydropteridin-2-yl)amino)-3-methoxybenzamido)piperidin-1-yl)-3-(4-(4-(2,6-dioxopiperidin-3-yl)phenyl)piperazin-1-yl)-1-oxopropan-2-yl)carbamate (180 mg, 189.45 μmol) in DCM (2 mL) was added TFA (3.08 g, 27.01 mmol, 2 mL) at 0° C. The mixture was stirred at 25° C. for 1 h. LCMS showed ~76% of desired mass. The reaction mixture was concentrated under reduced pressure to give a residue. The residue was purified by prep-HPLC (column: Phenomenex luna C18 150×40 mm×15 μm; mobile phase: [water (TFA)-ACN]; B %: 10%-40%, 10 min) and prep-HPLC(column: Waters Xbridge 150×25 mm×5 μm; mobile phase: [water (NH₄HCO₃)-ACN]; B %: 36%-66%, 10 min) followed by lyophilization to afford N-(1-(2-amino-3-(4-(4-(2,6-dioxopiperidin-3-yl)phenyl)piperazin-1-yl)propanoyl)piperidin-4-yl)-4-(((R)-8-cyclopentyl-7-ethyl-5-methyl-6-oxo-5,6,7,8-tetrahydropteridin-2-yl)amino)-3-methoxybenzamide (61.4 mg, 71.08 μmol, 37.52% yield, 98.4% purity) as a white solid. MS(M+H)⁺=850.2.

¹H NMR (400 MHz, DMSO-d₆) δ=10.84-10.67 (m, 1H), 8.41 (d, J=5.3 Hz, 1H), 8.13 (d, J=4.5 Hz, 1H), 7.84 (s, 1H), 7.60 (s, 1H), 7.47 (d, J=8.1 Hz, 2H), 7.06-7.03 (m, 2H), 6.89 (d, J=8.6 Hz, 2H), 4.48-4.32 (m, 2H), 4.23 (dd, J=3.4, 7.5 Hz, 1H), 4.12-4.00 (m, 2H), 3.93 (d, J=6.0 Hz, 4H), 3.75-3.67 (m, 1H), 3.24 (s, 3H), 3.15-3.08 (m, 5H), 2.75-2.56 (m, 6H), 2.47-2.31 (m, 4H), 2.17-2.10 (m, 1H), 2.00-1.96 (m, 2H), 1.90-1.74 (m, 8H), 1.72-1.48 (m, 5H), 1.45-1.33 (m, 1H), 0.76 (t, J=7.4 Hz, 3H).

### Example 12. Synthesis of 4-(((R)-8-cyclopentyl-7-ethyl-5-methyl-6-oxo-5,6,7,8-tetrahydropteridin-2-yl)amino)-N-((1r,4R)-4-(4-(((2-(2,6-dioxopiperidin-3-yl)-1-oxoisoindolin-4-yl)methyl)amino)piperidin-1-yl)cyclohexyl)-3-methoxybenzamide (Compound 12)

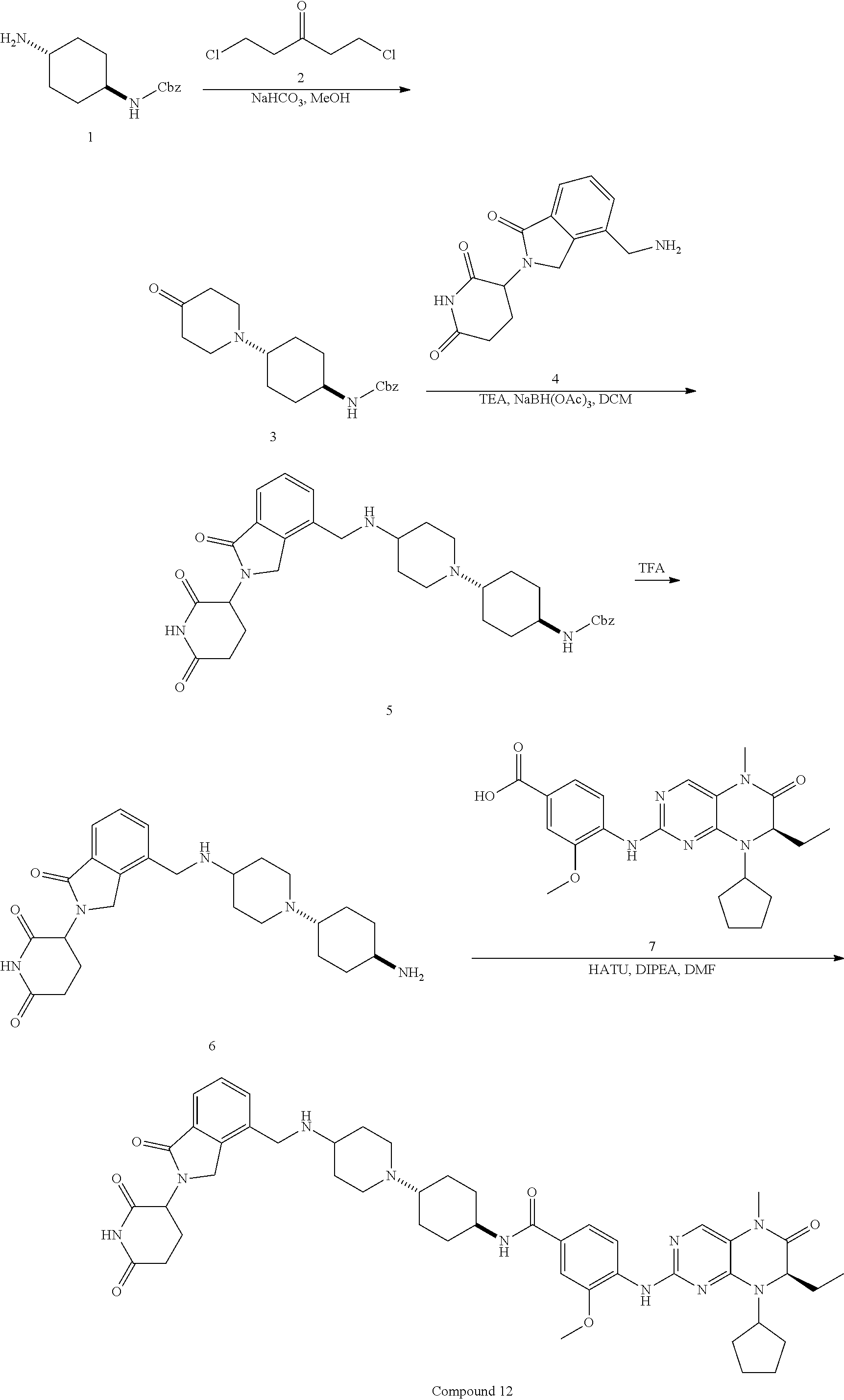

### Step 1. Synthesis of benzyl ((1r,4r)-4-(4-oxopiperidin-1-yl)cyclohexyl)carbamate (3)

To a solution of benzyl ((1r,4r)-4-aminocyclohexyl)carbamate (200 mg, 702.29 μmol HCl) in MeOH (2 mL) were added 1,5-dichloropentan-3-one (108.87 mg, 702.29 mol) and NaHCO₃ (294.99 mg, 3.51 mmol, 136.63 μL), the mixture was stirred at 75° C. for 16 h. LCMS showed overlapped peaks have desired mass. TLC (DCM:MeOH=10:1) showed a new spot was detected. The reaction mixture was diluted with H₂O (10 mL), extracted with EtOAc (10 mL×3). The combined organic layers were washed with brine (20 mL×2), dried over Na₂SO₄, filtered. The filtrate was concentrated in vacuum. The residue was purified by flash silica gel chromatography (Biotage; 10 g SepaFlash® Silica Flash Column, Eluent of 50~100% EtOAc/Petroleum ether to 10% MeOH/EtOAc gradient @100 mL/min) to afford benzyl ((1r,4r)-4-(4-oxopiperidin-1-yl)cyclohexyl)carbamate (140 mg, 423.70 μmol, 60.33% yield) as a yellow solid. MS(M+H)⁺=331.1.

### Step 2. Synthesis of benzyl ((1r,4r)-4-(4-(((2-(2,6-dioxopiperidin-3-yl)-1-oxoisoindolin-4-yl)methyl)amino)piperidin-1-yl)cyclohexyl)carbamate (5)

A solution of benzyl ((1r,4r)-4-(4-oxopiperidin-1-yl)cyclohexyl)carbamate (140 mg, 423.70 μmol), 3-(4-(aminomethyl)-1-oxoisoindolin-2-yl)piperidine-2,6-dione (115.79 mg, 423.70 μmol), TEA (128.62 mg, 1.27 mmol, 176.92 L) in DCM (2 mL) was stirred at 20° C. for 0.5 h, NaBH(OAc)₃ (269.40 mg, 1.27 mmol) was added, the mixture was stirred at 20° C. for 16 h. LCMS showed a main peak with desired mass. The mixture was filtered through a pad of celite. The filtrate was quenched with NaHCO₃ (5 mL), extracted with EtOAc (10 mL×5). The combined organic layers were dried over Na₂SO₄, filtered and the filtrate was concentrated in vacuum to afford benzyl ((1r,4r)-4-(4-(((2-(2,6-dioxopiperidin-3-yl)-1-oxoisoindolin-4-yl)methyl)amino)piperidin-1-yl)cyclohexyl)carbamate (100 mg, crude) as a yellow solid. MS(M+H)⁺=588.3.

### Step 3. Synthesis of 3-(4-(((1-((1r,4r)-4-aminocyclohexyl)piperidin-4-yl)amino)methyl)-1-oxoisoindolin-2-yl)piperidine-2,6-dione (6)

A mixture of benzyl ((1r,4r)-4-(4-(((2-(2,6-dioxopiperidin-3-yl)-1-oxoisoindolin-4-yl)methyl)amino)piperidin-1-yl)cyclohexyl)carbamate (100 mg, 170.15 μmol) in TFA (1 mL) was stirred at 60° C. for 4 h. LCMS showed a main peak with desired mass. The mixture was concentrated in vacuum to afford 3-(4-(((1-((1r,4r)-4-aminocyclohexyl)piperidin-4-yl)amino)methyl)-1-oxoisoindolin-2-yl)piperidine-2,6-dione (90 mg, 158.56 μmol, 93.19% yield, TFA) as a brown oil. MS(M+H)⁺=454.2.

### Step 4. Synthesis of 4-(((R)-8-cyclopentyl-7-ethyl-5-methyl-6-oxo-5,6,7,8-tetrahydropteridin-2-yl)amino)-N-((1r,4R)-4-(4-(((2-(2,6-dioxopiperidin-3-yl)-1-oxoisoindolin-4-yl)methyl)aminol)piperidin-1-yl)cyclohexyl)-3-methoxybenzamide (Compound 12)

To a solution of (R)-4-((8-cyclopentyl-7-ethyl-5-methyl-6-oxo-5,6,7,8-tetrahydropteridin-2-yl)amino)-3-methoxybenzoic acid (67.47 mg, 158.56 μmol) in DMF (1 mL) were added HATU (72.35 mg, 190.28 μmol) and DIPEA (122.96 mg, 951.38 μmol, 165.71 μL), the mixture was stirred at 20° C. for 0.5 h, 3-(4-(((1-((1r,4r)-4-aminocyclohexyl)piperidin-4-yl)amino)methyl)-1-oxoisoindolin-2-yl)piperidine-2,6-dione (90 mg, 158.56 μmol, TFA) was added, the mixture was stirred at 20° C. for 16 h. LCMS showed a overlapped peak (70%) with desired mass. The reaction mixture was diluted with H₂O (5 mL), extracted with EtOAc (10 mL×3). The combined organic layers were washed with brine (20 mL×2), dried over Na₂SO₄, filtered. The filtrate was concentrated in vacuum. The crude product was purified by reversed-phase HPLC (column: Waters Xbridge 150*25 mm*5 um; mobile phase: [water (NH₄HCO₃)-ACN]; B %: 37%-67%, 9 min) and lyophilized to afford 4-(((R)-8-cyclopentyl-7-ethyl-5-methyl-6-oxo-5,6,7,8-tetrahydropteridin-2-yl)amino)-N-((1r,4R)-4-(4-(((2-(2,6-dioxopiperidin-3-yl)-1-oxoisoindolin-4-yl)methyl)amino)piperidin-1-yl)cyclohexyl)-3-methoxybenzamide (18.1 mg, 19.13 μmol, 12.06% yield, 91% purity) as a white solid. MS(M+H)⁺=861.4.

¹H NMR (400 MHz, DMSO-d₆) δ=10.99 (s, 1H), 8.43-8.38 (m, 1H), 8.01 (d, J=7.7 Hz, 1H), 7.84 (s, 1H), 7.62-7.57 (m, 3H), 7.49-7.44 (m, 3H), 5.17-5.09 (m, 1H), 4.55-4.30 (m, 3H), 4.27-4.20 (m, 1H), 3.93 (s, 3H), 3.85-3.78 (m, 2H), 3.76-3.67 (m, 1H), 3.24 (s, 3H), 3.01-2.86 (m, 1H), 2.85-2.72 (m, 2H), 2.65-2.56 (m, 4H), 2.44-2.38 (m, 2H), 2.28-2.24 (m, 1H), 2.20-2.13 (m, 2H), 2.05-1.99 (m, 2H), 1.92-1.85 (m, 4H), 1.82-1.75 (m, 6H), 1.67-1.58 (m, 3H), 1.38-1.22 (m, 6H), 0.76 (t, J=7.4 Hz, 3H).

### Example 13. Synthesis of 4-(((R)-8-cyclopentyl-7-ethyl-5-methyl-6-oxo-5,6,7,8-tetrahydropteridin-2-yl)amino)-N-((1-((1-(4-((2,6-dioxopiperidin-3-yl)amino)phenyl)piperidin-4-yl)methyl)piperidin-4-yl)methyl)-3-methoxybenzamide (Compound 13)

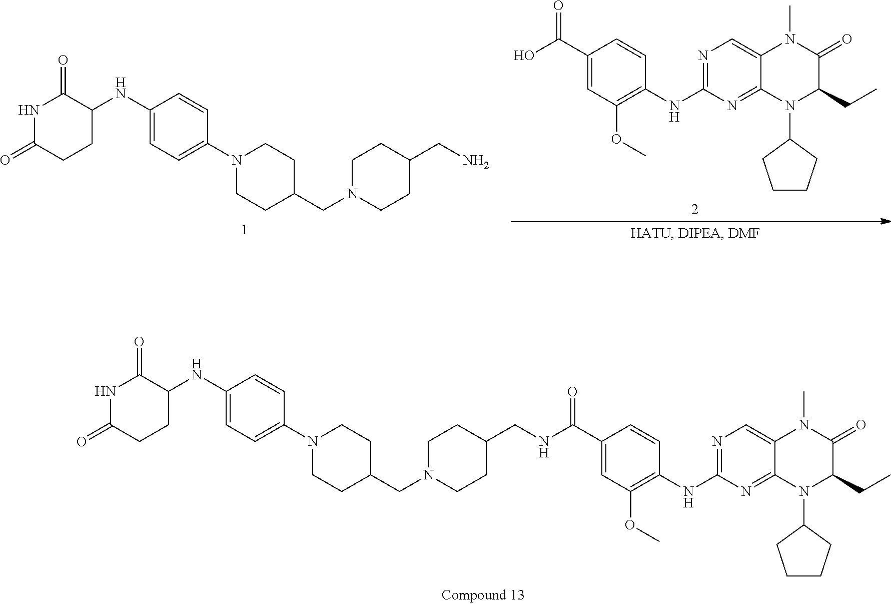 [SMILES: NCC1CCN(CC2CCN(c3ccc(NC4CCC(=O)NC4=O)cc3)CC2)CC1 ; CC[C@@H]1C(=O)N(C)c2cnc(Nc3ccc(C(=O)O)cc3OC)nc2N1C1CCCC1 ; CC[C@@H]1C(=O)N(C)c2cnc(Nc3ccc(C(=O)NCC4CCN(CC5CCN(c6ccc(NC7CCC(=O)NC7=O)cc6)CC5)CC4)cc3OC)nc2N1C1CCCC1]

### Step 1. Synthesis of 4-(((R)-8-cyclopentyl-7-ethyl-5-methyl-6-oxo-5,6,7,8-tetrahydropteridin-2-yl)amino)-N-((1-((1-(4-((2,6-dioxopiperidin-3-yl)amino)phenyl)piperidin-4-yl)methyl)piperidin-4-yl)methyl)-3-methoxybenzamide (Compound 13)

To a solution of (R)-4-((8-cyclopentyl-7-ethyl-5-methyl-6-oxo-5,6,7,8-tetrahydropteridin-2-yl)amino)-3-methoxybenzoic acid (61.29 mg, 144.05 μmol) in DMF (1 mL) were added HATU (65.73 mg, 172.87 μmol) and DIPEA (18.62 mg, 144.05 μmol, 25.09 L) at 20° C. After stirring 10 min, then a solution of 3-((4-(4-((4-(aminomethyl)piperidin-1-yl)methyl)piperidin-1-yl)phenyl)amino)piperidine-2,6-dione (114 mg, 216.08 μmol, TFA) in DMF (1 mL) and DIPEA (290.82 mg, 2.25 mmol, 391.94 L) was added at 20° C. and the resulting mixture was stirred at 20° C. for 16 h. LCMS showed starting material was consumed completely and 50% of peak with desired mass. The reaction mixture was diluted with H₂O (20 mL) and extracted with EtOAc (10 mL×3), the organic layers were washed with brine (10 mL×3), dried over Na₂SO₄, filtered and concentrated in vacuum to afford crude product. The crude product was purified by prep-TLC (SiO₂, DCM:MeOH=5:1) and the product was dissolved in mixture solution (20 mL, ACN:H₂O=1:3) and lyophilized to afford 4-(((R)-8-cyclopentyl-7-ethyl-5-methyl-6-oxo-5,6,7,8-tetrahydropteridin-2-yl)amino)-N-((1-((1-(4-((2,6-dioxopiperidin-3-yl)amino)phenyl)piperidin-4-yl)methyl)piperidin-4-yl)methyl)-3-methoxybenzamide (21 mg, 24.04 μmol, 16.45% yield, 94% purity) as an off-white solid. MS(M+H)⁺=821.2

¹H NMR (400 MHz, DMSO-d₆) δ=10.75 (s, 1H), 8.42 (d, J=8.8 Hz, 2H), 7.84 (s, 1H), 7.61 (s, 1H), 7.52-7.48 (m, 2H), 6.79-6.72 (m, 2H), 6.60 (d, J=8.9 Hz, 2H), 5.38 (d, J=6.4 Hz, 1H), 4.40-4.29 (m, 1H), 4.24 (dd, J=3.7, 7.6 Hz, 1H), 4.21-4.14 (m, 1H), 3.94 (s, 3H), 3.43-3.34 (m, 4H), 3.27-3.14 (m, 7H), 2.78-2.68 (m, 1H), 2.62-2.57 (m, 1H), 2.14-2.07 (m, 1H), 2.06-1.95 (m, 2H), 1.94-1.86 (m, 3H), 1.84-1.73 (m, 10H), 1.68-1.57 (m, 4H), 1.54-1.37 (m, 2H), 1.34-1.21 (m, 4H), 0.76 (t, J=7.5 Hz, 3H)

### Example 14. Synthesis of 4-(((R)-8-cyclopentyl-7-ethyl-5-methyl-6-oxo-5,6,7,8-tetrahydropteridin-2-yl)amino)-N-(1-(3-(4-(4-((2,6-dioxopiperidin-3-yl)amino)phenyl)piperazin-1-yl)propanoyl) piperidin-4-yl)-3-(trifluoromethoxy)benzamide (Compound 14)

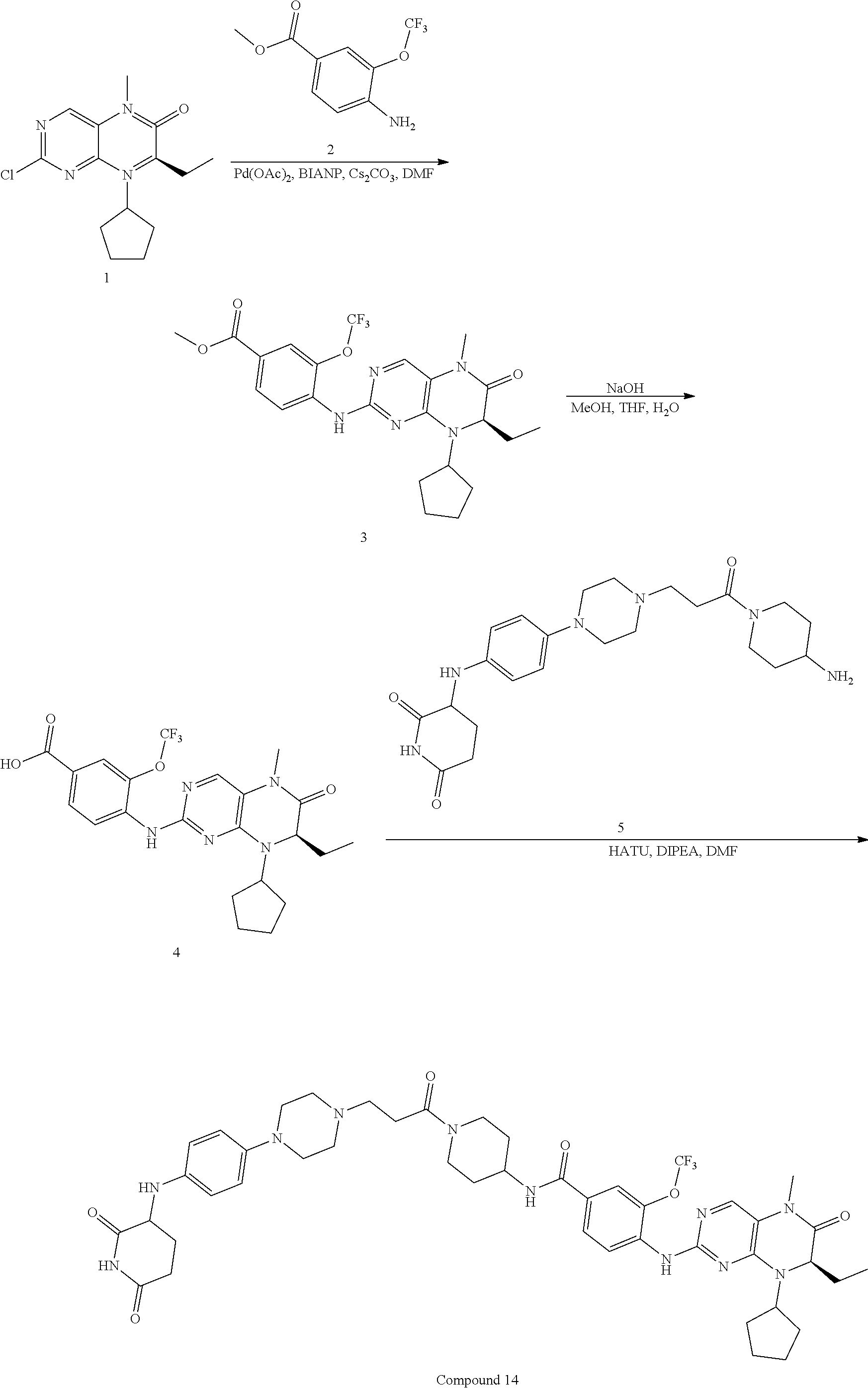

### Step 1. Synthesis of methyl (R)-4-((8-cyclopentyl-7-ethyl-5-methyl-6-oxo-5,6,7,8-tetrahydropteridin-2-yl)amino)-3-(trifluoromethoxy)benzoate (3)

To a solution of (R)-2-chloro-8-cyclopentyl-7-ethyl-5-methyl-7, 8-dihydropteridin-6(5H)-one (398.87 mg, 1.70 mmol) in DMF (8 mL) were added Pd(OAc)₂ (38.08 mg, 169.62 μmol) BINAP (211.23 mg, 339.24 μmol) and Cs₂CO₃ (1.11 g, 3.39 mmol), the reaction mixture was heated to 80° C. for 12 hr under N₂ atmosphere. LCMS showed a peak (57%) with desired mass. The reaction mixture was poured into 1N HCl solution to adjust PH-7, then extracted with EtOAc (50 mL×2). The combined organic layer was dried over Na₂SO₄, filtered. The filtrate was concentrated in vacuo. The residue was purified by flash silica gel chromatography (ISCO®; 40 g SepaFlash® Silica Flash Column, Eluent of 0~30% EtOAc/Petroleum ether gradient @60 mL/min) to afford methyl (R)-4-((8-cyclopentyl-7-ethyl-5-methyl-6-oxo-5,6,7,8-tetrahydropteridin-2-yl)amino)-3-(trifluoromethoxy)benzoate (330 mg, 668.72 μmol, 39.43% yield) as a yellow oil. MS(M+H)⁺=494.3.

### Step 2. Synthesis of (R)-4-((8-cyclopentyl-7-ethyl-5-methyl-6-oxo-5,6,7,8-tetrahydropteridin-2-yl)amino)-3-(trifluoromethoxy)benzoic acid (4)

To a solution of methyl (R)-4-((8-cyclopentyl-7-ethyl-5-methyl-6-oxo-5,6,7,8-tetrahydropteridin-2-yl)amino)-3-(trifluoromethoxy)benzoate (330 mg, 668.72 μmol) in THF (1.5 mL) and MeOH (1.5 mL) was added a solution of NaOH (40.12 mg, 1.00 mmol) in H₂O (0.5 mL) and the resulting mixture was stirred at 15° C. for 16 hr. LCMS showed 12% of methyl (R)-4-((8-cyclopentyl-7-ethyl-5-methyl-6-oxo-5,6,7,8-tetrahydropteridin-2-yl)amino)-3-(trifluoromethoxy)benzoate remained and a peak (59%) with desired mass. The reaction mixture was added con. HCl to adjust pH-6, then concentrated in vacuo to afford (R)-4-((8-cyclopentyl-7-ethyl-5-methyl-6-oxo-5,6,7,8-tetrahydropteridin-2-yl)amino)-3-(trifluoromethoxy)benzoic acid (320 mg, 667.43 μmol) a white solid, which was used in the next step without further purification. MS(M+H)⁺=480.2

### Step 3. Synthesis of 4-(((R)-8-cyclopentyl-7-ethyl-5-methyl-6-oxo-5,6,7,8-tetrahydropteridin-2-yl)amino)-N-(1-(3-(4-(4-((2,6-dioxopiperidin-3-yl)aminol)phenyl)piperazin-1-yl)propanoyl)piperidin-4-yl)-3-(trifluoromethoxy)benzamide (Compound 14)

To a solution of (R)-4-((8-cyclopentyl-7-ethyl-5-methyl-6-oxo-5,6,7,8-tetrahydropteridin-2-yl)amino)-3-(trifluoromethoxy)benzoic acid (97 mg, 168.75 μmol) in DMF (0.2 mL) were added HATU (133.22 mg, 350.36 μmol) and DIPEA (150.94 mg, 1.17 mmol, 203.42 L). The mixture was stirred at 25° C. for 0.5 h. Then 3-((4-(4-(3-(4-aminopiperidin-1-yl)-3-oxopropyl)piperazin-1-yl)phenyl)amino)piperidine-2,6-dione (130 mg, 233.57 μmol, TFA) was added and the resulting mixture was stirred at 25° C. for 16 hr. LCMS showed 3-((4-(4-(3-(4-aminopiperidin-1-yl)-3-oxopropyl)piperazin-1-yl)phenyl)amino)piperidine-2,6-dione was consumed completely and desired mass was detected. The reaction mixture was diluted with H₂O (10 mL) at 25° C., then extracted with EtOAc (80 mL). The organic layer was washed with brine (10 mL), dried over Na₂SO₄, filtered and concentrated under reduced pressure. The residue was purified by flash silica gel chromatography (ISCO®; 20 g SepaFlash® Silica Flash Column, Eluent of 0~15% MeOH/EtOAc @50 mL/min), then re-purified by prep-HPLC (column: Phenomenex luna C18 150*25 mm*10 um; mobile phase: [water (TFA)-ACN]; gradient:23%-53% B over 9 min). The eluent was lyophilized to afford 4-(((R)-8-cyclopentyl-7-ethyl-5-methyl-6-oxo-5,6,7,8-tetrahydropteridin-2-yl)amino)-N-(1-(3-(4-(4-((2,6-dioxopiperidin-3-yl)amino)phenyl)piperazin-1-yl)propanoyl)piperidin-4-yl)-3-(trifluoromethoxy)benzamide (53.8 mg, 45.62 μmol, 19.53% yield, 96% purity, 2 TFA) as a purple solid. MS(M+H)⁺=904.4.

¹H NMR (400 MHz, DMSO-d₆) δ=10.77 (s, 1H), 9.48-9.35 (m, 1H), 9.28-9.12 (m, 1H), 8.41 (br d, J=7.5 Hz, 1H), 8.09 (d, J=8.5 Hz, 1H), 7.95-7.87 (m, 2H), 7.79 (s, 1H), 6.83 (d, J=9.0 Hz, 2H), 6.65 (d, J=8.9 Hz, 2H), 4.41-4.34 (m, 2H), 4.27-4.19 (m, 2H), 4.14-4.06 (m, 3H), 3.92 (br d, J=3.5 Hz, 2H), 3.57 (br d, J=7.0 Hz, 2H), 3.40 (br d, J=6.3 Hz, 2H), 3.27-3.16 (m, 6H), 2.96-2.82 (m, 4H), 2.80-2.71 (m, 1H), 2.63-2.54 (m, 1H), 2.13-2.03 (m, 1H), 1.95-1.71 (m, 9H), 1.56-1.36 (m, 6H), 0.75 (t, J=7.4 Hz, 3H).

### Example 15. Synthesis of 3-(4-(4-(2-amino-3-(4-(((5-(4-(((R)-8-cyclopentyl-7-ethyl-5-methyl-6-oxo-5,6,7,8-tetrahydropteridin-2-yl)amino)-3-methoxyphenyl)-1,3,4-oxadiazol-2-yl)methyl)amino)piperidin-1-yl)-3-oxopropyl)piperazin-1-yl)phenyl)piperidine-2,6-dione (Compound 15)

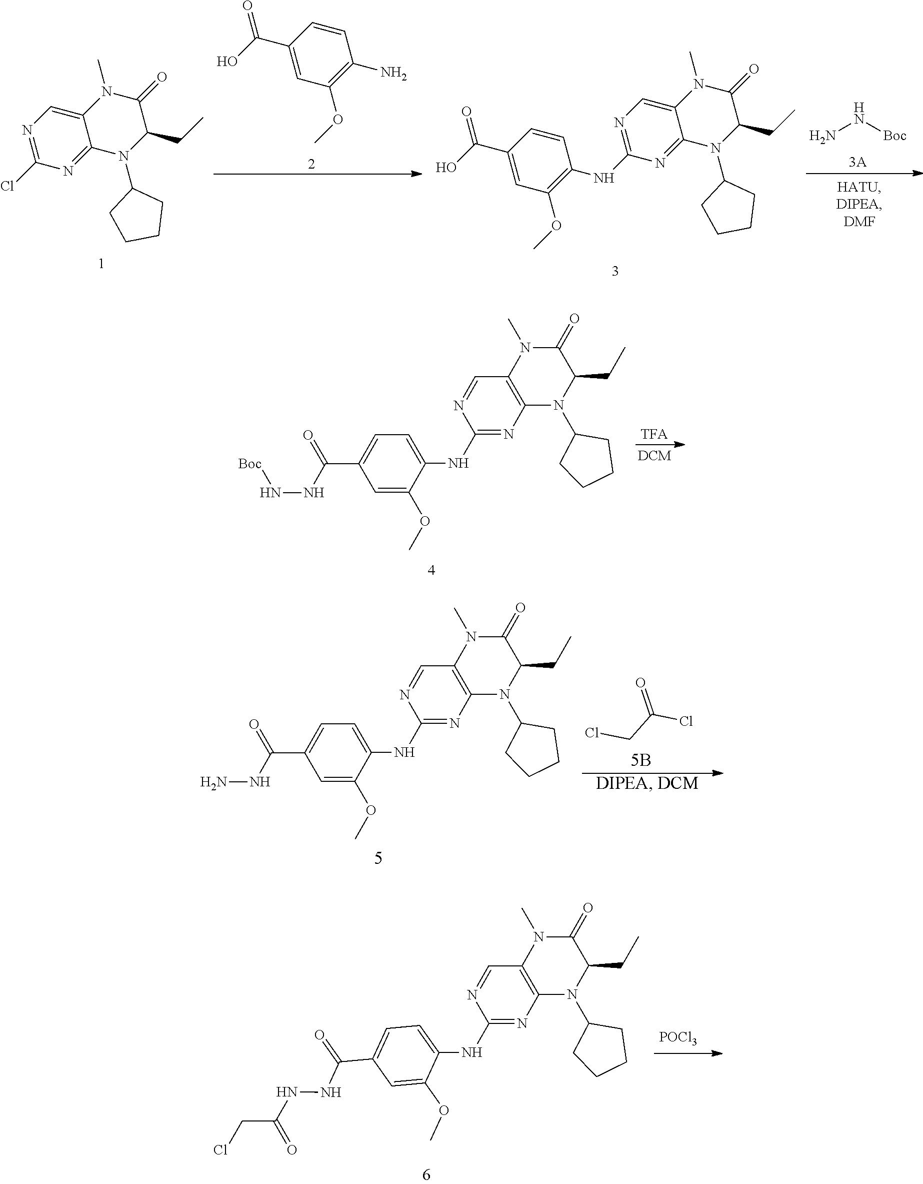

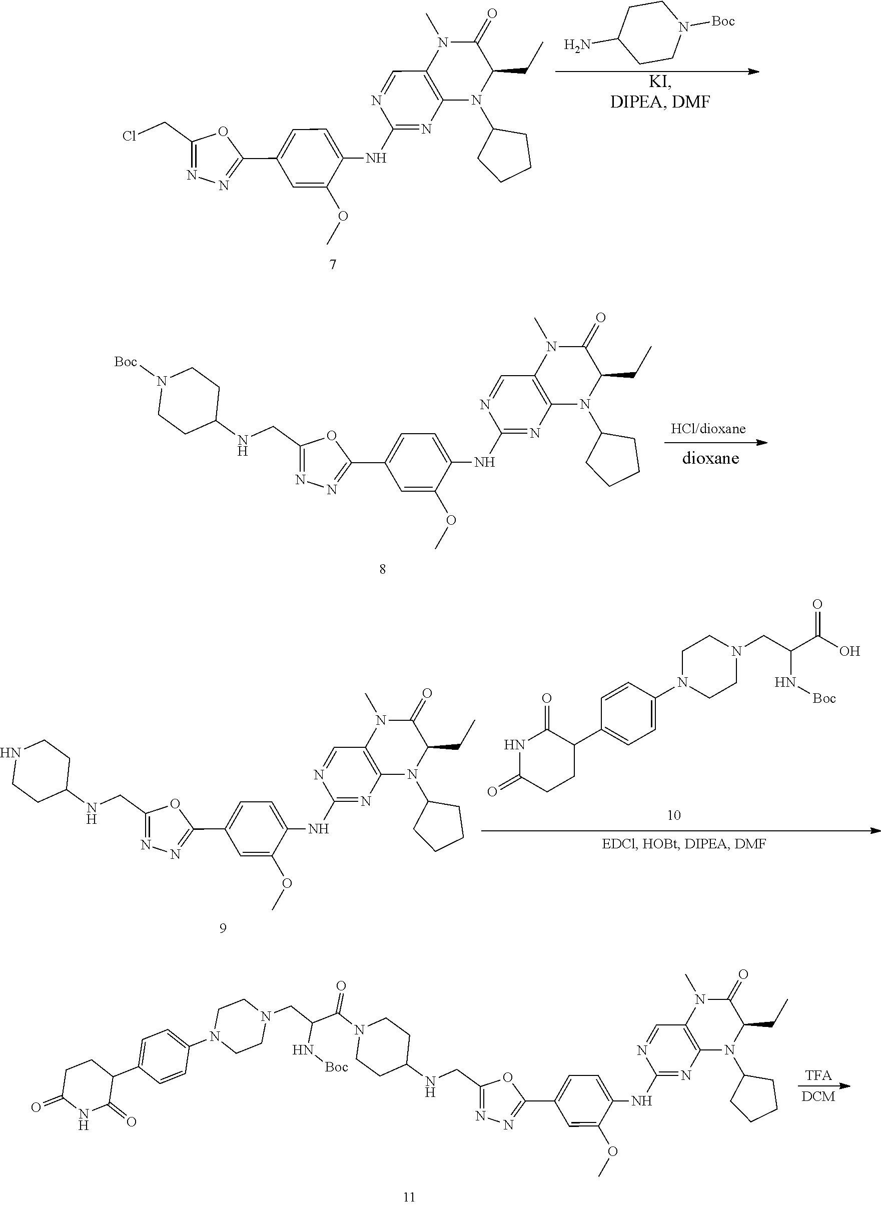

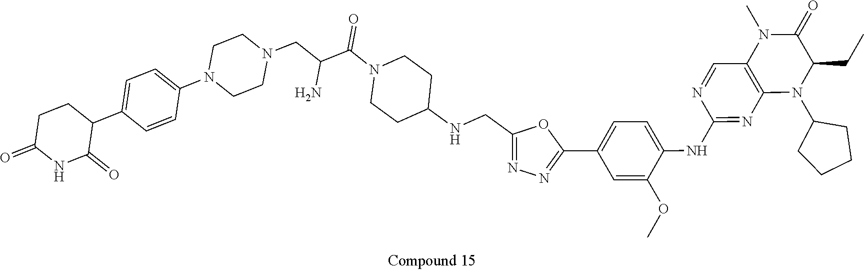 [SMILES: CC[C@@H]1C(=O)N(C)c2cnc(Nc3ccc(-c4nnc(CNC5CCN(C(=O)C(N)CN6CCN(c7ccc(C8CCC(=O)NC8=O)cc7)CC6)CC5)o4)cc3OC)nc2N1C1CCCC1]

### Step 1. Synthesis of (R)-4-((8-cyclopentyl-7-ethyl-5-methyl-6-oxo-5,6,7,8-tetrahydropteridin-2-yl)amino)-3-methoxybenzoic acid (3)

To a solution of (R)-2-chloro-8-cyclopentyl-7-ethyl-5-methyl-7,8-dihydropteridin-6(5H)-one (8 g, 27.14 mmol) in EtOH (100 mL) were added 4-amino-3-methoxybenzoic acid (4.4 g, 26.32 mmol) and a solution of HCl (12 M, 4.6 mL) in H₂O (400 mL) at 20° C. The mixture was stirred at 100° C. for 16 hr. LCMS showed a peak (50%) with the desired mass. The reaction mixture was stirred at 20° C. for 1 h, the mixture was filtered and the filter cake was dried under reduced pressure to afford (R)-4-((8-cyclopentyl-7-ethyl-5-methyl-6-oxo-5,6,7,8-tetrahydropteridin-2-yl)amino)-3-methoxybenzoic acid (6 g, crude) as a light yellow solid. MS(M+H)⁺=426.2

### Step 2. Synthesis of tert-butyl (R)-2-(4-((8-cyclopentyl-7-ethyl-5-methyl-6-oxo-5,6,7,8-tetrahydropteridin-2-yl)amino)-3-methoxybenzoyl)hydrazine-1-carboxylate (4)

To a solution of (R)-4-((8-cyclopentyl-7-ethyl-5-methyl-6-oxo-5,6,7,8-tetrahydropteridin-2-yl)amino)-3-methoxybenzoic acid (5 g, 11.75 mmol) in DMF (50 mL) were added HATU (6.75 g, 17.75 mmol), DIPEA (5.94 g, 45.93 mmol, 8 mL) and tert-butyl hydrazinecarboxylate (1.8 g, 13.62 mmol) at 20° C. The mixture was stirred at 20° C. for 16 h under N₂ atmosphere. LCMS showed a main peak with the desired mass. The reaction mixture was diluted with H₂O (100 mL), the mixture was extracted with EtOAc (150 mL×2). The combined organic layers were washed with brine (200 mL×3), dried over Na₂SO₄, filtered and concentrated under reduced pressure. The residue was triturated with solvent (EtOAc:MTBE=1:2; 90 mL) at 20° C. for 1 h. The mixture was filtered and the filter cake was dried under reduced pressure and afford tert-butyl (R)-2-(4-((8-cyclopentyl-7-ethyl-5-methyl-6-oxo-5,6,7,8-tetrahydropteridin-2-yl)amino)-3-methoxybenzoyl)hydrazine-1-carboxylate (5.2 g, 9.64 mmol, 82.00% yield) as a light yellow solid. MS(M+H)⁺=540.3

### Step 3. Synthesis of (R)-4-((8-cyclopentyl-7-ethyl-5-methyl-6-oxo-5,6,7,8-tetrahydropteridin-2-yl)amino)-3-methoxybenzohydrazide (5)

To a solution of tert-butyl (R)-2-(4-((8-cyclopentyl-7-ethyl-5-methyl-6-oxo-5,6,7,8-tetrahydropteridin-2-yl)amino)-3-methoxybenzoyl)hydrazine-1-carboxylate (2 g, 3.71 mmol) in DCM (15 mL) was added TFA (12.28 g, 107.70 mmol, 8 mL) at 20° C., The mixture was stirred at 20° C. for 2 hr. LCMS showed a main peak with desired mass. The reaction mixture was concentrated under reduced pressure to afford (R)-4-((8-cyclopentyl-7-ethyl-5-methyl-6-oxo-5,6,7,8-tetrahydropteridin-2-yl)amino)-3-methoxybenzohydrazide (2 g, crude, TFA) as a light yellow oil. MS(M+H)⁺=440.3

### Step 4. Synthesis of (R)—N′-(2-chloroacetyl)-4-((8-cyclopentyl-7-ethyl-5-methyl-6-oxo-5,6,7,8-tetrahydropteridin-2-yl)amino)-3-methoxybenzohydrazide (6)

To a solution of (R)-4-((8-cyclopentyl-7-ethyl-5-methyl-6-oxo-5,6,7,8-tetrahydropteridin-2-yl)amino)-3-methoxybenzohydrazide (2 g, crude, TFA) in DCM (40 mL) were added DIPEA (3.71 g, 28.71 mmol, 5 mL) and 2-chloroacetyl chloride (397.04 mg, 3.52 mmol, 280 μL) at 0° C. The mixture was stirred at 20° C. for 1 h under N₂ atmosphere. LCMS showed a peak (56%) with desired mass. The reaction mixture was diluted with H₂O (20 mL), the mixture was extracted with DCM (50 mL×2). The combined organic layers were washed with brine (60 mL×2), dried over Na₂SO₄, filtered and concentrated under reduced pressure. The residue was purified by flash silica gel chromatography (80 g SepaFlash® Silica Flash Column, Eluent of 75~100% EtOAc/Petroleum ether gradient @100 mL/min) to afford (R)—N′-(2-chloroacetyl)-4-((8-cyclopentyl-7-ethyl-5-methyl-6-oxo-5,6,7,8-tetrahydropteridin-2-yl)amino)-3-methoxybenzohydrazide (1.5 g, 2.91 mmol, 80.46% yield) as a light yellow oil. MS(M+H)⁺=516.2

### Step 5. Synthesis of (R)-2-((4-(5-(chloromethyl)-1,3,4-oxadiazol-2-yl)-2-methoxyphenyl)amino)-8-cyclopentyl-7-ethyl-5-methyl-7,8-dihydropteridin-6(5H)-one (7)

A solution of (R)—N′-(2-chloroacetyl)-4-((8-cyclopentyl-7-ethyl-5-methyl-6-oxo-5,6,7,8-tetrahydropteridin-2-yl)amino)-3-methoxybenzohydrazide (800 mg, 1.55 mmol) in POCl₃ (13.16 g, 85.83 mmol, 8.00 mL) was stirred at 80° C. for 32 h under N₂ atmosphere. LCMS showed (R)—N′-(2-chloroacetyl)-4-((8-cyclopentyl-7-ethyl-5-methyl-6-oxo-5,6,7,8-tetrahydropteridin-2-yl)amino)-3-methoxybenzohydrazide (7%) remained and a peak (65%) with the desired mass. The reaction mixture was concentrated under reduced pressure to remove the POCl₃. Then the crude product was diluted with DCM (30 mL), the organic layer was washed with NaHCO₃ (30 mL×2), dried over Na₂SO₄, filtered and concentrated under reduced pressure to afford (R)-2-((4-(5-(chloromethyl)-1,3,4-oxadiazol-2-yl)-2-methoxyphenyl)amino)-8-cyclopentyl-7-ethyl-5-methyl-7,8-dihydropteridin-6(5H)-one (770 mg, crude) as a yellow solid. MS(M+H)⁺=498.1

### Step 6. Synthesis of tert-butyl (R)-4-(((5-(4-((8-cyclopentyl-7-ethyl-5-methyl-6-oxo-5,6,7,8-tetrahydropteridin-2-yl)amino)-3-methoxyphenyl)-1,3,4-oxadiazol-2-yl)methyl)amino)piperidine-1-carboxylate

To a solution of (R)-2-((4-(5-(chloromethyl)-1,3,4-oxadiazol-2-yl)-2-methoxyphenyl)amino)-8-cyclopentyl-7-ethyl-5-methyl-7,8-dihydropteridin-6(5H)-one (200 mg, 401.63 μmol) in DMF (3 mL) were added tert-butyl 4-aminopiperidine-1-carboxylate (90 mg, 449.38 μmol), DIPEA (222.59 mg, 1.72 mmol, 299.99 μL) and KI (60.00 mg, 361.45 μmol) at 20° C. The mixture was stirred at 20° C. for 16 h under N₂ atmosphere. LCMS showed a peak with the desired mass. The reaction mixture was diluted with H₂O (10 mL), the mixture was extracted with EtOAc (25 mL×2). The combined organic layers were washed with brine (20 mL×3), dried over Na₂SO₄, filtered and concentrated under reduced pressure. The residue was purified by flash silica gel chromatography (10 g SepaFlash® Silica Flash Column, Eluent of 0~10% MeOH:EtOAc gradient, 100 mL/min) to afford tert-butyl (R)-4-(((5-(4-((8-cyclopentyl-7-ethyl-5-methyl-6-oxo-5,6,7,8-tetrahydropteridin-2-yl)amino)-3-methoxyphenyl)-1,3,4-oxadiazol-2-yl)methyl)amino)piperidine-1-carboxylate (300 mg, crude) as a light yellow solid. MS(M+H)⁺=662.4

### Step 7. Synthesis of (R)-8-cyclopentyl-7-ethyl-2-((2-methoxy-4-(5-((piperidin-4-ylamino)methyl)-1,3,4-oxadiazol-2-yl)phenyl)amino)-5-methyl-7,8-dihydropteridin-6(5H)-one (9)

To a solution of tert-butyl (R)-4-(((5-(4-((8-cyclopentyl-7-ethyl-5-methyl-6-oxo-5,6,7,8-tetrahydropteridin-2-yl)amino)-3-methoxyphenyl)-1,3,4-oxadiazol-2-yl)methyl)amino)piperidine-1-carboxylate (0.3 g, 453.31 μmol) in dioxane (4 mL) was added HCl/dioxane (4 M, 4 mL) at 20° C., The mixture was stirred at 20° C. for 2 h. LCMS showed a main peak with the desired mass. The reaction mixture was concentrated under reduced pressure to afford (R)-8-cyclopentyl-7-ethyl-2-((2-methoxy-4-(5-((piperidin-4-ylamino)methyl)-1,3,4-oxadiazol-2-yl)phenyl)amino)-5-methyl-7,8-dihydropteridin-6(5H)-one (270 mg, crude, HCl) as a light yellow solid. MS (M+H)⁺=562.3

### Step 8. Synthesis of tert-butyl (1-(4-(((5-(4-(((R)-8-cyclopentyl-7-ethyl-5-methyl-6-oxo-5,6,7,8-tetrahydropteridin-2-yl)amino)-3-methoxyphenyl)-1,3,4-oxadiazol-2-yl)methyl)amino)piperidin-1-yl)-3-(4-(4-(2,6-dioxopiperidin-3-yl)phenyl)piperazin-1-yl)-1-oxopropan-2-yl)carbamate (11)

To a solution of 2-((tert-butoxycarbonyl)amino)-3-(4-(4-(2,6-dioxopiperidin-3-yl)phenyl)piperazin-1-yl)propanoic acid (120 mg, 260.57 μmol) in DMF (2 mL) were added EDCI (100.00 mg, 521.65 μmol), HOBt (40.00 mg, 296.03 μmol), DIPEA (296.80 mg, 2.30 mmol, 400.00 μL) and (R)-8-cyclopentyl-7-ethyl-2-((2-methoxy-4-(5-((piperidin-4-ylamino)methyl)-1,3,4-oxadiazol-2-yl)phenyl)amino)-5-methyl-7,8-dihydropteridin-6(5H)-one (200.00 mg, crude, HCl) at 20° C. The mixture was stirred at 20° C. for 16 h under N₂ atmosphere. LCMS showed a peak (58%) with desired mass. The reaction mixture was diluted with H₂O (8 mL), the mixture was extracted with EtOAc (15 mL×2). The combined organic layers were washed with brine (10 mL×3), dried over Na₂SO₄, filtered and concentrated under reduced pressure. The residue was purified by prep-HPLC (column: Phenomene×luna C18 150×25 mmx 10 um; mobile phase: [water (TFA)-ACN]; gradient: 20%-50% B over 10 min; Column Temp: 30° C.) and re-purified by prep-HPLC (column: Waters Xbridge C18 150×50 mm×10 um; mobile phase: [water (NH₄HCO₃)-ACN]; gradient:35%-65% B over 10 min; Column Temp: 30° C.) to afford tert-butyl (1-(4-(((5-(4-(((R)-8-cyclopentyl-7-ethyl-5-methyl-6-oxo-5,6,7,8-tetrahydropteridin-2-yl)amino)-3-methoxyphenyl)-1,3,4-oxadiazol-2-yl)methyl)amino)piperidin-1-yl)-3-(4-(4-(2,6-dioxopiperidin-3-yl)phenyl)piperazin-1-yl)-1-oxopropan-2-yl)carbamate (210 mg, 209.13 μmol, 80.26% yield) as a light yellow solid. MS(M+H)⁺=1004.7

### Step 9. Synthesis of 3-(4-(4-(2-amino-3-(4-(((5-(4-(((R)-8-cyclopentyl-7-ethyl-5-methyl-6-oxo-5,6,7,8-tetrahydropteridin-2-yl)amino)-3-methoxyphenyl)-1,3,4-oxadiazol-2-yl)methyl)amino)piperidin-1-yl)-3-oxopropyl)piperazin-1-yl)phenyl)piperidine-2,6-dione (Compound 15)

To a solution of tert-butyl (1-(4-(((5-(4-(((R)-8-cyclopentyl-7-ethyl-5-methyl-6-oxo-5,6,7,8-tetrahydropteridin-2-yl)amino)-3-methoxyphenyl)-1,3,4-oxadiazol-2-yl)methyl)amino)piperidin-1-yl)-3-(4-(4-(2,6-dioxopiperidin-3-yl)phenyl)piperazin-1-yl)-1-oxopropan-2-yl)carbamate (170 mg, 169.29 μmol) in DCM (5 mL) was added TFA (6.14 g, 53.85 mmol, 4 mL) at 20° C., The mixture was stirred at 20° C. for 2 h. LCMS showed a main peak with desired mass. The reaction mixture was concentrated under reduced pressure. The residue was lyophilized to afford 3-(4-(4-(2-amino-3-(4-(((5-(4-(((R)-8-cyclopentyl-7-ethyl-5-methyl-6-oxo-5,6,7,8-tetrahydropteridin-2-yl)amino)-3-methoxyphenyl)-1,3,4-oxadiazol-2-yl)methyl)amino)piperidin-1-yl)-3-oxopropyl)piperazin-1-yl)phenyl)piperidine-2,6-dione (132.9 mg, crude, 3TFA) as a light yellow solid. MS(M+H)⁺=904.6

¹H NMR (400 MHz, DMSO-d₆) δ=10.78 (s, 1H), 9.33-9.22 (m, 1H), 8.25-8.07 (m, 2H), 7.86 (s, 1H), 7.68-7.60 (m, 2H), 7.12-7.03 (m, 2H), 6.97-6.89 (m, 2H), 4.81-4.71 (m, 4H), 4.61-4.42 (m, 5H), 4.24-4.17 (m, 1H), 4.03-3.92 (m, 4H), 3.78-3.69 (m, 1H), 3.62-3.51 (m, 1H), 3.26-3.14 (m, 7H), 2.83-2.56 (m, 7H), 2.49-2.42 (m, 1H), 2.28-2.06 (m, 3H), 1.98-1.74 (m, 6H), 1.60-1.43 (m, 5H), 0.77 (t, J=7.4 Hz, 3H).

### Example 16. Synthesis of 3-(4-(((1-((1r,4r)-4-(((5-(4-(((R)-8-cyclopentyl-7-ethyl-5-methyl-6-oxo-5,6,7,8-tetrahydropteridin-2-yl)amino)-3-methoxyphenyl)-1,3,4-oxadiazol-2-yl)methyl)amino) cyclohexyl)piperidin-4-yl)amino)methyl)phenyl)piperidine-2,6-dione (Compound 16)

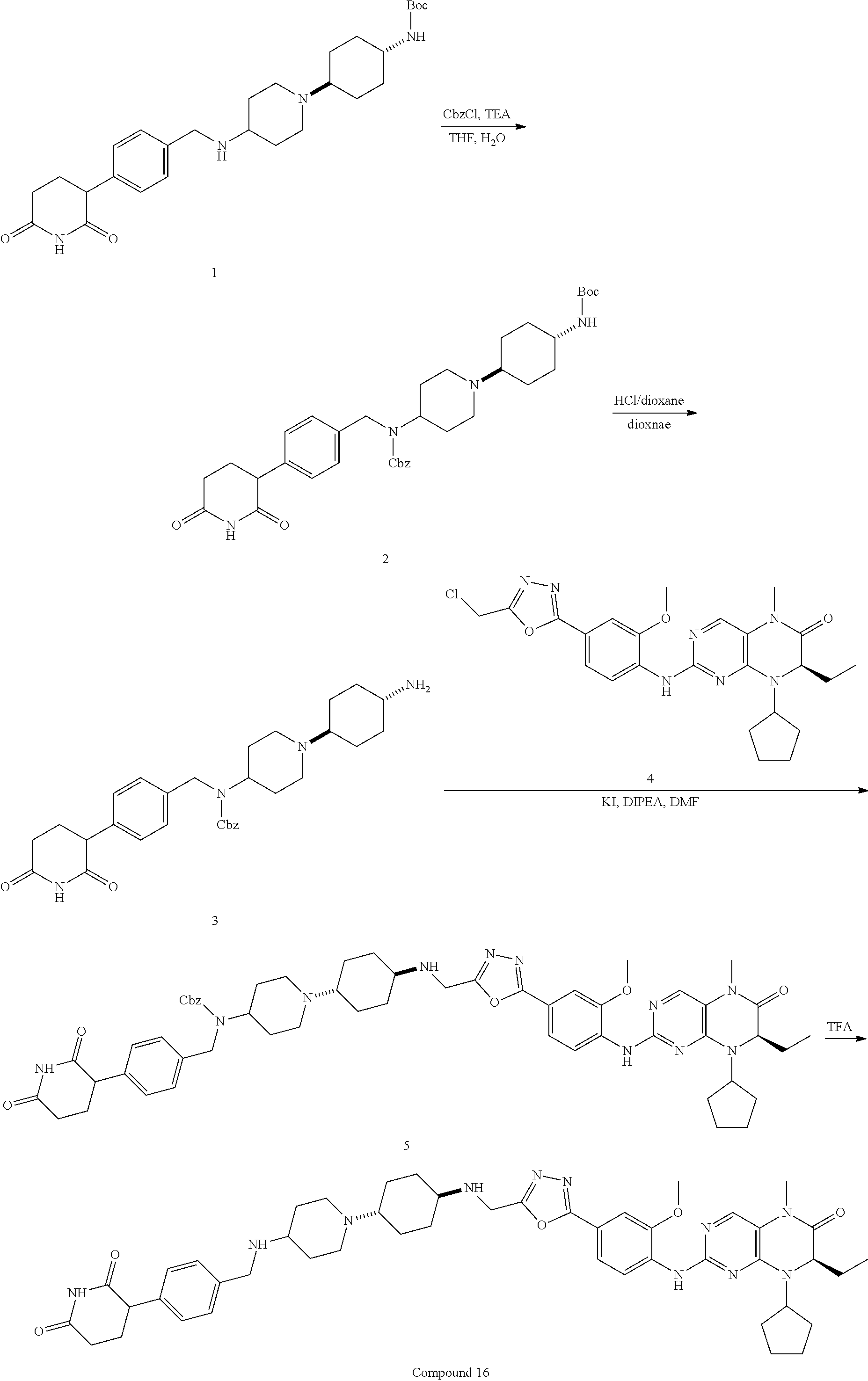

### Step 1. Synthesis of benzyl (1-((1r,4r)-4-((tert-butoxycarbonyl)amino)cyclohexyl)piperidin-4-yl)(4-(2,6-dioxopiperidin-3-yl)benzyl)carbamate (2)

To a solution of tert-butyl ((1r,4r)-4-(4-((4-(2,6-dioxopiperidin-3-yl)benzyl)amino)piperidin-1-yl)cyclohexyl)carb amate (500 mg, 1.00 mmol) in THF (5 mL) and H₂O (5 mL) was added TEA (304.39 mg, 3.01 mmol, 418.69 L). The reaction mixture was cooled to 0° C. Then CbzCl (342.10 mg, 2.01 mmol, 286.28 μL) was added slowly and the resulting mixture was stirred at 25° C. for 16 hr. LCMS showed a peak (~25%) with desired mass. The reaction mixture was diluted with water (20 mL) and extracted with EtOAc (30 mL×3). The combined organic layers were washed with water (30 mL), dried over Na₂SO₄, filtered. The filtrate was concentrated under reduced pressure to give a residue. The residue was purified by flash silica gel chromatography (ISCO®; 20 g SepaFlash® Silica Flash Column, Eluent of 30~40% MeOH/EtOAc gradient @80 mL/min) to afford benzyl (1-((1r,4r)-4-((tert-butoxycarbonyl)amino)cyclohexyl)piperidin-4-yl)(4-(2,6-dioxopiperidin-3-yl)benzyl)carbamate (410 mg, 647.93 μmol, 64.62% yield) as a yellow oil. MS(M+H)⁺=633.4.

### Step 2. Synthesis of benzyl (1-((1r,4r)-4-aminocyclohexyl)piperidin-4-yl)(4-(2,6-dioxopiperidin-3-yl)benzyl)carbamate (3)

To a solution of afford benzyl (1-((1r,4r)-4-((tert-butoxycarbonyl)amino)cyclohexyl)piperidin-4-yl)(4-(2,6-dioxopiperidin-3-yl)benzyl)carbamate (620 mg, 979.79 μmol) in dioxane (6 mL) was added HCl/dioxane (4 M, 6 mL). The mixture was stirred at 25° C. for 2 hr. LCMS showed benzyl (1-((1r,4r)-4-((tert-butoxycarbonyl)amino)cyclohexyl)piperidin-4-yl)(4-(2,6-dioxopiperidin-3-yl)benzyl)carbamate was consumed completely and one main peak with desired mass. The reaction mixture was concentrated under reduced pressure to afford benzyl (1-((1r,4r)-4-aminocyclohexyl)piperidin-4-yl)(4-(2,6-dioxopiperidin-3-yl)benzyl)carbamate (600 mg, crude, HCl salt) as a yellow solid. MS(M+H)⁺=533.4.

### Step 3. Synthesis of benzyl (1-((1r,4r)-4-(((5-(4-(((R)-8-cyclopentyl-7-ethyl-5-methyl-6-oxo-5,6,7,8-tetrahydropteridin-2-yl)amino)-3-methoxyphenyl)-1,3,4-oxadiazol-2-yl)methyl)amino)cyclohexyl)piperidin-4-yl)(4-(2,6-dioxopiperidin-3-yl)benzyl)carbamate (5)

To a solution of benzyl (1-((1r,4r)-4-aminocyclohexyl)piperidin-4-yl)(4-(2,6-dioxopiperidin-3-yl)benzyl)carbamate (300 mg, 527.12 μmol, HCl salt) and (R)-2-((4-(5-(chloromethyl)-1,3,4-oxadiazol-2-yl)-2-methoxyphenyl)amino)-8-cyclopentyl-7-ethyl-5-methyl-7,8-dihydropteridin-6(5H)-one (262.49 mg, 527.12 μmol) in DMF (5 mL) were added KI (8.75 mg, 52.71 μmol) and DIPEA (204.37 mg, 1.58 mmol, 275.44 μL). The mixture was stirred at 40° C. for 20 hr. LCMS showed a peak (~54%) with desired mass. The residue was diluted with water (20 mL) and extracted with EtOAc (20 mL×3). The combined organic layers were washed with brine (20 mL×3), dried over Na₂SO₄, filtered. The filtrate was concentrated under reduced pressure. The residue was purified by column chromatography by prep-TLC (SiO₂, EtOAc:MeOH=2:1) to afford benzyl (1-((1r,4r)-4-(((5-(4-(((R)-8-cyclopentyl-7-ethyl-5-methyl-6-oxo-5,6,7,8-tetrahydropteridin-2-yl)amino)-3-methoxyphenyl)-1,3,4-oxadiazol-2-yl)methyl)amino)cyclohexyl)piperidin-4-yl)(4-(2,6-dioxopiperidin-3-yl)benzyl)carbamate (125 mg, 125.73 μmol, 23.85% yield) as a yellow solid. MS(M+H)⁺=994.6.

### Step 4. Synthesis of 3-(4-(((1-((1r,4r)-4-(((5-(4-(((R)-8-cyclopentyl-7-ethyl-5-methyl-6-oxo-5,6,7,8-tetrahydropteridin-2-yl)amino)-3-methoxyphenyl-1,3,4-oxadiazol-2-yl)methyl)amino)cyclohexyl)piperidin-4-yl)amino)methyl)phenyl)piperidine-2,6-dione (Compound 16)

To a solution of benzyl (1-((1r,4r)-4-(((5-(4-(((R)-8-cyclopentyl-7-ethyl-5-methyl-6-oxo-5,6,7,8-tetrahydropteridin-2-yl)amino)-3-methoxyphenyl)-1,3,4-oxadiazol-2-yl)methyl)amino)cyclohexyl)piperidin-4-yl)(4-(2,6-dioxopiperidin-3-yl)benzyl)carbamate (125 mg, 125.73 μmol) was added TFA (4.61 g, 40.39 mmol, 3 mL). The mixture was stirred at 60° C. for 16 hr. LCMS showed one main peak with desired mass. The reaction mixture was concentrated under reduced pressure. The residue was purified by prep-HPLC (column: Phenomenex luna C18 150×40 mm×15 μm; mobile phase: [water (TFA)-ACN]; gradient: 10%-40% B over 10 min) the eluent was lyophilized to afford 3-(4-(((1-((1r,4r)-4-(((5-(4-(((R)-8-cyclopentyl-7-ethyl-5-methyl-6-oxo-5,6,7,8-tetrahydropteridin-2-yl)amino)-3-methoxyphenyl)-1,3,4-oxadiazol-2-yl)methyl)amino)cyclohexyl)piperidin-4-yl)amino)methyl)phenyl)piperidine-2,6-dione (113.3 mg, 88.31 μmol, 70.24% yield, 93.7% purity, 3TFA salt) as a white solid. MS(M+H)⁺=860.5.

¹H NMR (400 MHz, CD₃CN) δ=10.19-10.07 (m, 1H), 8.84 (br s, 1H), 7.97 (d, J=8.8 Hz, 1H), 7.65-7.61 (m, 2H), 7.49-7.44 (m, 3H), 7.27 (d, J=8.2 Hz, 2H), 4.48 (s, 2H), 4.33 (dd, J=3.4, 6.7 Hz, 1H), 4.20-4.13 (m, 3H), 3.93 (s, 3H), 3.82 (dd, J=5.3, 11.3 Hz, 1H), 3.60-3.54 (m, 2H), 3.49-3.44 (m, 1H), 3.22 (s, 3H), 3.18-3.16 (m, 1H), 2.99-2.95 (m, 2H), 2.66-2.59 (m, 4H), 2.43-2.37 (m, 3H), 2.34-2.28 (m, 3H), 2.26-2.22 (m, 2H), 2.20-2.15 (m, 4H), 1.87-1.76 (m, 4H), 1.63-1.55 (m, 4H), 1.53-1.40 (m, 5H), 0.80 (t, J=7.5 Hz, 3H).

### Example 17. Synthesis of 3-(4-(4-((((1r,4r)-4-(((5-(4-(((R)-8-cyclopentyl-7-ethyl-5-methyl-6-oxo-5,6,7,8-tetrahydropteridin-2-yl)amino)-3-methoxyphenyl)-1,3,4-oxadiazol-2-yl)methyl)amino) cyclohexyl)amino)methyl)piperidin-1-yl)-3-fluorophenyl)piperidine-2,6-dione (Compound 17)

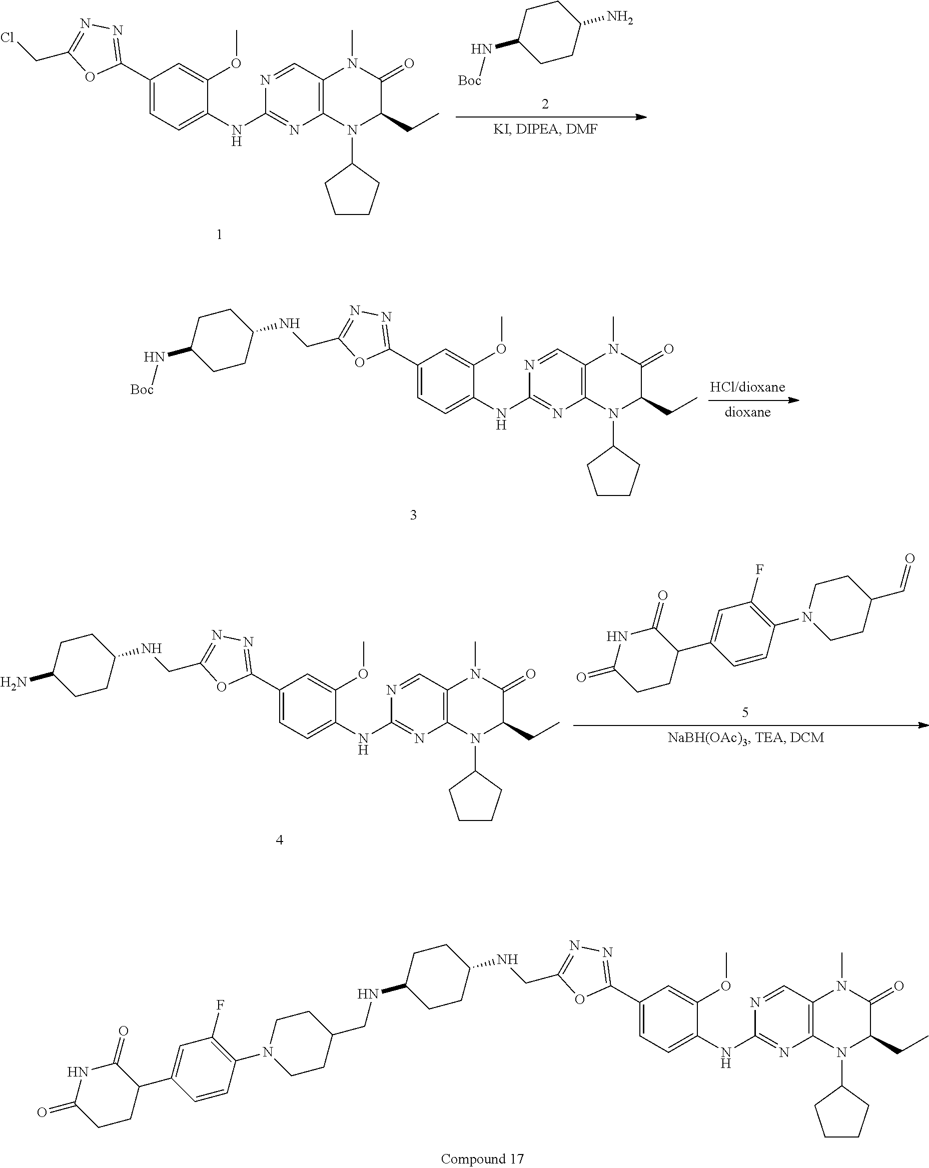

### Step 1. Synthesis of tert-butyl ((1r,4r)-4-(((5-(4-(((R)-8-cyclopentyl-7-ethyl-5-methyl-6-oxo-5,6,7,8-tetrahydropteridin-2-yl)amino)-3-methoxyphenyl)-1,3,4-oxadiazol-2-yl)methyl)amino)cyclohexyl)carbamate (3′)

To a solution of (R)-2-((4-(5-(chloromethyl)-1,3,4-oxadiazol-2-yl)-2-methoxyphenyl)amino)-8-cyclopentyl-7-ethyl-5-methyl-7,8-dihydropteridin-6(5H)-one (200 mg, 401.63 μmol) in DMF (4 mL) were added tert-butyl ((1r,4r)-4-aminocyclohexyl)carbamate (120 mg, 478.54 mol, HCl), DIPEA (267.12 mg, 2.07 mmol, 360.00 L) and KI (60.00 mg, 361.44 mol) at 20° C. The mixture was stirred at 20° C. for 20 hr under N₂ atmosphere. LCMS showed a peak (77%) with the desired mass. The reaction mixture was diluted with H₂O (10 mL), the mixture was extracted with EtOAc (30 mL×2). The combined organic layers were washed with brine (20 mL×3), dried over Na₂SO₄, filtered and concentrated under reduced pressure. The residue was purified by flash silica gel chromatography (Biotage; 10 g SepaFlash® Silica Flash Column, Eluent of 0~10% MeOH:EtOAc gradient, 100 mL/min) to afford tert-butyl ((1r,4r)-4-(((5-(4-(((R)-8-cyclopentyl-7-ethyl-5-methyl-6-oxo-5,6,7,8-tetrahydropteridin-2-yl)amino)-3-methoxyphenyl)-1,3,4-oxadiazol-2-yl)methyl)amino)cyclohexyl)carbamate (300 mg, crude) as a light yellow solid. MS(M+H)⁺=676.4

### Step 2. Synthesis of (R)-2-((4-(5-((((1r,4r)-4-aminocyclohexyl)amino)methyl)-1,3,4-oxadiazol-2-yl)-2-methoxyphenyl)amino)-8-cyclopentyl-7-ethyl-5-methyl-7,8-dihydropteridin-6(5H)-one (4)

To a solution of tert-butyl ((1r,4r)-4-(((5-(4-(((R)-8-cyclopentyl-7-ethyl-5-methyl-6-oxo-5,6,7,8-tetrahydropteridin-2-yl)amino)-3-methoxyphenyl)-1,3,4-oxadiazol-2-yl)methyl)amino)cyclohexyl)carbamate (300 mg, crude) in dioxane (5 mL) was added HCl/dioxane (4 M, 8 mL) at 20° C., The mixture was stirred at 20° C. for 2 hr. LCMS showed a main peak with the desired mass. The reaction mixture was concentrated under reduced pressure to afford (R)-2-((4-(5-((((1r,4r)-4-aminocyclohexyl)amino)methyl)-1,3,4-oxadiazol-2-yl)-2-methoxyphenyl)amino)-8-cyclopentyl-7-ethyl-5-methyl-7,8-dihydropteridin-6(5H)-one (270 mg, crude, HCl) as a light yellow solid. MS(M+H)⁺=576.3

### Step 3. Synthesis of 3-(4-(4-((((1r,4r)-4-(((5-(4-(((R)-8-cyclopentyl-7-ethyl-5-methyl-6-oxo-5,6,7,8-tetrahydropteridin-2-yl)amino)-3-methoxyphenyl)-1,3,4-oxadiazol-2-yl)methyl)amino)cyclohexyl)amino)methyl)piperidin-1-yl)-3-fluorophenyl)piperidine-2,6-dione (Compound 17)

To a solution of 1-(4-(2,6-dioxopiperidin-3-yl)-2-fluorophenyl)piperidine-4-carbaldehyde (120 mg, 376.95 μmol) in DCM (4 mL) were added (R)-2-((4-(5-((((1r,4r)-4-aminocyclohexyl)amino)methyl)-1,3,4-oxadiazol-2-yl)-2-methoxyphenyl)amino)-8-cyclopentyl-7-ethyl-5-methyl-7,8-dihydropteridin-6(5H)-one (210.00 mg, 343.05 μmol, HCl) and TEA (228.86 mg, 2.26 mmol, 314.80 μL) at 20° C., the mixture was stirred at 20° C. for 1 h. Then NaBH(OAc)₃ (300.00 mg, 1.42 mmol) was added to the mixture and the resulting mixture was stirred at 20° C. for 15 h. LCMS showed a peak (43%) with desired mass. To the reaction mixture was added NaHCO₃ (sat. aq, 4 mL) to adjust the water layers pH=9~10 at 0° C., then extracted with DCM (15 mL×2), then the combined organic layers were washed with brine (20 mL×2), dried over Na₂SO₄, filtered and concentrated under reduced pressure. The residue was purified by prep-HPLC (column: Phenomenex luna C18 150×25 mm×10 um; mobile phase: [water (TFA)-ACN]; gradient: 16%-46% B over 10 min; Column Temp: 30° C.). The eluent was lyophilized to afford 3-(4-(4-((((1r,4r)-4-(((5-(4-(((R)-8-cyclopentyl-7-ethyl-5-methyl-6-oxo-5,6,7,8-tetrahydropteridin-2-yl)amino)-3-methoxyphenyl)-1,3,4-oxadiazol-2-yl)methyl)amino)cyclohexyl)amino)methyl)piperidin-1-yl)-3-fluorophenyl)piperidine-2,6-dione (123.7 mg, 99.25 μmol, 26.33% yield, 97.9% purity, 3TFA) as a light yellow solid. MS(M+H)⁺=878.6

¹H NMR (400 MHz, DMSO-d₆) δ=10.77 (s, 1H), 9.96-9.78 (m, 1H), 9.10-8.95 (m, 1H), 8.59-8.47 (m, 1H), 8.25 (d, J=8.3 Hz, 1H), 7.86 (s, 1H), 7.68-7.61 (m, 2H), 7.05-6.94 (m, 3H), 4.73 (s, 2H), 4.46-4.38 (m, 1H), 4.28-4.18 (m, 1H), 3.98 (s, 3H), 3.85-3.76 (m, 1H), 3.42-3.33 (m, 2H), 3.25 (s, 2H), 3.12-3.03 (m, 1H), 2.99-2.83 (m, 2H), 2.72-2.62 (m, 2H), 2.50-2.49 (m, 3H), 2.30-2.13 (m, 5H), 2.02-1.73 (m, 10H), 1.66-1.33 (m, 11H), 0.77 (t, J=7.5 Hz, 3H).

### Example 18. Synthesis of 4-(((R)-8-cyclopentyl-7-ethyl-5-methyl-6-oxo-5,6,7,8-tetrahydropteridin-2-yl)amino)-N-((1r,4R)-4-(((1-(4-(2,6-dioxopiperidin-3-yl)-2-fluorophenyl)piperidin-4-yl)methyl)amino)cyclohexyl)-3-methoxybenzamide (Compound 18)

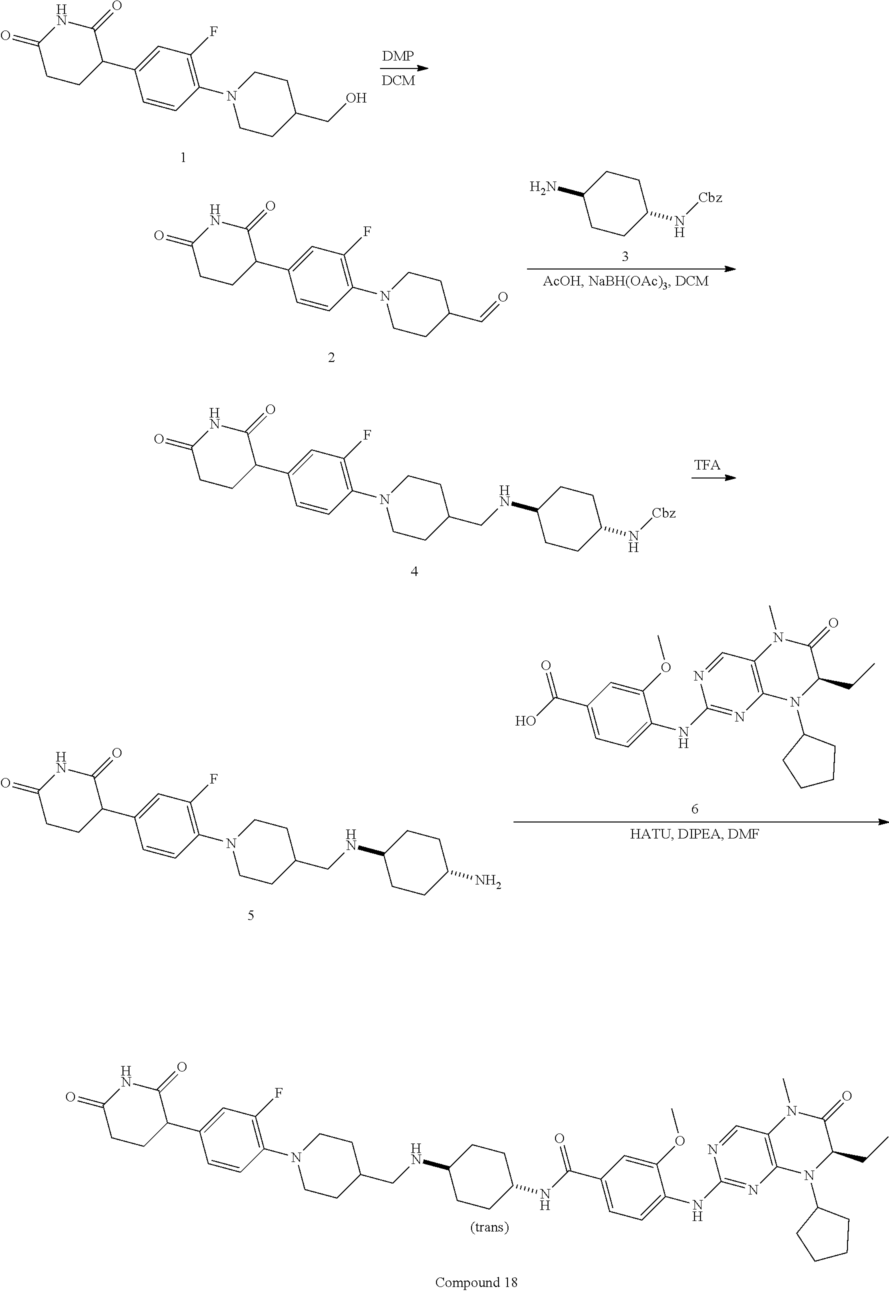

### Step 1. Synthesis of 1-(4-(2,6-dioxopiperidin-3-yl)-2-fluorophenyl)piperidine-4-carbaldehyde (2)

To a solution of 3-(3-fluoro-4-(4-(hydroxymethyl)piperidin-1-yl)phenyl)piperidine-2,6-dione (2 g, 6.24 mmol) in DCM (50 mL) was added DMP (3.97 g, 9.36 mmol) and the mixture was stirred at 20° C. for 2 h. TLC (Petroleum ether/EtOAc=1/1) showed 3-(3-fluoro-4-(4-(hydroxymethyl)piperidin-1-yl)phenyl)piperidine-2,6-dione was consumed completely and new spot was formed. The mixture was filtered and the filtrate was concentrated under reduced pressure to afford 1-(4-(2,6-dioxopiperidin-3-yl)-2-fluorophenyl)piperidine-4-carbaldehyde (2 g, crude) as yellow oil. MS(M+H)⁺=319.4

### Step 2. Synthesis of benzyl ((1r,4r)-4-(((1-(4-(2,6-dioxopiperidin-3-yl)-2-fluorophenyl)piperidin-4-yl)methyl)amino)cyclohexyl)carbamate (4)

To the solution of 1-(4-(2,6-dioxopiperidin-3-yl)-2-fluorophenyl)piperidine-4-carbaldehyde (2 g, 6.28 mmol) and benzyl ((1r,4r)-4-aminocyclohexyl)carbamate (1.56 g, 6.28 mmol) in DCM (20 mL) was added AcOH (377.28 mg, 6.28 mmol, 359.66 L) and the mixture was stirred at 20° C. for 1 h, then NaBH(OAc)₃ (1.60 g, 7.54 mmol) was added and the resulting mixture was stirred at 20° C. for 12 h. LCMS showed a peak (43%) with desired mass. The reaction mixture was diluted with water (100 mL) and extracted with DCM (50 mL×3). The combined organic layers were washed with saturated brine (200 mL), dried over anhydrous Na₂SO₄, filtered and concentrated under reduced pressure. The residue was purified by prep-HPLC (column: Phenomenex luna C18 150*40 mm*15 um; mobile phase: [water (TFA)-ACN]; gradient: 15%-45% B over 10 min) to afford benzyl ((1r,4r)-4-(((1-(4-(2,6-dioxopiperidin-3-yl)-2-fluorophenyl)piperidin-4-yl)methyl)amino)cyclohexyl)carbamate (0.9 g, 1.57 mmol, 24.97% yield, 96% purity) as yellow oil. MS(M+H)⁺=551.4

### Step 3. Synthesis of 3-(4-(4-((((1r,4r)-4-aminocyclohexyl)amino)methyl)piperidin-1-yl)-3-fluorophenyl)piperidine-2,6-dione (5)

The solution of benzyl ((1r,4r)-4-(((1-(4-(2,6-dioxopiperidin-3-yl)-2-fluorophenyl)piperidin-4-yl)methyl)amino)cyclohexyl)carbamate (450 mg, 817.20 μmol) in TFA (7.68 g, 67.31 mmol, 5 mL) was stirred at 40° C. for 3 h. LCMS showed a peak (79%) with desired mass. The mixture was concentrated under reduced pressure to afford 3-(4-(4-((((1r,4r)-4-aminocyclohexyl)amino)methyl)piperidin-1-yl)-3-fluorophenyl)piperidine-2,6-dione (0.4 g, 753.93 μmol, 92.26% yield, TFA) as yellow oil. MS(M+H)⁺=417.3

### Step 4. Synthesis of 4-(((R)-8-cyclopentyl-7-ethyl-5-methyl-6-oxo-5,6,7,8-tetrahydropteridin-2-yl)amino)-N-((1r,4R)-4-(((1-(4-(2,6-dioxopiperidin-3-yl)-2-fluorophenyl))piperidin-4-yl)methyl)amino)cyclohexyl)-3-methoxybenzamide (Compound 18)

To the solution of (R)-4-((8-cyclopentyl-7-ethyl-5-methyl-6-oxo-5,6,7,8-tetrahydropteridin-2-yl)amino)-3-methoxybenzoic acid (160.39 mg, 376.96 μmol) in DMF (5 mL) were added HATU (172.00 mg, 452.36 μmol) and DIPEA (194.88 mg, 1.51 mmol, 262.64 μL) and the mixture was stirred at 20° C. for 1 h, 3-(4-(4-((((1r,4r)-4-aminocyclohexyl)amino)methyl)piperidin-1-yl)-3-fluorophenyl)piperidine-2,6-dione (0.2 g, 376.96 μmol, TFA) was added and the mixture was stirred at 20° C. for 12 h. LCMS showed a peak (60%) with desired mass. The reaction mixture was diluted with water (50 mL) and extracted with EtOAc (30 mL×3). The combined organic layers were washed with saturated brine (100 mL), dried over anhydrous Na₂SO₄, filtered and concentrated under reduced pressure. The residue was purified by prep-HPLC (column: Phenomenex luna C18 150*40 mm*15 um; mobile phase: [water (TFA)-ACN]; gradient:15%-45% B over 10 min) and the eluent was adjusted pH=9 with K₂CO₃ solid, the resulting mixture was extracted with EtOAc (30 mL×3). The combined organic layers were washed with saturated brine (100 mL), dried over anhydrous Na₂SO₄, filtered and concentrated, the residue was lyophilized to afford 4-(((R)-8-cyclopentyl-7-ethyl-5-methyl-6-oxo-5,6,7,8-tetrahydropteridin-2-yl)amino)-N-((1r,4R)-4-(((1-(4-(2,6-dioxopiperidin-3-yl)-2-fluorophenyl)piperidin-4-yl)methyl)amino)cyclohexyl)-3-methoxybenzamide (121.3 mg, 139.85 μmol, 37.10% yield, 95% purity) as a white solid. MS(M+H)⁺=824.5.

¹H NMR (400 MHz, DMSO-d₆) δ=10.81 (s, 1H), 8.47-8.37 (m, 1H), 8.04 (br d, J=7.5 Hz, 1H), 7.85 (s, 1H), 7.60 (s, 1H), 7.54-7.43 (m, 2H), 7.08-6.89 (m, 3H), 4.41-4.31 (m, 1H), 4.24 (br dd, J=3.4, 7.3 Hz, 1H), 3.94 (s, 3H), 3.81-3.75 (m, 2H), 3.25 (s, 3H), 2.75-2.56 (m, 4H), 2.52-2.45 (m, 4H), 2.41-2.33 (m, 1H), 2.24-2.15 (m, 1H), 2.06-1.74 (m, 15H), 1.69-1.59 (m, 3H), 1.50-1.25 (m, 5H), 1.19-1.02 (m, 2H), 0.77 (br t, J=7.4 Hz, 3H).

### Example 19. Synthesis of N-(4-(1-amino-2-(4-(4-(2,6-dioxopiperidin-3-yl)-2-fluorophenyl)piperazin-1-yl)ethyl) piperidin-1-yl)-4-(((R)-8-cyclopentyl-7-ethyl-5-methyl-6-oxo-5,6,7,8-tetrahydropteridin-2-yl)amino)-3-methoxybenzamide (Compound 19)

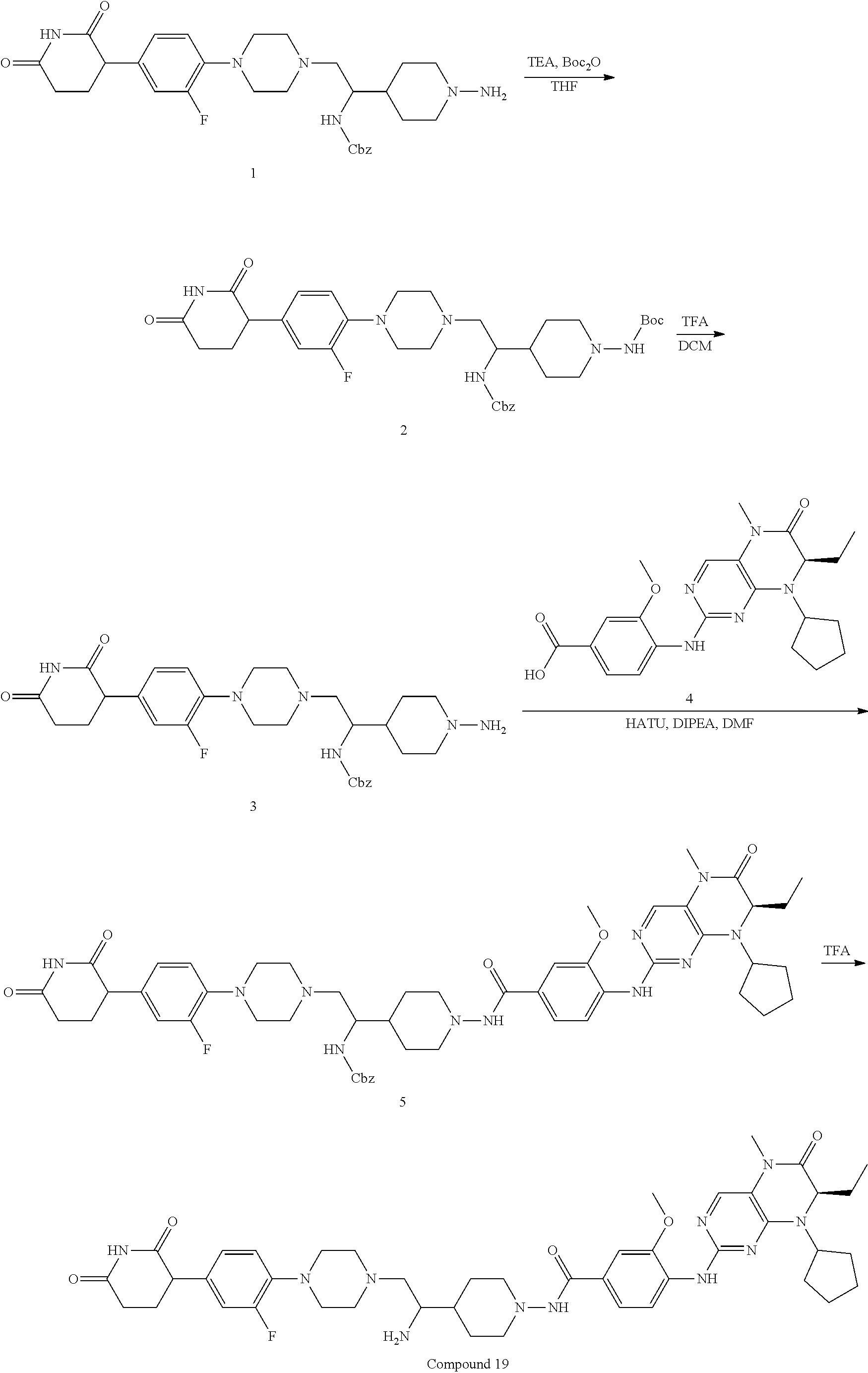

### Step 1. Synthesis of benzyl (1-(1-((tert-butoxycarbonyl)amino)piperidin-4-yl)-2-(4-(4-(2,6-dioxopiperidin-3-yl)-2-fluorophenyl)piperazin-1-yl))ethyl)carbamate (2)

To a solution of benzyl (1-(1-aminopiperidin-4-yl)-2-(4-(4-(2,6-dioxopiperidin-3-yl)-2-fluorophenyl)piperazin-1-yl)ethyl)carbamate (170 mg, 300.00 μmol) in THF (3 mL) were added TEA (212.50 mg, 2.10 mmol, 292.29 L) and Boc₂O (196.42 mg, 900.00 μmol, 206.76 L) and the mixture was stirred at 25° C. for 12 h. LCMS showed 38% of the desired mass. The mixture was diluted with water (10 mL) and extracted with EtOAc (10 mL×3), the combined organic layer was washed with water (10 mL), dried over Na₂SO₄ and filtered. The filtrate was concentrated under reduced pressure. The residue was purified by prep-TLC (DCM:MeOH=20:1) to afford benzyl (1-(1-((tert-butoxycarbonyl)amino)piperidin-4-yl)-2-(4-(4-(2,6-dioxopiperidin-3-yl)-2-fluorophenyl)piperazin-1-yl)ethyl)carbamate (40 mg, 59.99 μmol, 20.00% yield) as a yellow solid. MS(M+H)⁺=667.4

### Step 2. Synthesis of benzyl (1-(1-aminopiperidin-4-yl)-2-(4-(4-(2,6-dioxopiperidin-3-yl)-2-fluorophenyl)piperazin-1-yl)ethyl)carbamate (3)

To a solution of benzyl (1-(1-((tert-butoxycarbonyl)amino)piperidin-4-yl)-2-(4-(4-(2,6-dioxopiperidin-3-yl)-2-fluorophenyl)piperazin-1-yl)ethyl)carbamate (40 mg, 59.99 μmol) in DCM (0.5 mL) was added TFA (122.80 mg, 1.08 mmol, 80 L) and the mixture was stirred at 25° C. for 1 h. LCMS showed 82% of the desired mass. The mixture was concentrated under reduced pressure to afford benzyl (1-(1-aminopiperidin-4-yl)-2-(4-(4-(2,6-dioxopiperidin-3-yl)-2-fluorophenyl)piperazin-1-yl)ethyl)carbamate (40 mg, crude, TFA) as a yellow oil. MS(M+H)⁺=567.4

### Step 3. Synthesis of benzyl (1-(1-(4-(((R)-8-cyclopentyl-7-ethyl-5-methyl-6-oxo-5,6,7,8-tetrahydropteridin-2-yl)amino)-3-methoxybenzamido)piperidin-4-yl)-2-(4-(4-(2,6-dioxopiperidin-3-yl)-2-fluoro phenyl)piperazin-1-yl)ethyl)carbamate (5)

To a solution of (R)-4-((8-cyclopentyl-7-ethyl-5-methyl-6-oxo-5,6,7,8-tetrahydropteridin-2-yl)amino)-3-methoxybenzoic acid (30 mg, 70.51 μmol) and HATU (27 mg, 71.01 μmol) in DMF (1 mL) was added DIPEA (74.20 mg, 574.11 μmol, 0.1 mL) and the mixture was stirred at 25° C. for 15 min. Then a solution of benzyl (1-(1-aminopiperidin-4-yl)-2-(4-(4-(2,6-dioxopiperidin-3-yl)-2-fluorophenyl)piperazin-1-yl)ethyl)carbamate (40 mg, crude, TFA) was added at 0° C. and the mixture was stirred at 25° C. for 1 h. LCMS showed 36% of the desired mass. The mixture was diluted with water (10 mL) and extracted with EtOAc (10 mL×3), the combined organic layer was washed with brine (10 mL), dried over Na₂SO₄ and filtered. The filtrate was concentrated under reduced pressure. The residue was purified by prep-TLC (DCM:MeOH=10:1) to afford benzyl (1-(1-(4-(((R)-8-cyclopentyl-7-ethyl-5-methyl-6-oxo-5,6,7,8-tetrahydropteridin-2-yl)amino)-3-methoxybenzamido)piperidin-4-yl)-2-(4-(4-(2,6-dioxopiperidin-3-yl)-2-fluoro phenyl)piperazin-1-yl)ethyl)carbamate (40 mg, 40.24 μmol, 68.48% yield, 98% purity) as a yellow solid. MS(M+H)⁺=974.5

### Step 4. Synthesis of N-(4-(1-amino-2-(4-(4-(2,6-dioxopiperidin-3-yl)-2-fluorophenyl)piperazin-1-yl)ethyl)piperidin-1-yl)-4-(((R)-8-cyclopentyl-7-ethyl-5-methyl-6-oxo-5,6,7,8-tetrahydropteridin-2-ylamino)-3-methoxybenzamide (Compound 19)

A solution of benzyl (1-(1-(4-(((R)-8-cyclopentyl-7-ethyl-5-methyl-6-oxo-5,6,7,8-tetrahydropteridin-2-yl)amino)-3-methoxybenzamido)piperidin-4-yl)-2-(4-(4-(2,6-dioxopiperidin-3-yl)-2-fluoro phenyl)piperazin-1-yl)ethyl)carbamate (120 mg, 123.19 μmol) in TFA (7.68 g, 67.31 mmol, 5 mL) was stirred at 60° C. for 4.5 h. LCMS showed trace of the starting material remained and 52% the desired mass. The mixture was concentrated under reduced pressure and then purified by prep-HPLC (column: Phenomenex Luna C18 150*25 mm*10 um; mobile phase: [water (TFA)-ACN]; gradient: 17%-47% B over 10 min), the eluent was lyophilized to afford N-(4-(1-amino-2-(4-(4-(2,6-dioxopiperidin-3-yl)-2-fluorophenyl)piperazin-1-yl)ethyl)piperidin-1-yl)-4-(((R)-8-cyclopentyl-7-ethyl-5-methyl-6-oxo-5,6,7,8-tetrahydropteridin-2-yl)amino)-3-methoxybenzamide (37.3 mg, 30.92 μmol, 25.10% yield, 98% purity, 3TFA) as a white solid. MS(M+H)⁺=840.5

¹H NMR (400 MHz, DMSO-d₆) δ=10.82 (br s, 1H), 9.51 (br s, 1H), 9.24-9.01 (m, 1H), 7.99-7.91 (m, 1H), 7.79 (br s, 1H), 7.55-7.38 (m, 2H), 7.16-6.92 (m, 3H), 4.46-4.38 (m, 1H), 4.23-4.14 (m, 1H), 3.90 (s, 3H), 3.85-3.79 (m, 1H), 3.48-3.34 (m, 6H), 3.22 (s, 3H), 3.18-3.02 (m, 6H), 2.83-2.73 (m, 2H), 2.70-2.60 (m, 1H), 2.55-2.53 (m, 3H), 2.28-2.13 (m, 1H), 2.05-1.69 (m, 10H), 1.67-1.35 (m, 7H), 0.82-0.67 (m, 3H).

### Example 20. Synthesis of 4-(((R)-8-cyclopentyl-7-ethyl-5-methyl-6-oxo-5,6,7,8-tetrahydropteridin-2-yl)amino)-N-((1r,4R)-4-(4-(4-(2,6-dioxopiperidin-3-yl)phenethyl)piperazin-1-yl)cyclohexyl)-3-methoxybenzamide (Compound 20)

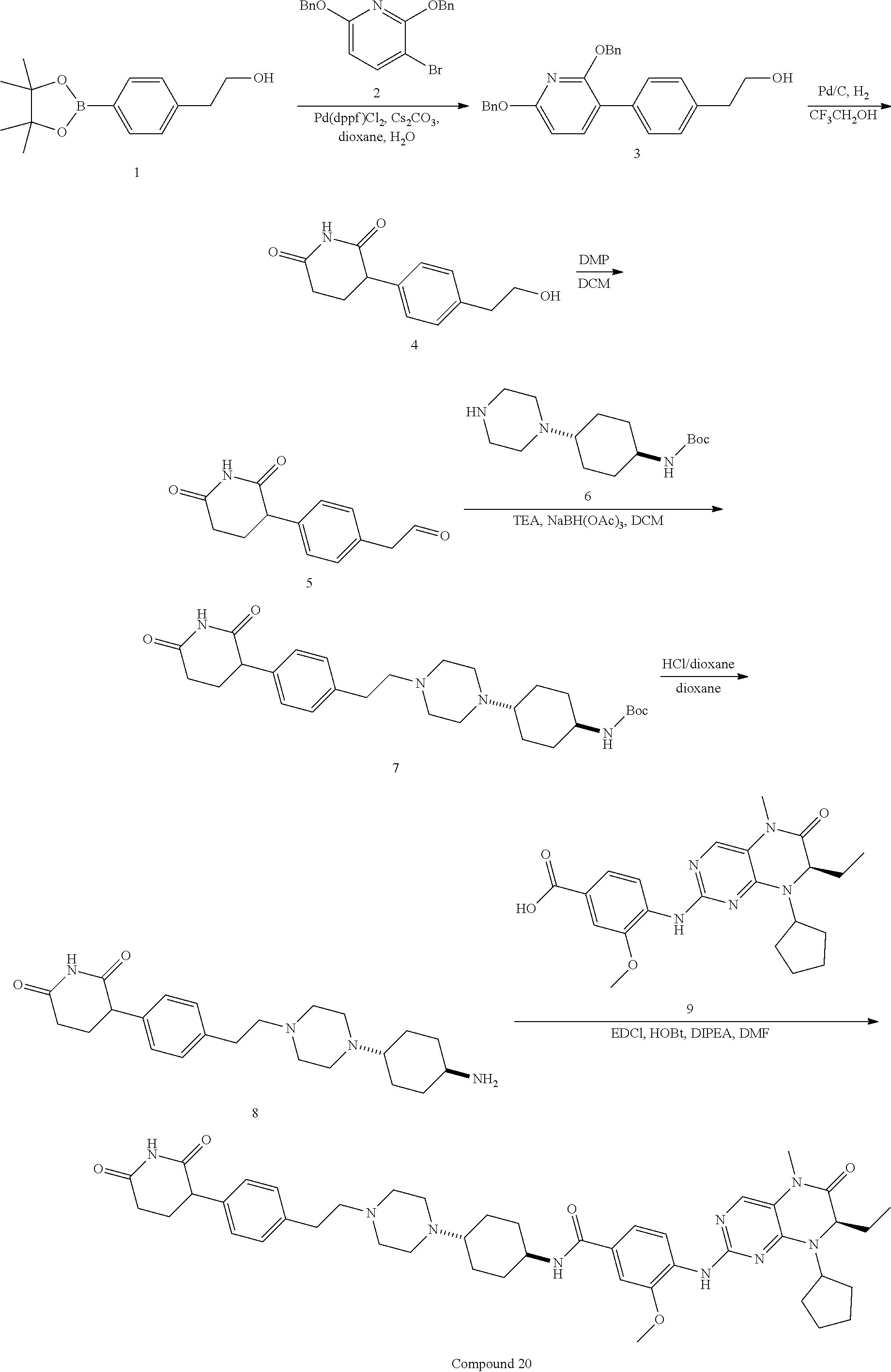

### Step 1. Synthesis of 2-(4-(2,6-bis(benzyloxy)pyridin-3-yl)phenyl)ethan-1-ol (3)

A mixture of 2-(4-(4,4,5,5-tetramethyl-1,3,2-dioxaborolan-2-yl)phenyl)ethan-1-ol (3 g, 12.09 mmol), 2,6-bis(benzyloxy)-3-bromopyridine (4.50 g, 12.15 mmol), Cs₂CO₃ (7.88 g, 24.18 mmol and Pd(dppf)Cl₂ (900.00 mg, 1.23 mmol) in dioxane (6 mL) and H₂O (1.2 mL) was degassed and purged with N₂ for 3 times, then the mixture was stirred at 100° C. for 16 hr under N₂ atmosphere. LCMS showed a main peak with desired mass. The reaction mixture was filtered. The filtrate was diluted with H₂O (80 mL), then extracted with EtOAc (150 mL×2). The combined organic lavers were washed with brine (100 mL×2), dried over Na₂SO₄, filtered and concentrated under reduced pressure to give a residue, which was purified by flash silica gel chromatography (Biotage; 40 g SepaFlash® Silica Flash Column, Eluent of 15~25% EtOAc:Petroleum ether gradient, 100 mL/min) to afford 2-(4-(2,6-bis(benzyloxy)pyridin-3-yl)phenyl)ethan-1-ol (4.9 g, 11.91 mmol, 98.49% yield) as a light yellow oil. MS(M+H)⁺=412.2

### Step 2. Synthesis of 3-(4-(2-hydroxyethyl)phenyl)piperidine-2,6-dione (4)

To a solution of 2-(4-(2,6-bis(benzyloxy)pyridin-3-yl)phenyl)ethan-1-ol (1.9 g, 4.62 mmol) in CF₃CH₂OH (50 mL) was added Pd/C (4.91 g, 4.62 mmol, 10% purity) under N₂ atmosphere, the suspension was degassed and purged with H₂ for 3 times, then the mixture was stirred at 20° C. for 32 hr under H₂ atmosphere (15 Psi). LCMS showed 2-(4-(2,6-bis(benzyloxy)pyridin-3-yl)phenyl)ethan-1-ol was consumed completely and a peak with desired mass. The reaction mixture was filtered. The filtrate was concentrated under reduced pressure. The compound was triturated with MTBE (15 mL) at 20° C. for 1 hr. The mixture was filtered. The filter cake was dried in vacuo to afford 3-(4-(2-hydroxyethyl)phenyl)piperidine-2,6-dione (800 mg, 3.43 mmol, 74.28% yield) as a light yellow solid. MS(M+H)⁺=234.1

### Step 3. Synthesis of 2-(4-(2,6-dioxopiperidin-3-yl))phenyl)acetaldehyde (5)

To a solution of 3-(4-(2-hydroxyethyl)phenyl)piperidine-2,6-dione (500 mg, 2.14 mmol) in DCM (10 mL) was added DMP (1.09 g, 2.57 mmol, 796.92 L) at 25° C. The mixture was stirred at 25° C. for 2 hr. LCMS showed a main peak with desired mass. The reaction mixture was diluted with H₂O (10 mL) at 20° C., then extracted with DCM (30 mL×2). The combined organic layers were washed with NaHCO₃ (sat, aq, 30 mL×3), dried over Na₂SO₄, filtered and concentrated under reduced pressure to afford 2-(4-(2,6-dioxopiperidin-3-yl)phenyl)acetaldehyde (490 mg, crude) as a light yellow oil. MS(M+H)⁺=232.1

### Step 4. Synthesis of tert-butyl ((1r,4r)-4-(4-(4-(2,6-dioxopiperidin-3-yl)phenethyl)piperazin-1-yl)cyclohexyl)carbamate (7)

To a solution of 2-(4-(2,6-dioxopiperidin-3-yl)phenyl)acetaldehyde (490 mg, 2.12 mmol) in DCM (20 mL) were added tert-butyl ((1r,4r)-4-(piperazin-1-yl)cyclohexyl)carbamate (544.44 mg, 1.92 mmol) and TEA (1.29 g, 12.71 mmol, 1.77 mL) at 20° C., the mixture was stirred at 20° C. for 1 h. Then NaBH(OAc)₃ (1.35 g, 6.36 mmol) was added and the resulting mixture was stirred at 20° C. for another 15 hr. LCMS showed a peak (28%) with the desired mass. The reaction mixture was adjusted pH around 9~10 with NaHCO₃ (sat. aq. 20 mL) at 0° C., then extracted with DCM (30 m×2). Then combined organic layers were washed with brine (30 mL×2), dried over Na₂SO₄, filtered and concentrated under reduced pressure. The residue was diluted with DMF (6 mL), then filtered. The filter cake was concentrated under reduced pressure to afford 350 mg of product. The filtrate was purified by prep-HPLC (column: Phenomenex luna C18 150×40 mm×15 um; mobile phase: [water (TFA)-ACN]; gradient:5%-35% B over 10 min, Column Temp: 30° C.). The eluent was lyophilized to afford 90 mg of product. Combined 2 batches to afford tert-butyl ((1r,4r)-4-(4-(4-(2,6-dioxopiperidin-3-yl)phenethyl)piperazin-1-yl)cyclohexyl)carbamate (440 mg, 882.37 μmol, 41.64% yield) as a light yellow solid. MS(M+H)⁺=499.3

¹H NMR (400 MHz, DMSO-d₆) δ=10.81 (s, 1H), 7.22-7.05 (m, 4H), 6.71-6.63 (m, 1H), 3.85-3.77 (m, 1H), 3.18-3.07 (m, 1H), 2.72-2.59 (m, 3H), 2.50-2.35 (m, 11H), 2.19-2.00 (m, 3H), 1.84-1.72 (m, 4H), 1.37 (s, 9H), 1.24-1.09 (m, 4H).

### Step 5. Synthesis of 3-(4-(2-(4-((1r,4r)-4-aminocyclohexyl)piperazin-1-yl))ethyl)phenyl)piperidine-2,6-dione (8)

To a solution of tert-butyl ((1r,4r)-4-(4-(4-(2,6-dioxopiperidin-3-yl)phenethyl)piperazin-1-yl)cyclohexyl)carbamate (300 mg, 601.62 μmol) in dioxane (2 mL) was added HCl/dioxane (2 M, 12 mL) at 20° C. and the mixture was stirred at 20° C. for 2 hr. LCMS showed a main peak with the desired mass. The reaction mixture was concentrated under reduced pressure to afford 3-(4-(2-(4-((1r,4r)-4-aminocyclohexyl)piperazin-1-yl)ethyl)phenyl)piperidine-2,6-dione (260 mg, crude, HCl salt) as a white solid. MS(M+H)⁺=399.3

### Step 6. Synthesis of 4-(((R)-8-cyclopentyl-7-ethyl-5-methyl-6-oxo-5,6,7,8-tetrahydropteridin-2-yl)amino)-N-((1r,4R)-4-(4-(4-(2,6-dioxopiperidin-3-yl)phenethyl)piperazin-1-yl)cyclohexyl)-3-methoxybenzamide (Compound 20)

To a solution of (R)-4-((8-cyclopentyl-7-ethyl-5-methyl-6-oxo-5,6,7,8-tetrahydropteridin-2-yl)amino)-3-methoxybenzoic acid (260 mg, 611.07 μmol) in DMF (5 mL) were added EDCI (200 mg, 1.04 mmol), HOBt (120 mg, 888.08 μmol), DIPEA (519.40 mg, 4.02 mmol, 700 μL) and 3-(4-(2-(4-((1r,4r)-4-aminocyclohexyl)piperazin-1-yl)ethyl)phenyl)piperidine-2,6-dione (260 mg, 597.70 μmol, HCl) at 20° C. The mixture was stirred at 20° C. for 16 hr under N₂ atmosphere. LCMS showed a main peak with the desired mass. The reaction mixture was diluted with H₂O (15 mL), then extracted with EtOAc (50 mL×2). The combined organic layers were washed with brine (30 mL×3), dried over Na₂SO₄, filtered and concentrated under reduced pressure. The compound was triturated with (EtOAc:MTBE=1:1; 40 mL) at 20° C. for 1 hr. The mixture was filtered and the filter cake was concentrated under reduced pressure followed by lyophilization to afford 4-(((R)-8-cyclopentyl-7-ethyl-5-methyl-6-oxo-5,6,7,8-tetrahydropteridin-2-yl)amino)-N-((1r,4R)-4-(4-(4-(2,6-dioxopiperidin-3-yl)phenethyl)piperazin-1-yl)cyclohexyl)-3-methoxybenzamide (251.3 mg, 296.51 μmol, 48.52% yield, 95.1% purity) as a white solid. MS(M+H)⁺=806.6

¹H NMR (400 MHz, DMSO-d₆) δ=10.87-10.74 (m, 1H), 8.47 (s, 1H), 8.08-7.94 (m, 1H), 7.85 (s, 1H), 7.63-7.55 (m, 1H), 7.52-7.42 (m, 2H), 7.23-7.14 (m, 2H), 7.14-7.08 (m, 2H), 4.40-4.31 (m, 1H), 4.28-4.18 (m, 1H), 3.94 (s, 3H), 3.84-3.68 (m, 2H), 3.25 (s, 3H), 2.74-2.62 (m, 3H), 2.61-2.52 (m, 6H), 2.47-2.36 (m, 4H), 2.35-2.12 (m, 3H), 2.07-1.98 (m, 2H), 1.93-1.71 (m, 10H), 1.67-1.55 (m, 3H), 1.41-1.24 (m, 4H), 0.77 (t, J=7.2 Hz, 3H).

The filtrate was concentrated, then purified by prep-HPLC (column: Phenomene×luna C18 150×40 mmx 15 um; mobile phase: [water (TFA)-ACN]; gradient: 10%-40% B over 10 min, Column Temp: 30° C.). The eluent was lyophilized to afford 4-(((R)-8-cyclopentyl-7-ethyl-5-methyl-6-oxo-5,6,7,8-tetrahydropteridin-2-yl)amino)-N-((1r,4R)-4-(4-(4-(2,6-dioxopiperidin-3-yl)phenethyl)piperazin-1-yl)cyclohexyl)-3-methoxybenzamide (114 mg, 106.61 μmol, 17.45% yield, 96.7% purity, 2TFA) as a white solid. MS(M+H)⁺=806.6

¹H NMR (400 MHz, DMSO-d₆) δ=0.83 (s, 1H), 9.51-9.33 (m, 1H), 8.30 (d, J=7.7 Hz, 1H), 7.85 (d, J=8.3 Hz, 1H), 7.78 (s, 1H), 7.59-7.50 (m, 2H), 7.29-7.22 (m, 2H), 7.22-7.16 (m, 2H), 4.49-4.42 (m, 2H), 4.16-4.12 (m, 2H), 3.90 (s, 3H), 3.86-3.82 (m, 2H), 3.22-3.18 (m, 3H), 2.97-2.90 (m, 2H), 2.70-2.60 (m, 1H), 2.51-2.36 (m, 10H), 2.19-1.77 (m, 12H), 1.62-1.39 (m, 8H), 0.75 (t, J=7.5 Hz, 3H).

### Example 21. Synthesis of 4-(((R)-8-cyclopentyl-7-ethyl-5-methyl-6-oxo-5,6,7,8-tetrahydropteridin-2-yl)amino)-N-(4-(((4-(2,6-dioxopiperidin-3-yl)phenyl)amino)methyl)-[1,4′-bipiperidin]-1′-yl)-3-methoxybenzamide (Compound 21)

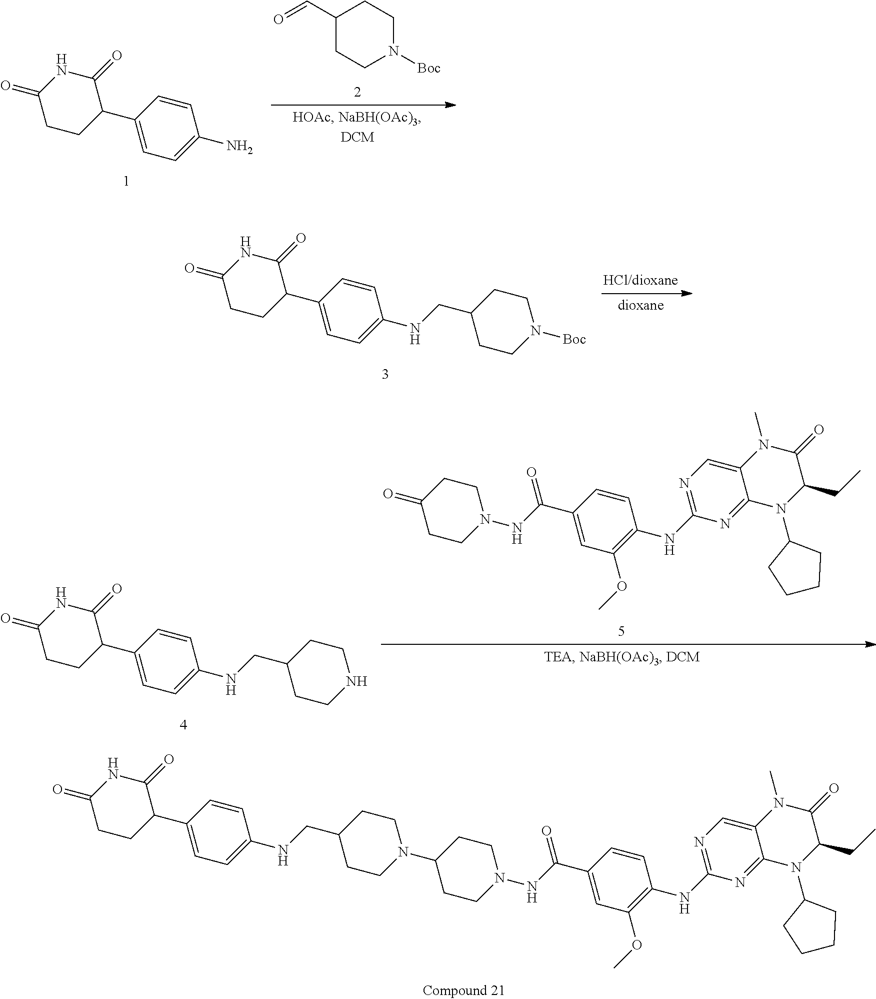 [SMILES: Nc1ccc(C2CCC(=O)NC2=O)cc1 ; CC(C)(C)OC(=O)N1CCC(C=O)CC1 ; CC(C)(C)OC(=O)N1CCC(CNc2ccc(C3CCC(=O)NC3=O)cc2)CC1 ; O=C1CCC(c2ccc(NCC3CCNCC3)cc2)C(=O)N1 ; CC[C@@H]1C(=O)N(C)c2cnc(Nc3ccc(C(=O)NN4CCC(=O)CC4)cc3OC)nc2N1C1CCCC1 ; CC[C@@H]1C(=O)N(C)c2cnc(Nc3ccc(C(=O)NN4CCC(N5CCC(CNc6ccc(C7CCC(=O)NC7=O)cc6)CC5)CC4)cc3OC)nc2N1C1CCCC1]

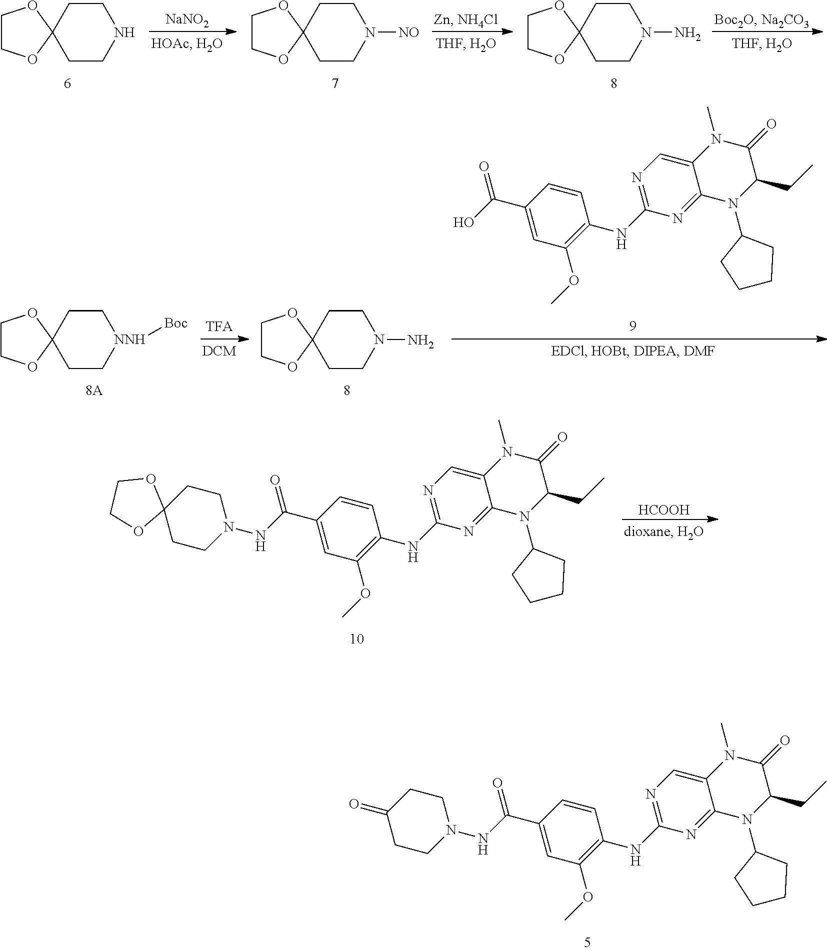

### Step 1. Synthesis of tert-butyl 4-(((4-(2,6-dioxopiperidin-3-yl)phenyl)amino)methyl)piperidine-1-carboxylate (3)

To a solution of 3-(4-aminophenyl) piperidine-2,6-dione (800 mg, 3.92 mmol) in DCM (30 mL) were added tert-butyl 4-formylpiperidine-1-carboxylate (960.00 mg, 4.50 mmol) and HOAc (314.70 mg, 5.24 mmol, 300 μL) at 20° C., the mixture was stirred at 20° C. for 1 h. Then NaBH(OAc)₃ (2.40 g, 11.32 mmol) was added and the resulting mixture was stirred at 20° C. for 15 hr. LCMS showed a main peak with desired mass. The reaction mixture was adjusted pH=8-9 at 0° C. with NaHCO₃ (sat. aq), then extracted with DCM (30 mL×3). The combined organic layers were washed with brine (30 mL×2), dried over Na₂SO₄, filtered and concentrated under reduced pressure. The product was purified by prep-HPLC (column: Phenomenex luna C18 150×40 mm×15 um; mobile phase: [water (TFA)-ACN]; gradient:15%-45% B over 10 min; Column Temp: 30° C.) to afford tert-butyl 4-(((4-(2,6-dioxopiperidin-3-yl)phenyl)amino)methyl)piperidine-1-carboxylate (1.4 g, 3.49 mmol, 89.01% yield) as a light yellow solid. MS(M+Na)⁺=424.2

### Step 2. Synthesis of 3-(4-((piperidin-4-ylmethyl)amino)phenyl)piperidine-2,6-dione (4)

To a solution of tert-butyl 4-(((4-(2,6-dioxopiperidin-3-yl)phenyl)amino)methyl)piperidine-1-carboxylate (1 g, 2.49 mmol) in dioxane (2 mL) was added HCl/dioxane (2 M, 20 mL) at 20° C., the mixture was stirred at 20° C. for 2 hr. LCMS showed a main peak with desired mass. The reaction mixture was concentrated under reduced pressure to afford 3-(4-((piperidin-4-ylmethyl)amino)phenyl)piperidine-2,6-dione (800 mg, crude, HCl salt) as a light yellow solid. MS(M+H)⁺=302.2

### Step 3. Synthesis of 8-nitroso-1,4-dioxa-8-azaspiro[4.5]decane (7)

To a solution of 1,4-dioxa-8-azaspiro[4.5]decane (2 g, 13.97 mmol, 1.79 mL) in H₂O (20 mL) was added NaNO₂ (2.9 g, 42.03 mmol) in portions at 0° C., then HOAc (3.36 g, 55.87 mmol, 3.20 mL) was added dropwise at 0° C. and the resulting mixture was stirred at 20° C. for another 16 hr. LCMS showed a main peak with desired mass. The reaction mixture was diluted with H₂O (20 mL) at 0° C., then adjusted pH=9 with NaHCO₃ (sat. aq) at 0° C., then extracted with EtOAc (50 mL×3). The combined organic layers were washed with brine (60 mL×2), dried over Na₂SO₄, filtered and the filtrate was concentrated under reduced pressure to afford 8-nitroso-1,4-dioxa-8-azaspiro[4.5]decane (2.4 g, crude) as a light yellow solid. MS(M+H)⁺=173.0

### Step 4. Synthesis of 1,4-dioxa-8-azaspiro[4.5]decan-8-amine (8)

To a solution of 8-nitroso-1,4-dioxa-8-azaspiro[4.5]decane (2 g, 11.62 mmol) in THF (20 mL) and H₂O (10 mL) was added NH₄Cl (3.20 g, 59.82 mmol) at 0° C. under N₂ atmosphere, then Zn (4.10 g, 62.70 mmol) was added in portions at 0° C., then the suspension was stirred at 20° C. for 4 hr under N₂ atmosphere. LCMS showed starting material was consumed completely and a main peak with desired mass. The reaction mixture was filtered. The filter cake was washed with THF (50 mL). The filtrate of 1,4-dioxa-8-azaspiro[4.5]decan-8-amine (1.8 g, crude, light yellow oil) was used into the next step without further purification. MS(M+H)⁺=159.1

### Step 5. Synthesis of tert-butyl (1,4-dioxa-8-azaspiro[4.5]decan-8-yl)carbamate (8A)

To a solution of 1,4-dioxa-8-azaspiro[4.5]decan-8-amine (1.8 g, 11.38 mmol) in THF (10 mL) and H₂O (10 mL) was added Boc₂O (7.60 g, 34.82 mmol, 8 mL) at 0° C. under N₂ atmosphere, then Na₂CO₃ (5 g, 47.17 mmol) was added into the mixture in portions at 0° C., then the mixture was stirred at 20° C. for 14 hr under N₂ atmosphere. LCMS showed 1,4-dioxa-8-azaspiro[4.5]decan-8-amine was consumed completely and a main peak with desired mass. The reaction mixture was filtered. The filtrate was diluted with H₂O (30 mL), then extracted with EtOAc (80 mL×2). The combined organic layers were washed with brine (80 mL×2), dried over Na₂SO₄, filtered and the filtrate was concentrated under reduced pressure. The residue was purified by flash silica gel chromatography (Biotage; 20 g SepaFlash® Silica Flash Column, Eluent of 30~50% EtOAc:Petroleum ether gradient, 100 mL/min) to afford tert-butyl (1,4-dioxa-8-azaspiro[4.5]decan-8-yl)carbamate (900 mg, 3.48 mmol, 30.62% yield) as a light yellow solid. MS(M+H-56)⁺=203.1

### Step 6. Synthesis of 1,4-dioxa-8-azaspiro[4.5]decan-8-amine (8)

To a solution of tert-butyl (1,4-dioxa-8-azaspiro[4.5]decan-8-yl)carbamate (600 mg, 2.32 mmol) in DCM (5 mL) was added TFA (4.61 g, 40.39 mmol, 3.00 mL) at 20° C., the mixture was stirred at 20° C. for 2 hr. LCMS showed a main peak with desired mass. The reaction mixture was concentrated under reduced pressure to afford 1,4-dioxa-8-azaspiro[4.5]decan-8-amine (600 mg, crude, TFA salt) as a light yellow oil. MS(M+H)⁺=159.1

### Step 7. Synthesis of (R)-4-((8-cyclopentyl-7-ethyl-5-methyl-6-oxo-5,6,7,8-tetrahydropteridin-2-yl)amino)-3-methoxy-N-(1,4-dioxa-8-azaspiro[4.5]decan-8-yl)benzamide (10)

To a solution of (R)-4-((8-cyclopentyl-7-ethyl-5-methyl-6-oxo-5,6,7,8-tetrahydropteridin-2-yl)amino)-3-methoxybenzoic acid (600 mg, 1.41 mmol) in DMF (8 mL) were added EDCI (360.00 mg, 1.88 mmol), HOBt (240.00 mg, 1.78 mmol), DIPEA (1.11 g, 8.61 mmol, 1.50 mL) and 1,4-dioxa-8-azaspiro[4.5]decan-8-amine (600.00 mg, 2.20 mmol, TFA salt) at 20° C. The mixture was stirred at 20° C. for 16 hr under N₂ atmosphere. LCMS showed a peak (59%) with the desired mass. The reaction mixture was diluted with H₂O (30 mL), then extracted with EtOAc (50 mL×2). The combined organic layers were washed with brine (50 mL×3), dried over Na₂SO₄, filtered and concentrated under reduced pressure. The residue was purified by flash silica gel chromatography (Biotage; 10 g SepaFlash® Silica Flash Column, Eluent of 60~100% EtOAc:Petroleum ether gradient, 80 mL/min) followed by prep-HPLC (column: Phenomenex luna C18 150×40 mm×15 um; mobile phase: [water (TFA)-ACN]; gradient: 15%-45% B over 10 min, Column Temp: 30° C.). The eluent was lyophilized to afford (R)-4-((8-cyclopentyl-7-ethyl-5-methyl-6-oxo-5,6,7,8-tetrahydropteridin-2-yl)amino)-3-methoxy-N-(1,4-dioxa-8-azaspiro[4.5]decan-8-yl)benzamide (420 mg, 742.49 μmol, 52.65% yield) as a light yellow solid. MS(M+H)⁺=566.3

### Step 8. Synthesis of (R)-4-((8-cyclopentyl-7-ethyl-5-methyl-6-oxo-5,6,7,8-tetrahydropteridin-2-yl)amino)-3-methoxy-N-(4-oxopiperidin-1-yl)benzamide (5)

To a solution of (R)-4-((8-cyclopentyl-7-ethyl-5-methyl-6-oxo-5,6,7,8-tetrahydropteridin-2-yl)amino)-3-methoxy-N-(1,4-dioxa-8-azaspiro[4.5]decan-8-yl)benzamide (300 mg, 530.35 μmol) in H₂O (2 mL) and dioxane (2 mL) was added HCOOH (12 mL). The mixture was stirred at 20° C. for 32 hr. LCMS showed a main peak with desired mass. The reaction mixture was washed with EtOAc (20 mL). The aqueous phase was adjusted pH=8~9 with Na₂CO₃ (sat. aq) at 0° C., then extracted with EtOAc (30 mL×3), the combined organic layers were washed with brine (30 mL×2), dried over Na₂SO₄, filtered and the filtrate was concentrated under reduced pressure to afford (R)-4-((8-cyclopentyl-7-ethyl-5-methyl-6-oxo-5,6,7,8-tetrahydropteridin-2-yl)amino)-3-methoxy-N-(4-oxopiperidin-1-yl)benzamide (276 mg, crude) as a light yellow oil. MS(M+H)⁺=522.3

### Step 9. Synthesis of 4-(((R)-8-cyclopentyl-7-ethyl-5-methyl-6-oxo-5,6,7,8-tetrahydropteridin-2-yl)amino)-N-(4-(((4-(2,6-dioxopiperidin-3-yl)phenyl)amino)methyl)-[1,4′-bipiperidin]-1′-yl)-3-methoxybenzamide (Compound 21)

To a solution of (R)-4-((8-cyclopentyl-7-ethyl-5-methyl-6-oxo-5,6,7,8-tetrahydropteridin-2-yl)amino)-3-methoxy-N-(4-oxopiperidin-1-yl)benzamide (240 mg, 460.11 μmol) in DCM (8 mL) were added 3-(4-((piperidin-4-ylmethyl)amino)phenyl)piperidine-2,6-dione (200 mg, 591.99 μmol, HCl salt) and TEA (436.20 mg, 4.31 mmol, 600.00 μL) at 20° C., the mixture was stirred at 20° C. for 6 h. Then NaBH(OAc)₃ (420.00 mg, 1.98 mmol) was added and the resulting mixture was stirred at 20° C. for another 15 hr. LCMS showed a peak (25%) with the desired mass. The reaction mixture was adjusted the pH=9-10 with NaHCO₃ (sat. aq) at 0° C., then extracted with DCM (20 mL×2). The combined organic layers were washed with brine (20 mL×2), dried over Na₂SO₄, filtered and concentrated under reduced pressure. The residue was purified by flash silica gel chromatography (Biotage; 10 g SepaFlash® Silica Flash Column, Eluent of 0~10% EtOH:DCM gradient, 60 mL/min) followed by prep-HPLC (column: Phenomenex luna C18 150×25 mm×10 um; mobile phase: [water (TFA)-ACN]; gradient:14%-44% B over 10 min, Column Temp: 30° C.). The eluent was lyophilized to afford 4-(((R)-8-cyclopentyl-7-ethyl-5-methyl-6-oxo-5,6,7,8-tetrahydropteridin-2-yl)amino)-N-(4-(((4-(2,6-dioxopiperidin-3-yl)phenyl)amino)methyl)-[1,4′-bipiperidin]-1′-yl)-3-methoxybenzamide (91.7 mg, 85.05 μmol, 18.48% yield, 96.0% purity, 2TFA) as a white solid. MS(M+H)⁺=807.6

¹H NMR (400 MHz, DMSO-d₆) δ=10.73 (s, 1H), 9.63-9.54 (m, 1H), 9.35-9.22 (m, 2H), 7.95-7.86 (m, 1H), 7.78 (s, 1H), 7.50-7.43 (m, 2H), 6.95-6.89 (m, 2H), 6.58-6.51 (m, 2H), 4.45-4.42 (m, 1H), 4.18-4.13 (m, 1H), 3.89 (s, 4H), 3.66-3.63 (m, 2H), 3.56-3.54 (m, 1H), 3.22 (s, 3H), 3.17-3.08 (m, 3H), 2.97-2.86 (m, 5H), 2.64-2.56 (m, 1H), 2.10-1.97 (m, 6H), 1.92-1.74 (m, 10H), 1.54-1.38 (m, 6H), 0.75 (t, J=7.4 Hz, 3H).

### Example 22. Synthesis of 4-(((R)-8-cyclopentyl-7-ethyl-5-methyl-6-oxo-5,6,7,8-tetrahydropteridin-2-yl)amino)-N-(1′-(4-(2,6-dioxopiperidin-3-yl)phenethyl)-[1,4′-bipiperidin]-4-yl)-3-methoxy benzamide (Compound 22)

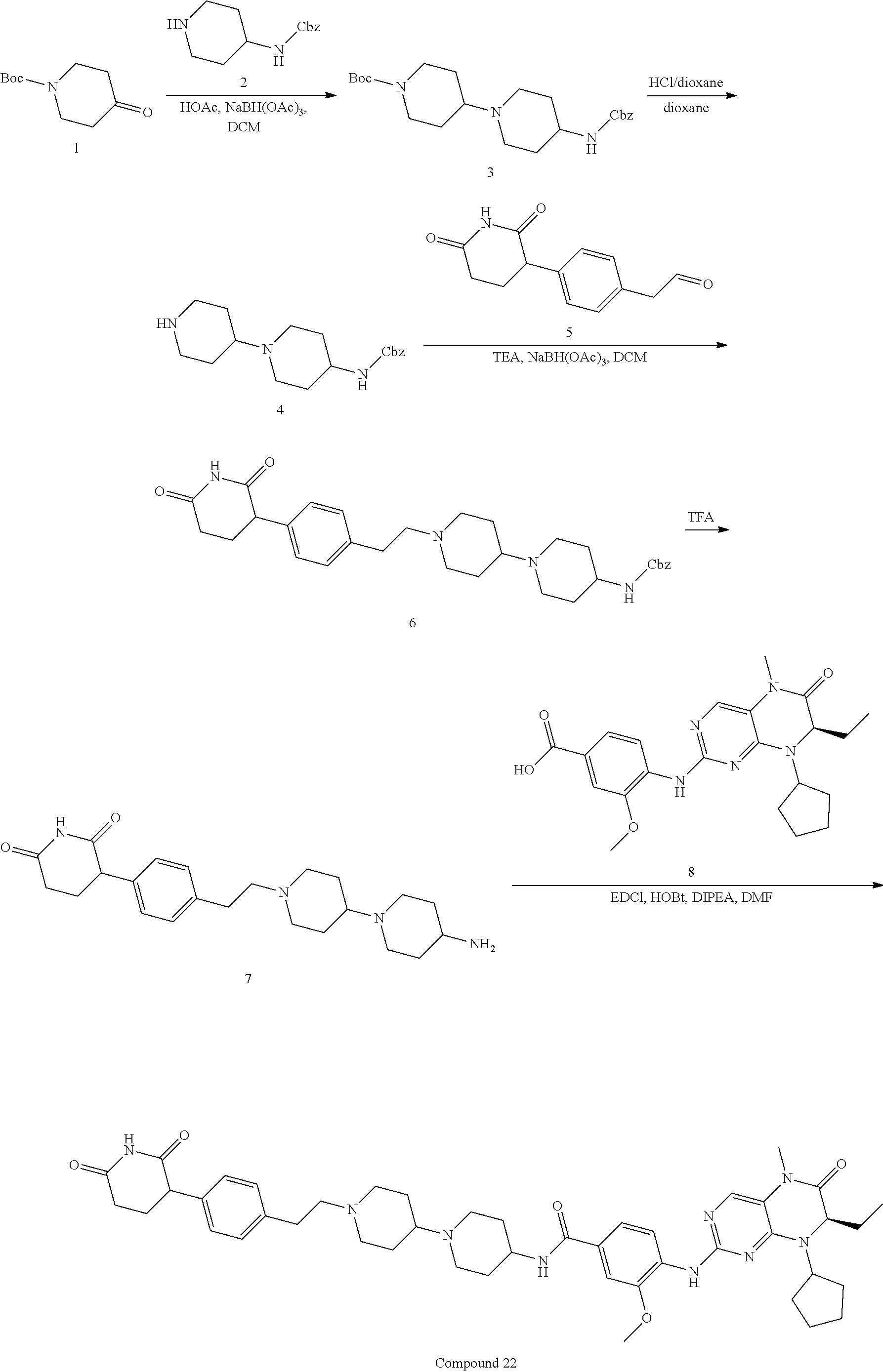

### Step 1. Synthesis of tert-butyl 4-((benzyloxy)carbonyl)amino)-[1,4′-bipiperidine]-1-carboxylate (3)

To a solution of tert-butyl 4-oxopiperidine-1-carboxylate (2 g, 10.04 mmol) in DCM (30 mL) was added benzyl piperidin-4-ylcarbamate (2.5 g, 10.67 mmol) and HOAc (629.40 mg, 10.48 mmol, 600 μL). The mixture was stirred at 20° C. for 2 hr. Then NaBH(OAc)₃ (4.67 g, 22.02 mmol) was added at 20° C. and the resulting mixture was stirred at 20° C. for 14 hr. LCMS showed a peak (59%) with desired mass. The reaction mixture was diluted with H₂O (10 mL) at 0° C., adjusted pH=8-9 with NaHCO₃ (sat. aq) at 0° C., then extracted with DCM (30 mL×3). The combined organic layers were washed with brine (30 mL×2), dried over Na₂SO₄, filtered and the filtrate was concentrated under reduced pressure. The residue was purified by reversed-phase HPLC (ID.95 mm×H 365 mm Column Welch Ultimate XB_C₁₈ 20-40 μm; 120 A; Mobile phase MeCN/H₂O; Gradient B %: 30-50% 45 min: % min: Flow rate 100 mL/min; 0.1% FA condition). The eluent was lyophilized to afford tert-butyl 4-(((benzyloxy)carbonyl)amino)-[1,4′-bipiperidine]-1′-carboxylate (2.6 g, 6.23 mmol, 62.03% yield) as a light yellow solid. MS(M+H)⁺=418.2

### Step 2. Synthesis of benzyl [1,4′-bipiperidin]-4-ylcarbamate (4)

To a solution of tert-butyl 4-(((benzyloxy)carbonyl)amino)-[1,4′-bipiperidine]-1′-carboxylate (1.0 g, 2.39 mmol) in dioxane (2 mL) was added HCl/dioxane (2 M, 10 mL) at 20° C., the mixture was stirred at 20° C. for 2 hr. LCMS showed tert-butyl 4-(((benzyloxy)carbonyl)amino)-[1,4′-bipiperidine]-1′-carboxylate was consumed completely and a main peak with desired mass. The reaction mixture was concentrated under reduced pressure to afford benzyl [1,4′-bipiperidin]-4-ylcarbamate (800 mg, crude, HCl salt) as a light yellow solid. MS(M+H)⁺=318.2

### Step 3. Synthesis of benzyl (1′-(4-(2,6-dioxopiperidin-3-yl)phenethyl)-[1,4′-bipiperidin]-4-yl)carbamate (6)

To a solution of 2-(4-(2,6-dioxopiperidin-3-yl)phenyl)acetaldehyde (450 mg, 1.95 mmol) in DCM (25 mL) were added benzyl [1,4′-bipiperidin]-4-ylcarbamate (510 mg, 1.44 mmol, HCl salt) and TEA (1.65 g, 16.33 mmol, 2.27 mL) at 20° C., the mixture was stirred at 20° C. for 1 h. Then NaBH(OAc)₃ (1.36 g, 6.43 mmol) was added and the resulting mixture was stirred at 20° C. for 15 hr. LCMS showed a peak (24%) with the desired mass. The reaction mixture was to be adjusted the pH=9-10 with NaHCO₃ (sat. aq) at 0° C., then extracted with DCM (40 mL×2). The combined organic layers were washed with brine (40 mL×2), dried over Na₂SO₄, filtered and concentrated under reduced pressure. The residue was diluted with DMF (7 mL), then filtered. The filter cake was dried in vacuo to afford 150 mg of the product. The filtrate was purified by prep-HPLC (column: Phenomenex luna C18 150×40 mm×15 um; mobile phase: [water (TFA)-ACN]; gradient: 0%-30% B over 10 min, Column Temp: 30° C., Column Temp: 30° C.). The eluent was lyophilized to afford another 450 mg of product. Combined 2 batches to afford benzyl (1′-(4-(2,6-dioxopiperidin-3-yl)phenethyl)-[1,4′-bipiperidin]-4-yl)carbamate (600 mg, 1.13 mmol, 57.88% yield) as a light yellow solid. MS(M+H)⁺=533.3

### Step 4. Synthesis of 3-(4-(2-(4-amino-[1,4′-bipiperidin]-1′-yl)ethyl)phenyl)piperidine-2,6-dione (7)

A mixture of benzyl (1′-(4-(2,6-dioxopiperidin-3-yl)phenethyl)-[1,4′-bipiperidin]-4-yl)carbamate (560 mg, 1.05 mmol) in TFA (10 mL) was stirred at 60° C. for 3 hr. LCMS showed the starting material was consumed completely and a peak with desired mass. The reaction mixture was concentrated under reduced pressure to afford 3-(4-(2-(4-amino-[1,4′-bipiperidin]-1′-yl)ethyl)phenyl)piperidine-2,6-dione (540 mg, crude, TFA salt) as a light yellow oil. MS(M+H)⁺=399.3

### Step 5. Synthesis of 4-(((R)-8-cyclopentyl-7-ethyl-5-methyl-6-oxo-5,6,7,8-tetrahydropteridin-2-yl)amino)-N-(1′-(4-(2,6-dioxopiperidin-3-yl)phenethyl)-[1,4′-bipiperidin]-4-yl)-3-methoxybenzamide (Compound 22)

To a solution of (R)-4-((8-cyclopentyl-7-ethyl-5-methyl-6-oxo-5,6,7,8-tetrahydropteridin-2-yl)amino)-3-methoxybenzoic acid (400 mg, 940.12 μmol) in DMF (6 mL) were added EDCI (400 mg, 2.09 mmol), HOBt (200 mg, 1.48 mmol), DIPEA (742.00 mg, 5.74 mmol, 1 mL) and 3-(4-(2-(4-amino-[1,4′-bipiperidin]-1′-yl)ethyl)phenyl)piperidine-2,6-dione (540.00 mg, 1.05 mmol, TFA salt) at 20° C. The mixture was stirred at 20° C. for 16 hr under N₂ atmosphere. LCMS showed a peak (45%) with the desired mass. The reaction mixture was diluted with H₂O (15 mL), then with EtOAc (40 mL×2). The combined organic layers were washed with brine (30 mL×3), dried over Na₂SO₄, filtered and concentrated under reduced pressure. The residue was purified by prep-HPLC (column: Phenomenex luna C18 150×40 mm×15 um; mobile phase: [water (TFA)-ACN]; gradient:10%-40% B over 10 min, Column Temp: 30° C.). The eluent was lyophilized to afford 4-(((R)-8-cyclopentyl-7-ethyl-5-methyl-6-oxo-5,6,7,8-tetrahydropteridin-2-yl)amino)-N-(1′-(4-(2,6-dioxopiperidin-3-yl)phenethyl)-[1,4′-bipiperidin]-4-yl)-3-methoxybenzamide (194.2 mg, 227.21 μmol, 24.17% yield, 94.3% purity, 2TFA) as a light yellow solid and another 90 mg of impure product, which was repurified by prep-HPLC (column: Waters Xbridge C18 150×50 mm×10 um; mobile phase: [water (NH₄HCO₃) ACN]; gradient: 25%-55% B over 10 min Column Temp: 30° C.), The eluent was lyophilized to afford 4-(((R)-8-cyclopentyl-7-ethyl-5-methyl-6-oxo-5,6,7,8-tetrahydropteridin-2-yl)amino)-N-(1′-(4-(2,6-dioxopiperidin-3-yl)phenethyl)-[1,4′-bipiperidin]-4-yl)-3-methoxybenzamide (46.7 mg, 57.19 μmol, 6.08% yield, 98.7% purity) as a light yellow solid. MS(M+H)⁺=806.6

¹H NMR (400 MHz, DMSO-d₆) δ=10.86-10.77 (m, 1H), 8.45-8.36 (m, 1H), 8.09-8.01 (m, 1H), 7.89-7.81 (m, 1H), 7.62-7.56 (m, 1H), 7.51-7.44 (m, 2H), 7.21-7.15 (m, 2H), 7.14-7.08 (m, 2H), 4.41-4.32 (m, 1H), 4.28-4.19 (m, 1H), 3.94 (s, 3H), 3.84-3.71 (m, 2H), 3.30-3.22 (m, 5H), 2.99-2.85 (m, 4H), 2.74-2.58 (m, 4H), 2.26-2.15 (m, 4H), 2.04-1.90 (m, 5H), 1.82-1.41 (m, 16H), 0.77 (t, J=7.5 Hz, 3H).

### Example 23. Synthesis of 4-(((R)-8-cyclopentyl-7-ethyl-5-methyl-6-oxo-5,6,7,8-tetrahydropteridin-2-yl)amino)-N-(1′-(4-(2,6-dioxopiperidin-3-yl)phenethyl)-[4,4′-bipiperidin]-1-yl)-3-methoxy benzamide (Compound 23)

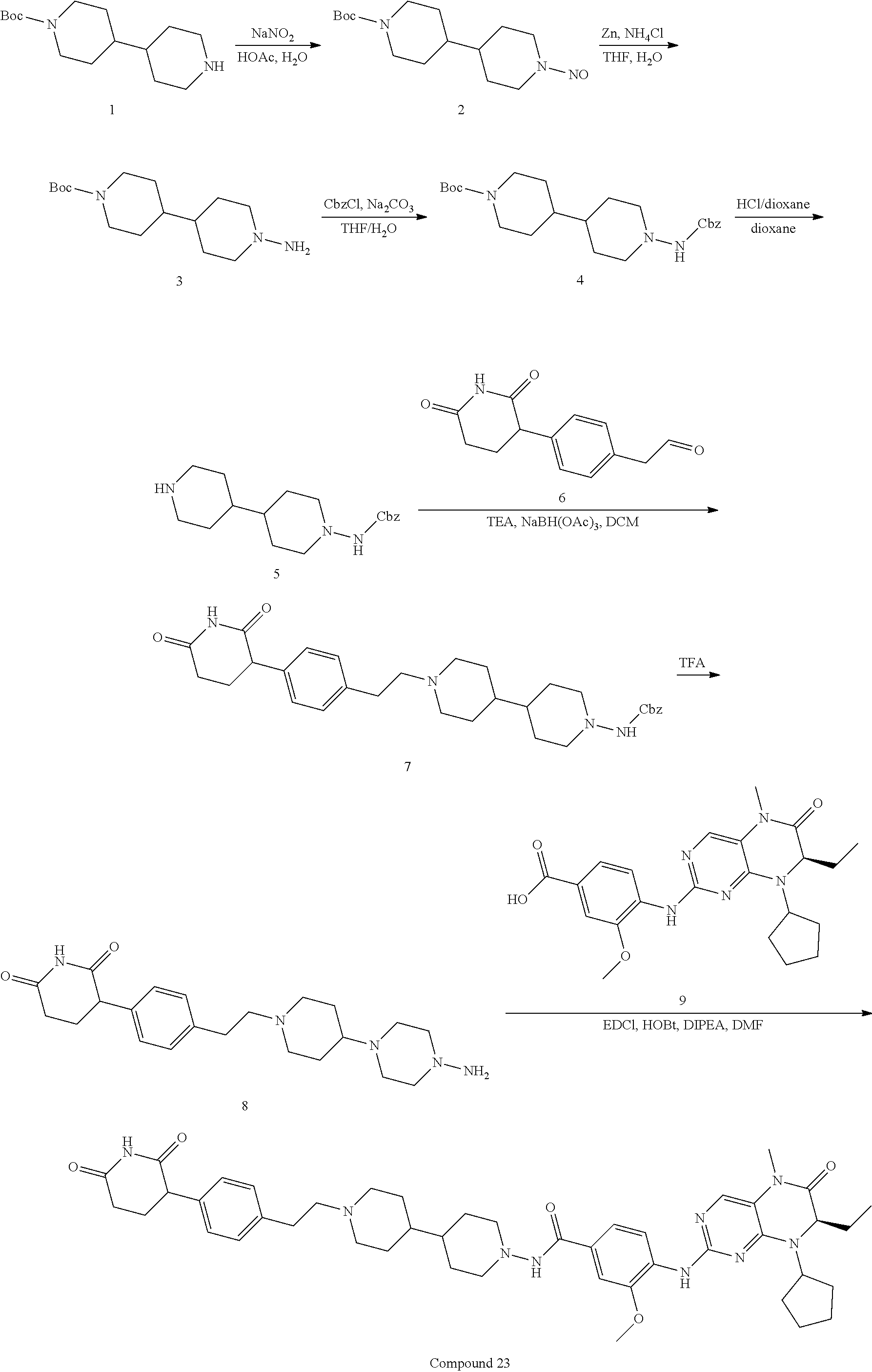

### Step 1. Synthesis of tert-butyl 1′-nitroso-[4,4′-bipiperidine]-1-carboxylate (2)

To a solution of tert-butyl [4,4′-bipiperidine]-1-carboxylate (2 g, 7.45 mmol) in H₂O (20 mL) was added NaNO₂ (1.54 g, 22.36 mmol) in portions at 0° C., then HOAc (2.10 g, 34.94 mmol, 2 mL) was added dropwise at 0° C., the mixture was stirred at 20° C. for 16 hr. LCMS showed a main peak with desired mass. The reaction mixture was diluted with H₂O (20 mL) at 0° C., NaHCO₃ (sat. aq, 60 mL) was added at 0° C. to adjust pH=9, then extracted with EtOAc (60 mL×3). The combined organic layers were washed with brine 160 mL (80 mL×2), dried over Na₂SO₄, filtered. The filtrate was concentrated under reduced pressure to afford tert-butyl 1′-nitroso-[4,4′-bipiperidine]-1-carboxylate (2.2 g, crude) as a light yellow oil. MS(M+H-56)⁺=242.1

¹H NMR (400 MHz, DMSO-d₆) δ=4.87-4.80 (m, 1H), 4.69-4.58 (m, 1H), 4.01-3.86 (m, 2H), 3.73-3.64 (m, 1H), 2.79-2.53 (m, 3H), 1.96-1.89 (m, 1H), 1.78-1.69 (m, 1H), 1.68-1.59 (m, 2H), 1.56-1.46 (m, 1H), 1.39 (s, 9H), 1.33-1.23 (m, 2H), 1.09-0.91 (m, 3H).

### Step 2. Synthesis of tert-butyl 1′-amino-[4,4′-bipiperidine]-1-carboxylate (3)

To a solution of tert-butyl 1′-nitroso-[4,4′-bipiperidine]-1-carboxylate (2.1 g, 7.06 mmol) in THF (20 mL) and H₂O (10 mL) was added NH₄Cl (2 g, 37.39 mmol) at 0° C. under N₂ atmosphere, Then Zn (2.6 g, 39.76 mmol) was added into the mixture in portions at 0° C. and the resulting mixture was stirred at 20° C. for 3 hr under N₂ atmosphere. LCMS showed a peak with desired mass. The reaction mixture was filtered. The filter cake was washed with THF (50 mL). The filtrate of tert-butyl 1′-amino-[4,4′-bipiperidine]-1-carboxylate (2 g, crude, light yellow oil) was used into the next step without further purification. MS(M+H)⁺=284.2

### Step 3. Synthesis of tert-butyl 1′-(((benzyloxy)carbonyl)amino)-[4,4′-bipiperidine]-1-carboxylate (4)

To a solution of tert-butyl 1′-amino-[4,4′-bipiperidine]-1-carboxylate (2 g, 7.06 mmol) in THF (40 mL) and H₂O (20 mL) was added Na₂CO₃ (4 g, 37.74 mmol) at 0° C. under N₂ atmosphere, then CbzCl (4.80 g, 28.16 mmol, 4.02 mL) was added dropwise at 0° C., then the mixture was stirred at 20° C. for 16 hr under N₂ atmosphere. LCMS showed tert-butyl 1′-amino-[4,4′-bipiperidine]-1-carboxylate was consumed completely and a peak with desired mass. The reaction mixture was filtered. The filtrate was diluted with H₂O (30 mL), then extracted with EtOAc (60 mL×2). The combined organic layers were washed with brine (50 mL×3), dried over Na₂SO₄, filtered. The filtrate was concentrated under reduced pressure. The residue was purified by flash silica gel chromatography (Biotage; 20 g SepaFlash® Silica Flash Column, Eluent of 25~33% EtOAc:Petroleum ether gradient, 80 mL/min) followed by prep-HPLC (column: Waters Xbridge C18 150×50 mmx 10 um; mobile phase: [water (NH₄HCO₃)-ACN]; gradient:44%-74% B over 10 min, Column Temp: 30° C.). The eluent was lyophilized to afford tert-butyl 1′-(((benzyloxy)carbonyl)amino)-[4,4′-bipiperidine]-1-carboxylate (1.8 g, 4.31 mmol, 61.09% yield) as a white solid. MS(M+H)⁺=418.2

### Step 4. Synthesis of benzyl [4,4′-bipiperidin]-1-ylcarbamate (5)

To a solution of tert-butyl 1′-(((benzyloxy)carbonyl)amino)-[4,4′-bipiperidine]-1-carboxylate (1 g, 2.39 mmol) in dioxane (6 mL) was added HCl/dioxane (2 M, 10 mL) at 20° C., the mixture was stirred at 20° C. for 2 hr. LCMS showed a main peak with desired mass. The reaction mixture was concentrated under reduced pressure to afford benzyl [4,4′-bipiperidin]-1-ylcarbamate (820 mg, crude, HCl salt) as a light yellow solid. MS(M+H)⁺=318.2

### Step 5. Synthesis of benzyl (1′-(4-(2,6-dioxopiperidin-3-yl)phenethyl)-[4,4′-bipiperidin]-1-yl)carbamate (7)

To a solution of 2-(4-(2,6-dioxopiperidin-3-yl)phenyl)acetaldehyde (550 mg, 2.38 mmol) in DCM (25 mL) were added benzyl [4,4′-bipiperidin]-1-ylcarbamate (700 mg, 1.98 mmol, HCl salt) and TEA (2.04 g, 20.12 mmol, 2.8 mL) at 20° C., the mixture was stirred at 20° C. for 1 h. Then NaBH(OAc)₃ (1.53 g, 7.21 mmol) was added and the resulting mixture was stirred at 20° C. for 15 hr. LCMS showed a peak (56%) with the desired mass. The reaction mixture was treated with NaHCO₃ (sat. aq) to adjust the pH=9-10 at 0° C., then extracted with DCM (40 mL×2). The combined organic layers were washed with brine (30 mL×2), dried over Na₂SO₄, filtered and concentrated under reduced pressure. The residue was diluted with DMF (6 mL), filtered. The filter cake was dried under reduced pressure to afford 550 mg of product. The filtrate was purified by prep-HPLC (column: Phenomenex luna C18 150×25 mm×10 um; mobile phase: [water (TFA)-ACN]; gradient:16%-46% B over 12 min). The eluent was lyophilized to afford another 120 mg of product. Combined 2 batches to afford benzyl (1′-(4-(2,6-dioxopiperidin-3-yl)phenethyl)-[4,4′-bipiperidin]-1-yl)carbamate (670 mg, 1.26 mmol, 52.88% yield) as a light yellow solid. MS(M+H)⁺=533.3

### Step 6. Synthesis of 3-(4-(2-(1′-amino-[4,4′-bipiperidin]-1-yl)ethyl)phenyl)piperidine-2,6-dione (8)

A mixture of benzyl (1′-(4-(2,6-dioxopiperidin-3-yl)phenethyl)-[4,4′-bipiperidin]-1-yl)carbamate (150 mg, 281.60 μmol) in TFA (5 mL) was stirred at 60° C. for 3 hr. LCMS showed the starting material was consumed completely and a peak with desired mass. The reaction mixture was concentrated under reduced pressure to afford 3-(4-(2-(1′-amino-[4,4′-bipiperidin]-1-yl)ethyl)phenyl)piperidine-2,6-dione (144 mg, crude, TFA salt) as a light yellow oil. MS(M+H)⁺=399.3

### Step 7. Synthesis of 4-(((R)-8-cyclopentyl-7-ethyl-5-methyl-6-oxo-5,6,7,8-tetrahydropteridin-2-yl)amino)-N-(1′-(4-(2,6-dioxopiperidin-3-yl)phenethyl)-[4,4′-bipiperidin]-1-yl)-3-methoxybenzamide (Compound 23)

To a solution of (R)-4-((8-cyclopentyl-7-ethyl-5-methyl-6-oxo-5,6,7,8-tetrahydropteridin-2-yl)amino)-3-methoxybenzoic acid (110 mg, 258.53 μmol) in DMF (3 mL) were added EDCI (100 mg, 521.65 μmol), HOBt (60 mg, 444.04 μmol), DIPEA (296.80 mg, 2.30 mmol, 400 μL) and 3-(4-(2-(1′-amino-[4,4′-bipiperidin]-1-yl)ethyl)phenyl)piperidine-2,6-dione (144 mg, 280.94 μmol, TFA salt) at 20° C. The mixture was stirred at 20° C. for 16 hr under N₂ atmosphere. LCMS showed a peak (57%) with desired mass. The reaction mixture was diluted with H₂O (5 mL), then extracted with EtOAc (15 mL×2). The combined organic layers were washed with brine (20 mL×3), dried over Na₂SO₄, filtered and concentrated under reduced pressure. The compound was purified by prep-HPLC (column: Phenomenex luna C18 150×25 mmx 10 um; mobile phase: [water (TFA)-ACN]; gradient:15%-45% B over 12 min, Column Temp: 30° C.). The eluent was lyophilized to afford 4-(((R)-8-cyclopentyl-7-ethyl-5-methyl-6-oxo-5,6,7,8-tetrahydropteridin-2-yl)amino)-N-(1′-(4-(2,6-dioxopiperidin-3-yl)phenethyl)-[4,4′-bipiperidin]-1-yl)-3-methoxybenzamide (79.2 mg, 70.92 μmol, 27.43% yield, 92.6% purity, 2TFA) as a light yellow solid. MS(M+H)⁺=806.6

¹H NMR (400 MHz, DMSO-d₆) δ=10.84 (s, 1H), 9.94-9.76 (m, 1H), 9.66-9.32 (m, 2H), 7.92-7.83 (m, 1H), 7.79 (s, 1H), 7.57-7.45 (m, 2H), 7.33-7.18 (m, 4H), 4.48-4.38 (m, 1H), 4.24-4.08 (m, 1H), 3.98-3.76 (m, 4H), 3.67-3.36 (m, 2H), 3.30-3.10 (m, 6H), 3.02-2.79 (m, 5H), 2.73-2.62 (m, 1H), 2.49-2.36 (m, 2H), 2.26-2.10 (m, 1H), 2.06-1.65 (m, 11H), 1.63-1.06 (m, 11H), 0.76 (t, J=7.4 Hz, 3H).

### Example 24. Synthesis of 4-(((R)-8-cyclopentyl-7-ethyl-5-methyl-6-oxo-5,6,7,8-tetrahydropteridin-2-yl)amino)-N-(1-(3-(4-(4-((2,6-dioxopiperidin-3-yl)amino)phenyl)piperazin-1-yl)propanoyl) piperidin-4-yl)-3-methoxybenzamide (Compound 24)

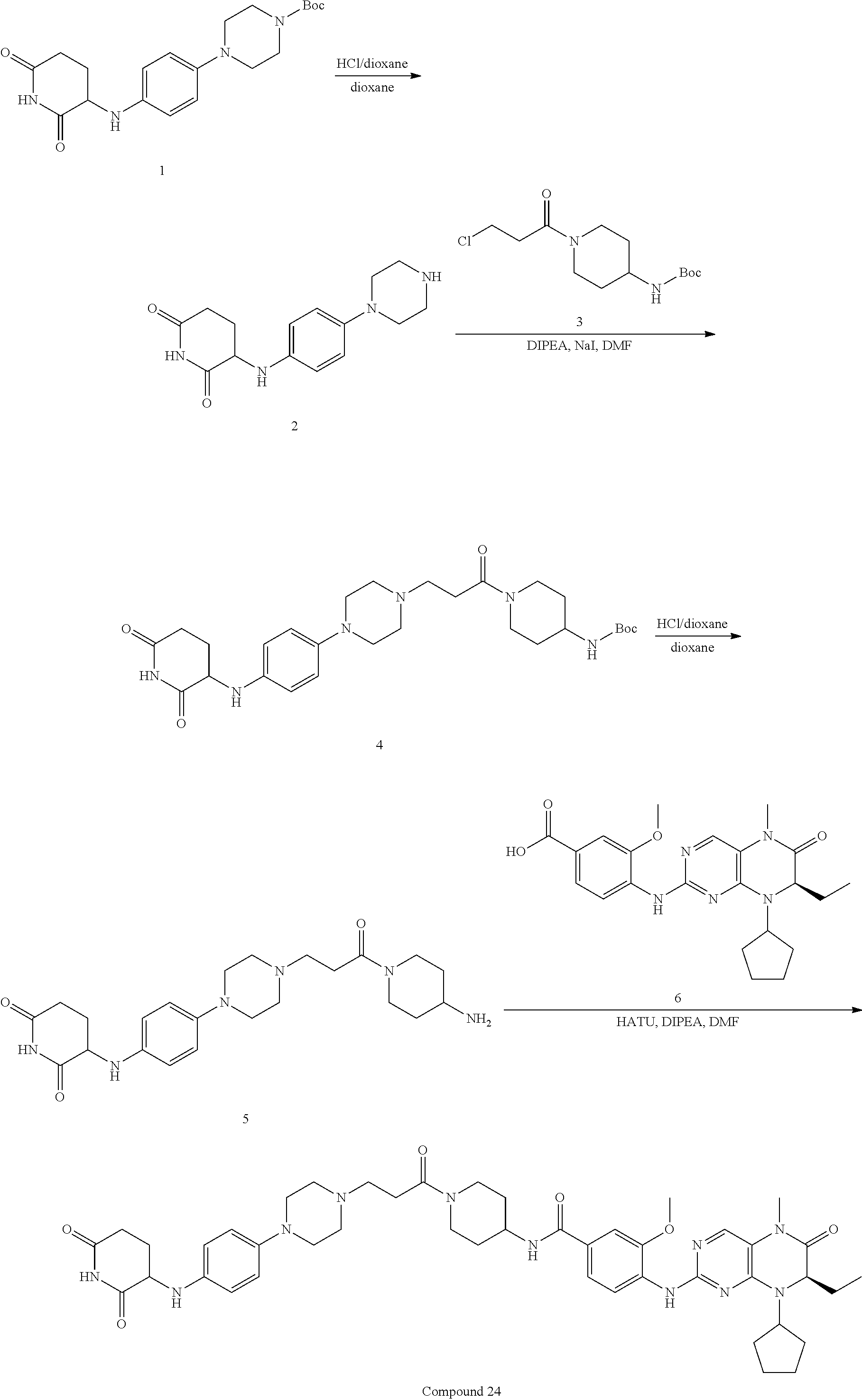

### Step 1. Synthesis of 3-((4-(piperazin-1-yl)phenyl)amino)piperidine-2,6-dione (2)

To a solution of tert-butyl 4-(4-((2,6-dioxopiperidin-3-yl)amino)phenyl)piperazine-1-carboxylate (1 g, 2.57 mmol) in dioxane (20 mL) was added HCl/dioxane (2 M, 7.72 mL). The mixture was stirred at 25° C. for 8.5 hr. LCMS showed 3% of tert-butyl 4-(4-((2,6-dioxopiperidin-3-yl)amino)phenyl)piperazine-1-carboxylate remained and 79% of desired compound. The mixture reaction concentrated under reduced pressure to afford 3-((4-(piperazin-1-yl)phenyl)amino)piperidine-2,6-dione (1.45 g, crude, HCl) as a blue solid. MS(M+H)⁺=289.2.

### Step 2. Synthesis of tert-butyl (1-(3-(4-(4-((2,6-dioxopiperidin-3-yl))amino)phenyl)piperazin-1-yl)propanoyl)piperidin-4-yl)carbamate (4)

To a solution of 3-((4-(piperazin-1-yl)phenyl)amino)piperidine-2,6-dione (1.35 g, 4.16 mmol, HCl) and tert-butyl (1-(3-chloropropanoyl)piperidin-4-yl)carbamate (1.09 g, 3.74 mmol) in DMF (20 mL) were added NaI (747.61 mg, 4.99 mmol) and DIPEA (1.61 g, 12.47 mmol, 2.17 mL). The mixture was stirred at 80° C. for 16 hr. LCMS showed 3-((4-(piperazin-1-yl)phenyl)amino)piperidine-2,6-dione was consumed completely and 48% of desired compound. The residue was diluted with H₂O (100 mL) and extracted with EtOAc (100 mL×3). The combined organic layers were washed with brine (200 mL), dried over Na₂SO₄, filtered and concentrated under reduced pressure. The residue was purified by flash silica gel chromatography (20 g SepaFlash® Silica Flash Column, Eluent of 100~50% EtOAc/MeOH @45 mL/min) to afford tert-butyl (1-(3-(4-(4-((2,6-dioxopiperidin-3-yl)amino)phenyl)piperazin-1-yl)propanoyl)piperidin-4-yl)carbamate (0.65 g, 1.08 mmol, 25.94% yield, 90% purity) as a purple solid. MS(M+H)⁺=543.4.

### Step 3. Synthesis of 3-((4-(4-(3-(4-aminopiperidin-1-yl))-3-oxopropyl)piperazin-1-yl)phenyl)amino)piperidine-2,6-dione (5)

To a solution of tert-butyl (1-(3-(4-(4-((2,6-dioxopiperidin-3-yl)amino)phenyl)piperazin-1-yl)propanoyl)piperidin-4-yl)carbamate (650 mg, 1.12 mmol, HCl) in dioxane (10 mL) was added HCl/dioxane (2 M, 10 mL). The mixture was stirred at 25° C. for 2 hr. LCMS showed 63% of desired compound. The reaction mixture was concentrated to afford 3-((4-(4-(3-(4-aminopiperidin-1-yl)-3-oxopropyl)piperazin-1-yl)phenyl)amino)piperidine-2,6-dione (790 mg, crude, HCl) as a blue solid. MS(M+H)⁺=443.3.

### Step 4. Synthesis of 4-(((R)-8-cyclopentyl-7-ethyl-5-methyl-6-oxo-5,6,7,8-tetrahydropteridin-2-yl)amino)-N-(1-(3-(4-(4-((2,6-dioxopiperidin-3-yl)aminol)phenyl)piperazin-1-yl)propanoyl)piperidin-4-yl)-3-methoxybenzamide (Compound 24)

To a solution of 3-((4-(4-(3-(4-aminopiperidin-1-yl)-3-oxopropyl)piperazin-1-yl)phenyl)amino)piperidine-2,6-dione (790 mg, 1.65 mmol HCl) and (R)-4-((8-cyclopentyl-7-ethyl-5-methyl-6-oxo-5,6,7,8-tetrahydropteridin-2-yl)amino)-3-methoxybenzoic acid (467.81 mg, 1.10 mmol) in DMF (10 mL) were added HATU (543.47 mg, 1.43 mmol) and DIPEA (710.50 mg, 5.50 mmol, 957.55 L). The mixture was stirred at 25° C. for 2 hr. LCMS showed 42% of desired compound. The reaction mixture was poured into water (50 mL) and extracted with EtOAc (60 mL×3). The combined organic layers were washed with brine (200 mL), dried over Na₂SO₄ and concentrated to afford the crude product. The residue was purified by flash silica gel chromatography (ISCO®; 20 g SepaFlash® Silica Flash Column, Eluent of 100~30% DCM:MeOH @50 mL/min) to afford the crude product. The crude product was purified by prep-HPLC (column: Waters Xbridge C18 150×50 mm×10 um; mobile phase: [water(NH₄HCO₃)-ACN]; gradient: 25%-55% B over 10 min) and the eluent was lyophilized to afford 4-(((R)-8-cyclopentyl-7-ethyl-5-methyl-6-oxo-5,6,7,8-tetrahydropteridin-2-yl)amino)-N-(1-(3-(4-(4-((2,6-dioxopiperidin-3-yl)amino)phenyl)piperazin-1-yl)propanoyl)piperidin-4-yl)-3-methoxybenzamide (203.5 mg, 220.25 μmol, 20.03% yield, 92% purity) as a blue solid. MS(M+H)⁺=850.5

¹H NMR (400 MHz, DMSO-d₆) δ=10.75 (br s, 1H), 8.42 (d, J=8.9 Hz, 1H), 8.11 (d, J=7.9 Hz, 1H), 7.84 (s, 1H), 7.66-7.55 (m, 1H), 7.52-7.37 (m, 2H), 6.75 (d, J=9.0 Hz, 2H), 6.60 (d, J=8.9 Hz, 2H), 5.37 (d, J=7.1 Hz, 1H), 4.47-4.31 (m, 2H), 4.28-4.13 (m, 2H), 4.13-3.99 (m, 1H), 3.94 (s, 3H), 3.44-3.36 (m, 2H), 3.24 (s, 3H), 3.17-3.08 (m, 1H), 3.00-2.82 (m, 4H), 2.80-2.63 (m, 2H), 2.62-2.52 (m, 8H), 2.16-1.97 (m, 2H), 1.96-1.71 (m, 9H), 1.69-1.54 (m, 3H), 1.54-1.28 (m, 2H), 0.76 (t, J=7.5 Hz, 3H)

### Example 25. Synthesis of 4-(((R)-8-cyclopentyl-7-ethyl-5-methyl-6-oxo-5,6,7,8-tetrahydropteridin-2-yl)amino)-N-((1r,4R)-4-(((1-(6-(2,6-dioxopiperidin-3-yl)pyridin-3-yl)piperidin-4-yl)methyl)amino)cyclohexyl)-3-methoxybenzamide (Compound 25)

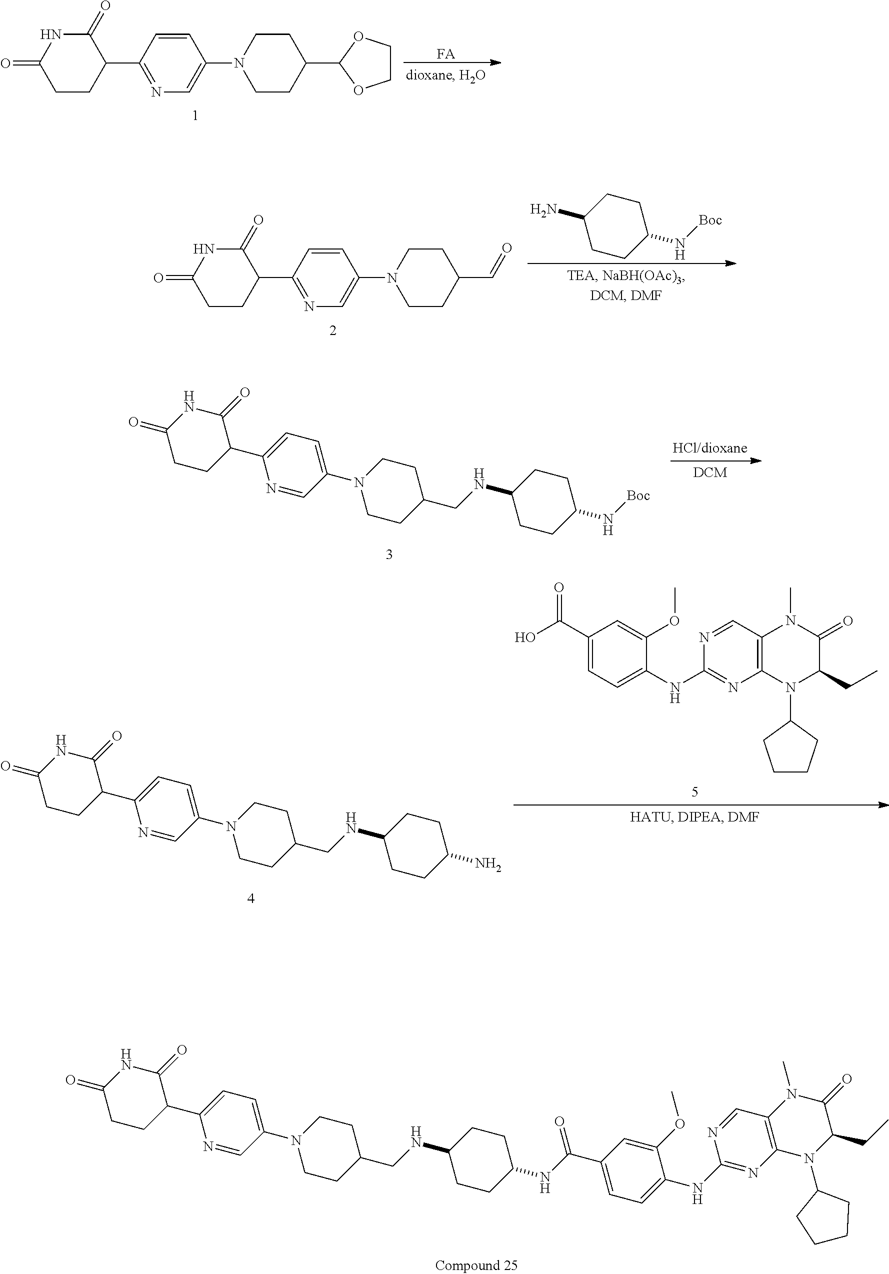

### Step 1. Synthesis of 1-(6-(2,6-dioxopiperidin-3-yl)pyridin-3-yl)piperidine-4-carbaldehyde (2)

To a solution of 3-(5-(4-(1,3-dioxolan-2-yl)piperidin-1-yl)pyridin-2-yl)piperidine-2,6-dione (0.5 g, 1.45 mmol) in dioxane (2 mL) and H₂O (2 mL) was added FA (69.55 mg, 1.45 mmol, 6 mL) and the mixture was stirred at 25° C. for 38 h. The mixture was adjusted the pH=8 with saturated Na₂CO₃ solution at 0° C. and extracted with EtOAc (10 mL×3), the combined organic layer was washed with water (20 mL), dried over Na₂SO₄ and filtered. The filtrate was concentrated under reduced pressure to afford 1-(6-(2,6-dioxopiperidin-3-yl)pyridin-3-yl)piperidine-4-carbaldehyde (380 mg, crude) as a yellow oil. MS(M+H)⁺=302.2

### Step 2. Synthesis of tert-butyl ((1r,4r)-4-(((1-(6-(2,6-dioxopiperidin-3-yl)pyridin-3-yl)piperidin-4-yl)methyl)amino)cyclohexyl)carbamate (trans) (3)

To a solution of 1-(6-(2,6-dioxopiperidin-3-yl)pyridin-3-yl)piperidine-4-carbaldehyde (380 mg, 1.26 mmol), tert-butyl ((1r,4r)-4-aminocyclohexyl)carbamate (trans) (0.3 g, 1.40 mmol) in DCM (6 mL) and DMF (3 mL) was added TEA (261.72 mg, 2.59 mmol, 360 μL) and the mixture was stirred at 25° C. for 15 min. NaBH(OAc)₃ (534.53 mg, 2.52 mmol) was added and the mixture was stirred at 25° C. for 14 h. LCMS showed the desired mass was detected. The mixture was diluted with water (20 mL) and then extracted with EtOAc (10 mL×3), the combined organic layer was washed with water (20 mL), dried over Na₂SO₄ and filtered. The filtrate was concentrated under reduced pressure. The residue was purified by flash silica gel chromatography (5 g SepaFlash® Silica Flash Column, Eluent of 15% MeOH/EtOAc gradient @50 mL/min) to afford tert-butyl ((1r,4r)-4-(((1-(6-(2,6-dioxopiperidin-3-yl)pyridin-3-yl)piperidin-4-yl)methyl)amino)cyclohexyl)carbamate(trans) (250 mg, 480.34 μmol, 38.09% yield, 96% purity) as a yellow solid. MS(M+H)⁺=500.4

### Step 3. Synthesis of 3-(5-(4-((((1r,4r)-4-aminocyclohexyl)amino)methyl)piperidin-1-yl)pyridin-2-yl)piperidine-2,6-dione (trans) (4)

To a solution of tert-butyl ((1r,4r)-4-(((1-(6-(2,6-dioxopiperidin-3-yl)pyridin-3-yl)piperidin-4-yl)methyl)amino)cyclohexyl)carbamate (trans) (250 mg, 500.36 μmol) in DCM (2.5 mL) was added HCl/dioxane (2 M, 5 mL) and the mixture was stirred at 25° C. for 1 h. LCMS showed 93% of the desired mass. The mixture was concentrated under reduced pressure to afford 3-(5-(4-((((1r,4r)-4-aminocyclohexyl)amino)methyl)piperidin-1-yl)pyridin-2-yl)piperidine-2,6-dione (trans) (220 mg, crude, HCl) as a yellow solid. MS(M+H)⁺=400.3

### Step 4. Synthesis of 4-(((R)-8-cyclopentyl-7-ethyl-5-methyl-6-oxo-5,6,7,8-tetrahydropteridin-2-yl)amino)-N-((1r,4R)-4-(((1-(6-(2,6-dioxopiperidin-3-yl)pyridin-3-yl)piperidin-4-yl)methyl)amino)cyclohexyl)-3-methoxybenzamide (trans) (Compound 25)

To a solution of (R)-4-((8-cyclopentyl-7-ethyl-5-methyl-6-oxo-5,6,7,8-tetrahydropteridin-2-yl)amino)-3-methoxybenzoic acid (210 mg, 493.56 μmol) and HATU (206.43 mg, 542.92 μmol) in DMF (3 mL) was added DIPEA (318.95 mg, 2.47 mmol, 429.85 μL) and the mixture was stirred at 25° C. for 15 min. 3-(5-(4-((((1r,4r)-4-aminocyclohexyl)amino)methyl)piperidin-1-yl)pyridin-2-yl)piperidine-2,6-dione (trans) (220 mg, 504.60 μmol, HCl) in DMF (3 mL) was added and the mixture was stirred at 25° C. for 1 h. LCMS showed 53% of the desired mass. The mixture was diluted with EtOAc (10 mL) and saturated NaHCO₃ solution (10 mL) and then extracted with EtOAc (10 mL×3) and DCM (10 mL×3), the combined organic layer was washed with brine (30 mL×2), dried over Na₂SO₄ and filtered. The filtrate was concentrated under reduced pressure and then purified by prep-HPLC (column: Phenomenex luna C18 150*25 mm*10 um; mobile phase: [water (TFA)-ACN]; gradient: 14%-44% B over 10 min) and the eluent was lyophilized. The product was re-purified by prep-HPLC (column: Waters Xbridge 150*25 mm*5 um; mobile phase: [water (NH₄HCO₃)-ACN]; gradient: 30%-50% B over 10 min) and the eluent was lyophilized to afford 4-(((R)-8-cyclopentyl-7-ethyl-5-methyl-6-oxo-5,6,7,8-tetrahydropteridin-2-yl)amino)-N-((1r,4R)-4-(((1-(6-(2,6-dioxopiperidin-3-yl)pyridin-3-yl)piperidin-4-yl)methyl)amino)cyclohexyl)-3-methoxybenzamide (trans) (57.80 mg, 67.33 μmol, 13.64% yield, 94% purity) as a white solid. MS(M+H)⁺=807.7

¹H NMR (400 MHz, DMSO-d₆) δ=10.83-10.70 (m, 1H), 8.40 (d, J=8.8 Hz, 1H), 8.20-8.17 (m, 1H), 8.02 (d, J=8.1 Hz, 1H), 7.84 (s, 1H), 7.59 (s, 1H), 7.49-7.44 (m, 2H), 7.31 (dd, J=2.8, 8.7 Hz, 1H), 7.15 (d, J=8.7 Hz, 1H), 4.36 (t, J=8.0 Hz, 1H), 4.23 (dd, J=3.4, 7.5 Hz, 1H), 3.94 (s, 3H), 3.87 (dd, J=5.4, 8.7 Hz, 1H), 3.78-3.67 (m, 3H), 3.24 (s, 3H), 2.67 (t, J=11.2 Hz, 2H), 2.60-2.52 (m, 1H), 2.48-2.43 (m, 3H), 2.37-2.28 (m, 1H), 2.22-2.15 (m, 1H), 2.13-2.07 (m, 1H), 2.03-1.99 (m, 1H), 1.97-1.71 (m, 13H), 1.68-1.56 (m, 3H), 1.54-1.45 (m, 1H), 1.43-1.31 (m, 2H), 1.28-1.17 (m, 2H), 1.14-1.03 (m, 2H), 0.76 (t, J=7.4 Hz, 3H).

SFC Method details: “Column: Chiralpak AD-3 50×4.6 mm I. D., 3 um Mobile phase: Phase A for CO₂, and Phase B for IPA+ACN (0.05% DEA); Gradient elution: 60% IPA+ACN (0.05% DEA) in CO₂ Flow rate: 3 mL/min; Detector: PDA Column Temp: 35 C; Back Pressure: 100 Bar”

### Example 26. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(3-(4-(4-((2,6-dioxopiperidin-3-yl)amino)-2-fluorophenyl)piperazin-1-yl)propanoyl)piperidin-4-yl)-3-methoxybenzamide (Compound 26)

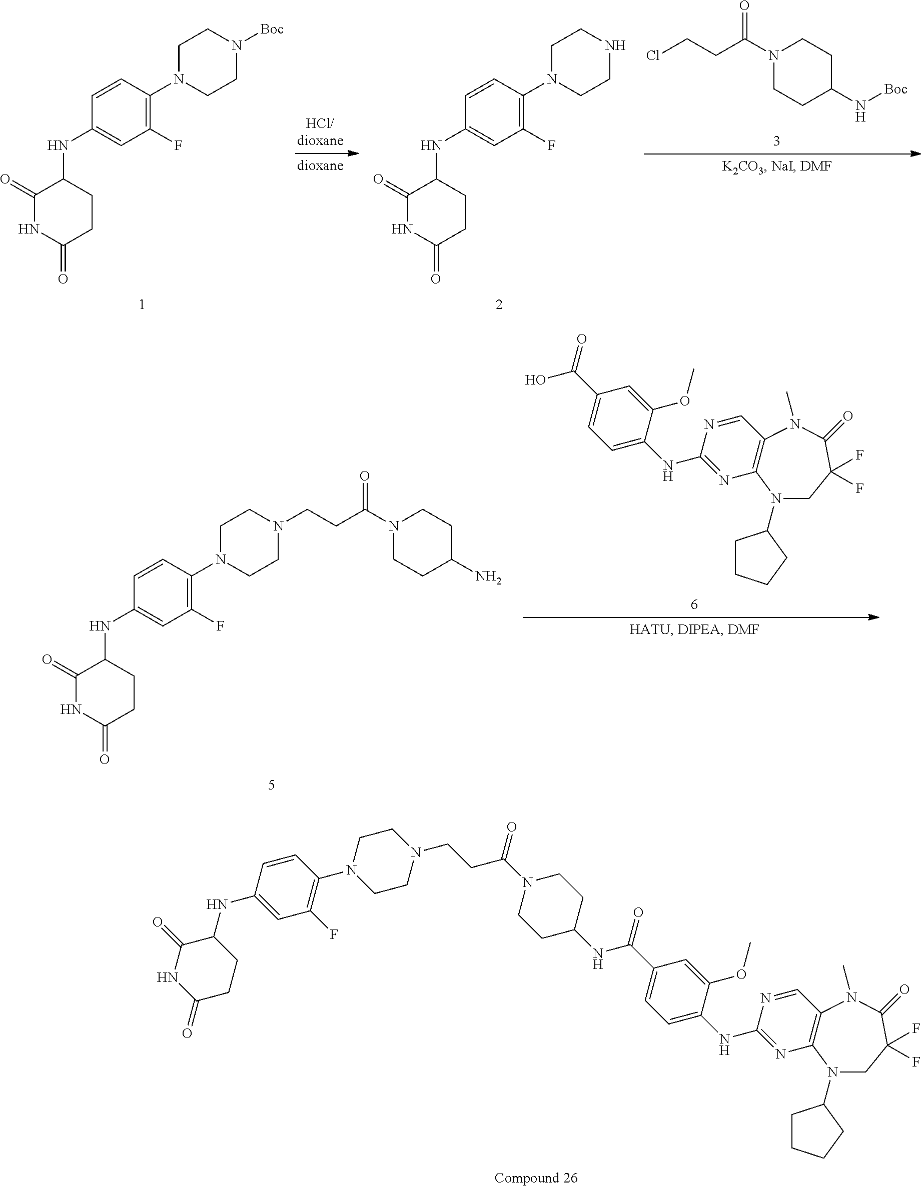 [SMILES: CC(C)(C)OC(=O)N1CCN(c2ccc(NC3CCC(=O)NC3=O)cc2F)CC1 ; O=C1CCC(Nc2ccc(N3CCNCC3)c(F)c2)C(=O)N1 ; CC(C)(C)OC(=O)NC1CCN(C(=O)CCCl)CC1 ; NC1CCN(C(=O)CCN2CCN(c3ccc(NC4CCC(=O)NC4=O)cc3F)CC2)CC1 ; COc1cc(C(=O)O)ccc1Nc1ncc2c(n1)N(C1CCCC1)CC(F)(F)C(=O)N2C ; COc1cc(C(=O)NC2CCN(C(=O)CCN3CCN(c4ccc(NC5CCC(=O)NC5=O)cc4F)CC3)CC2)ccc1Nc1ncc2c(n1)N(C1CCCC1)CC(F)(F)C(=O)N2C]

### Step 1. Synthesis of 3-((3-fluoro-4-(piperazin-1-yl)phenyl)amino)piperidine-2,6-dione (2)

To a solution of tert-butyl 4-(4-((2,6-dioxopiperidin-3-yl)amino)-2-fluorophenyl)piperazine-1-carboxylate (500 mg, 1.23 mmol) in dioxane (5 mL) was added HCl/dioxane (4 M, 5 mL), the mixture was stirred at 20° C. for 1 h. LCMS showed the starting material was consumed completely and a main peak with desired mass. The mixture was concentrated in vacuum to afford 3-((3-fluoro-4-(piperazin-1-yl)phenyl)amino)piperidine-2,6-dione (420 mg, crude, HCl) as a gray solid. MS(M+H)⁺=307.1.

### Step 2. Synthesis of tert-butyl (1-(3-(4-(4-((2,6-dioxopiperidin-3-yl)amino)-2-fluorophenyl)piperazin-1-yl)propanoyl piperidin-4-yl)carbamate (4)

To a solution of 3-((3-fluoro-4-(piperazin-1-yl)phenyl)amino)piperidine-2,6-dione (420 mg, crude, HCl), tert-butyl (1-(3-chloropropanoyl)piperidin-4-yl)carbamate (1.78 g, 6.13 mmol) in DMF (10 mL) were added K₂CO₃ (677.33 mg, 4.90 mmol), NaI (18.37 mg, 122.52 μmol), the mixture was stirred at 90° C. for 16 h. LCMS showed the starting material was consumed completely and a main peak with desired mass. The reaction mixture was diluted with brine (30 mL), extracted with EtOAc (20 mL×3). The combined organic layers were washed with brine (80 mL×3), dried over Na₂SO₄, filtered. The filtrate was concentrated in vacuum to give a residue. The residue was purified by flash silica gel chromatography (Biotage; 20 g SepaFlash® Silica Flash Column, Eluent of 50~100% EtOAc/Petroleum ether to 10% MeOH/EtOAc gradient @100 mL/min) to afford tert-butyl (1-(3-(4-(4-((2,6-dioxopiperidin-3-yl)amino)-2-fluorophenyl)piperazin-1-yl)propanoyl) piperidin-4-yl)carbamate (630 mg, 1.12 mmol, 91.71% yield) as a gray solid. MS(M+H)⁺=561.3.

### Step 3. Synthesis of 3-((4-(4-(3-(4-aminopiperidin-1-yl))-3-oxopropyl)piperazin-1-yl)-3-fluorophenyl)amino)piperidine-2,6-dione (5)

To a solution of tert-butyl (1-(3-(4-(4-((2,6-dioxopiperidin-3-yl)amino)-2-fluorophenyl)piperazin-1-yl)propanoyl) piperidin-4-yl)carbamate (630 mg, 1.12 mmol) in dioxane (10 mL) was added HCl/dioxane (4 M, 10 mL), the mixture was stirred at 20° C. for 0.5 h. LCMS showed the starting material was consumed completely and a main peak with desired mass. The mixture was concentrated in vacuum to afford 3-((4-(4-(3-(4-aminopiperidin-1-yl)-3-oxopropyl)piperazin-1-yl)-3-fluorophenyl)amino)piperidine-2,6-dione (550 mg, crude, HCl) as a yellow solid. MS(M+H)⁺=461.2.

### Step 4. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(3-(4-(4-((2,6-dioxopiperidin-3-yl)amino)-2-fluorophen yl)piperazin-1-yl)propanoyl)piperidin-4-yl)-3-methoxybenzamide (Compound 26)

To a solution of 4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzoic acid (270.08 mg, 603.62 μmol) in DMF (6 mL) were added HATU (344.27 mg, 905.42 μmol), DIPEA (390.07 mg, 3.02 mmol, 525.70 μL), the mixture was stirred at 20° C. for 0.5 h, 3-((4-(4-(3-(4-aminopiperidin-1-yl)-3-oxopropyl)piperazin-1-yl)-3-fluorophenyl)amino)piperidine-2,6-dione (300 mg, crude, HCl) was added, the resulting mixture was stirred at 20° C. for 16 h. LCMS showed the starting material was consumed completely and a main peak with desired mass. The reaction mixture was diluted with brine (15 mL), extracted with EtOAc (20 mL×3). The combined organic layers were washed with brine (80 mL×3), dried over Na₂SO₄, filtered. The filtrate was concentrated in vacuum to give a residue. The residue was purified by flash silica gel chromatography (Biotage; 10 g SepaFlash® Silica Flash Column, Eluent of 50~100% EtOAc/Petroleum ether to 10% MeOH/EtOAc gradient @200 mL/min) and re-purified by reversed-phase HPLC (column: Waters Xbridge 150*25 mm*5 um; mobile phase: [water (NH₄HCO₃)-ACN]; B %:36%-66%, 10 min). The eluent was lyophilized to afford 4-((9-cyclopentyl-7,7-difluoro-5-methyl-6-oxo-6,7,8,9-tetrahydro-5H-pyrimido[4,5-b][1,4]diazepin-2-yl)amino)-N-(1-(3-(4-(4-((2,6-dioxopiperidin-3-yl)amino)-2-fluorophen yl)piperazin-1-yl)propanoyl)piperidin-4-yl)-3-methoxybenzamide (200.9 mg, 218.97 mol, 36.28% yield, 97% purity) as a gray solid. MS(M+H)⁺=890.3.

¹H NMR (400 MHz, DMSO-d₆) δ=10.76 (s, 1H), 8.30-8.23 (m, 2H), 8.14 (d, J=7.5 Hz, 1H), 7.96 (s, 1H), 7.52-7.45 (m, 2H), 6.87-6.77 (m, 1H), 6.54-6.47 (m, 1H), 6.44-6.38 (m, 1H), 5.79 (d, J=7.7 Hz, 1H), 4.83-4.70 (m, 1H), 4.44-4.33 (m, 1H), 4.29-4.21 (m, 1H), 4.09-4.00 (m, 3H), 3.96-3.90 (m, 4H), 3.29 (s, 3H), 3.16-3.09 (m, 1H), 2.89-2.81 (m, 4H), 2.76-2.54 (m, 11H), 2.11-2.06 (m, 1H), 1.96-1.81 (m, 5H), 1.74-1.67 (m, 2H), 1.64-1.56 (m, 4H), 1.51-1.36 (m, 2H).

### Example 27. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-((1-((1-(4-(2,6-dioxopiperidin-3-yl)phenyl)piperidin-4-yl)methyl)piperidin-4-yl)methyl)-3-methoxybenzamide (Compound 27)

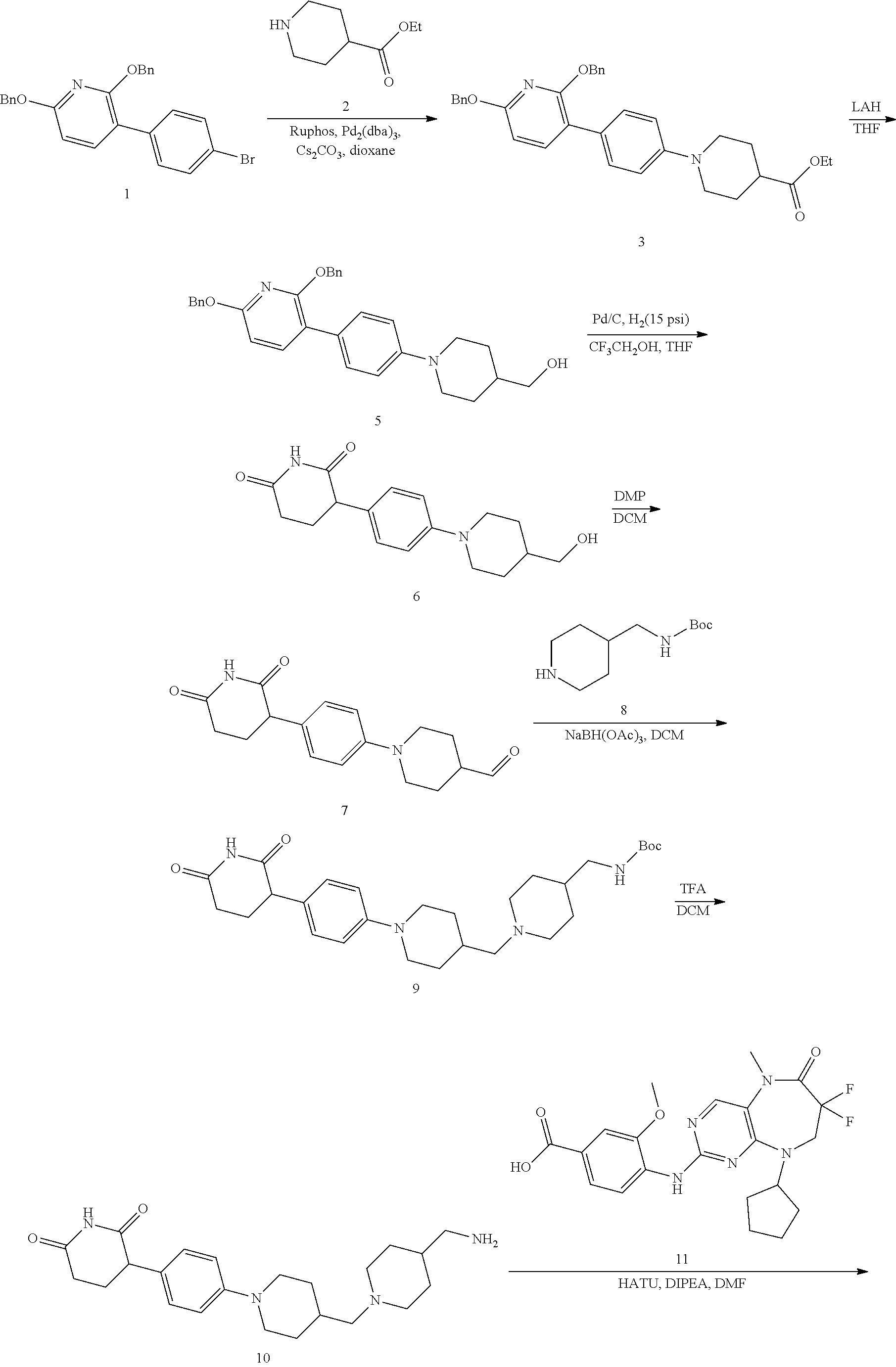

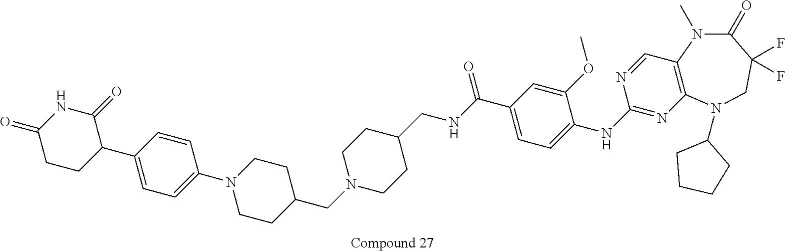 [SMILES: COc1cc(C(=O)NCC2CCN(CC3CCN(c4ccc(C5CCC(=O)NC5=O)cc4)CC3)CC2)ccc1Nc1ncc2c(n1)N(C1CCCC1)CC(F)(F)C(=O)N2C]

### Step 1. Synthesis of ethyl 1-(4-(2,6-bis(benzyloxy)pyridin-3-yl)phenyl)piperidine-4-carboxylate (3)

A mixture of 2,6-bis(benzyloxy)-3-(4-bromophenyl)pyridine (1.9 g, 4.26 mmol), ethyl piperidine-4-carboxylate (803.07 mg, 5.11 mmol), RuPhos (198.64 mg, 425.69 mol), Pd₂(dba)₃ (389.81 mg, 425.69 μmol) and Cs₂CO₃ (4.16 g, 12.77 mmol) in dioxane (40 mL) was degassed and purged with N₂ for 3 times, the resulting mixture was stirred at 100° C. for 16 h under N₂ atmosphere. LCMS showed a peak (75%) with desired mass. The mixture was filtered through a pad of celite. The filtrate was concentrated in vacuum. The residue was purified by flash silica gel chromatography (Biotage; 20 g SepaFlash® Silica Flash Column, Eluent of 0~20% EtOAc/Petroleum ether gradient @100 mL/min) to afford ethyl 1-(4-(2,6-bis(benzyloxy)pyridin-3-yl)phenyl)piperidine-4-carboxylate (1.9 g, 3.64 mmol, 85.40% yield) as a white solid. MS(M+H)⁺=523.2

### Step 2. Synthesis of (1-(4-(2,6-bis(benzyloxypyridin-3-yl)phenyl)piperidin-4-yl)methanol (5)

To a solution of ethyl 1-(4-(2,6-bis(benzyloxy)pyridin-3-yl)phenyl)piperidine-4-carboxylate (1.1 g, 2.10 mmol) in THF (20 mL) was added LAH (160 mg, 4.22 mmol) at 0° C., the mixture was stirred at 25° C. for 2 hours. LCMS showed a main peak with desired mass. The mixture was quenched by addition H₂O (0.5 mL), NaOH (15% aq, 0.5 mL), H₂O (1.5 mL), dried over Na₂SO₄ and filtered. The filtrate was concentrated in vacuum. The residue was purified by flash silica gel chromatography (Biotage; 20 g SepaFlash® Silica Flash Column, Eluent of 0~50% EtOAc/Petroleum ether gradient @100 mL/min) to afford (1-(4-(2,6-bis(benzyloxy)pyridin-3-yl)phenyl)piperidin-4-yl)methanol (0.9 g, 1.87 mmol, 88.97% yield) as a white solid. MS(M+H)⁺=481.2

### Step 3. Synthesis of 3-(4-(4-(hydroxymethyl)piperidin-1-yl)phenyl)piperidine-2,6-dione (6)

A mixture of (1-(4-(2,6-bis(benzyloxy)pyridin-3-yl)phenyl)piperidin-4-yl)methanol (800 mg, 1.66 mmol) and Pd/C (200 mg, 1.66 mmol, 10% purity) in CF₃CH₂OH (10 mL) and THF (2 mL) was degassed and purged with H₂ (15 Psi) for 3 times, and the mixture was stirred at 20° C. for 16 h. LCMS showed a main peak with desired mass. The mixture was filtered through a pad of celite. The filtrate was concentrated in vacuum. The residue was purified by flash silica gel chromatography (Biotage; 20 g SepaFlash® Silica Flash Column, Eluent of 50~100% EtOAc/Petroleum ether gradient @100 mL/min) to afford 3-(4-(4-(hydroxymethyl)piperidin-1-yl)phenyl)piperidine-2,6-dione (480 mg, 1.59 mmol, 95.37% yield) as a white solid. MS(M+H)⁺=303.1

### Step 4. Synthesis of 1-(4-(2,6-dioxopiperidin-3-yl)phenyl)piperidine-4-carbaldehyde

To a solution of 3-(4-(4-(hydroxymethyl)piperidin-1-yl)phenyl)piperidine-2,6-dione (200 mg, 661.45 μmol) in DCM (1 mL) was added DMP (336.66 mg, 793.74 μmol), the mixture was stirred at 20° C. for 1 h. LCMS showed the desired mass was detected. The mixture was filtered through a pad of celite. The filtrate was concentrated in vacuum to afford 1-(4-(2,6-dioxopiperidin-3-yl)phenyl)piperidine-4-carbaldehyde (200 mg, crude) as a brown oil. MS(M+H)⁺=301.1

### Step 5. Synthesis of tert-butyl ((1-((1-(4-(2,6-dioxopiperidin-3-yl)phenyl)piperidin-4-yl)methyl)piperidin-4-yl)methyl)carbamate (9)

A solution of 1-(4-(2,6-dioxopiperidin-3-yl)phenyl)piperidine-4-carbaldehyde (200 mg, crude), tert-butyl (piperidin-4-ylmethyl)carbamate (142.70 mg, 665.89 μmol) in DCM (2 mL) was stirred at 20° C. for 0.5 h, NaBH(OAc)₃ (423.39 mg, 2.00 mmol) was added, the mixture was stirred at 20° C. for 16 h. LCMS showed a peak (98%) with desired mass. The reaction mixture was quenched by NaHCO₃ (5 mL), extracted with EtOAc (10 mL×3). The combined organic layers was washed with brine (20 mL×2), dried over Na₂SO₄, filtered and the filtrate was concentrated in vacuum. The residue was purified by flash silica gel chromatography (Biotage; 10 g SepaFlash® Silica Flash Column, Eluent of 50~100% EtOAc/Petroleum ether to 10% MeOH/EtOAc gradient @100 mL/min) and then triturated with a mixture solution (6 mL, DMF:ACN=5:1) at 20° C. for 0.5 h and filtered, the filter cake was dried in vacuum to afford tert-butyl ((1-((1-(4-(2,6-dioxopiperidin-3-yl)phenyl)piperidin-4-yl)methyl)piperidin-4-yl)methyl)carbamate (74 mg, 148.40 μmol, 37.00% yield, 78% purity) as a purple solid. The filtrate was re-purified by prep-HPLC(column: Phenomenex Synergi Polar-RP 100*25 mm*4 um; mobile phase: [water(TFA)-ACN]; B %: 10%-40%, 9 min), the eluent was lyophilized to afford tert-butyl ((1-((1-(4-(2,6-dioxopiperidin-3-yl)phenyl)piperidin-4-yl)methyl)piperidin-4-yl)methyl)carbamate (130 mg, 260.70 μmol, 65.00% yield, 93% purity) as a white solid. MS(M+H)⁺=499.3

### Step 6. Synthesis of 3-(4-(4-((4-(aminomethyl)piperidin-1-yl)methyl)piperidin-1-yl)phenyl)piperidine-2,6-dione (10)

To a solution of tert-butyl ((1-((1-(4-(2,6-dioxopiperidin-3-yl)phenyl)piperidin-4-yl)methyl)piperidin-4-yl)methyl)carbamate (130 mg, 212.18 μmol, TFA salt) in DCM (2 mL) was added TFA (241.94 mg, 2.12 mmol) at 20° C. The mixture was stirred at 20° C. for 20 min. LCMS showed starting material was consumed completely and desired mass was detected. The reaction mixture was concentrated in vacuum to afford 3-(4-(4-((4-(aminomethyl)piperidin-1-yl)methyl)piperidin-1-yl)phenyl)piperidine-2,6-dione (109 mg, crude, TFA) as a yellow oil. MS(M+H)⁺=399.3

### Step 7. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-((1-((1-(4-(2,6-dioxopiperidin-3-yl)phenyl)piperidin-4-yl) methyl)piperidin-4-yl)methyl)-3-methoxybenzamide (Compound 27)

To a solution of 4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzoic acid (70 mg, 156.45 μmol) in DMF (2 mL) were added HATU (65.43 mg, 172.09 μmol) and DIPEA (60.66 mg, 469.34 μmol) at 20° C., after stirring for 10 min, then a solution of 3-(4-(4-((4-(aminomethyl)piperidin-1-yl)methyl)piperidin-1-yl)phenyl)piperidine-2,6-dione (109 mg, crude, TFA salt) in DMF (2 mL) and DIPEA (121.32 mg, 938.69 mol) was added at 20° C. and the resulting mixture was stirred at 20° C. for 1 h. LCMS showed starting material was consumed completely and a main peak (90%) with desired mass. The reaction mixture was diluted with H₂O (15 mL) and extracted with EtOAc (10 mL×3). The combined organic layer was washed with brine (10 mL×3), dried over Na₂SO₄, filtered and concentrated. The residue was purified by prep-TLC (SiO₂, DCM:MeOH=10:1) to afford a residue. The residue was combined with another batch (30 mg scale) for lyophilization to afford 4-((9-cyclopentyl-7,7-difluoro-5-methyl-6-oxo-6,7,8,9-tetrahydro-5H-pyrimido[4,5-b][1,4]diazepin-2-yl)amino)-N-((1-((1-(4-(2,6-dioxopiperidin-3-yl)phenyl)piperidin-4-yl) methyl)piperidin-4-yl)methyl)-3-methoxybenzamide (113 mg, 136.48 μmol, 87.24% yield) as a white solid. MS(M+H)⁺=828.2

¹H NMR (400 MHz, DMSO-d₆) δ=10.76 (s, 1H), 8.48-8.35 (m, 1H), 8.31-8.23 (m, 2H), 7.97 (s, 1H), 7.55-7.45 (m, 2H), 7.03 (d, J=8.6 Hz, 2H), 6.88 (d, J=8.7 Hz, 2H), 4.84-4.70 (m, 1H), 4.05 (t, J=14.1 Hz, 2H), 3.93 (s, 3H), 3.76-3.60 (m, 3H), 3.33-3.30 (m, 5H), 3.17 (s, 2H), 3.04-2.71 (m, 2H), 2.68-2.58 (m, 3H), 2.48-2.41 (m, 1H), 2.21-2.07 (m, 2H), 2.05-1.86 (m, 4H), 1.82-1.54 (m, 12H), 1.34-1.08 (m, 4H).

### Example 28. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-((1-((1-(4-((2,6-dioxopiperidin-3-yl)amino)phenyl) piperidin-4-yl)methyl)piperidin-4-yl)methyl)-3-methoxybenzamide (Compound 28)

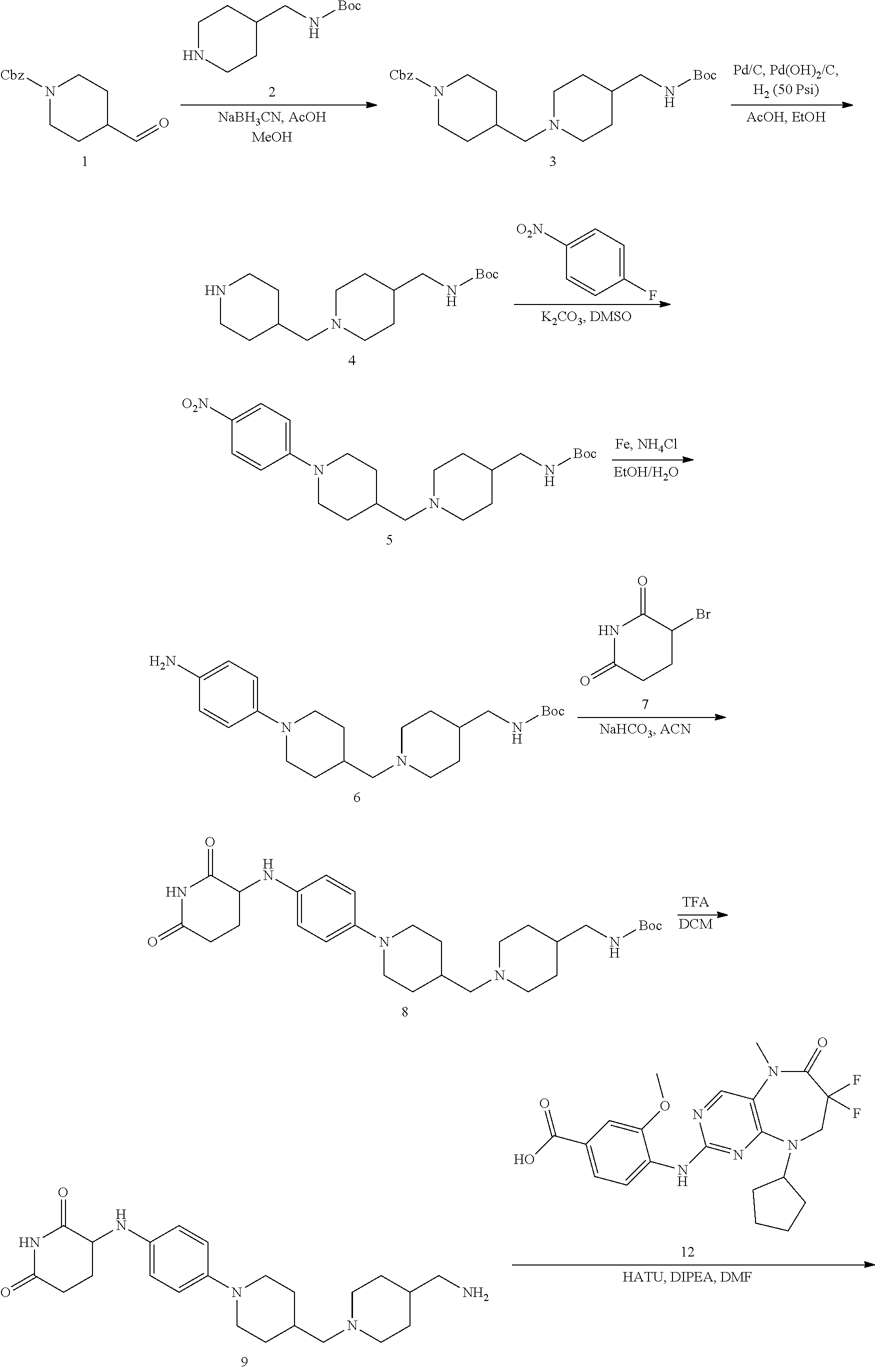

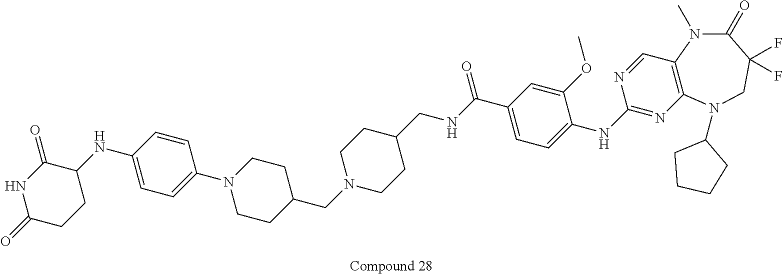 [SMILES: COc1cc(C(=O)NCC2CCN(CC3CCN(c4ccc(NC5CCC(=O)NC5=O)cc4)CC3)CC2)ccc1Nc1ncc2c(n1)N(C1CCCC1)CC(F)(F)C(=O)N2C]

### Step 1. Synthesis of benzyl 4-((4-(((tert-butoxycarbonyl)amino)methyl)piperidin-1-yl)methyl)piperidine-1-carboxylate (3)

To a solution of benzyl 4-formylpiperidine-1-carboxylate (10 g, 40.44 mmol) and tert-butyl (piperidin-4-ylmethyl) carbamate (8.67 g, 40.44 mmol) in MeOH (200 mL) was added AcOH (2.43 g, 40.44 mmol, 2.31 mL) at 20° C., after stirring 30 min, then NaBH₃CN (2.54 g, 40.44 mmol) was added slowly at 20° C. and the resulting reaction mixture was stirred at 20° C. for 16 h. LCMS showed starting material was consumed completely and a peak (62%) with desired mass. The reaction mixture was diluted with H₂O (300 mL) and extracted with EtOAc (150 mL×3). The combined organic layer was washed with NaHCO₃ (100 mL×3), dried over Na₂SO₄, filtered. The filtrate was concentrated in vacuum. The residue was purified by flash silica gel chromatography (80 g SepaFlash® Silica Flash Column, Eluent of 0~100% EtOAc/Petroleum ether gradient @200 mL/min) followed by flash silica gel chromatography (ISCO®; 80 g SepaFlash® Silica Flash Column, Eluent of 0~100% EtOAc/Petroleum ether gradient @200 mL/min) to afford benzyl 4-((4-(((tert-butoxycarbonyl)amino)methyl)piperidin-1-yl)methyl)piperidine-1-carboxylate (14 g, 31.42 mmol, 77.70% yield) as a white solid. MS(M+H)⁺=446.4.

### Step 2. Synthesis of tert-butyl ((1-(piperidin-4-ylmethyl)piperidin-4-yl)methyl)carbamate (4)

To a solution of benzyl 4-((4-(((tert-butoxycarbonyl)amino)methyl)piperidin-1-yl)methyl)piperidine-1-carboxylate (2 g, 4.49 mmol) in EtOH (20 mL) were added Pd/C (1 g, 10% purity) and AcOH (2.10 g, 34.94 mmol, 2 mL) under N₂ atmosphere. The suspension was degassed and purged with H₂ for 3 times. The mixture was stirred at 25° C. for 16 h under H₂ (50 Psi). LCMS showed most of starting material remained and the reaction mixture was stirred at 20° C. for another 16 h. LCMS showed half of starting material remained. Additional Pd(OH)₂/C (1 g, 20% purity), AcOH (1 mL) were added and the suspension was degassed and purged with H₂ for 3 times. The mixture was stirred at 25° C. for 16 h under H₂ (50 Psi). LCMS showed starting material was consumed completely and a peak (86%) with desired mass. The reaction mixture was filtered. The filter cake was washed with EtOH (40 mL×3), the filtrate was concentrated in vacuum to afford tert-butyl ((1-(piperidin-4-ylmethyl)piperidin-4-yl)methyl)carbamate (1.5 g, crude) as a colorless oil. MS(M+H)⁺=312.5.

### Step 3. Synthesis of tert-butyl ((1-((1-(4-nitrophenyl)piperidin-4-yl)methyl)piperidin-4-yl)methyl)carbamate (5)

To a solution of tert-butyl ((1-(piperidin-4-ylmethyl)piperidin-4-yl)methyl)carbamate (1.5 g, 4.04 mmol) and 1-fluoro-4-nitrobenzene (455.76 mg, 3.23 mmol, 342.67 L) in DMSO (40 mL) was added K₂CO₃ (2.23 g, 16.15 mmol) at 20° C. The mixture was stirred at 20° C. for 16 h. LCMS showed a peak (26% peak) with desired mass. The reaction mixture was diluted with H₂O (80 mL) and extracted with EtOAc (30 mL×3), the organic layer was washed with brine (30 mL×3), dried over Na₂SO₄, filtered. The filtrate was concentrated in vacuum. The residue was purified by flash silica gel chromatography (80 g SepaFlash® Silica Flash Column, Eluent of 0~100% EtOAc/Petroleum ether gradient @200 mL/min) to afford tert-butyl ((1-((1-(4-nitrophenyl)piperidin-4-yl)methyl)piperidin-4-yl)methyl)carbamate (400 mg, 924.74 μmol, 22.90% yield) as a yellow oil. MS(M+H)⁺=433.3.

### Step 4. Synthesis of tert-butyl ((1-((1-(4-aminophenyl)piperidin-4-yl)methyl)piperidin-4-yl)methyl)carbamate (6)

To a solution of tert-butyl ((1-((1-(4-nitrophenyl)piperidin-4-yl)methyl)piperidin-4-yl)methyl)carbamate (400 mg, 924.74 μmol) in EtOH (10 mL) and H₂O (2 mL) were added Fe (258.23 mg, 4.62 mmol) and NH₄Cl (247.32 mg, 4.62 mmol) at 20° C. The mixture was stirred at 80° C. for 2 h. LCMS showed most of the starting material remained, additional NH₄Cl (247.33 mg, 4.62 mmol) was added and the reaction mixture was stirred at 80° C. for another 1 h. LCMS showed 11% of the starting material remained and a peak (83%) with desired mass. The reaction mixture was diluted with H₂O (40 mL) and adjusted pH=10 with Na₂CO₃, then extracted with EtOAc (30 mL×3). The combined organic layer was dried over Na₂SO₄, filtered. The filtrate was concentrated in vacuum to afford tert-butyl ((1-((1-(4-aminophenyl)piperidin-4-yl)methyl)piperidin-4-yl)methyl)carbamate (370 mg, crude) as a brown solid. MS(M+H)⁺=403.3.

### Step 5. Synthesis of tert-butyl ((1-((1-(4-((2,6-dioxopiperidin-3-yl)amino)phenyl)piperidin-4-yl)methyl)piperidin-4-yl)methyl)carbamate (8)

To a solution of tert-butyl ((1-((1-(4-aminophenyl)piperidin-4-yl)methyl)piperidin-4-yl)methyl)carbamate (185 mg, 459.55 μmol) and 3-bromopiperidine-2,6-dione (264.71 mg, 1.38 mmol) in ACN (1.5 mL) was added NaHCO₃ (231.63 mg, 2.76 mmol, 107.29 μL) at 20° C. The mixture was stirred at 80° C. for 16 h. LCMS showed the starting material was consumed completely and a peak (36%) with desired mass. The reaction mixture was filtered, the filter cake was washed with EtOAc (20 mL). The filtrate was concentrated in vacuum to afford crude product. The crude product was combined with another batch (350 mg scale) for purification. The residue was purified by flash silica gel chromatography (12 g SepaFlash® Silica Flash Column, Eluent of 0~100% EtOAc/Petroleum ether to 0~50% DCM/MeOH gradient @100 mL/min) to afford product A (248 mg). The product A was re-purified by prep-TLC (SiO₂, DCM:MeOH=5:1) to afford impure product, which was dissolved in mixture of solvent (20 mL, ACN:H₂O=1:3) to afford tert-butyl ((1-((1-(4-((2,6-dioxopiperidin-3-yl)amino)phenyl)piperidin-4-yl)methyl)piperidin-4-yl)methyl)carbamate (170 mg, 330.95 μmol, 72.02% yield) as a white solid. MS(M+H)⁺=514.3.

### Step 6. Synthesis of 3-((4-(4-((4-(aminomethyl)piperidin-1-yl)methyl)piperidin-1-yl)phenyl)amino)piperidine-2,6-dione (9)

To a solution of tert-butyl ((1-((1-(4-((2,6-dioxopiperidin-3-yl)amino)phenyl)piperidin-4-yl)methyl)piperidin-4-yl)methyl)carbamate (138 mg, 268.65 μmol) in DCM (1 mL) was added TFA (307.00 mg, 2.69 mmol, 0.2 mL), the mixture was stirred at 20° C. for 1 h. LCMS showed starting material was consumed completely and desired mass was detected. The reaction mixture was concentrated in vacuum to afford 3-((4-(4-((4-(aminomethyl)piperidin-1-yl)methyl)piperidin-1-yl)phenyl)amino)piperidine-2,6-dione (228 mg, crude, TFA salt) as a dark green solid. MS(M+H)⁺=414.4.

### Step 7. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-((1-((1-(4-((2,6-dioxopiperidin-3-yl)amino)phenyl)piperidin-4-yl)methyl)piperidin-4-yl)methyl)-3-methoxybenzamide (Compound 28)

To a solution of 4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzoic acid (96.68 mg, 216.08 μmol) in DMF (1 mL) were added HATU (98.59 mg, 259.30 μmol) and DIPEA (74.20 mg, 574.11 μmol, 0.1 mL) at 20° C., after stirring 10 min, then a solution of 3-((4-(4-((4-(aminomethyl)piperidin-1-yl)methyl)piperidin-1-yl)phenyl)amino)piperidine-2,6-dione (114 mg, 216.08 μmol, TFA salt) in DMF (1 mL) with DIPEA (222.60 mg, 1.72 mmol, 0.3 mL) was added at 20° C., the reaction mixture was stirred at 20° C. for 1 h. LCMS showed starting material was consumed completely and a peak (26%) with desired mass. The reaction mixture was diluted with H₂O (20 mL) and extracted with EtOAc (10 mL×3). The combined organic layer was washed with brine (10 mL×3), dried over Na₂SO₄, filtered and concentrated in vacuum. The crude product was purified by prep-TLC (SiO₂, DCM:MeOH=5:1) and re-purify by prep-HPLC (column: Phenomenex luna C18 150*25 mm*10 um; mobile phase: [water (TFA)-ACN]; gradient:18%-48% B over 9 min), the eluent was lyophilized to afford 4-((9-cyclopentyl-7,7-difluoro-5-methyl-6-oxo-6,7,8,9-tetrahydro-5H-pyrimido[4,5-b][1,4]diazepin-2-yl)amino)-N-((1-((1-(4-((2,6-dioxopiperidin-3-yl)amino)phenyl)piperidin-4-yl)methyl)piperidin-4-yl)methyl)-3-methoxybenzamide (31.1 mg, 27.91 μmol, 12.91% yield, 96.2% purity, 2TFA) as a white solid. MS(M+H)⁺=843.4.

¹H NMR (400 MHz, DMSO-d₆) δ=10.82 (s, 1H), 9.17-9.00 (m, 1H), 8.57-8.47 (m, 1H), 8.32-8.22 (m, 2H), 8.09 (s, 1H), 7.56-7.48 (m, 2H), 7.36-7.30 (m, 1H), 6.77 (br d, J=8.8 Hz, 2H), 4.83-4.72 (m, 1H), 4.38 (br dd, J=3.7, 6.5 Hz, 1H), 4.14-4.00 (m, 2H), 3.93 (s, 3H), 3.53-3.63 (m, 8H), 3.33 (s, 3H), 3.28-3.19 (m, 2H), 3.13-2.98 (m, 2H), 2.80-2.68 (m, 1H), 2.67-2.50 (m, 1H), 2.28-2.10 (m, 1H), 2.09-2.00 (m, 2H), 1.97-1.82 (m, 6H), 1.75-1.44 (m, 10H).

### Example 29. Synthesis of 4-((9′-cyclopentyl-5′-methyl-6′-oxo-5′,6′,8′,9′-tetrahydrospiro[cyclopropane-1,7′-pyrimido[4,5-b][1,4]diazepin]-2′-yl)amino)-N-(1-(3-(4-(4-((2,6-dioxopiperidin-3-yl)amino)phenyl)piperazin-1-yl)propanoyl)piperidin-4-yl)-3-methoxybenzamide (Compound 29)

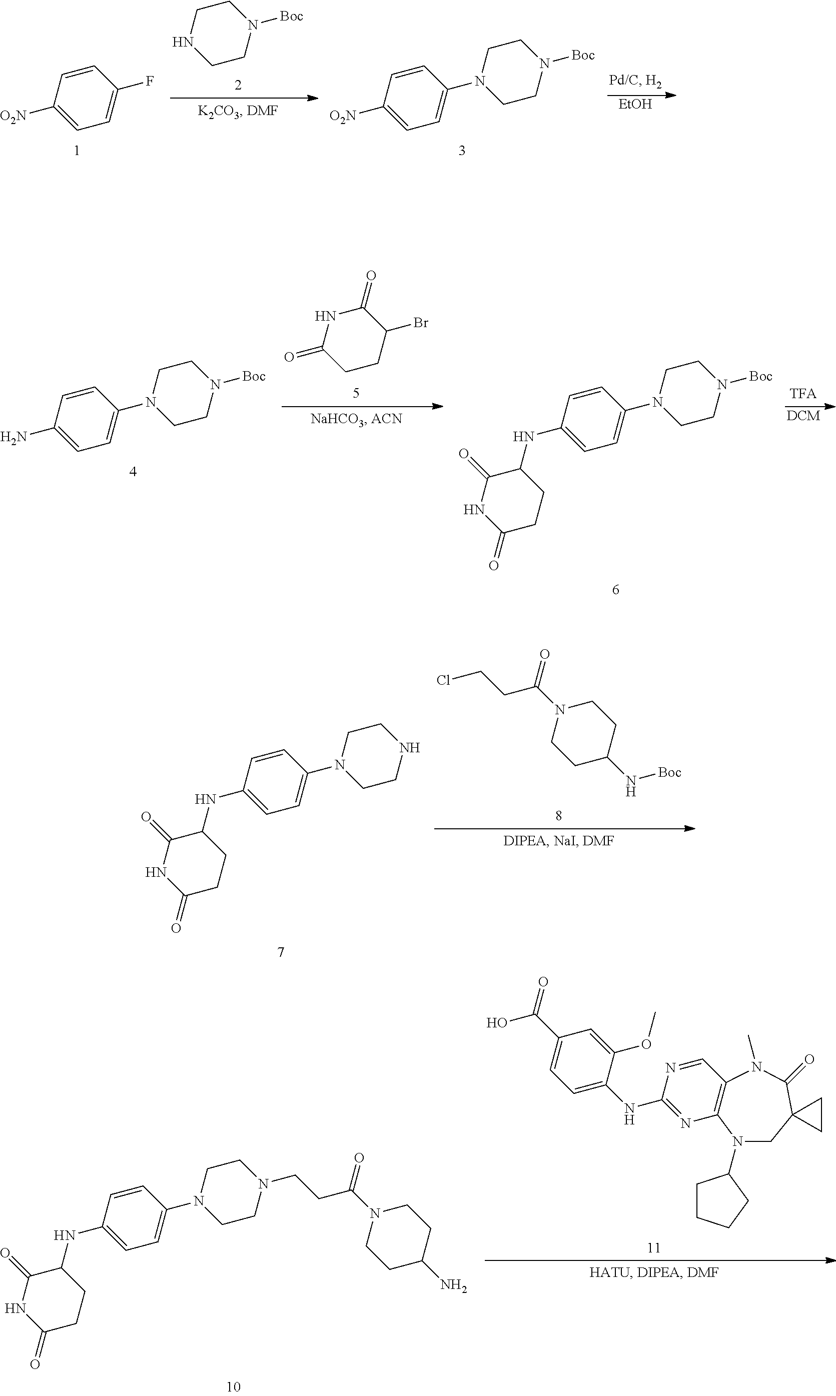

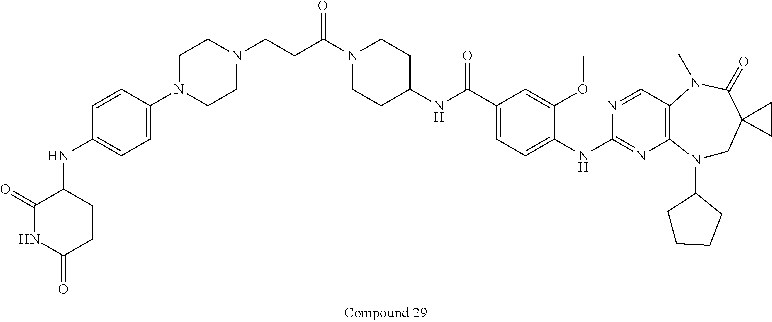 [SMILES: COc1cc(C(=O)NC2CCN(C(=O)CCN3CCN(c4ccc(NC5CCC(=O)NC5=O)cc4)CC3)CC2)ccc1Nc1ncc2c(n1)N(C1CCCC1)CC1(CC1)C(=O)N2C]

### Step 1. Synthesis of tert-butyl 4-(4-nitrophenyl)piperazine-1-carboxylate (3)

To a solution of 1-fluoro-4-nitrobenzene (10 g, 70.87 mmol, 7.52 mL) and tert-butyl piperazine-1-carboxylate (26.40 g, 141.74 mmol) in DMF (100 mL) was added K₂CO₃ (29.38 g, 212.62 mmol). The mixture was stirred at 0° C. for 1 h and at 20° C. for 16 h. LCMS showed 1-fluoro-4-nitrobenzene was consumed completely and desired mass was detected. The reaction mixture was diluted with H₂O (100 mL) at 0° C. The mixture was filtered and washed with water (100 mL) and Petroleum ether (100 mL) to afford tert-butyl 4-(4-nitrophenyl)piperazine-1-carboxylate (25.7 g, crude) as a yellow solid. MS(M+H)⁺=308.0.

### Step 2. Synthesis of tert-butyl 4-(4-aminophenyl)piperazine-1-carboxylate (4)

To solution of tert-butyl 4-(4-nitrophenyl)piperazine-1-carboxylate (10 g, crude) in EtOH (100 mL) was added Pd/C (3 g, 10% purity). The suspension was degassed and purged with H₂ for 3 times. The mixture was stirred under H₂ (15 Psi) at 25° C. for 16 h. LCMS showed tert-butyl 4-(4-nitrophenyl)piperazine-1-carboxylate was consumed completely and desired mass was detected. The mixture was filtered and concentrated under reduced pressure to afford tert-butyl 4-(4-aminophenyl)piperazine-1-carboxylate (9.18 g, crude) as a red solid. The crude product was used to the next step without further purification. MS(M+H)⁺=278.2.

### Step 3. Synthesis of tert-butyl 4-(4-((2,6-dioxopiperidin-3-yl)amino)phenyl))piperazine-1-carboxylate (6)

To a solution of tert-butyl 4-(4-aminophenyl)piperazine-1-carboxylate (2.5 g, 9.01 mmol) and 3-bromopiperidine-2,6-dione (3.46 g, 18.02 mmol) in ACN (10 mL) was added NaHCO₃ (3.51 g, 41.78 mmol, 1.63 mL). The mixture was stirred at 80° C. for 16 hr. LCMS showed tert-butyl 4-(4-aminophenyl)piperazine-1-carboxylate was consumed completely and desired mass was detected. The reaction mixture was quenched with H₂O (40 mL) at 25° C., extracted with EtOAc (160 mL). The organic layer was dried over Na₂SO₄, filtered and concentrated under reduced pressure to give a residue. The residue was purified by flash silica gel chromatography (ISCO®; 80 g SepaFlash® Silica Flash Column, Eluent of 0~40% EtOAc/Petroleum ether gradient @50 mL/min) to afford tert-butyl 4-(4-((2,6-dioxopiperidin-3-yl)amino)phenyl)piperazine-1-carboxylate (1.7 g, 4.38 mmol, 48.55% yield) as a blue solid. MS(M+H)⁺=389.2.

### Step 4. Synthesis of 3-((4-(piperazin-1-yl)phenyl)amino)piperidine-2,6-dione (7)

To a solution of tert-butyl 4-(4-((2,6-dioxopiperidin-3-yl)amino)phenyl)piperazine-1-carboxylate (0.6 g, 1.54 mmol) in DCM (6 mL) was added TFA (3.07 g, 26.92 mmol, 2 mL). The mixture was stirred at 20° C. for 1 hr. LCMS showed tert-butyl 4-(4-((2,6-dioxopiperidin-3-yl)amino)phenyl)piperazine-1-carboxylate was consumed completely and desired mass was detected. The reaction mixture was concentrated under reduced pressure to afford 3-((4-(piperazin-1-yl)phenyl)amino)piperidine-2,6-dione (0.62 g, 1.54 mmol, 99.76% yield, TFA) as a blue oil. MS (M+H)⁺=289.3.

### Step 5. Synthesis of tert-butyl (1-(3-(4-(4-((2,6-dioxopiperidin-3-yl)amino)phenyl)piperazin-1-yl)propanoyl)piperidin-4-yl)carbamate (9)

To a solution of 3-((4-(piperazin-1-yl)phenyl)amino)piperidine-2,6-dione (0.77 g, 1.91 mmol, TFA) and tert-butyl (1-(3-chloropropanoyl)piperidin-4-yl)carbamate (1.11 g, 3.83 mmol) in DMF (8 mL) were added DIPEA (2.23 g, 17.22 mmol, 3.00 mL) and NaI (57.37 mg, 382.73 μmol). The mixture was stirred at 40° C. for 16 h. LCMS showed 23% of 3-((4-(piperazin-1-yl)phenyl)amino)piperidine-2,6-dione remained and 16% of desired mass was detected. The reaction mixture was quenched with H₂O (20 mL) at 25° C., and then extracted with EtOAc (180 mL). The organic layer was washed with brine (20 mL), dried over Na₂SO₄, filtered and concentrated under reduced pressure to give a residue. The residue was purified by flash silica gel chromatography (ISCO®; 40 g SepaFlash® Silica Flash Column, Eluent of 0~10% MeOH/EtOAc @50 mL/min) to afford tert-butyl (1-(3-(4-(4-((2,6-dioxopiperidin-3-yl)amino)phenyl)piperazin-1-yl)propanoyl)piperidin-4-yl)carbamate (0.34 g, 626.53 μmol, 32.74% yield) as a black solid. MS(M+H)⁺=543.4.

### Step 6. Synthesis of 3-((4-(4-(3-(4-aminopiperidin-1-yl)-3-oxopropyl)piperazin-1-yl)phenyl)amino)piperidine-2,6-dione (10)

To a solution of tert-butyl (1-(3-(4-(4-((2,6-dioxopiperidin-3-yl)amino)phenyl)piperazin-1-yl)propanoyl)piperidin-4-yl)carbamate (300 mg, 552.82 μmol) in DCM (0.4 mL) was added TFA (1.15 g, 10.10 mmol, 750.00 μL). The mixture was stirred at 25° C. for 0.5 hr. LCMS showed tert-butyl (1-(3-(4-(4-((2,6-dioxopiperidin-3-yl)amino)phenyl)piperazin-1-yl)propanoyl)piperidin-4-yl)carbamate was consumed completely and desired mass was detected. The reaction mixture was concentrated under reduced pressure to afford 3-((4-(4-(3-(4-aminopiperidin-1-yl)-3-oxopropyl)piperazin-1-yl)phenyl)amino)piperidine-2,6-dione (300 mg, 539.01 μmol, 97.50% yield, TFA) a green oil. MS(M+H)⁺=443.2.

### Step 7. Synthesis of 4-((9′-cyclopentyl-5′-methyl-6′-oxo-5′,6′,8′,9′-tetrahydrospiro[cyclopropane-1,7′-pyrimido[4,5-b][1,4]diazepin]-2′-yl)amino)-N-(1-(3-(4-(4-((2,6-dioxopiperidin-3-yl)amino)phenyl)piperazin-1-yl)propanoyl)piperidin-4-yl)-3-methoxybenzamide (Compound 29)

To a solution of 4-((9′-cyclopentyl-5′-methyl-6′-oxo-5′,6′,8′,9′-tetrahydrospiro[cyclopropane-1,7′-pyrimido[4,5-b][1,4]diazepin]-2′-yl)amino)-3-methoxybenzoic acid (235.81 mg, 539.01 mol) in DMF (3 mL) were added HATU (307.42 mg, 808.51 μmol) and DIPEA (348.32 mg, 2.70 mmol, 469.43 μL). The mixture was stirred at 25° C. for 0.5 h. And then 3-((4-(4-(3-(4-aminopiperidin-1-yl)-3-oxopropyl)piperazin-1-yl)phenyl)amino)piperidine-2,6-dione (300 mg, 539.01 μmol, TFA) was added and stirred at 25° C. for another 16 h. LCMS showed 11% of 3-((4-(4-(3-(4-aminopiperidin-1-yl)-3-oxopropyl)piperazin-1-yl)phenyl)amino)piperidine-2,6-dione remained and 24% of desired compound. DIPEA (371.00 mg, 2.87 mmol, 0.5 mL) was added and stirred at 25° C. for another 2 hr. LCMS showed 3-((4-(4-(3-(4-aminopiperidin-1-yl)-3-oxopropyl)piperazin-1-yl)phenyl)amino)piperidine-2,6-dione was consumed completely and desired mass was detected. The reaction mixture was quenched by addition H₂O (20 mL) at 25° C., and then extracted with EtOAc (40 mL×3). The combined organic layers were washed with brine (20 mL), dried over Na₂SO₄, filtered and concentrated under reduced pressure to give a residue. The residue was purified by flash silica gel chromatography (ISCO®; 20 g SepaFlash® Silica Flash Column, Eluent of 0~20% MeOH/EtOAc @50 mL/min) and prep-HPLC (column: Waters Xbridge 150*25 mm*5 um; mobile phase: [water (NH₄HCO₃)-ACN]; gradient:30%-60% B over 10 min). The eluent was lyophilized to afford 4-((9′-cyclopentyl-5′-methyl-6′-oxo-5′,6′,8′,9′-tetrahydrospiro[cyclopropane-1,7′-pyrimido[4,5-b][1,4]diazepin]-2′-yl)amino)-N-(1-(3-(4-(4-((2,6-dioxopiperidin-3-yl)amino)phenyl)piperazin-1-yl)propanoyl)piperidin-4-yl)-3-methoxybenzamide (73 mg, 79.60 mol, 14.77% yield, 94% purity) and 4-((9′-cyclopentyl-5′-methyl-6′-oxo-5′,6′,8′,9′-tetrahydrospiro[cyclopropane-1,7′-pyrimido[4,5-b][1,4]diazepin]-2′-yl)amino)-N-(1-(3-(4-(4-((2,6-dioxopiperidin-3-yl)amino)phenyl)piperazin-1-yl)propanoyl)piperidin-4-yl)-3-methoxybenzamide (50.2 mg, 56.49 mol, 10.48% yield, 94% purity) as a purple solid. MS(M+H)⁺=862.7.

¹H NMR (400 MHz, DMSO-d₆) δ=10.75 (s, 1H), 8.40 (d, J=8.1 Hz, 1H), 8.12 (d, J=7.6 Hz, 1H), 7.98 (s, 1H), 7.69 (s, 1H), 7.55-7.39 (m, 2H), 6.75 (d, J=9.0 Hz, 2H), 6.60 (d, J=8.9 Hz, 2H), 5.37 (d, J=7.3 Hz, 1H), 4.94-4.77 (m, 1H), 4.39 (d, J=13.0 Hz, 1H), 4.26-4.13 (m, 1H), 4.12-4.00 (m, 1H), 3.94 (s, 4H), 3.47 (s, 2H), 3.16 (s, 3H), 3.15-3.08 (m, 1H), 2.97-2.86 (m, 4H), 2.78-2.63 (m, 2H), 2.60-2.50 (m, 9H), 2.17-2.03 (m, 1H), 1.95-1.76 (m, 5H), 1.73-1.64 (m, 2H), 1.63-1.56 (m, 2H), 1.54-1.36 (m, 4H), 0.95-0.83 (m, 2H), 0.72-0.61 (m, 2H)

### Example 30. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-((1r,4r)-4-(((1-(4-(2,6-dioxopiperidin-3-yl)-2-fluoro phenyl)piperidin-4-yl)methyl)amino)cyclohexyl)-3-methoxybenzamide (Compound 30)

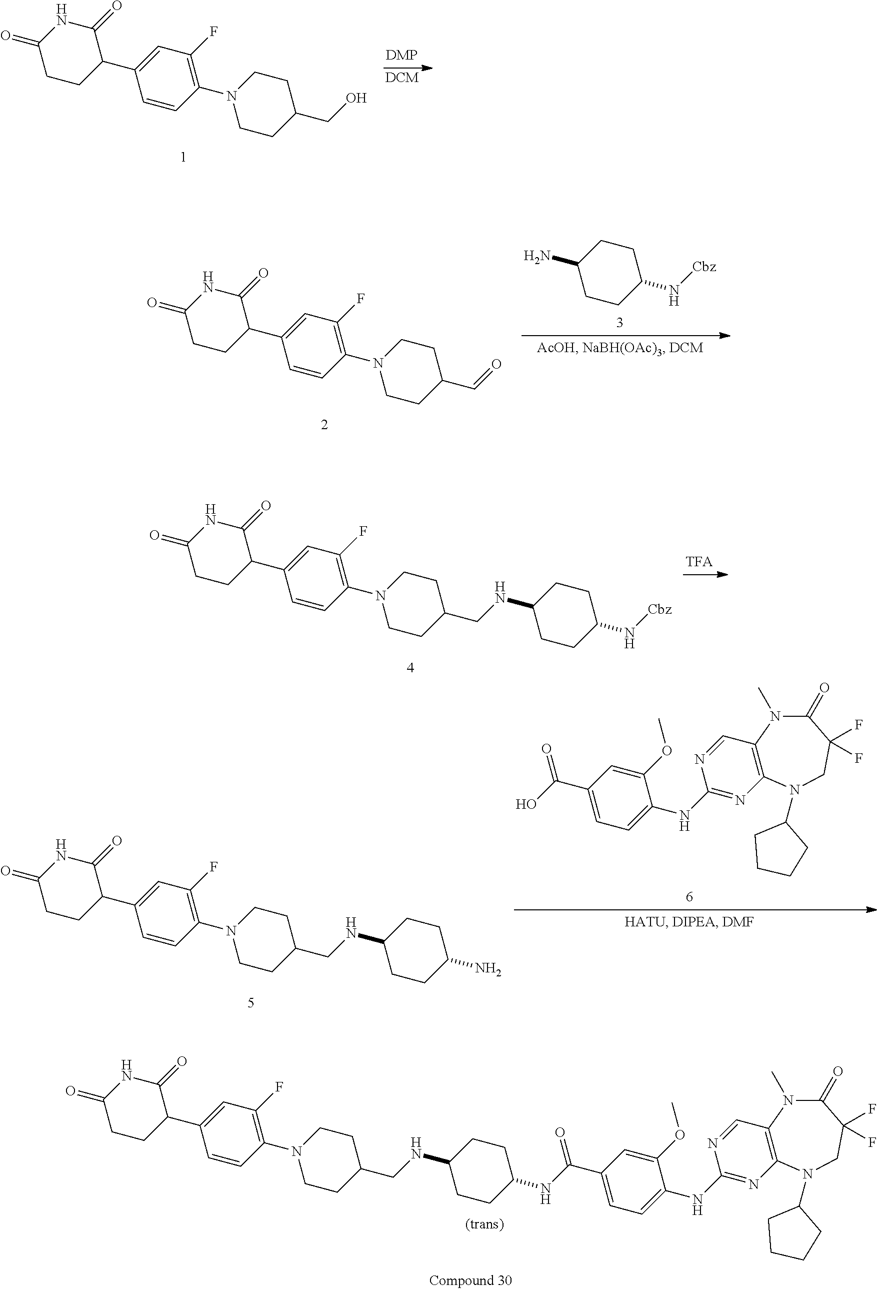

### Step 1. Synthesis of 1-(4-(2,6-dioxopiperidin-3-yl)-2-fluorophenyl)piperidine-4-carbaldehyde (2)

To a solution of 3-(3-fluoro-4-(4-(hydroxymethyl)piperidin-1-yl)phenyl)piperidine-2,6-dione (2 g, 6.24 mmol) in DCM (50 mL) was added DMP (3.97 g, 9.36 mmol) and the mixture was stirred at 20° C. for 2 h. TLC (Petroleum ether/EtOAc=1/1) showed 3-(3-fluoro-4-(4-(hydroxymethyl)piperidin-1-yl)phenyl)piperidine-2,6-dione was consumed completely and new spot was formed. The mixture was filtered and the filtrate was concentrated under reduced pressure to afford 1-(4-(2,6-dioxopiperidin-3-yl)-2-fluorophenyl)piperidine-4-carbaldehyde (2 g, crude) as yellow oil. MS(M+H)⁺=319.4

### Step 2. Synthesis of benzyl ((1r,4r)-4-(((1-(4-(2,6-dioxopiperidin-3-yl)-2-fluorophenyl)piperidin-4-yl)methyl)amino)cyclohexyl)carbamate (4)

To the solution of 1-(4-(2,6-dioxopiperidin-3-yl)-2-fluorophenyl)piperidine-4-carbaldehyde (2 g, 6.28 mmol) and benzyl ((1r,4r)-4-aminocyclohexyl)carbamate (1.56 g, 6.28 mmol) in DCM (20 mL) was added AcOH (377.28 mg, 6.28 mmol, 359.66 μL) and the mixture was stirred at 20° C. for 1 h, then NaBH(OAc)₃ (1.60 g, 7.54 mmol) was added and the resulting mixture was stirred at 20° C. for 12 h. LCMS showed a peak (43%) with desired mass. The reaction mixture was diluted with water (100 mL) and extracted with DCM (50 mL×3). The combined organic layers were washed with saturated brine (200 mL), dried over anhydrous Na₂SO₄, filtered and concentrated under reduced pressure. The residue was purified by prep-HPLC (column: Phenomenex luna C18 150*40 mm*15 um; mobile phase: [water (TFA)-ACN]; gradient: 15%-45% B over 10 min) to afford benzyl ((1r,4r)-4-(((1-(4-(2,6-dioxopiperidin-3-yl)-2-fluorophenyl)piperidin-4-yl)methyl)amino)cyclohexyl)carbamate (0.9 g, 1.57 mmol, 24.97% yield, 96% purity) as yellow oil. MS(M+H)⁺=551.4

### Step 3. Synthesis of 3-(4-(4-((((1r,4r)-4-aminocyclohexyl)amino)methyl)piperidin-1-yl)-3-fluorophenyl)piperidine-2,6-dione (5)

The solution of benzyl ((1r,4r)-4-(((1-(4-(2,6-dioxopiperidin-3-yl)-2-fluorophenyl)piperidin-4-yl)methyl)amino)cyclohexyl)carbamate (450 mg, 817.20 μmol) in TFA (7.68 g, 67.31 mmol, 5 mL) was stirred at 40° C. for 3 h. LCMS showed a peak (79%) with desired mass. The mixture was concentrated under reduced pressure to afford 3-(4-(4-((((1r,4r)-4-aminocyclohexyl)amino)methyl)piperidin-1-yl)-3-fluorophenyl)piperidine-2,6-dione (0.4 g, 753.93 μmol, 92.26% yield, TFA) as yellow oil. MS(M+H)⁺=417.3

### Step 4. Synthesis of 4-((9-cyclopentyl-7,7-difluoro-5-methyl-6-oxo-6,7,8,9-tetrahydro-5H-pyrimido[4,5-b][1.4]diazepin-2-yl)amino)-N-((1r,4r)-4-(((1-(4-(2,6-dioxopiperidin-3-yl)-2-fluorophenyl)piperidin-4-yl)methyl)amino)cyclohexyl)-3-methoxybenzamide (Compound 30)

To the solution of 4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzoic acid (168.67 mg, 376.96 μmol) in DMF (5 mL) were added HATU (172.00 mg, 452.36 μmol) and DIPEA (194.88 mg, 1.51 mmol, 262.64 μL) and the mixture was stirred at 20° C. for 1 h, 3-(4-(4-((((1r,4r)-4-aminocyclohexyl)amino)methyl)piperidin-1-yl)-3-fluorophenyl)piperidine-2,6-dione (0.2 g, 376.96 μmol, TFA salt) was added and the resulting mixture was stirred at 20° C. for 12 h. LCMS showed a peak (16%) with desired mass. The reaction mixture was diluted with water (50 mL) and extracted with EtOAc (30 mL×3). The combined organic layers were washed with saturated brine (100 mL), dried over anhydrous Na₂SO₄, filtered. The filtrate was concentrated under reduced pressure. The residue was purified by prep-HPLC (column: Phenomenex luna C18 150*25 mm*10 um; mobile phase: [water (FA)-ACN]; gradient: 17%-47% B over 10 min) and the eluent was lyophilized to afford 4-((9-cyclopentyl-7,7-difluoro-5-methyl-6-oxo-6,7,8,9-tetrahydro-5H-pyrimido[4,5-b][1,4]diazepin-2-yl)amino)-N-((1r,4r)-4-(((1-(4-(2,6-dioxopiperidin-3-yl)-2-fluorophenyl)piperidin-4-yl)methyl)amino)cyclohexyl)-3-methoxybenzamide (17.5 mg, 20.25 mol, 5.37% yield, 97.9% purity) as a light yellow solid. MS(M+H)⁺=846.4.

¹H NMR (400 MHz, DMSO-d₆) δ=10.83 (s, 1H), 8.26 (s, 1H), 8.26-8.17 (m, 4H), 7.54-7.43 (m, 2H), 7.10-6.93 (m, 3H), 4.88-4.68 (m, 1H), 4.14-4.04 (m, 2H), 3.94 (s, 3H), 3.85-3.75 (m, 2H), 3.33 (s, 3H), 3.10-3.04 (m, 1H), 3.00-2.92 (m, 2H), 2.73-2.62 (m, 5H), 2.27-2.07 (m, 4H), 2.05-1.82 (m, 8H), 1.81-1.70 (m, 3H), 1.65-1.55 (m, 4H), 1.50-1.36 (m, 6H).

### Example 31. Synthesis of N-(4-(1-amino-2-(4-(4-(2,6-dioxopiperidin-3-yl)-2-fluorophenyl)piperazin-1-yl)ethyl) piperidin-1-yl)-4-((7,7-difluoro-9-isopropyl-5-methyl-6-oxo-6,7,8,9-tetrahydro-5H-pyrimido[4,5-b][1,4]diazepin-2-yl)amino)-3-methoxybenzamide (Compound 31)

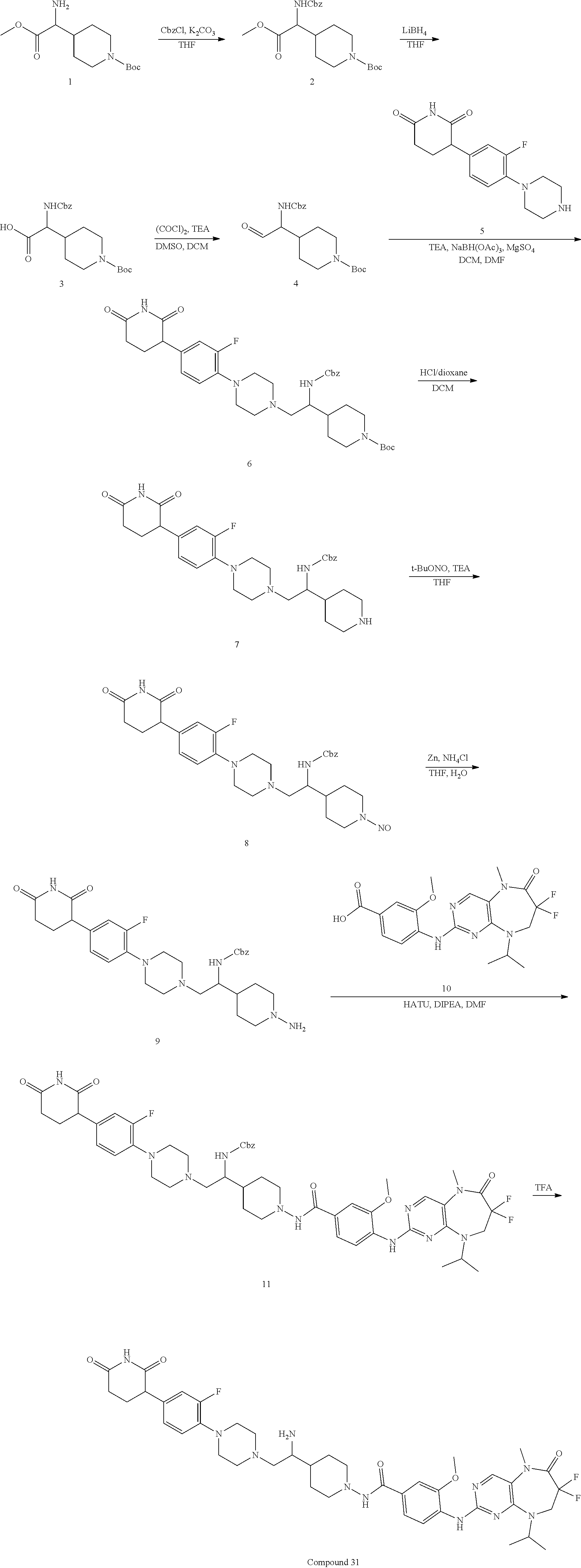

### Step 1. Synthesis of tert-butyl 4-(1-(((benzyloxy)carbonyl)amino)-2-methoxy-2-oxoethyl)piperidine-1-carboxylate

To a solution of tert-butyl 4-(1-amino-2-methoxy-2-oxoethyl)piperidine-1-carboxylate (4.5 g, 16.52 mmol) in THF (50 mL) were added CbzCl (3.38 g, 19.83 mmol, 2.83 mL) and K₂CO₃ (6.85 g, 49.57 mmol). The mixture was stirred at 20° C. for 16 h. LCMS showed the starting material was consumed completely and 15% of the desired mass. The reaction mixture was quenched by addition water (30 mL) at 0° C., extracted with EtOAc (50 mL×2). The combined organic layers were washed with brine (40 mL), dried over Na₂SO₄ and filtered. The filtrate was concentrated under reduced pressure and then purified by flash silica gel chromatography (40 g SepaFlash® Silica Flash Column, Eluent of 0~30% EtOAc/Petroleum ether gradient @60 mL/min) to afford tert-butyl 4-(1-(((benzyloxy)carbonyl)amino)-2-methoxy-2-oxoethyl)piperidine-1-carboxylate (3.9 g, 9.59 mmol, 58.07% yield) as a colorless oil. MS(M−100+H)⁺=307.2

### Step 2. Synthesis of tert-butyl 4-(1-(((benzyloxy)carbonyl)amino)-2-hydroxyethyl)piperidine-1-carboxylate (3)

To a solution of tert-butyl 4-(1-(((benzyloxy)carbonyl)amino)-2-methoxy-2-oxoethyl)piperidine-1-carboxylate (3.9 g, 9.59 mmol) in THF (100 mL) was added LiBH₄ (2 M, 9.59 mL) at 0° C. The mixture was stirred at 20° C. for 16 h. LCMS showed the starting material was consumed completely and 37% of the desired mass. The reaction mixture was quenched by addition aq. sat. NH₄Cl (30 mL) at 0° C., and then extracted with EtOAc (40 mL×2). The combined organic layers were washed with brine (30 mL), dried over Na₂SO₄, filtered and concentrated under reduced pressure to give a residue. The residue was purified by flash silica gel chromatography (40 g SepaFlash® Silica Flash Column, Eluent of 0~60% EtOAc/Petroleum ether gradient @60 mL/min) to afford tert-butyl 4-(1-(((benzyloxy)carbonyl)amino)-2-hydroxyethyl)piperidine-1-carboxylate (1.5 g, 3.73 mmol, 38.83% yield, 94% purity) as a colorless oil. MS(M−100+H)⁺=279.2

### Step 3. Synthesis of tert-butyl 4-(1-(((benzyloxy)carbonyl)amino)-2-oxoethyl)piperidine-1-carboxylate (4)

To a solution of DMSO (420.00 mg, 5.38 mmol, 420 L) in DCM (9 mL) was slowly added (COCl)₂ (609.00 mg, 4.80 mmol, 420 L) at −70° C. and the mixture was stirred at −70° C. for 15 min. Then a solution of tert-butyl 4-(1-(((benzyloxy)carbonyl)amino)-2-hydroxyethyl)piperidine-1-carboxylate (850 mg, 2.25 mmol) in DCM (9 mL) was added at −70° C. and the mixture was stirred at −70° C. for 20 min. TEA (1.36 g, 13.44 mmol, 1.87 mL) was added and the mixture was stirred at −70° C. for 30 min. ¹H NMR showed the desired product after work up. The mixture was diluted with water (50 mL) and then extracted with DCM (20 mL×2), the combined organic layers was washed with water (20 mL), dried over Na₂SO₄, filtered and concentrated under reduced pressure to afford tert-butyl 4-(1-(((benzyloxy)carbonyl)amino)-2-oxoethyl)piperidine-1-carboxylate (860 mg, crude) as a yellow oil. MS(M+H)⁺=377.5

### Step 4. Synthesis of tert-butyl 4-(1-(((benzyloxy)carbonyl)amino)-2-(4-(4-(2,6-dioxopiperidin-3-yl)-2-fluorophenyl)piperazin-1-ylethyl)piperidine-1-carboxylate (6)

To a solution of 3-(3-fluoro-4-(piperazin-1-yl)phenyl)piperidine-2,6-dione (650 mg, 1.98 mmol, HCl) in DCM (10 mL) were added TEA (625.22 mg, 6.18 mmol, 860 L) and MgSO₄ (716.08 mg, 5.95 mmol) and then followed by tert-butyl 4-(1-(((benzyloxy)carbonyl)amino)-2-oxoethyl)piperidine-1-carboxylate (860 mg, 2.28 mmol) in DMF (10 mL) and the mixture was stirred at 25° C. for 15 min. NaBH(OAc)₃ (840.67 mg, 3.97 mmol) was added and the mixture was stirred at 25° C. for 14 h. LCMS showed 41% of the desired mass. The mixture was combined with other batch (150 mg scale) and then diluted with water (30 mL) and extracted with EtOAc (10 mL×2), the combined organic layer was washed with water (10 mL×2), dried over Na₂SO₄ and filtered. The filtrate was concentrated under reduced pressure. The residue was purified by flash silica gel chromatography (5 g SepaFlash® Silica Flash Column, Eluent of 0~10% MeOH/EtOAc gradient @50 mL/min) to afford tert-butyl 4-(1-(((benzyloxy)carbonyl)amino)-2-(4-(4-(2,6-dioxopiperidin-3-yl)-2-fluorophenyl)piperazin-1-yl)ethyl)piperidine-1-carboxylate (720 mg, 994.22 μmol, 50.14% yield, 90% purity) as a yellow solid. MS(M+H)⁺=652.4

### Step 5. Synthesis of benzyl (2-(4-(4-(2,6-dioxopiperidin-3-yl)-2-fluorophenyl)piperazin-1-yl)-1-(piperidin-4-yl)ethyl)carbamate (7)

To a solution of tert-butyl 4-(1-(((benzyloxy)carbonyl)amino)-2-(4-(4-(2,6-dioxopiperidin-3-yl)-2-fluorophenyl)piperazin-1-yl)ethyl)piperidine-1-carboxylate (720 mg, 1.10 mmol) in DCM (5 mL) was added HCl/dioxane (4 M, 10 mL) and the mixture was stirred at 25° C. for 1 h. LCMS showed 87% of the desired mass. The mixture was concentrated under reduced pressure to afford benzyl (2-(4-(4-(2,6-dioxopiperidin-3-yl)-2-fluorophenyl)piperazin-1-yl)-1-(piperidin-4-yl)ethyl)carbamate (660 mg, crude, HCl) as a yellow solid. MS(M+H)⁺=552.4

### Step 6. Synthesis of benzyl (2-(4-(4-(2,6-dioxopiperidin-3-yl)-2-fluorophenyl)piperazin-1-yl)-1-(1-nitrosopiperidin-4-yl)ethyl)carbamate (8)

To a solution of benzyl (2-(4-(4-(2,6-dioxopiperidin-3-yl)-2-fluorophenyl)piperazin-1-yl)-1-(piperidin-4-yl)ethyl)carbamate (0.3 g, crude, HCl) in THF (6 mL) were added TEA (174.48 mg, 1.72 mmol, 240.00 μL) and t-BuONO (208.08 mg, 2.02 mmol, 240.00 μL) and the mixture was stirred at 60° C. under N₂ for 14 h. LCMS showed 50% of the desired mass. The mixture was concentrated under reduced pressure. The residue was purified by flash silica gel chromatography (5 g SepaFlash® Silica Flash Column, Eluent of 0~20% EtOAc/Petroleum ether gradient @50 mL/min) to afford benzyl (2-(4-(4-(2,6-dioxopiperidin-3-yl)-2-fluorophenyl)piperazin-1-yl)-1-(1-nitrosopiperidin-4-yl)ethyl)carbamate (0.5 g, 861.11 μmol, 84.40% yield) as a yellow solid. MS(M+H)⁺=581.4

### Step 7. Synthesis of benzyl (1-(1-aminopiperidin-4-yl)-2-(4-(4-(2,6-dioxopiperidin-3-yl)-2-fluorophenyl)piperazin-1-yl)ethyl)carbamate (9)

To a solution of benzyl (2-(4-(4-(2,6-dioxopiperidin-3-yl)-2-fluorophenyl)piperazin-1-yl)-1-(1-nitrosopiperidin-4-yl)ethyl)carbamate (100 mg, 172.22 μmol) and NH₄Cl (28.00 mg, 523.45 μmol) in THF (1 mL) and H₂O (0.3 mL) was added Zn (56 mg, 856.40 μmol) slowly and the mixture was stirred at 25° C. for 1 h. LCMS showed 75% of the desired mass. The mixture was combined with other batch (50 mg scale) and then diluted with THF (5 mL). The mixture was filtered and the filter cake was washed with THF (15 mL). The filtrate was concentrated under reduced pressure to afford benzyl (1-(1-aminopiperidin-4-yl)-2-(4-(4-(2,6-dioxopiperidin-3-yl)-2-fluorophenyl)piperazin-1-yl)ethyl)carbamate (190 mg, crude) as a yellow solid. MS(M+H)⁺=567.4

### Step 8. Synthesis of benzyl (1-(1-(4-((7,7-difluoro-9-isopropyl-5-methyl-6-oxo-6,7,8,9-tetrahydro-5H-pyrimido[4, 5-b][1,4]diazepin-2-yl)amino)-3-methoxybenzamido)piperidin-4-yl)-2-(4-(4-(2,6-dioxopiperidin-3-yl)-2-fluorophenyl)piperazin-1-yl)ethyl)carbamate (11)

To a solution of 4-((7,7-difluoro-9-isopropyl-5-methyl-6-oxo-6,7,8,9-tetrahydro-5H-pyrimido[4,5-b][1, 4]diazepin-2-yl)amino)-3-methoxybenzoic acid (140 mg, 332.23 μmol) and HATU (151.59 mg, 398.67 μmol) in DMF (1 mL) was added DIPEA (222.60 mg, 1.72 mmol, 0.3 mL) and the mixture was stirred at 25° C. for 15 min. Then a solution of benzyl (1-(1-aminopiperidin-4-yl)-2-(4-(4-(2,6-dioxopiperidin-3-yl)-2-fluorophenyl)piperazin-1-yl)ethyl)carbamate (190 mg, crude) in DMF (3 mL) was added and the mixture was stirred at 25° C. for 1 h. LCMS showed 51% the desired mass. The mixture was diluted with water (10 mL) and then extracted with EtOAc (10 mL×3), the combined organic layers was washed with brine (10 mL×3), dried over Na₂SO₄, filtered and concentrated under reduced pressure and then purified by prep-TLC (EtOAc:MeOH=10:1) to afford benzyl (1-(1-(4-((7,7-difluoro-9-isopropyl-5-methyl-6-oxo-6,7,8,9-tetrahydro-5H-pyrimido[4, 5-b][1,4]diazepin-2-yl)amino)-3-methoxybenzamido)piperidin-4-yl)-2-(4-(4-(2,6-dioxopiperidin-3-yl)-2-fluorophenyl)piperazin-1-yl)ethyl)carbamate (0.2 g, 173.19 μmol, 52.13% yield, 84% purity) as a yellow solid. MS(M+H)⁺=970.5

### Step 9. Synthesis of N-(4-(1-amino-2-(4-(4-(2,6-dioxopiperidin-3-yl)-2-fluorophenyl)piperazin-1-yl)ethyl)piperidin-1-yl)-4-((7,7-difluoro-9-isopropyl-5-methyl-6-oxo-6,7,8,9-tetrahydro-5H-pyrimido[4,5-b][1,4]diazepin-2-yl)amino)-3-methoxybenzamide (Compound 31)

A solution of benzyl (1-(1-(4-((7,7-difluoro-9-isopropyl-5-methyl-6-oxo-6,7,8,9-tetrahydro-5H-pyrimido[4, 5-b][1,4]diazepin-2-yl)amino)-3-methoxybenzamido)piperidin-4-yl)-2-(4-(4-(2,6-dioxopiperidin-3-yl)-2-fluorophenyl)piperazin-1-yl)ethyl)carbamate (170 mg, 147.21 mol) in TFA (12.28 g, 107.70 mmol, 8 mL) was stirred at 60° C. for 6 h. LCMS showed 55% the desired mass. The mixture was concentrated under reduced pressure and then purified by prep-HPLC (column: Phenomenex luna C18 150*25 mm*10 um; mobile phase: [water (TFA)-ACN]; gradient: 17%-47% B over 10 min) and the eluent was lyophilized to afford N-(4-(1-amino-2-(4-(4-(2,6-dioxopiperidin-3-yl)-2-fluorophenyl)piperazin-1-yl)ethyl)piperidin-1-yl)-4-((7,7-difluoro-9-isopropyl-5-methyl-6-oxo-6,7,8,9-tetrahydro-5H-pyrimido[4,5-b][1,4]diazepin-2-yl)amino)-3-methoxybenzamide (77.4 mg, 59.79 μmol, 40.62% yield, 91% purity, 3TFA) as a white solid. MS(M+H)⁺=836.5

¹H NMR (400 MHz, DMSO-d₆) δ=10.83 (s, 1H), 9.48-9.38 (m, 1H), 8.30-8.02 (m, 3H), 7.49-7.40 (m, 2H), 7.12-6.93 (m, 3H), 4.95-4.80 (m, 1H), 4.08 (t, J=12.9 Hz, 2H), 3.93 (s, 3H), 3.85-3.79 (m, 1H), 3.58-3.62 (m, 6H), 3.32 (s, 3H), 3.20-3.08 (m, 6H), 3.02-2.91 (m, 1H), 2.81-2.72 (m, 2H), 2.71-2.58 (m, 1H), 2.50-2.47 (m, 3H), 2.26-2.14 (m, 1H), 2.04-1.94 (m, 1H), 1.82-1.46 (m, 5H), 1.30-1.16 (m, 6H).

### Experimental Example

### 1. Luciferase Assay for PLK1

### Preparation and Culture of HeLa LgBit (PLK1-HiBit KI) Cell Line

A HeLa cell line in which the LgBit vector was transfected and expressed stably was prepared. Then, after constructing gRNA and donor to express the HiBit amino acid sequence behind the C-terminal of the PLK1 gene, which was inherent in the cell, it was inserted into the cell together with a vector capable of expressing CRISPR/Cas9. Only the cells in which the insertion was completed and knock-in had progressed were selected, sub-cultured and used.

For cell culture, DMEM (Gibco, Cat. No. 11995-065; Lot. No. 2646135), FBS (Gibco, Cat. No. 16000-044; Lot. No. 2634213P), Penicillin/Streptomycin (PS)(Gibco, Cat. No. 15140-122; Lot. No. 2441887), 100 mm² cell culture dish (SPL, Cat. No. 20100), 150 mm² cell culture dish (SPL, Cat. No. 20150), 96-well culture plate (SPL, Cat. No. 30196), PBS pH 7.4 (Gibco, Cat. No. 10010-023; Lot. No. 2509235), TrypLE™ Express (Gibco, Cat. No. 12605-010; Lot. No. 2323417), Cell Counter (Hematocytometer)(As one, Cat. No. 4-458-01) and 0.4% Trypan Blue Solution (DYNEBIO, Cat. No. CBT3710; Lot. No. 20221111) were used.

### Luciferase Assay

The compounds of Examples were completely dissolved in DMSO (Sigma-Aldrich Cat. No. D2438, Lot. No. RNBL5438) and used in the experiment.

In the case of HeLa LgBit (Plk1-HiBit KI), the compounds were treated after being released after thymidine block, and the process was as follows. Thymidine (Sigma-Aldrich Cat. No. T9250-5G) was completely dissolved in ddH₂O and used in the experiment. For thymidine block, the products were treated with 2 mM of thymidine and then incubated for 24 hours. For release and chemical treatment, the medium was suctioned and washed with 1×PBS. TrypLE™ was added and incubated in 37° C. CO₂ incubator (Thermo Fisher Science, Cat. No. 4111) for 5 mins. Cells neutralized by adding complete media were counted through a counter. For each well of a 96-well culture plate (SPL), 3.3×10⁴ cells and a total medium volume of 150 μl were seeded and incubated in a CO₂ incubator.

Each cell line was incubated in a CO₂ incubator for 18 hours, and Endurazine (Promega, Cat. No. N257B) was added to each well to make up 4% of the total volume. After adding compounds of Examples in a 96-well white plate (SPL) to a concentration of 300 nM, the wavelength of the plate reader (BMG Labtech, CLARIOstar Plus) was set to 470-480 nM, and then the luminescence was tracked in real time. After 9 hours, the luminescence value was obtained and displayed as a bar graph through an Excel program.

The results are shown in the table below.

TABLE 3

| Compound | Value | Compound | Value |
|---|---|---|---|
| 1 | 0.139 | 2 | 0.129 |
| 3 | 0.253 | 4 | 0.157 |
| 5 | 0.376 | 6 | 0.286 |
| 7 | 0.214 | 8 | 0.279 |
| 9 | 0.377 | 10 | 0.282 |
| 11 | 0.234 | 12 | 0.372 |
| 13 | 0.165 | 14 | 0.269 |
| 15 | 0.345 | 16 | 0.263 |
| 17 | 0.172 | 18 | 0.111 |
| 19 | 0.132 | 20 | 0.154 |
| 21 | 0.144 | 22 | 0.145 |
| 23 | 0.186 | 24 | 0.206 |
| 25 | 0.144 | 26 | 0.269 |
| 27 | 0.295 | 28 | 0.259 |
| 29 | 0.267 | 30 | 0.250 |
| 31 | 0.241 | | |

### 2. Cell Viability Assay for NCI-H69, NCI-H526 Cell Lines

### Culture of NCI-H69, NCI-H526 Cell Lines

The NCI-H69 (hereafter H69) and NCI-H526 (hereafter H526) cell lines were purchased from Korea Cell Line Bank (KCLB, Seoul, Korea). For cell culture, RPMI 1640 (Gibco, Cat. No. 22400-089; Lot. No. 2537146), FBS (Gibco, Cat. No. 16000-044; Lot. No. 2511328P), Penicillin/Streptomycin (PS) (Gibco, Cat. No. 15140-122; Lot. No. 2441887), 75T cell culture flask (SPL, Cat. No. 71075), 175T cell culture flask (SPL, Cat. No. 71175), 96-well cell culture plate (SPL, Cat. No. 30096), PBS pH 7.4 (Gibco, Cat. No. 10010-023; Lot. No. 2509235), TrypLE™ Express (Gibco, Cat. No. 12605-010; Lot. No. 2323417), Cell Counter (Hematocytometer)(As one, Cat. No. 4-458-01), and 0.4% Trypan Blue Solution (DYNEBIO, Cat. No. CBT3710; Lot. No. 20221111) were used.

### Cell Viability Assay

The compounds of Examples were completely dissolved in DMSO (Sigma-Aldrich Cat. No. D2438, Lot. No. RNBL7287) and used in the experiment. 3×10⁴ cells were seeded for each well of a 96-well plate (SPL), and the cells were cultured in total volume of 150 μl. Each compound was diluted 3-folds from the highest concentration of 3000 nM to the lowest concentration of 0.46 nM. After treating the compound to each well to make the total volume of 200 μl, it was cultured in a CO₂ incubator (Thermo Fisher Science, Cat. No. 4111) for 5 days. Then, after treating EZ-Cytox (DOGEN, Cat.NO. EZ-3000, Lot. No. DLS2308) 20 μl in each well, it was cultured in CO₂ incubator for 4 hours. The absorbance of the completely cultured sample was measured by setting the wavelength of a plate reader (BMG Labtech, CLARIOstar Plus) to 450 nM, and was measured after shaking for 3 minutes in a plate reader before measurement. The final measured value was arranged with Excel program, a graph was displayed through Prism-GraphPad program, and the IC₅₀ value was measured.

The results of cell viability for H69 and H526 cell line are shown in Table 4 and 5, respectively. (A: IC₅₀ is less than 20 nM, B: IC₅₀ is 50 nM or less, C: IC₅₀ is 100 nM or less)

TABLE 4

| Compound | Activity | Compound | Activity |
|---|---|---|---|
| 1 | A | 2 | A |
| 3 | A | 4 | A |
| 5 | A | 6 | B |
| 7 | B | 8 | A |
| 9 | C | 10 | C |
| 11 | B | 12 | C |
| 13 | C | 14 | B |
| 15 | C | 16 | C |
| 17 | B | 18 | A |
| 19 | B | 20 | A |
| 21 | A | 22 | B |
| 23 | A | 24 | A |
| 25 | A | 26 | B |
| 27 | B | 28 | C |
| 29 | C | 30 | B |
| 31 | B | | |

TABLE 5

| Compound | Activity | Compound | Activity |
|---|---|---|---|
| 1 | A | 2 | A |
| 3 | A | 4 | A |
| 5 | A | 6 | A |
| 7 | A | 8 | A |
| 9 | A | 10 | B |
| 11 | A | 12 | B |
| 13 | A | 14 | A |
| 15 | B | 16 | B |
| 17 | A | 18 | A |
| 19 | B | 20 | A |
| 21 | A | 22 | A |
| 23 | A | 24 | A |
| 25 | A | 26 | A |
| 27 | B | 28 | B |
| 29 | B | 30 | B |
| 31 | B | | |

1. A compound represented by the following Formula I: 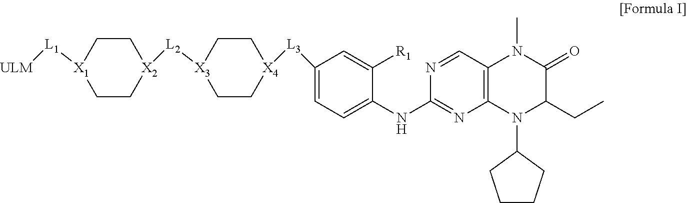
wherein:
L₁ is a covalent bond or —(X₅)₁₋₃— wherein X₅ is each independently —CH₂— or —NH— provided that —NH— is absent or present once at most in L₁;
L₂ is a covalent bond or —(X₆)₁₋₃— wherein X₆ is each independently —CH₂—, —C(O)—, —NH— or —CH(NH₂)— provided that —C(O)—, —NH—, —N(CH₃)— and —CH(NH₂) are each independently absent or present once at most in L₂;
L₃ is 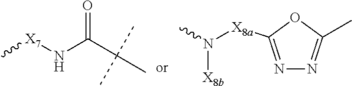
wherein L₃ is covalently bonded into X₄ through [symbol] ;
X₁, X₂, X₃ and X₄ are each independently CH or N;
X₇ and X₈ₐ are each independently a covalent bond or —CH₂—;
X_{8b} is —H or —CH₃; and
R₁ is —OCH₃ or —OCF₃;
wherein ULM is a moiety represented by the following Formula II-1, II-2 or II-3: 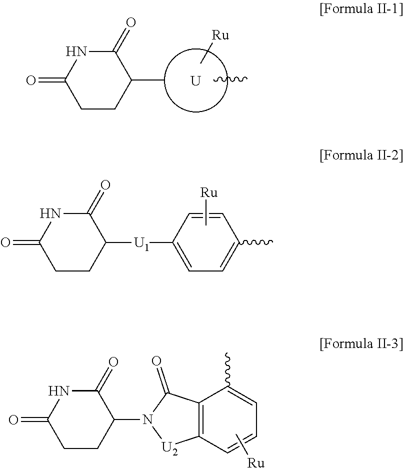
wherein:
Ring U is phenyl or 5- or 6-membered heteroaryl;
U₁ is —NH—, —NHCH₂—, —NHCH₂CH₂—, —CH₂NH—, —CH₂CH₂NH—, —NHCO—, —CONH— or —O—;
U₂ is CH₂ or C(O); and
R_{U} is halo.

2. The compound according to claim 1, wherein Formula I is the following Formula I-1: 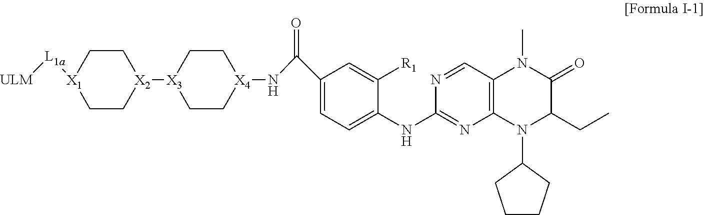
wherein L₁ₐ is —CH₂—, —NH—, —CH₂CH₂—, —NHCH₂—, —CH₂NH—, —CH₂CH₂CH₂—, —NHCH₂CH₂—, —CH₂NHCH₂— or —CH₂CH₂NH—.

3. The compound according to claim 1, wherein Formula I is the following Formula I-2: 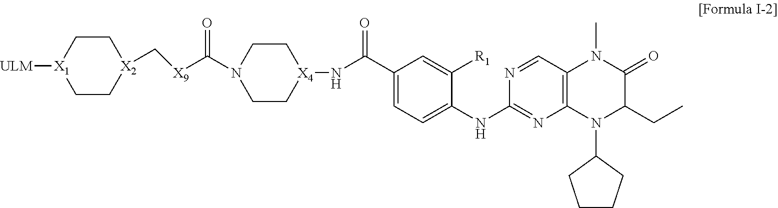
wherein:
X₉ is —CH₂— or —CH(NH₂)—.

4. The compound according to claim 1, wherein Formula I is the following Formula I-3: 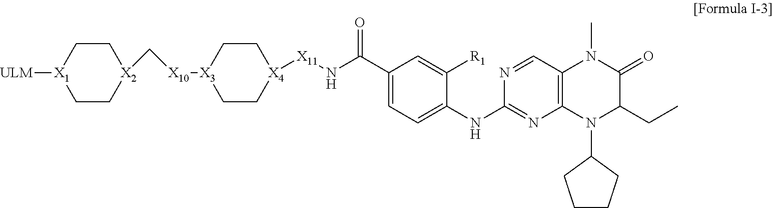
wherein X₁₀ is a covalent bond, —NH— or —CH(NH₂)—; and
X₁₁ is a covalent bond or —CH₂—.

5. The compound according to claim 1, wherein Formula I is represented by the following Formula I-4: 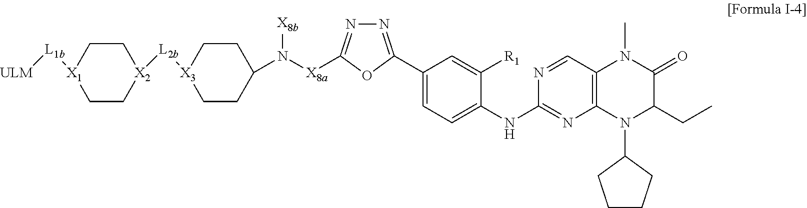
wherein:
L_{1b} is a covalent bond or —CH₂NH—;
L_{2b} is a covalent bond, —CH₂NH—, —CH₂N(CH₃)— or —CH₂CH(NH₂)C(O)—;
X₈ₐ is a covalent bond or —CH₂—; and
X_{8b} is —H or —CH₃.

6. The compound according to claim 1, wherein the compound is selected from Compounds 1 to 25:

| Compound | Structure |
|---|---|
| 1 | 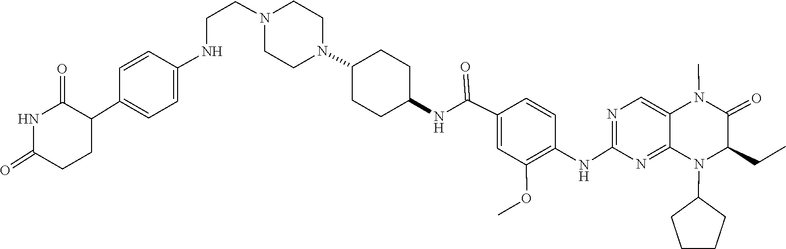 [SMILES: CC[C@@H]1C(=O)N(C)c2cnc(Nc3ccc(C(=O)N[C@H]4CC[C@H](N5CCN(CCNc6ccc(C7CCC(=O)NC7=O)cc6)CC5)CC4)cc3OC)nc2N1C1CCCC1] |
| 2 | 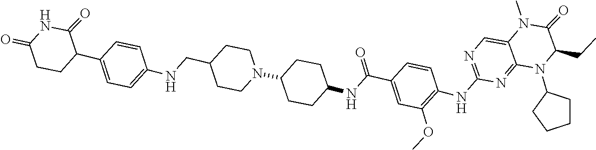 [SMILES: CC[C@@H]1C(=O)N(C)c2cnc(Nc3ccc(C(=O)N[C@H]4CC[C@H](N5CCC(CNc6ccc(C7CCC(=O)NC7=O)cc6)CC5)CC4)cc3OC)nc2N1C1CCCC1] |
| 3 | 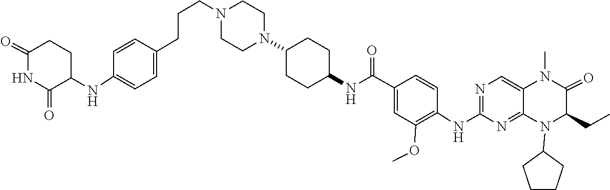 [SMILES: CC[C@@H]1C(=O)N(C)c2cnc(Nc3ccc(C(=O)N[C@H]4CC[C@H](N5CCN(CCCc6ccc(NC7CCC(=O)NC7=O)cc6)CC5)CC4)cc3OC)nc2N1C1CCCC1] |
| 4 | 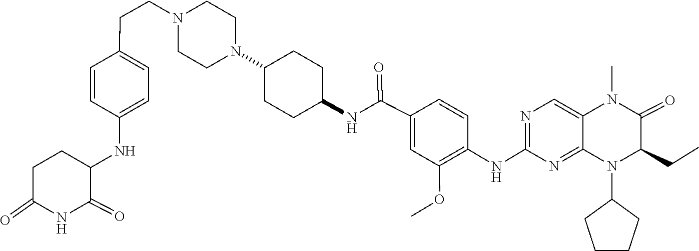 [SMILES: CC[C@@H]1C(=O)N(C)c2cnc(Nc3ccc(C(=O)N[C@H]4CC[C@H](N5CCN(CCc6ccc(NC7CCC(=O)NC7=O)cc6)CC5)CC4)cc3OC)nc2N1C1CCCC1] |
| 5 | 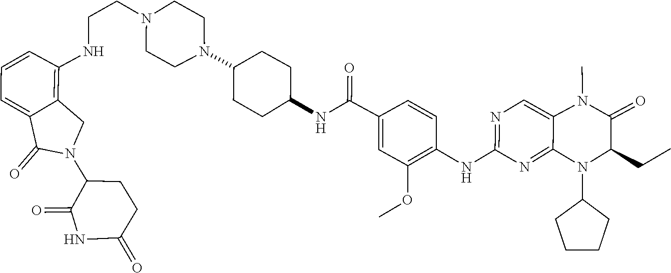 [SMILES: CC[C@@H]1C(=O)N(C)c2cnc(Nc3ccc(C(=O)N[C@H]4CC[C@H](N5CCN(CCNc6cccc7c6CN(C6CCC(=O)NC6=O)C7=O)CC5)CC4)cc3OC)nc2N1C1CCCC1] |
| 6 | 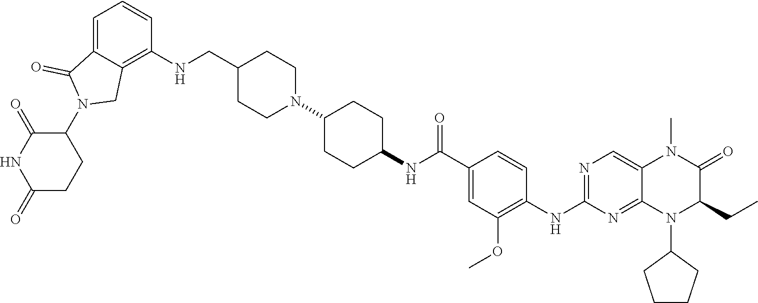 |
| 7 | 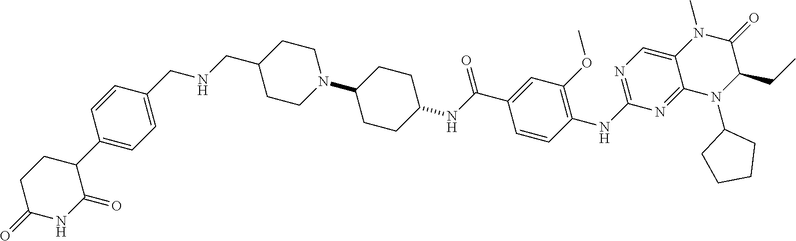 |
| 8 | 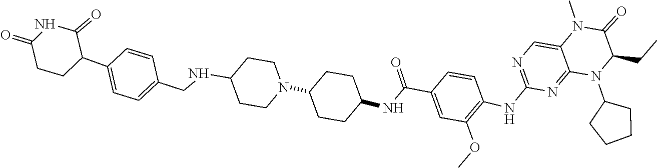 [SMILES: CC[C@@H]1C(=O)N(C)c2cnc(Nc3ccc(C(=O)N[C@H]4CC[C@H](N5CCC(NCc6ccc(C7CCC(=O)NC7=O)cc6)CC5)CC4)cc3OC)nc2N1C1CCCC1] |
| 9 | 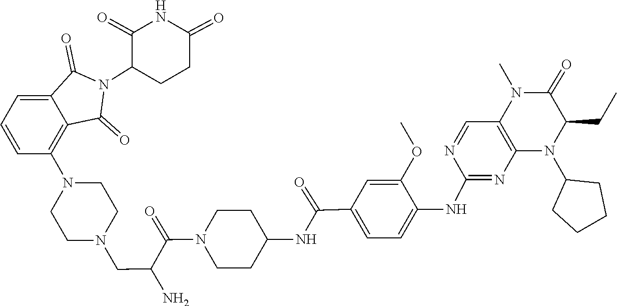 [SMILES: CC[C@@H]1C(=O)N(C)c2cnc(Nc3ccc(C(=O)NC4CCN(C(=O)C(N)CN5CCN(c6cccc7c6C(=O)N(C6CCC(=O)NC6=O)C7=O)CC5)CC4)cc3OC)nc2N1C1CCCC1] |
| 10 | 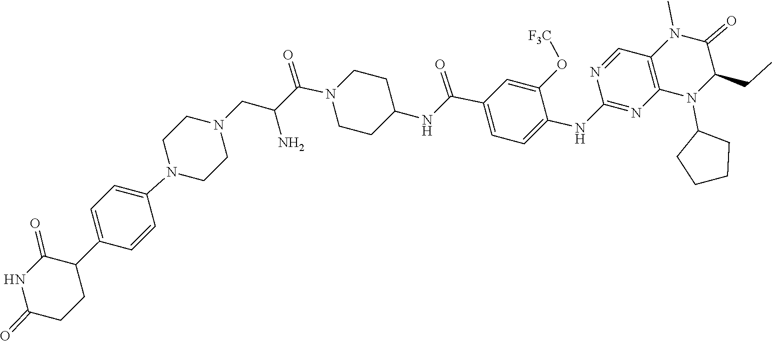 [SMILES: CC[C@@H]1C(=O)N(C)c2cnc(Nc3ccc(C(=O)NC4CCN(C(=O)C(N)CN5CCN(c6ccc(C7CCC(=O)NC7=O)cc6)CC5)CC4)cc3OC(F)(F)F)nc2N1C1CCCC1] |
| 11 | 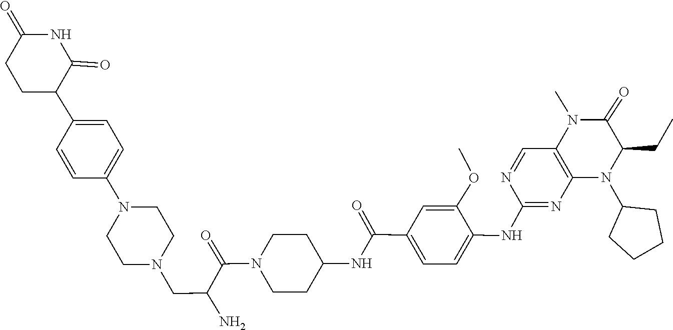 [SMILES: CC[C@@H]1C(=O)N(C)c2cnc(Nc3ccc(C(=O)NC4CCN(C(=O)C(N)CN5CCN(c6ccc(C7CCC(=O)NC7=O)cc6)CC5)CC4)cc3OC)nc2N1C1CCCC1] |
| 12 | 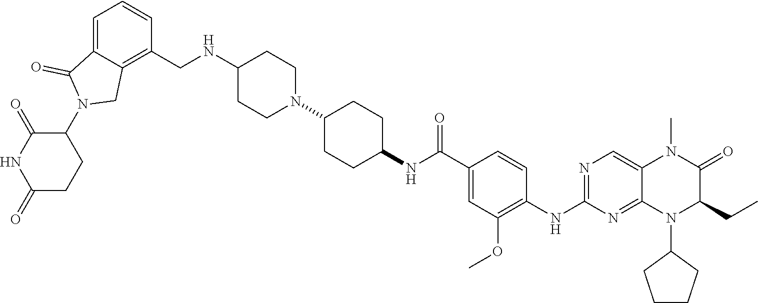 |
| 13 | 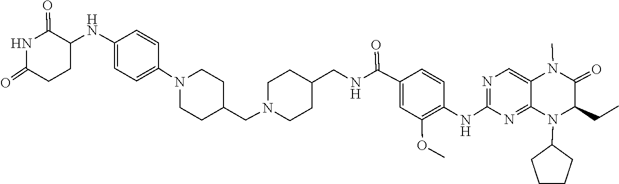 [SMILES: CC[C@@H]1C(=O)N(C)c2cnc(Nc3ccc(C(=O)NCC4CCN(CC5CCN(c6ccc(NC7CCC(=O)NC7=O)cc6)CC5)CC4)cc3OC)nc2N1C1CCCC1] |
| 14 | 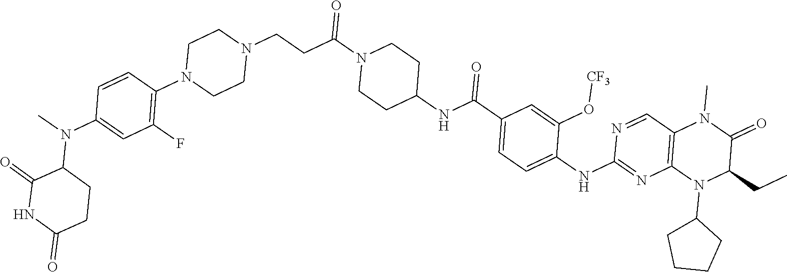 [SMILES: CC[C@@H]1C(=O)N(C)c2cnc(Nc3ccc(C(=O)NC4CCN(C(=O)CCN5CCN(c6ccc(N(C)C7CCC(=O)NC7=O)cc6F)CC5)CC4)cc3OC(F)(F)F)nc2N1C1CCCC1] |
| 15 | 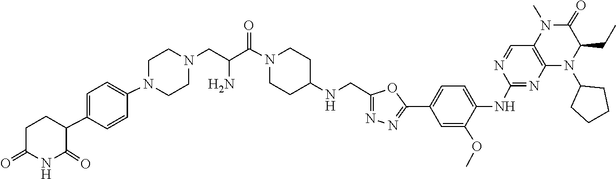 [SMILES: CC[C@@H]1C(=O)N(C)c2cnc(Nc3ccc(-c4nnc(CNC5CCN(C(=O)C(N)CN6CCN(c7ccc(C8CCC(=O)NC8=O)cc7)CC6)CC5)o4)cc3OC)nc2N1C1CCCC1] |
| 16 | 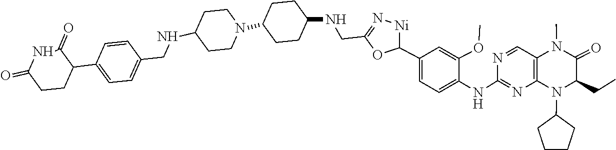 |
| 17 | 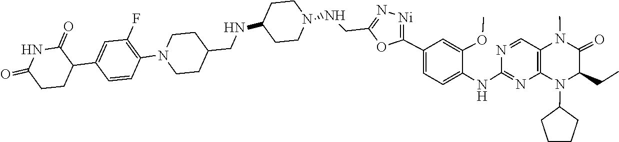 |
| 18 | 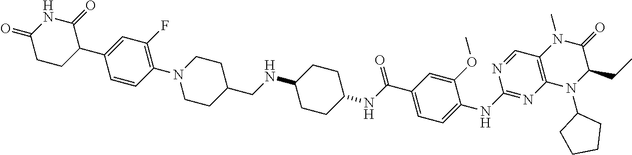 |
| 19 | 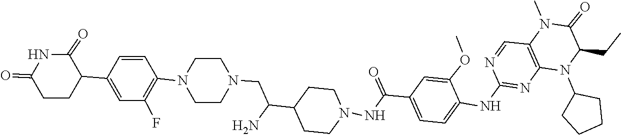 [SMILES: CC[C@@H]1C(=O)N(C)c2cnc(Nc3ccc(C(=O)NN4CCC(C(N)CN5CCN(c6ccc(C7CCC(=O)NC7=O)cc6F)CC5)CC4)cc3OC)nc2N1C1CCCC1] |
| 20 | 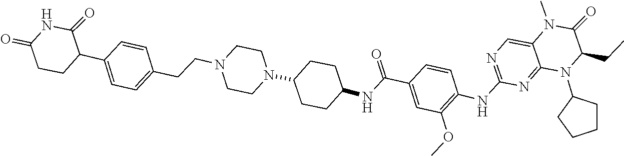 [SMILES: CC[C@@H]1C(=O)N(C)c2cnc(Nc3ccc(C(=O)N[C@H]4CC[C@H](N5CCN(CCc6ccc(C7CCC(=O)NC7=O)cc6)CC5)CC4)cc3OC)nc2N1C1CCCC1] |
| 21 | 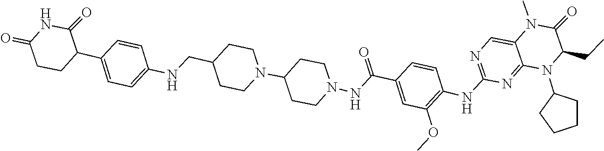 [SMILES: CC[C@@H]1C(=O)N(C)c2cnc(Nc3ccc(C(=O)NN4CCC(N5CCC(CNc6ccc(C7CCC(=O)NC7=O)cc6)CC5)CC4)cc3OC)nc2N1C1CCCC1] |
| 22 | 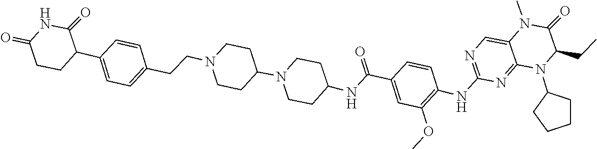 [SMILES: CC[C@@H]1C(=O)N(C)c2cnc(Nc3ccc(C(=O)NC4CCN(C5CCN(CCc6ccc(C7CCC(=O)NC7=O)cc6)CC5)CC4)cc3OC)nc2N1C1CCCC1] |
| 23 | 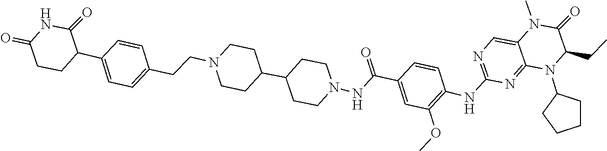 [SMILES: CC[C@@H]1C(=O)N(C)c2cnc(Nc3ccc(C(=O)NN4CCC(C5CCN(CCc6ccc(C7CCC(=O)NC7=O)cc6)CC5)CC4)cc3OC)nc2N1C1CCCC1] |
| 24 | 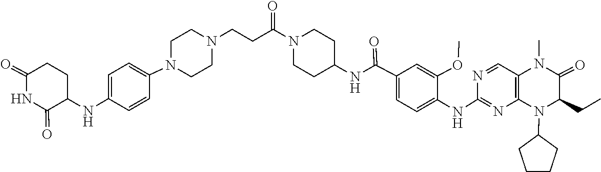 [SMILES: CC[C@@H]1C(=O)N(C)c2cnc(Nc3ccc(C(=O)NC4CCN(C(=O)CCN5CCN(c6ccc(NC7CCC(=O)NC7=O)cc6)CC5)CC4)cc3OC)nc2N1C1CCCC1] |
| 25 | 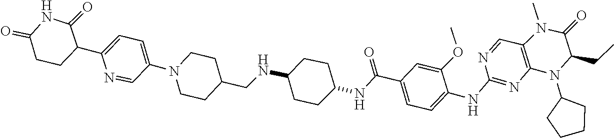 [SMILES: CC[C@@H]1C(=O)N(C)c2cnc(Nc3ccc(C(=O)N[C@H]4CC[C@H](NCC5CCN(c6ccc(C7CCC(=O)NC7=O)nc6)CC5)CC4)cc3OC)nc2N1C1CCCC1] |

7. A compound represented by the following Formula III: 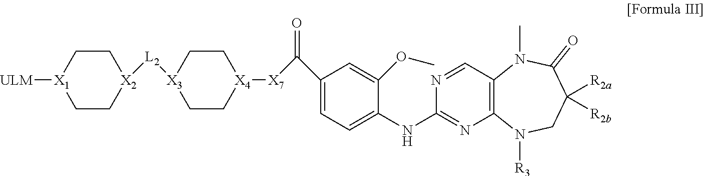
wherein:
L₂ is —CH₂—, —NHCH₂—, —CH₂NH—, —CH(NH₂)CH₂—, —CH₂CH(NH₂)—, —C(O)CH₂CH₂— or —CH₂CH₂C(O)—;
X₁, X₂, X₃ and X₄ are each independently CH or N;
X₇ is a covalent bond or —CH₂—;
R₂ₐ and R_{2b} are -halo, or R₂ₐ and R_{2b} are linked each other to form 3- to 6-membered ring; and
R₃ is —C₁₋₃alkyl or 3- to 7-membered cycloalkyl;
wherein ULM is a moiety represented by the following Formula II-4: 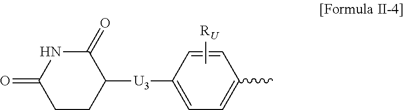
wherein U₃ is a covalent bond or —NH—; and R_{U} is halo.

8. The compound according to claim 7, wherein the compound is selected from Compounds 26 to 31:

| Compound | Structure |
|---|---|
| 26 | 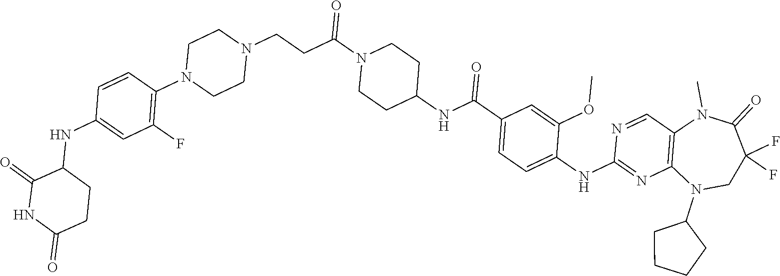 [SMILES: COc1cc(C(=O)NC2CCN(C(=O)CCN3CCN(c4ccc(NC5CCC(=O)NC5=O)cc4F)CC3)CC2)ccc1Nc1ncc2c(n1)N(C1CCCC1)CC(F)(F)C(=O)N2C] |
| 27 | 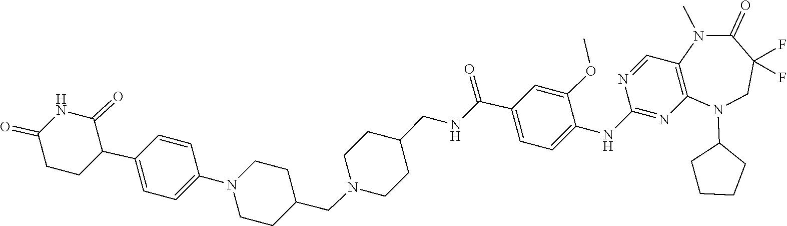 [SMILES: COc1cc(C(=O)NCC2CCN(CC3CCN(c4ccc(C5CCC(=O)NC5=O)cc4)CC3)CC2)ccc1Nc1ncc2c(n1)N(C1CCCC1)CC(F)(F)C(=O)N2C] |
| 28 | 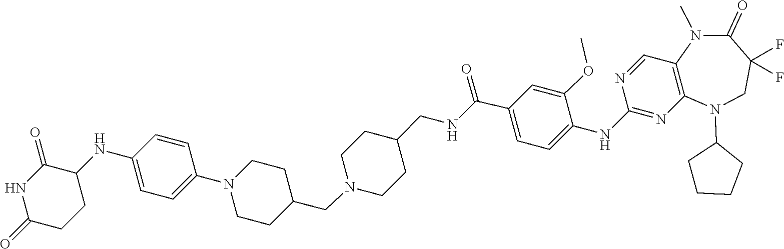 [SMILES: COc1cc(C(=O)NCC2CCN(CC3CCN(c4ccc(NC5CCC(=O)NC5=O)cc4)CC3)CC2)ccc1Nc1ncc2c(n1)N(C1CCCC1)CC(F)(F)C(=O)N2C] |
| 29 | 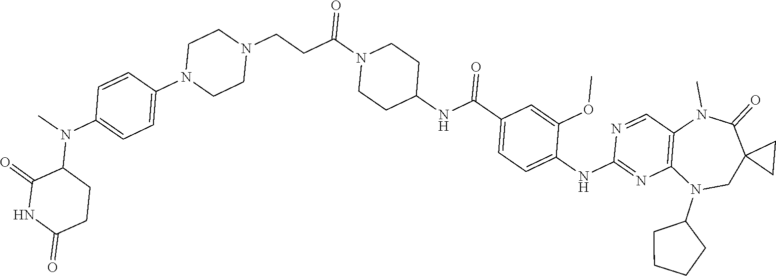 [SMILES: COc1cc(C(=O)NC2CCN(C(=O)CCN3CCN(c4ccc(N(C)C5CCC(=O)NC5=O)cc4)CC3)CC2)ccc1Nc1ncc2c(n1)N(C1CCCC1)CC1(CC1)C(=O)N2C] |
| 30 | 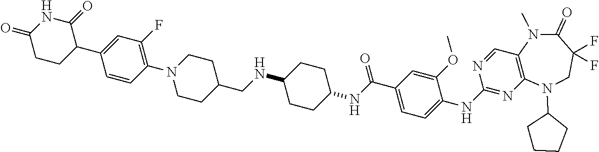 |
| 31 | 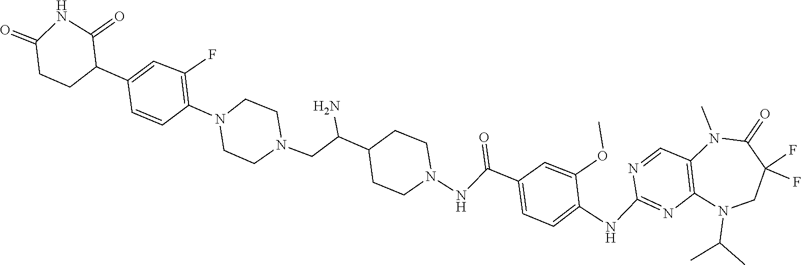 [SMILES: COc1cc(C(=O)NN2CCC(C(N)CN3CCN(c4ccc(C5CCC(=O)NC5=O)cc4F)CC3)CC2)ccc1Nc1ncc2c(n1)N(C(C)C)CC(F)(F)C(=O)N2C] |

9. The compound according to claim 1, wherein the compound induces polo-like kinase 1 (PLK1) protein degradation.

10. The pharmaceutical composition comprising the compound according to claim 1, and at least one pharmaceutically acceptable carrier.

11. The pharmaceutical composition comprising the compound according to claim 1, wherein the compound is conjugated to an antibody or an antigen binding fragment thereof via a linker.

12. The pharmaceutical composition comprising the compound according to claim 1 for the treatment of a cancer, a benign tumor or a neurological disease.

13. An antibody-drug conjugate comprising an antibody or an antigen binding fragment thereof and the compound according to claim 1, wherein the compound is conjugated to the antibody or antigen binding fragment thereof via a linker.
